(19) United States
(12) Patent Application Publication
(10) Pub. No.: US 2022/0242879 A1
(43) Pub. Date: Aug. 4, 2022

(54) FUSED HETEROCYCLE DERIVATIVES

(71) Applicant: Janssen Sciences Ireland Unlimited Company, Co Cork (IE)

(72) Inventors: Scott D. KUDUK, Harleysville, PA (US); Christelle Catherine Cecile DOEBELIN, IllKIRCH (FR); Abdellah TAHRI, Anderlecht (BE); Sandrine Céline GROSSE, Turnhout (BE); Stefaan Julien LAST, Beveren (BE); Lindsey Graham DERATT, North Wales, PA (US); Koen VANDYCK, Paal-Beringen (BE); Pierre Jean-Marie Bernard RABOISSON, Wavre (BE); Jan Martin BERKE, Düsseldorf (DE); Wim Gaston VERSCHUEREN, Berchem (BE); Michel OBRINGER, Achen (FR)

(21) Appl. No.: 17/595,804

(22) PCT Filed: May 27, 2020
(86) PCT No.: PCT/US2020/034667

§ 371 Date: Nov. 24, 2021

Related U.S. Application Data
(60) Provisional application No. 62/853,528, filed on May 28, 2019.

(30) Foreign Application Priority Data
May 28, 2019 (EP) 19177009.8

(51) Int. Cl. C07D 498/22; A61K 45/06; A61K 31/4375; C07D 471/22; C07D 513/22; A61K 31/55; A61P 31/20

(52) U.S. Cl. CPC C07D 498/22; A61K 45/06; A61K 31/4375; A61P 31/20; C07D 513/22; A61K 31/55; C07D 471/22

(57) ABSTRACT

The application describes fused heterocycle derivative compounds, pharmaceutical compositions comprising these compounds, chemical processes for preparing these compounds and their use in the treatment of diseases associated with HBV infection.

### FIELD

The application relates to fused heterocyclic derivative compounds, pharmaceutical compositions comprising these compounds, chemical processes for preparing these compounds and their use in the treatment of HBV or diseases associated with HBV infection.

### RELATED APPLICATIONS

This application claims priority to European Application No. 19177009.8 filed on May 28, 2019 and U.S. Provisional Application No. 62/853,528 filed on May 28, 2019, the contents of which are hereby incorporated in their entireties.

### BACKGROUND

Chronic hepatitis B virus (HBV) infection is a significant global health problem, affecting over 5% of the world population (over 350 million people worldwide and 1.25 million individuals in the U.S.).

Despite the availability of a prophylactic HBV vaccine, the burden of chronic HBV infection continues to be a significant unmet worldwide medical problem, due to suboptimal treatment options and sustained rates of new infections in most parts of the developing world. Current treatments do not provide a cure and are limited to only two classes of agents (interferon alpha and nucleoside analogues/inhibitors of the viral polymerase); drug resistance, low efficacy, and tolerability issues limit their impact. The low cure rates of HBV are attributed at least in part to the fact that complete suppression of virus production is difficult to achieve with a single antiviral agent. However, persistent suppression of HBV DNA slows liver disease progression and helps to prevent hepatocellular carcinoma. Current therapy goals for HBV-infected patients are directed to reducing serum HBV DNA to low or undetectable levels, and to ultimately reducing or preventing the development of cirrhosis and hepatocellular carcinoma.

The HBV capsid protein plays essential functions during the viral life cycle. HBV capsid/core proteins form metastable viral particles or protein shells that protect the viral genome during intercellular passage, and also play a central role in viral replication processes, including genome encapsidation, genome replication, and virion morphogenesis and egress. Capsid structures also respond to environmental cues to allow un-coating after viral entry. Consistently, the appropriate timing of capsid assembly and dis-assembly, the appropriate capsid stability and the function of core protein have been found to be critical for viral infectivity.

The crucial function of HBV capsid proteins imposes stringent evolutionary constraints on the viral capsid protein sequence, leading to the observed low sequence variability and high conservation. Consistently, mutations in HBV capsid that disrupt its assembly are lethal, and mutations that perturb capsid stability severely attenuate viral replication. The high functional constraints on the multi-functional HBV core/capsid protein is consistent with a high sequence conservation, as many mutations are deleterious to function. Indeed, the core/capsid protein sequences are >90% identical across HBV genotypes and show only a small number of polymorphic residues. Resistance selection to HBV core/capsid protein binding compounds may therefore be difficult to select without large impacts on virus replication fitness.

Reports describing compounds that bind viral capsids and inhibit replication of HIV, rhinovirus and HBV provide strong pharmacological proof of concept for viral capsid proteins as antiviral drug targets.

WO2018/005881 and WO2018/005883 disclose fused tricyclic derivatives for the treatment of HBV.

There is a need in the art for therapeutic agents that can increase the suppression of virus production and that can treat, ameliorate, and/or prevent HBV infection. Administration of such therapeutic agents to an HBV infected patient, either as monotherapy or in combination with other HBV treatments or ancillary treatments, will lead to significantly reduced virus burden, improved prognosis, diminished progression of the disease and enhanced seroconversion rates.

In view of the clinical importance of HBV, the identification of compounds that can increase the suppression of virus production and that can treat, ameliorate, and/or prevent HBV infection represents an attractive avenue into the development of new therapeutic agents. Such compounds are provided herein.

### SUMMARY

The present disclosure is directed to the general and preferred embodiments defined, respectively, by the independent and dependent claims appended hereto, which are incorporated by reference herein. The present invention is directed to compounds capable of capsid assembly modulation. The compounds of the present invention may provide a beneficial balance of properties with respect to prior art compounds. In particular, the present disclosure is directed to compounds of Formula (I):

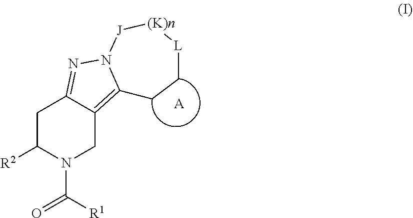

or a stereoisomer or tautomer thereof, wherein

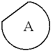

is a 5-membered heteroaryl comprising one, two or three heteroatoms, the heteroatoms being independently selected from the group consisting of N, O and S, wherein the 5-membered heteroaryl is substituted with one or more substituents each independently selected from the group consisting of H, C₁₋₄alkyl, CF₃, CF₂H, NH₂, NH(CH₃), N(CH₃)₂ and phenyl;

R¹ is a 5- to 10-membered monocyclic or bicyclic ring, more particularly a 5- to 9-membered monocyclic or bicyclic ring, wherein the 5- to 10-membered monocyclic or bicyclic ring, more particularly the 5- to 9-membered monocyclic or bicyclic ring:
optionally contains 1 to 3 heteroatoms, the heteroatoms each independently being selected from N, O and S; and/or
is optionally substituted with one or more substituents each independently selected from the group consisting of hydrogen, halogens, CN, CF₃, CF₂H, CFH₂, CF₂CH₃, C₁₋₆alkyl, OC₁₋₆alkyl, OCF₃, OCF₂H and C₃₋₄cycloalkyl;

more particularly R¹ is phenyl substituted with one or more substituents each independently selected from the group consisting of CN, F, CF₃, CF₂H, CN, and C₁₋₄alkyl;

R² is selected from the group consisting of H, C₁₋₄alkyl and C₁₋₄alkyl substituted with one or more F;

J is CHR³:

R³ is selected from the group consisting of H, CH₂OH, and C(═O)N(R⁴)(R⁵);

R⁴ and R⁵ are each independently selected from the group consisting of H, C₁₋₄alkyl, and C₃₋₄cycloalkyl, wherein C₁₋₄alkyl is optionally substituted with one or more substituents each independently selected from the group consisting of OH and F;

K is selected from the group consisting of C(R⁶)(R⁷), C═CH₂ and C(═O);

R⁶ and R⁷ are each independently selected from the group consisting of H, F, OH, OCH, CH₂OH, C(═O)R⁴ and C(═O)N(R⁹)(R¹⁰);

R⁸ is OH or morpholine;

R⁹ and R¹⁰ are each independently selected from the group consisting of H, phenyl, C₁₋₄alkyl and C₁₋₄cycloalkyl, wherein C₁₋₄alkyl is optionally substituted with one or more substituents each independently selected from the group consisting of OH and F;

n is an integer of 0 or 1:

L is C(R¹¹)(R¹²), NH, O;

R¹¹ and R¹² are each independently selected from the group consisting of H and C(═O)N(R¹³)(R¹⁴); and

R¹³ and R¹⁴ are each independently selected from the group consisting of H, C₁₋₄alkyl and C₃₋₄cycloalkyl, wherein C₁₋₄alkyl is optionally substituted with one or more substituents each independently selected from the group consisting of OH and F,

or a pharmaceutically acceptable salt thereof.

Further embodiments include pharmaceutically acceptable salts of compounds of Formula (I), pharmaceutically acceptable prodrugs of compounds of Formula (I), pharmaceutically active metabolites of compounds of Formula (I), and enantiomers and diastereomers of the compounds of Formula (I), as well as pharmaceutically acceptable salts thereof.

In embodiments, the compounds of Formula (I) are compounds selected from those species described or exemplified in the detailed description below.

The present disclosure is also directed to pharmaceutical compositions comprising one or more compounds of Formula (I), pharmaceutically acceptable salts of compounds of Formula (I), pharmaceutically acceptable prodrugs of compounds of Formula (I), and pharmaceutically active metabolites of Formula (I). Pharmaceutical compositions may further comprise one or more pharmaceutically acceptable excipients or one or more other agents or therapeutics.

The present disclosure is also directed to methods of using or uses of compounds of Formula (I). In embodiments, compounds of Formula (I) are used to prevent, treat or ameliorate hepatitis B viral (HBV) infection, increase the suppression of HBV production, interfere with HBV capsid assembly or other HBV viral replication steps or products thereof. The methods comprise administering to a subject in need of such method an effective amount of at least one compound of Formula (I), pharmaceutically acceptable salts of compounds of Formula (I), pharmaceutically acceptable prodrugs of compounds of Formula (I), and pharmaceutically active metabolites of compounds of Formula (I). Additional embodiments of methods of treatment are set forth in the detailed description. Any of the methods provided herein can further comprise administering to the individual at least one additional therapeutic agent, more particularly at least one other HBV inhibitor.

The present disclosure is also directed to compounds of Formula (Ia):

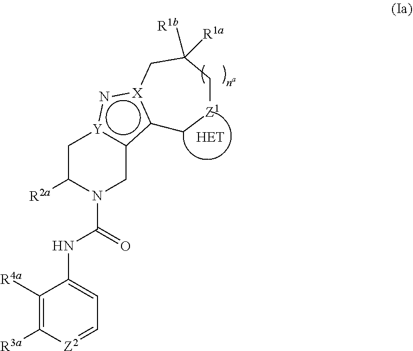

and pharmaceutically acceptable salts, stereoisomers, isotopic variants. N-oxides, or solvates of compounds of Formula (Ia);

wherein

R^{1b} is independently selected from the group consisting of: hydrogen, C₁₋₄alkyl, hydroxy, hydroxymethyl, (2,2-difluoroethoxy)methyl, OC₁₋₄alkyl, and fluoro;

R^{1a} is independently hydrogen or taken together with R^{1b} to form methylenyl:

n^{a} is an integer that is 0, 1, or 2;

R^{2a} is independently selected from the group consisting of hydrogen and C₁₋₆alkyl;

R^{3a} is selected from the group consisting of: Cl, CN, and C₁₋₄haloalkyl;

R^{4a} is H, or F:

HET is a 5- or 6-membered heteroaryl, optionally independently substituted with one to two substituents selected from the group consisting of: C₁₋₄alkyl, bromo, choro, fluoro, and hydroxy(C₁₋₄)alkyl;

X and Y are each independently selected from: N or C, such that only one of X and Y is N in any instance:

Z¹ is N or C; and

Z² is N or CF.

Further embodiments include pharmaceutically acceptable salts of compounds of Formula (Ia), pharmaceutically acceptable prodrugs of compounds of Formula (Ia), pharmaceutically active metabolites of compounds of Formula (Ia), and enantiomers and diastereomers of the compounds of Formula (Ia), as well as pharmaceutically acceptable salts thereof.

In embodiments, the compounds of Formula (Ia) are compounds selected from those species described or exemplified in the detailed description below.

The present disclosure is also directed to pharmaceutical compositions comprising one or more compounds of Formula (Ia), pharmaceutically acceptable salts of compounds of Formula (Ia), pharmaceutically acceptable prodrugs of compounds of Formula (Ia), and pharmaceutically active metabolites of Formula (Ia). Pharmaceutical compositions may further comprise one or more pharmaceutically acceptable excipients or one or more other agents or therapeutics.

The present disclosure is also directed to methods of using or uses of compounds of Formula (Ia). In embodiments, compounds of Formula (Ia) are used to treat or ameliorate hepatitis B viral (HBV) infection, increase the suppression of HBV production, interfere with HBV capsid assembly or other HBV viral replication steps or products thereof. The methods comprise administering to a subject in need of such method an effective amount of at least one compound of Formula (Ia), pharmaceutically acceptable salts of compounds of Formula (Ia), pharmaceutically acceptable prodrugs of compounds of Formula (Ia), and pharmaceutically active metabolites of compounds of Formula (Ia). Additional embodiments of methods of treatment are set forth in the detailed description.

An object of the present disclosure is to overcome or ameliorate at least one of the disadvantages of the conventional methodologies and/or prior art, or to provide a useful alternative thereto. Additional embodiments, features, and advantages of the present disclosure will be apparent from the following detailed description and through practice of the disclosed subject matter.

### DETAILED DESCRIPTION

Additional embodiments, features, and advantages of the subject matter of the present disclosure will be apparent from the following detailed description of such disclosure and through its practice. For the sake of brevity, the publications, including patents, cited in this specification are herein incorporated by reference.

Provided herein are compounds of Formula (I), and their pharmaceutically acceptable salts, pharmaceutically acceptable prodrugs, and pharmaceutically active metabolites of the disclosed compounds. These compounds may provide an advantageous balance of properties compared to prior art compounds.

In one aspect, provided herein are compounds of Formula (I),

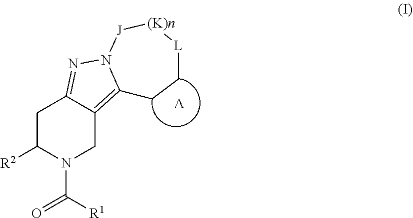

or a stereoisomer or tautomer thereof, or a pharmaceutically acceptable salt thereof, wherein

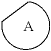

is a 5-membered heteroaryl comprising one, two or three heteroatoms, the heteroatoms being independently selected from the group consisting of N, O and S, wherein the 5-membered heteroaryl is substituted with one or more substituents selected from the group consisting of H, C₁₋₄alkyl, CF₃, CF₂H, NH₂, NH(CH₃), N(CH₃)₂ and phenyl;

R¹ is phenyl substituted with one or more substituents selected from the group consisting of Cl, F, CF₃, CF₂H, CN, and C₁₋₄alkyl;

R² is selected from the group consisting of H, C₁₋₄alkyl and C₁₋₄alkyl substituted with one or more F;

J is CHR³;

R³ is selected from the group consisting of H, CH₂OH, and C(═O)N(R⁴)(R⁵);

R⁴ and R⁵ are independently selected from the group consisting of H, C₁₋₄alkyl, and C₃₋₄cycloalkyl, wherein C₁₋₄alkyl is optionally substituted with one or more substituents selected from the group consisting of OH and F;

K is selected from the group consisting of C(R⁶)(R⁷), C═CH₂ and C(═O);

R⁶ and R⁷ are independently selected from the group consisting of H, F, OH, OCH₃, CH₂OH, C(═O)R⁸ and C(═O)N(R⁹)(R¹⁰);

R⁸ is OH or morpholine:

R⁹ and R¹⁰ are independently selected from the group consisting of H, phenyl, C₁₋₄alkyl and C₁₋₄cycloalkyl, wherein C₁₋₄alkyl is optionally substituted with one or more substituents selected from the group consisting of OH and F;

n is an integer of 0 or 1;

L is C(R¹¹)(R¹²), NH, O;

R¹¹ and R¹² are independently selected from the group consisting of H and C(═O)N(R¹³)(R¹⁴); and

R¹³ and R¹⁴ are independently selected from the group consisting of H, C₁₋₄alkyl and C₃₋₄cycloalkyl, wherein C₁₋₄alkyl is optionally substituted with one or more substituents selected from the group consisting of OH and F.

In embodiments, the compound of Formula (I) is a compound wherein R¹ is phenyl substituted with one or more Cl substituents, more particularly wherein R¹ is dichlorophenyl.

In embodiments, the compound of Formula (I) is a compound wherein R² is H or methyl.

In embodiments, the compound of Formula (I) is a compound wherein R³ is H.

In embodiments, the compound of Formula (I) is a compound wherein K is C(R⁶)(R⁷) or C═CH₂.

In embodiments, the compound of Formula (I) is a compound wherein K is C(R⁶)(R⁷) or C═CH₂, wherein

R⁶ and R⁷ are each independently selected from the group consisting of H, F, OH, OCH₃, CH₂OH, C(═O)R⁸ and C(═O)N(R⁹)(R¹⁰);

R⁸ is OH or morpholine; and

R⁹ and R¹⁰ are each independently selected from the group consisting of H, phenyl, C₁₋₄alkyl and C₃₋₄cycloalkyl, wherein C₁₋₄alkyl is optionally substituted with one or more substituents each independently selected from the group consisting of OH and F.

In embodiments, the compound of Formula (I) is a compound wherein R⁶ and R⁷ are independently selected from the group consisting of H, F, OH, CH₂OH and C(═O)N(R⁹)(R¹⁰).

In embodiments, the compound of Formula (I) is a compound wherein R⁶ and R⁷ are independently selected from the group consisting of H, OH and C(═O)N(R⁹)(R¹⁰).

In embodiments, the compound of Formula (I) is a compound wherein K is C(R⁶)(R⁷) and wherein R⁶ and R⁷ are each independently selected from the group consisting of H, F, OH, CH₂OH and C(═O)N(R⁹)(R¹⁰), more in particular, wherein R⁶ is H or OH, and R⁷ is selected from the group consisting of H, F, OH, CH₂OH and C(═O)N(R⁹)(R¹⁰); and wherein R⁹ and R¹⁰ are each independently selected from the group consisting of H, phenyl, C₁₋₄alkyl and C₃₋₄cycloalkyl, wherein C₁₋₄alkyl is optionally substituted with one or more substituents selected from the group consisting of OH and F.

In embodiments, the compound of Formula (I) is a compound R⁹ and R¹⁰ are independently selected from the group consisting of C₁₋₄alkyl and C₃₋₄cycloalkyl.

In embodiments, the compound of Formula (I) is a compound wherein each of R¹¹ and R¹² is hydrogen. In embodiments, the compound of Formula (I) is a compound wherein

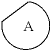

is selected from the group consisting of isoxazole, pyrazole, imidazole, oxazole and thiazole, and wherein

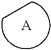

is optionally substituted with one or more substituents selected from the group consisting of H C₁₋₄alkyl, CF₃, CF₂H, NH₂, NH(CH₃), N(CH₃)₂ and phenyl.

In embodiments, the compound of Formula (I) is a compound wherein

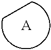

is an isoxazole, optionally substituted with a substituent selected from C₁₋₄alkyl and NH₂.

In embodiments, the compound of Formula (I) is a compound wherein

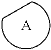

is a pyrazole.

In embodiments, the compound of Formula (I) is a compound wherein n is 0.

In embodiments, the compound of Formula (I) is a compound wherein n is 1.

In embodiments, the compound of Formula (I) is a compound which shows an EC₅₀ of less than 0.10 μM for the inhibition of HBV DNA in the hepG2.117 cell line.

A further embodiment of the present disclosure is a compound selected from the group consisting of the compounds described below (cf. Table 1), a stereoisomer or tautomer thereof, or a pharmaceutically acceptable salt thereof.

TABLE 1

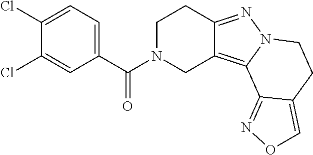 [SMILES: O=C(c1ccc(Cl)c(Cl)c1)N1CCc2nn3c(c2C1)-c1nocc1CC3]

1

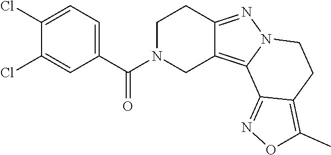 [SMILES: Cc1onc2c1CCn1nc3c(c1-2)CN(C(=O)c1ccc(Cl)c(Cl)c1)CC3]

2

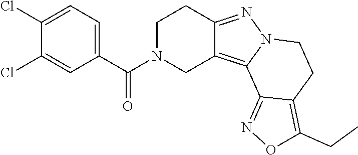 [SMILES: CCc1onc2c1CCn1nc3c(c1-2)CN(C(=O)c1ccc(Cl)c(Cl)c1)CC3]

3

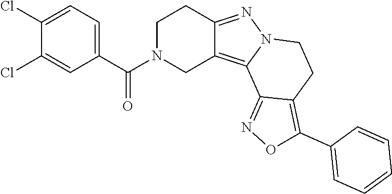 [SMILES: O=C(c1ccc(Cl)c(Cl)c1)N1CCc2nn3c(c2C1)-c1noc(-c2ccccc2)c1CC3]

4

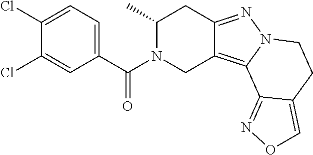 [SMILES: C[C@@H]1Cc2nn3c(c2CN1C(=O)c1ccc(Cl)c(Cl)c1)-c1nocc1CC3]

5

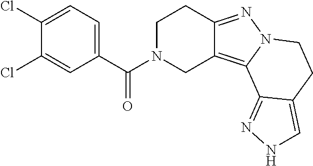 [SMILES: O=C(c1ccc(Cl)c(Cl)c1)N1CCc2nn3c(c2C1)-c1n[nH]cc1CC3]

6

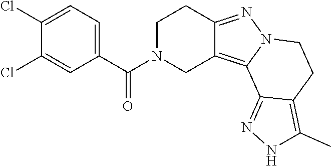 [SMILES: Cc1[nH]nc2c1CCn1nc3c(c1-2)CN(C(=O)c1ccc(Cl)c(Cl)c1)CC3]

7

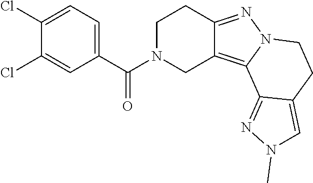 [SMILES: Cn1cc2c(n1)-c1c3c(nn1CC2)CCN(C(=O)c1ccc(Cl)c(Cl)c1)C3]

8

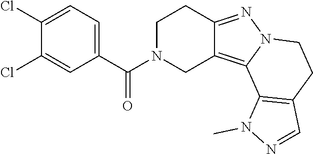 [SMILES: Cn1ncc2c1-c1c3c(nn1CC2)CCN(C(=O)c1ccc(Cl)c(Cl)c1)C3]

9

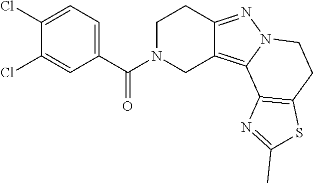 [SMILES: Cc1nc2c(s1)CCn1nc3c(c1-2)CN(C(=O)c1ccc(Cl)c(Cl)c1)CC3]

10

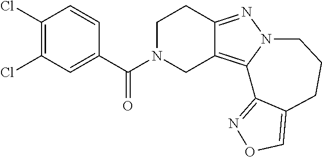 [SMILES: O=C(c1ccc(Cl)c(Cl)c1)N1CCc2nn3c(c2C1)-c1nocc1CCC3]

11

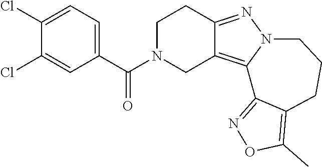 [SMILES: Cc1onc2c1CCCn1nc3c(c1-2)CN(C(=O)c1ccc(Cl)c(Cl)c1)CC3]

12

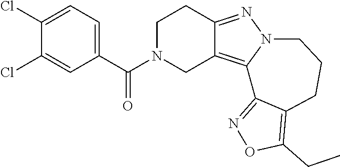 [SMILES: CCc1onc2c1CCCn1nc3c(c1-2)CN(C(=O)c1ccc(Cl)c(Cl)c1)CC3]

13

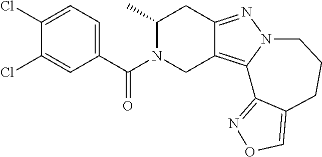 [SMILES: C[C@@H]1Cc2nn3c(c2CN1C(=O)c1ccc(Cl)c(Cl)c1)-c1nocc1CCC3]

14

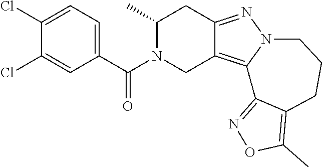 [SMILES: Cc1onc2c1CCCn1nc3c(c1-2)CN(C(=O)c1ccc(Cl)c(Cl)c1)[C@H](C)C3]

15

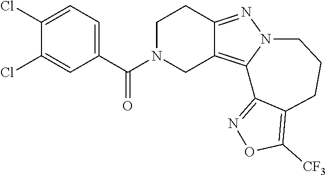 [SMILES: O=C(c1ccc(Cl)c(Cl)c1)N1CCc2nn3c(c2C1)-c1noc(C(F)(F)F)c1CCC3]

16

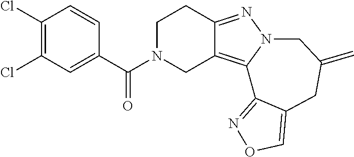 [SMILES: C=C1Cc2conc2-c2c3c(nn2C1)CCN(C(=O)c1ccc(Cl)c(Cl)c1)C3]

17

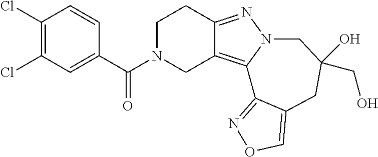 [SMILES: O=C(c1ccc(Cl)c(Cl)c1)N1CCc2nn3c(c2C1)-c1nocc1CC(O)(CO)C3]

18

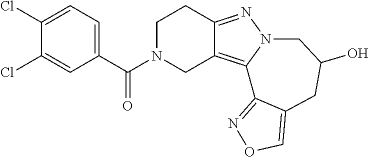 [SMILES: O=C(c1ccc(Cl)c(Cl)c1)N1CCc2nn3c(c2C1)-c1nocc1CC(O)C3]

19

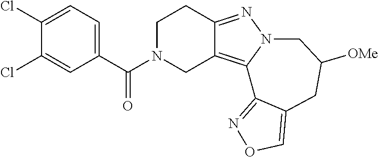 [SMILES: COC1Cc2conc2-c2c3c(nn2C1)CCN(C(=O)c1ccc(Cl)c(Cl)c1)C3]

20

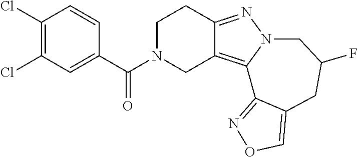 [SMILES: O=C(c1ccc(Cl)c(Cl)c1)N1CCc2nn3c(c2C1)-c1nocc1CC(F)C3]

21

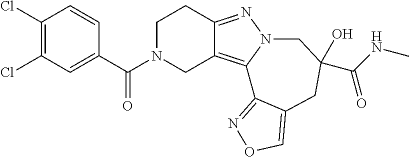 [SMILES: CNC(=O)C1(O)Cc2conc2-c2c3c(nn2C1)CCN(C(=O)c1ccc(Cl)c(Cl)c1)C3]

22

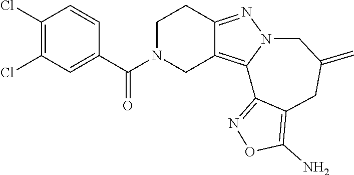 [SMILES: C=C1Cc2c(noc2N)-c2c3c(nn2C1)CCN(C(=O)c1ccc(Cl)c(Cl)c1)C3]

23

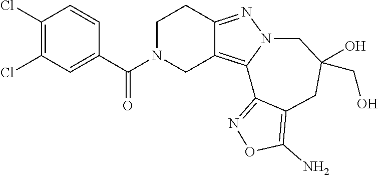 [SMILES: Nc1onc2c1CC(O)(CO)Cn1nc3c(c1-2)CN(C(=O)c1ccc(Cl)c(Cl)c1)CC3]

24

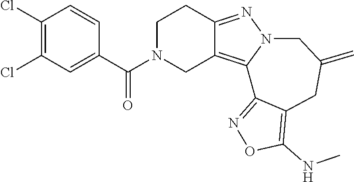 [SMILES: C=C1Cc2c(noc2NC)-c2c3c(nn2C1)CCN(C(=O)c1ccc(Cl)c(Cl)c1)C3]

25

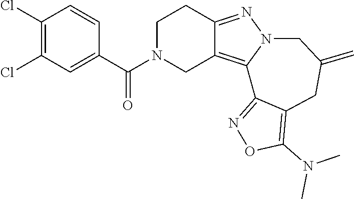 [SMILES: C=C1Cc2c(noc2N(C)C)-c2c3c(nn2C1)CCN(C(=O)c1ccc(Cl)c(Cl)c1)C3]

26

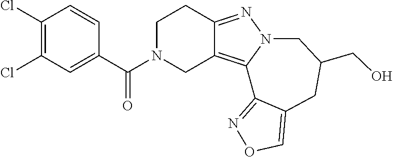 [SMILES: O=C(c1ccc(Cl)c(Cl)c1)N1CCc2nn3c(c2C1)-c1nocc1CC(CO)C3]

27

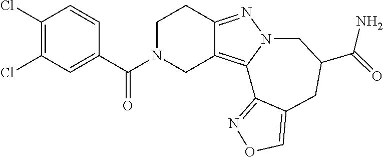 [SMILES: NC(=O)C1Cc2conc2-c2c3c(nn2C1)CCN(C(=O)c1ccc(Cl)c(Cl)c1)C3]

28

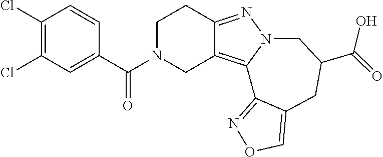 [SMILES: O=C(O)C1Cc2conc2-c2c3c(nn2C1)CCN(C(=O)c1ccc(Cl)c(Cl)c1)C3]

29

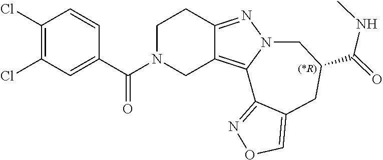 [SMILES: CNC(=O)[C@@H]1Cc2conc2-c2c3c(nn2C1)CCN(C(=O)c1ccc(Cl)c(Cl)c1)C3]

30

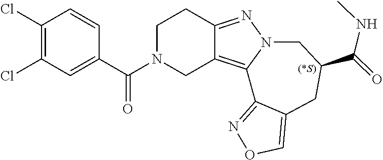 [SMILES: CNC(=O)[C@H]1Cc2conc2-c2c3c(nn2C1)CCN(C(=O)c1ccc(Cl)c(Cl)c1)C3]

31

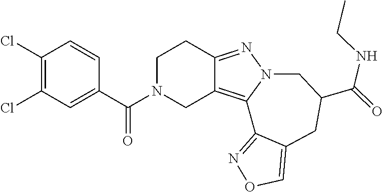 [SMILES: CCNC(=O)C1Cc2conc2-c2c3c(nn2C1)CCN(C(=O)c1ccc(Cl)c(Cl)c1)C3]

32

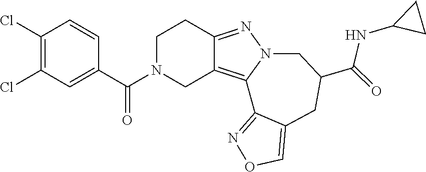 [SMILES: O=C(NC1CC1)C1Cc2conc2-c2c3c(nn2C1)CCN(C(=O)c1ccc(Cl)c(Cl)c1)C3]

33

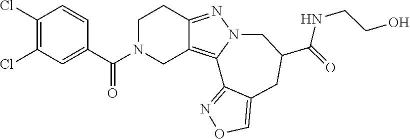 [SMILES: O=C(NCCO)C1Cc2conc2-c2c3c(nn2C1)CCN(C(=O)c1ccc(Cl)c(Cl)c1)C3]

34

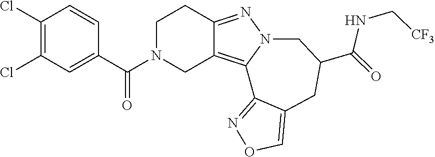 [SMILES: O=C(NCC(F)(F)F)C1Cc2conc2-c2c3c(nn2C1)CCN(C(=O)c1ccc(Cl)c(Cl)c1)C3]

35

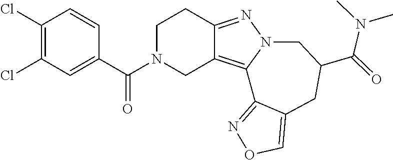 [SMILES: CN(C)C(=O)C1Cc2conc2-c2c3c(nn2C1)CCN(C(=O)c1ccc(Cl)c(Cl)c1)C3]

36

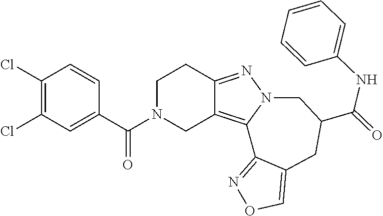 [SMILES: O=C(Nc1ccccc1)C1Cc2conc2-c2c3c(nn2C1)CCN(C(=O)c1ccc(Cl)c(Cl)c1)C3]

37

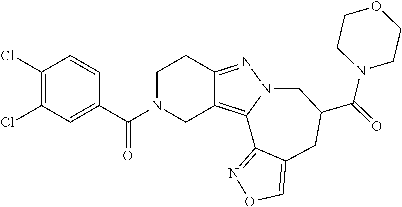 [SMILES: O=C(c1ccc(Cl)c(Cl)c1)N1CCc2nn3c(c2C1)-c1nocc1CC(C(=O)N1CCOCC1)C3]

38

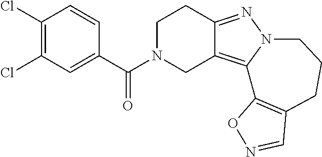 [SMILES: O=C(c1ccc(Cl)c(Cl)c1)N1CCc2nn3c(c2C1)-c1oncc1CCC3]

39

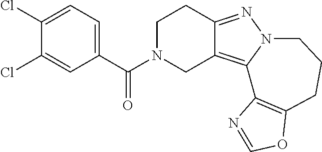 [SMILES: O=C(c1ccc(Cl)c(Cl)c1)N1CCc2nn3c(c2C1)-c1ncoc1CCC3]

40

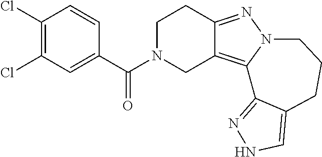 [SMILES: O=C(c1ccc(Cl)c(Cl)c1)N1CCc2nn3c(c2C1)-c1n[nH]cc1CCC3]

41

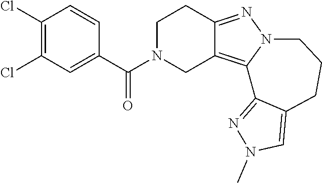 [SMILES: Cn1cc2c(n1)-c1c3c(nn1CCC2)CCN(C(=O)c1ccc(Cl)c(Cl)c1)C3]

42

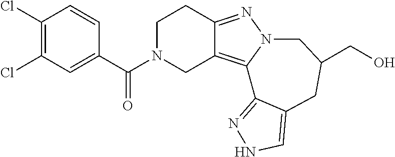 [SMILES: O=C(c1ccc(Cl)c(Cl)c1)N1CCc2nn3c(c2C1)-c1n[nH]cc1CC(CO)C3]

43

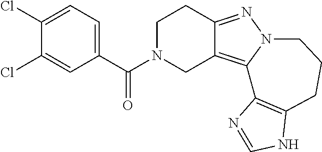 [SMILES: O=C(c1ccc(Cl)c(Cl)c1)N1CCc2nn3c(c2C1)-c1nc[nH]c1CCC3]

44

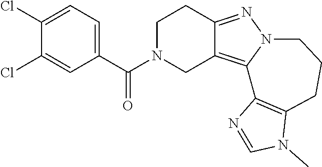 [SMILES: Cn1cnc2c1CCCn1nc3c(c1-2)CN(C(=O)c1ccc(Cl)c(Cl)c1)CC3]

45

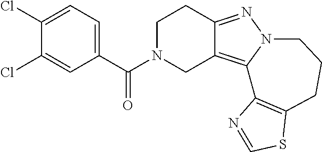 [SMILES: O=C(c1ccc(Cl)c(Cl)c1)N1CCc2nn3c(c2C1)-c1ncsc1CCC3]

46

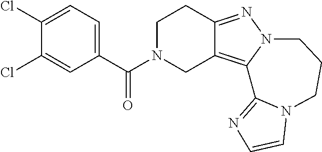 [SMILES: O=C(c1ccc(Cl)c(Cl)c1)N1CCc2nn3c(c2C1)-c1nccn1CCC3]

47

In one aspect, provided herein are compounds of Formula (Ia):

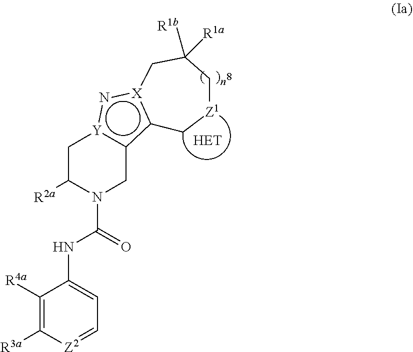

and pharmaceutically acceptable salts, stereoisomers, isotopic variants. N-oxides, or solvates of compounds of Formula (Ia);

wherein
R^{1b} is selected from the group consisting of: hydrogen, C₁₋₄alkyl, hydroxy, hydroxymethyl, (2,2-difluoroethoxy)methyl, OC₁₋₄alkyl, and fluoro;
R^{1a} is hydrogen or taken together with R^{1b} to form methylenyl;
n^{a} is an integer that is 0, 1, or 2;
R^{2a} is selected from the group consisting of: hydrogen and C₁₋₆alkyl;
R^{3a} is selected from the group consisting of: Cl, CN, and C₁₋₄haloalkyl;
R⁴ is H, or F;
HET is a 5- or 6-membered heteroaryl, optionally independently substituted with one to two substituents selected from C₁₋₄alkyl, bromo, chloro, fluoro, and hydroxy(C₁₋₄)alkyl;
X and Y are each independently selected from: N or C, such that only one of X and Y is N in any instance;
Z¹ is N or C; and
Z² is N or CF.

In embodiments, the compound of Formula (Ia) is a compound wherein R^{1b} is hydrogen, C₁₋₄alkyl, hydroxy, hydroxymethyl, (2,2-difluoroethoxy)methyl, OC₁₋₄alkyl, or fluoro.

In embodiments, the compound of Formula (Ia) is a compound wherein R^{1b} and R^{1a} are taken together with R^{1b} to form methylenyl.

In embodiments, the compound of Formula (Ia) is a compound wherein n^{a} is 1.

In embodiments, the compound of Formula (Ia) is a compound wherein n^{a} is 0.

In embodiments, the compound of Formula (Ia) is a compound wherein n^{a} is 2.

In embodiments, the compound of Formula (Ia) is a compound wherein R^{2a} is H or CH₂.

In embodiments, the compound of Formula (Ia) is a compound wherein R^{2a} is H.

In embodiments, the compound of Formula (Ia) is a compound wherein R^{2a} is CH₃.

In embodiments, the compound of Formula (Ia) is a compound wherein R^{3a} is Cl, CN, or CF₃.

In embodiments, the compound of Formula (Ia) is a compound wherein R^{4a} is H.

In embodiments, the compound of Formula (Ia) is a compound wherein R^{4a} is F.

In embodiments, the compound of Formula (Ia) is a compound wherein Y is N and X is C.

In embodiments, the compound of Formula (Ia) is a compound wherein Y is C and X is N.

In embodiments, the compound of Formula (Ia) is a compound wherein Z¹ is N.

In embodiments, the compound of Formula (Ia) is a compound wherein Z¹ is C.

In embodiments, the compound of Formula (Ia) is a compound wherein Z² is N.

In embodiments, the compound of Formula (Ia) is a compound wherein Z² is CF.

In embodiments, the compound of Formula (Ia) is a compound wherein

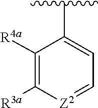

is 3-cyano-4-fluorophenyl, 4-fluoro-3-(trifluoromethyl)phenyl, or 3-chloro-4-fluorophenyl.

In embodiments, the compound of Formula (Ia) is a compound wherein

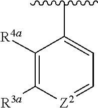

is 3-cyano-4-fluorophenyl.

In embodiments, the compound of Formula (a) is a compound wherein HET is a heteroaryl independently selected from the group consisting of isoxazolyl, pyridinyl, triazolyl, 3-methyl-triazolyl, pyridazinyl, pyrazolyl, or 1-methylpyrazolyl.

In embodiments, the compound of Formula (Ia) is a compound wherein HET is a heteroaryl independently selected from the group consisting of isoxazolyl and pyrazolyl.

A further embodiment of the present disclosure is a compound selected from the group consisting of:

TABLE 2

| Ex # | Structure | Name |
|---|---|---|
| 1a | 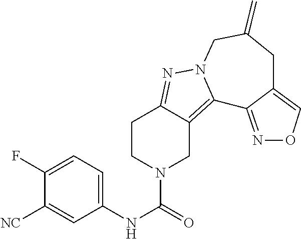 [SMILES: C=C1Cc2conc2-c2c3c(nn2C1)CCN(C(=O)Nc1ccc(F)c(C#N)c1)C3] | N-(3-Cyano-4-fluorophenyl)-5- methylene-5,6,9,10-tetrahydro- 4H-isoxazolo[3,4-c]pyrido- [4′,3′:3,4]pyrazolo[1,5-a]- azepine-11(12H)-carboxamide; |
| 2a | 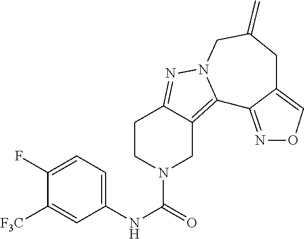 [SMILES: C=C1Cc2conc2-c2c3c(nn2C1)CCN(C(=O)Nc1ccc(F)c(C(F)(F)F)c1)C3] | N-(4-Fluoro-3-(trifluoromethyl)- phenyl)-5-methylene-5,6,9,10- tetrahydro-4H-isoxazolo[3,4- c]pyrido[4′,3′:3,4]pyrazolo[1,5- a]azepine-11(12H)-carboxamide; |
| 3a | 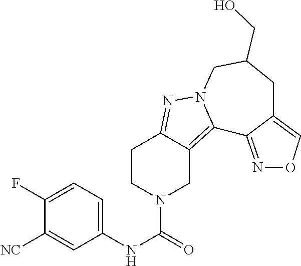 [SMILES: N#Cc1cc(NC(=O)N2CCc3nn4c(c3C2)-c2nocc2CC(CO)C4)ccc1F] | N-(3-Cyano-4-fluorophenyl)-5- (hydroxymethyl)-5,6,9,10- tetrahydro-4H-isoxazolo[3,4- c]pyrido[4′,3′:3,4]pyrazolo[1,5- a]azepine-11(12H)-carboxamide; |
| 4a | 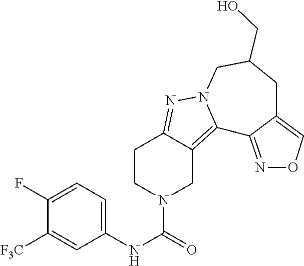 [SMILES: O=C(Nc1ccc(F)c(C(F)(F)F)c1)N1CCc2nn3c(c2C1)-c1nocc1CC(CO)C3] | N-(4-Fluoro-3-(trifluoromethyl)- phenyl)-5-(hydroxymethyl)- 5,6,9,10-tetrahydro-4H- isoxazolo[3,4-c]pyrido[4′,3′:3,4]- pyrazolo[1,5-a]azepine-11(12H)- carboxamide; |
| 5a | 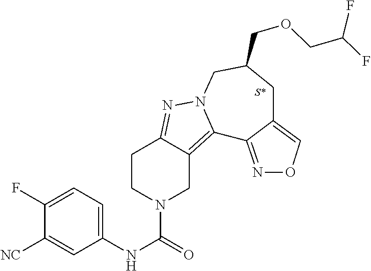 [SMILES: N#Cc1cc(NC(=O)N2CCc3nn4c(c3C2)-c2nocc2C[C@H](COCC(F)F)C4)ccc1F] | (5S*)-N-(3-Cyano-4-fluoro- phenyl)-5-((2,2-difluoroethoxy)- methyl)-5,6,9,10-tetrahydro-4H- isoxazolo[3,4-c]pyrido[4′,3′:3,4]- pyrazolo[1,5-a]azepine-11(12H)- carboxamide; |
| 6a | 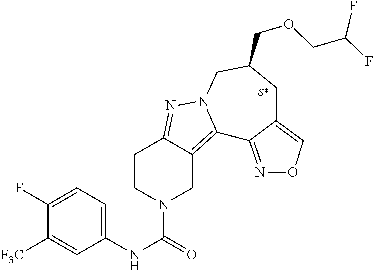 [SMILES: O=C(Nc1ccc(F)c(C(F)(F)F)c1)N1CCc2nn3c(c2C1)-c1nocc1C[C@H](COCC(F)F)C3] | (5S*)-5-((2,2-Difluoroethoxy)- methyl)-N-(4-fluoro-3- (trifluoromethyl)phenyl)- 5,6,9,10-tetrahydro-4H- isoxazolo[3,4-c]pyrido- [4′,3′:3,4]pyrazolo[1,5-a]azepine- 11(12H)-carboxamide; |
| 7a | 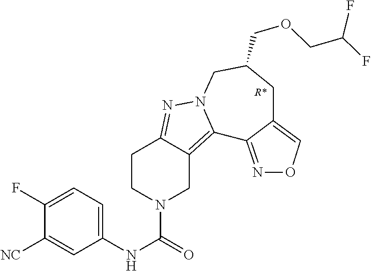 [SMILES: N#Cc1cc(NC(=O)N2CCc3nn4c(c3C2)-c2nocc2C[C@@H](COCC(F)F)C4)ccc1F] | (5R*)-N-(3-Cyano-4- fluorophenyl)-5-((2,2- difluoroethoxy)methyl)-5,6,9,10- tetrahydro-4H-isoxazolo[3,4- c]pyrido[4′,3′:3,4]pyrazolo[1,5- a]azepine-11(12H)-carboxamide; |
| 8a | 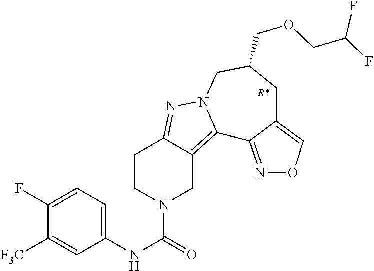 [SMILES: O=C(Nc1ccc(F)c(C(F)(F)F)c1)N1CCc2nn3c(c2C1)-c1nocc1C[C@@H](COCC(F)F)C3] | (5R*)-5-((2,2- Difluoroethoxy)methyl)-N-(4- fluoro-3- (trifluoromethyl)phenyl)- 5,6,9,10-tetrahydro-4H- isoxazolo[3,4- c]pyrido[4′,3′:3,4]pyrazolo[1,5- a]azepine-11(12H)-carboxamide; |
| 9a | 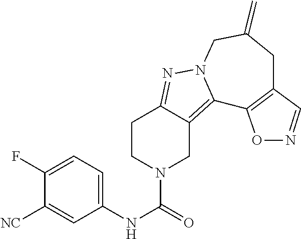 [SMILES: C=C1Cc2cnoc2-c2c3c(nn2C1)CCN(C(=O)Nc1ccc(F)c(C#N)c1)C3] | N-(3-Cyano-4-fluorophenyl)-5- methylene-5,6,9,10-tetrahydro- 4H-isoxazolo[5,4- c]pyrido[4′,3′:3,4]pyrazolo[1,5- a]azepine-11(12H)-carboxamide; |
| 10a | 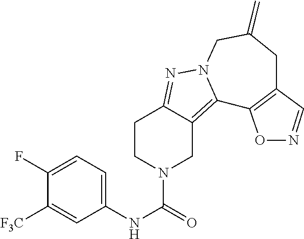 [SMILES: C=C1Cc2cnoc2-c2c3c(nn2C1)CCN(C(=O)Nc1ccc(F)c(C(F)(F)F)c1)C3] | N-(4-Fluoro-3- (trifluoromethyl)phenyl)-5- methylene-5,6,9,10-tetrahydro- 4H-isoxazolo[5,4- c]pyrido]4′,3′:3,4]pyrazolo[1,5- a]azepine-11(12H)-carboxamide; |
| 11a | 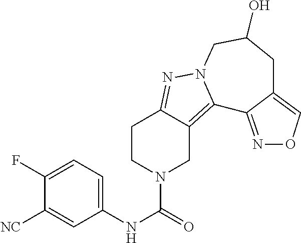 [SMILES: N#Cc1cc(NC(=O)N2CCc3nn4c(c3C2)-c2nocc2CC(O)C4)ccc1F] | N-(3-Cyano-4-fluorophenyl)-5- hydroxy-5,6,9,10-tetrahydro-4H- isoxazolo[3,4- c]pyrido[4′,3′:3,4]pyrazolo[1,5- a]azepine-11(12H)-carboxamide; |
| 12a | 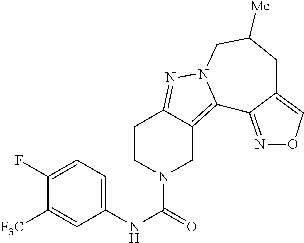 [SMILES: CC1Cc2conc2-c2c3c(nn2C1)CCN(C(=O)Nc1ccc(F)c(C(F)(F)F)c1)C3] | N-(4-Fluoro-3- (trifluoromethyl)phenyl)-5- methyl-5,6,9,10-tetrahydro-4H- isoxazolo[3,4- c]pyrido[4′,3′:3,4]pyrazolo[1,5- a]azepine-11(12H)-carboxamide; |
| 13a | 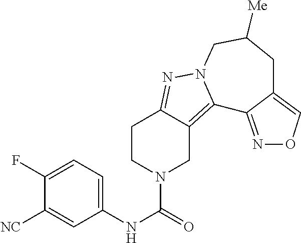 [SMILES: CC1Cc2conc2-c2c3c(nn2C1)CCN(C(=O)Nc1ccc(F)c(C#N)c1)C3] | N-(3-Cyano-4-fluorophenyl)-5- methyl-5,6,9,10-tetrahydro-4H- isoxazolo[3,4- c]pyrido[4′,3′:3,4]pyrazolo[1,5- a]azepine-11(12H)-carboxamide; |
| 14a | 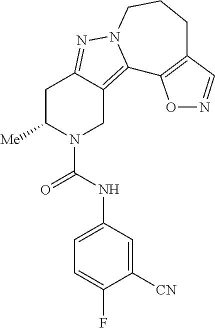 [SMILES: C[C@@H]1Cc2nn3c(c2CN1C(=O)Nc1ccc(F)c(C#N)c1)-c1oncc1CCC3] | (10R)-N-(3-Cyano-4- fluorophenyl)-10-methyl- 5,6,9,10-tetrahydro-4H- isoxazolo[5,4- c]pyrido[4′,3′:3,4]pyrazolo[1,5- a]azepine-11(12H)-carboxamide; |
| 15a | 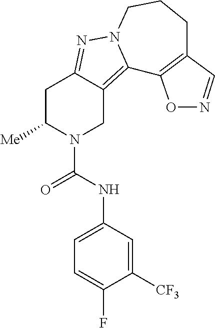 [SMILES: C[C@@H]1Cc2nn3c(c2CN1C(=O)Nc1ccc(F)c(C(F)(F)F)c1)-c1oncc1CCC3] | (10R)-N-(4-Fluoro-3- (trifluoromethyl)phenyl)-10- methyl-5,6,9,10-tetrahydro-4H- isoxazolo[5,4- c]pyrido[4′,3′:3,4]pyrazolo[1,5- a]azepine-11(12H)-carboxamide; |
| 16a | 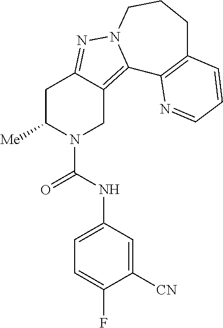 [SMILES: C[C@@H]1Cc2nn3c(c2CN1C(=O)Nc1ccc(F)c(C#N)c1)-c1ncccc1CCC3] | (11R)-N-(3-Cyano-4- fluorophenyl)-11-methyl- 6,7,10,11-tetrahydro-5H- pyrido[2,3- c]pyrido[4′,3′:3,4]pyrazolo[1,5- a]azepine-12(13H)-carboxamide; |
| 17a | 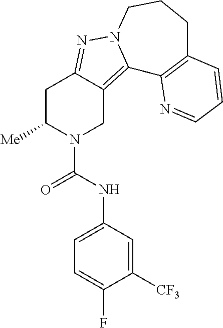 [SMILES: C[C@@H]1Cc2nn3c(c2CN1C(=O)Nc1ccc(F)c(C(F)(F)F)c1)-c1ncccc1CCC3] | (11R)-N-(4-Fluoro-3- (trifluoromethyl)phenyl)-11- methyl-6,7,10,11-tetrahydro-5H- pyrido[2,3- c]pyrido[4′,3′:3,4]pyrazolo[1,5- a]azepine-12(13H)-carboxamide; |
| 18a | 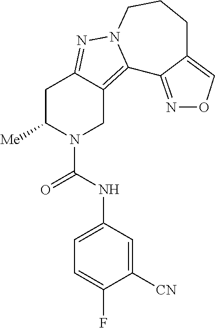 [SMILES: C[C@@H]1Cc2nn3c(c2CN1C(=O)Nc1ccc(F)c(C#N)c1)-c1nocc1CCC3] | (10R)-N-(3-Cyano-4- fluorophenyl)-10-methyl- 5,6,9,10-tetrahydro-4H- isoxazolo[3,4- c]pyrido[4′,3′:3,4]pyrazolo[1,5- a]azepine-11(12H)-carboxamide; |
| 19a | 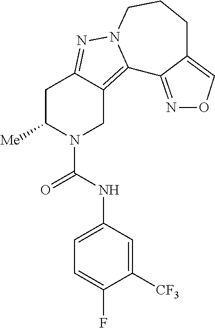 [SMILES: C[C@@H]1Cc2nn3c(c2CN1C(=O)Nc1ccc(F)c(C(F)(F)F)c1)-c1nocc1CCC3] | (10R)-N-(4-Fluoro-3- (trifluoromethyl)phenyl)-10- methyl-5,6,9,10-tetrahydro-4H- isoxazolo[3,4- c]pyrido[4′,3′:3,4]pyrazolo[1,5- a]azepine-11(12H)-carboxamide; |
| 20a | 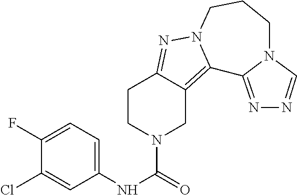 [SMILES: O=C(Nc1ccc(F)c(Cl)c1)N1CCc2nn3c(c2C1)-c1nncn1CCC3] | N-(3-Chloro-4-fluorophenyl)- 6,7,10,11-tetrahydro-5H- pyrido[4′,3′:3,4]pyrazolo[1,5- a][1,2,4]triazolo[3,4- c][1,4]diazepine-12(13H)- carboxamide; |
| 21a | 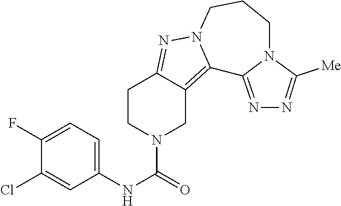 [SMILES: Cc1nnc2n1CCCn1nc3c(c1-2)CN(C(=O)Nc1ccc(F)c(Cl)c1)CC3] | N-(3-Chloro-4-fluorophenyl)-3- methyl-6,7,10,11-tetrahydro-5H- pyrido[4′,3′:3,4]pyrazolo[1,5- a][1,2,4]triazolo[3,4- c][1,4]diazepine-12(13H)- carboxamide; |
| 22a | 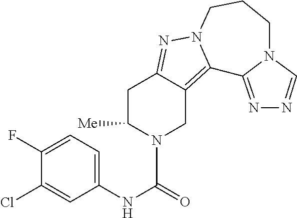 [SMILES: C[C@@H]1Cc2nn3c(c2CN1C(=O)Nc1ccc(F)c(Cl)c1)-c1nncn1CCC3] | (11R)-N-(3-Chloro-4- fluorophenyl)-11-methyl- 6,7,10,11-tetrahydro-5H- pyrido[4′,3′:3,4]pyrazolo[1,5- a][1,2,4]triazolo[3,4- c][1,4]diazepine-12(13H)- carboxamide; |
| 23a | 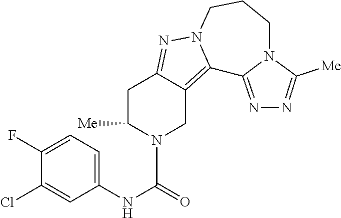 [SMILES: Cc1nnc2n1CCCn1nc3c(c1-2)CN(C(=O)Nc1ccc(F)c(Cl)c1)[C@H](C)C3] | (11R)-N-(3-Chloro-4- fluorophenyl)-11-methyl- 6,7,10,11-tetrahydro-5H- pyrido[4′,3′:3,4]pyrazolo[1,5- a][1,2,4]triazolo[3,4- c][1,4]diazepine-12(13H)- carboxamide; |
| 24a | 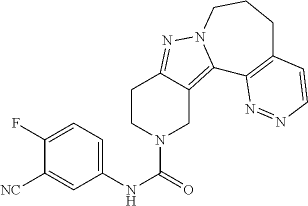 [SMILES: N#Cc1cc(NC(=O)N2CCc3nn4c(c3C2)-c2nnccc2CCC4)ccc1F] | N-(3-Cyano-4-fluorophenyl)- 6,7,10,11-tetrahydro-5H- pyridazino[4′,3′:3,4]pyrazolo[1,5- a]azepine-12(13H)-carboxamide; |
| 25a | 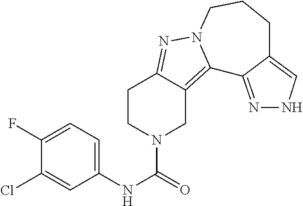 [SMILES: O=C(Nc1ccc(F)c(Cl)c1)N1CCc2nn3c(c2C1)-c1n[nH]cc1CCC3] | N-(3-Chloro-4-fluorophenyl)- 4,5,6,9,10,12- hexahydropyrazolo[3,4- c]pyrido[4′,3′:3,4]pyrazolo[1,5- a]azepine-11(2H)-carboxamide; |
| 26a | 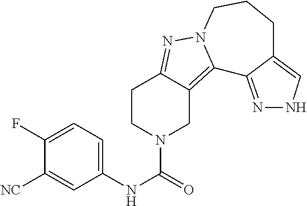 [SMILES: N#Cc1cc(NC(=O)N2CCc3nn4c(c3C2)-c2n[nH]cc2CCC4)ccc1F] | N-(3-Cyano-4-fluorophenyl)- 4,5,6,9,10,12- hexahydropyrazolo[3,4- c]pyrido[4′,3′:3,4]pyrazolo[1,5- a]azepine-11(2H)-carboxamide; |
| 27a | 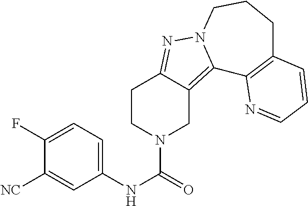 [SMILES: N#Cc1cc(NC(=O)N2CCc3nn4c(c3C2)-c2ncccc2CCC4)ccc1F] | N-(3-Cyano-4-fluorophenyl)- 6,7,10,11-tetrahydro-5H- pyrido[4′,3′:3,4]pyrazolo[1,5- a]azepine-12(13H)-carboxamide; |
| 28a | 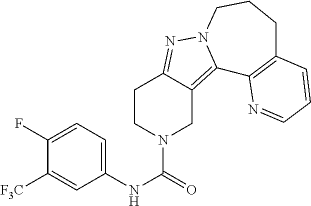 [SMILES: O=C(Nc1ccc(F)c(C(F)(F)F)c1)N1CCc2nn3c(c2C1)-c1ncccc1CCC3] | N-(4Fluoro-3- (trifluoromethyl)phenyl)- 6,7,10,11-tetrahydro-5H- pyrido[2,3-c]pyrido[4′,3′:3,4] pyrazolo[1,5-a]azepine- 12(13H)-carboxamide; |
| 29a | 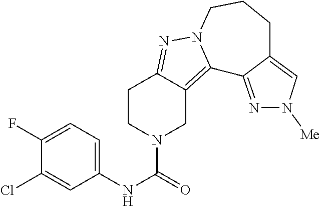 [SMILES: Cn1cc2c(n1)-c1c3c(nn1CCC2)CCN(C(=O)Nc1ccc(F)c(Cl)c1)C3] | N-(3-Chloro-4-fluorophenyl)-2- methyl-4,5,6,9,10,12- hexahydropyrazolo[3,4- c]pyrido[4′,3′:3,4]pyrazolo[1,5- a]azepine-11(2H)-carboxamide; |
| 30a | 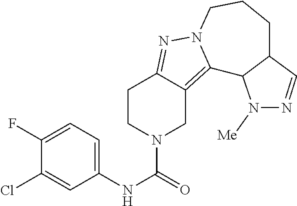 [SMILES: CN1N=CC2CCCn3nc4c(c3C21)CN(C(=O)Nc1ccc(F)c(Cl)c1)CC4] | N-(3-Chloro-4-fluorophenyl)-1- methyl-4,5,6,9,10,12- hexahydropyrazolo[3,4- c]pyrido[4′,3′:3,4]pyrazolo[1,5- a]azepine-11(1H)-carboxamide; |
| 31a | 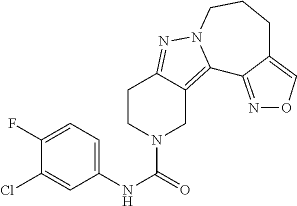 [SMILES: O=C(Nc1ccc(F)c(Cl)c1)N1CCc2nn3c(c2C1)-c1nocc1CCC3] | N-(3-Chloro-4-fluorophenyl)- 5,6,9,10-tetrahydro-4H- isoxazolo[3,4- c]pyrido[4′,3′:3,4]pyrazolo[1,5- a]azepine-11(12H)-carboxamide; |
| 32a | 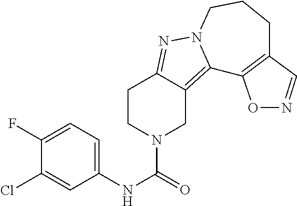 [SMILES: O=C(Nc1ccc(F)c(Cl)c1)N1CCc2nn3c(c2C1)-c1oncc1CCC3] | N-(3Chloro-4-fluorophenyl)- 5,6,9,10-tetrahydro-4H- isoxazolo[5,4- c]pyrido[4′,3′:3,4]pyrazolo[1,5- a]azepine-11(12H)-carboxamide; |
| 33a | 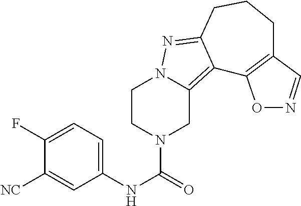 [SMILES: N#Cc1cc(NC(=O)N2CCn3nc4c(c3C2)-c2oncc2CCC4)ccc1F] | N-(3-Cyano-4-fluorophenyl)- 5,6,9,10-tetrahydro-4H- isoxazolo[5″,4″:3′,4′]cyclo- hepta[1′,2′:3,4]pyrazolo[1,5- a]pyrazine-11(12H)- carboxamide; |
| 34a | 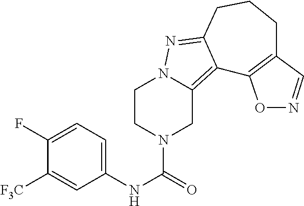 [SMILES: O=C(Nc1ccc(F)c(C(F)(F)F)c1)N1CCn2nc3c(c2C1)-c1oncc1CCC3] | N-(3-Cyano-4-fluorophenyl)- 5,6,9,10-tetrahydro-4H- isoxazolo[5″,4″:3′,4′]cyclo- hepta[1′,2′:3,4]pyrazolo[1,5- a]pyrazine-11(12H)- carboxamide; |
| 35a | 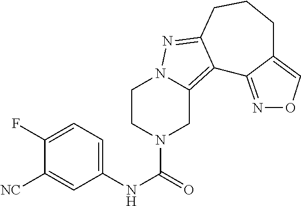 [SMILES: N#Cc1cc(NC(=O)N2CCn3nc4c(c3C2)-c2nocc2CCC4)ccc1F] | N-(3-Cyano-4-fluorophenyl)- 5,6,9,10-tetrahydro-4H- isoxazolo[3″,4″:3′,4′]cyclo- hepta[1′,2′:3,4]pyrazolo[1,5- a]pyrazine-11(12H)- carboxamide; and |
| 36a | 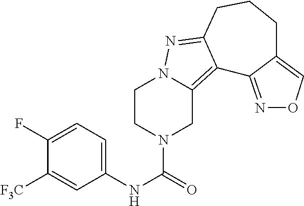 [SMILES: O=C(Nc1ccc(F)c(C(F)(F)F)c1)N1CCn2nc3c(c2C1)-c1nocc1CCC3] | N-(4-Fluoro-3- (trifluoromethyl)phenyl)- 5,6,9,10-tetrahydro-4H- isoxazolo[3″,4″:3′,4′]cyclo- hepta[1′,2′:3,4]pyrazolo[1,5- a]pyrazine-11(12H)- carboxamide; |

and pharmaceutically acceptable salts, N-oxides, or solvates thereof.

### Pharmaceutical Compositions

Also disclosed herein are pharmaceutical compositions comprising a compound according to the invention, or a pharmaceutically acceptable salt thereof, and at least one pharmaceutically acceptable carrier.

An embodiment of the present disclosure is a pharmaceutical composition comprising at least one pharmaceutically acceptable excipient and at least one compound selected from the group consisting of the compounds described below (cf. Table 3), or a stereoisomer or tautomer thereof, or a pharmaceutically acceptable salt thereof.

Therefore, also disclosed herein are pharmaceutical compositions comprising

(A) at least one compound of Formula (I):

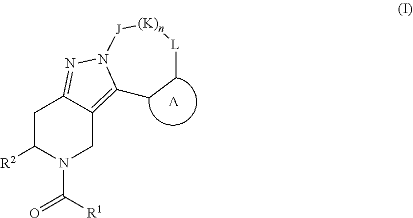

or a stereoisomer or tautomer thereof, or a pharmaceutically acceptable salt thereof, wherein

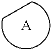

is a 5-membered heteroalkyl comprising one, two or three heteroatoms, the heteroatoms being independently selected from the group consisting of N, O and S, wherein the 5-membered heteroaryl is substituted with one or more substituents selected from the group consisting of H, C₁₋₄alkyl, CF₃, CF₂H, NH₂, NH(CH₃), N(CH₃)₂ and phenyl;

R¹ is phenyl substituted with one or more substituents selected from the group consisting of Cl, F, CF₃, CF₂H, CN, and C₁₋₄alkyl;

R² is selected from the group consisting of H, C₁₋₄alkyl and C₁₋₄alkyl substituted with one or more F;

J is CHR³:

R³ is selected from the group consisting of H, CH₂OH, and C(═O)N(R⁴)(R⁵);

R⁴ and R⁵ are independently selected from the group consisting of H, C₁₋₄alkyl, and C₃₋₄cycloalkyl, wherein C₁₋₄alkyl is optionally substituted with one or more substituents selected from the group consisting of OH and F;

K is selected from the group consisting of C(R⁶)(R⁷), C═CH₂ and C(═O);

R⁶ and R⁷ are independently selected from the group consisting of H, F, OH, OCH₃, CH₂OH, C(═O)R⁸ and C(═O)N(R⁹)(R¹⁰);

R⁸ is OH or morpholine;

R⁹ and R¹⁰ are independently selected from the group consisting of H, phenyl, C₁₋₄alkyl and C₃₋₄cycloalkyl, wherein C₁₋₄alkyl is optionally substituted with one or more substituents selected from the group consisting of OH and F;

n is an integer of 0 or 1;

L is C(R¹¹)(R¹²), NH, O;

R¹¹ and R¹² are independently selected from the group consisting of H and C(═O)N(R¹³)(R¹⁴); and

R¹³ and R¹⁴ are independently selected from the group consisting of H, C₁₋₄alkyl and C₃₋₄cycloalkyl, wherein C₁₋₄alkyl is optionally substituted with one or more substituents selected from the group consisting of OH and F. and

(B) at least one pharmaceutically acceptable excipient.

An embodiment of the present disclosure is a pharmaceutical composition comprising at least one pharmaceutically acceptable excipient and at least one compound selected from the group consisting of the compounds described below (cf. Table 3), or a stereoisomer or tautomer thereof, or a pharmaceutically acceptable salt thereof.

TABLE 3

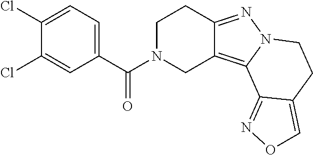 [SMILES: O=C(c1ccc(Cl)c(Cl)c1)N1CCc2nn3c(c2C1)-c1nocc1CC3]

1

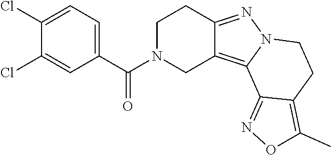 [SMILES: Cc1onc2c1CCn1nc3c(c1-2)CN(C(=O)c1ccc(Cl)c(Cl)c1)CC3]

2

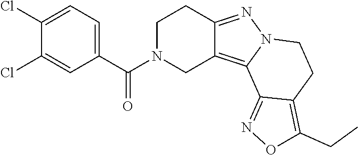 [SMILES: CCc1onc2c1CCn1nc3c(c1-2)CN(C(=O)c1ccc(Cl)c(Cl)c1)CC3]

3

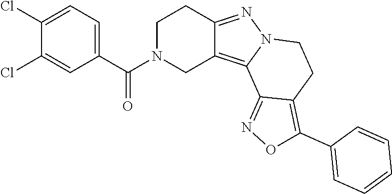 [SMILES: O=C(c1ccc(Cl)c(Cl)c1)N1CCc2nn3c(c2C1)-c1noc(-c2ccccc2)c1CC3]

4

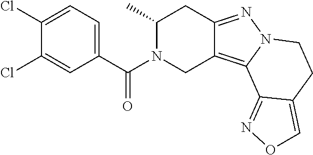 [SMILES: C[C@@H]1Cc2nn3c(c2CN1C(=O)c1ccc(Cl)c(Cl)c1)-c1nocc1CC3]

5

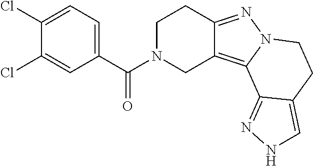 [SMILES: O=C(c1ccc(Cl)c(Cl)c1)N1CCc2nn3c(c2C1)-c1n[nH]cc1CC3]

6

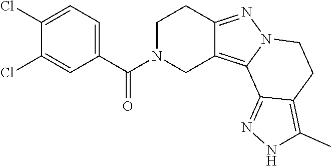 [SMILES: Cc1[nH]nc2c1CCn1nc3c(c1-2)CN(C(=O)c1ccc(Cl)c(Cl)c1)CC3]

7

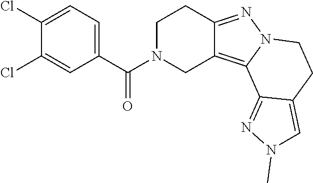 [SMILES: Cn1cc2c(n1)-c1c3c(nn1CC2)CCN(C(=O)c1ccc(Cl)c(Cl)c1)C3]

8

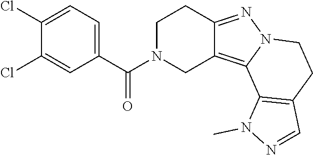 [SMILES: Cn1ncc2c1-c1c3c(nn1CC2)CCN(C(=O)c1ccc(Cl)c(Cl)c1)C3]

9

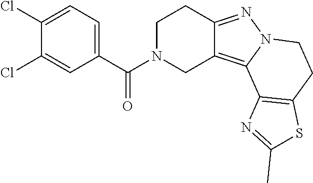 [SMILES: Cc1nc2c(s1)CCn1nc3c(c1-2)CN(C(=O)c1ccc(Cl)c(Cl)c1)CC3]

10

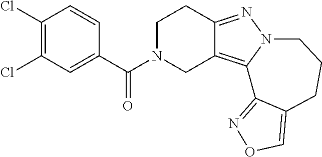 [SMILES: O=C(c1ccc(Cl)c(Cl)c1)N1CCc2nn3c(c2C1)-c1nocc1CCC3]

11

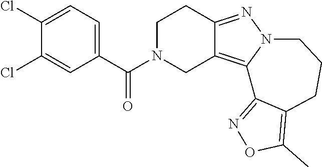 [SMILES: Cc1onc2c1CCCn1nc3c(c1-2)CN(C(=O)c1ccc(Cl)c(Cl)c1)CC3]

12

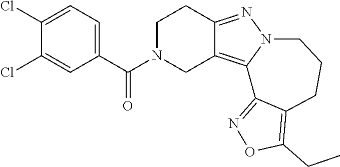 [SMILES: CCc1onc2c1CCCn1nc3c(c1-2)CN(C(=O)c1ccc(Cl)c(Cl)c1)CC3]

13

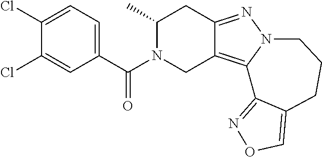 [SMILES: C[C@@H]1Cc2nn3c(c2CN1C(=O)c1ccc(Cl)c(Cl)c1)-c1nocc1CCC3]

14

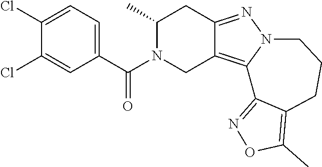 [SMILES: Cc1onc2c1CCCn1nc3c(c1-2)CN(C(=O)c1ccc(Cl)c(Cl)c1)[C@H](C)C3]

15

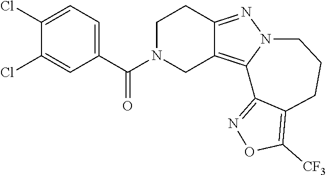 [SMILES: O=C(c1ccc(Cl)c(Cl)c1)N1CCc2nn3c(c2C1)-c1noc(C(F)(F)F)c1CCC3]

16

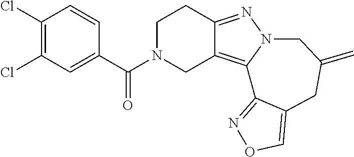 [SMILES: C=C1Cc2conc2-c2c3c(nn2C1)CCN(C(=O)c1ccc(Cl)c(Cl)c1)C3]

17

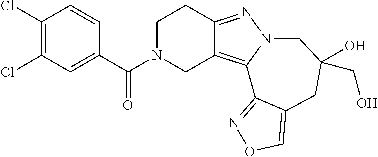 [SMILES: O=C(c1ccc(Cl)c(Cl)c1)N1CCc2nn3c(c2C1)-c1nocc1CC(O)(CO)C3]

18

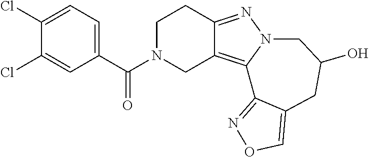 [SMILES: O=C(c1ccc(Cl)c(Cl)c1)N1CCc2nn3c(c2C1)-c1nocc1CC(O)C3]

19

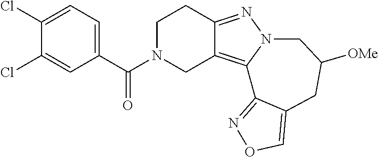 [SMILES: COC1Cc2conc2-c2c3c(nn2C1)CCN(C(=O)c1ccc(Cl)c(Cl)c1)C3]

20

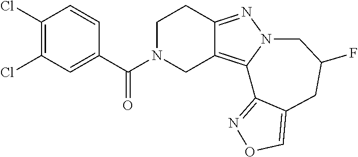 [SMILES: O=C(c1ccc(Cl)c(Cl)c1)N1CCc2nn3c(c2C1)-c1nocc1CC(F)C3]

21

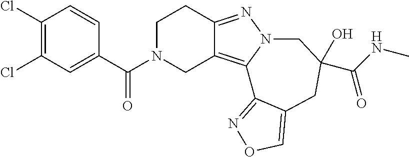 [SMILES: CNC(=O)C1(O)Cc2conc2-c2c3c(nn2C1)CCN(C(=O)c1ccc(Cl)c(Cl)c1)C3]

22

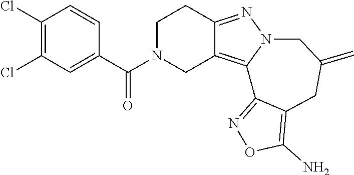 [SMILES: C=C1Cc2c(noc2N)-c2c3c(nn2C1)CCN(C(=O)c1ccc(Cl)c(Cl)c1)C3]

23

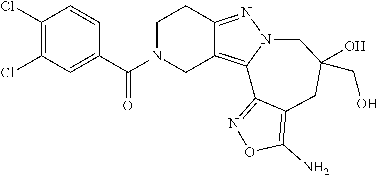 [SMILES: Nc1onc2c1CC(O)(CO)Cn1nc3c(c1-2)CN(C(=O)c1ccc(Cl)c(Cl)c1)CC3]

24

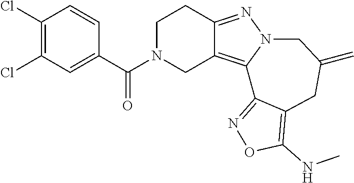 [SMILES: C=C1Cc2c(noc2NC)-c2c3c(nn2C1)CCN(C(=O)c1ccc(Cl)c(Cl)c1)C3]

25

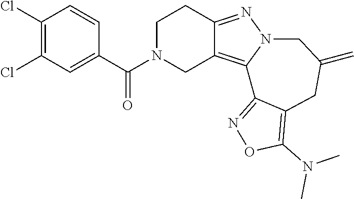 [SMILES: C=C1Cc2c(noc2N(C)C)-c2c3c(nn2C1)CCN(C(=O)c1ccc(Cl)c(Cl)c1)C3]

26

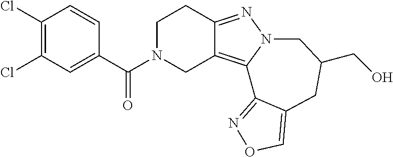 [SMILES: O=C(c1ccc(Cl)c(Cl)c1)N1CCc2nn3c(c2C1)-c1nocc1CC(CO)C3]

27

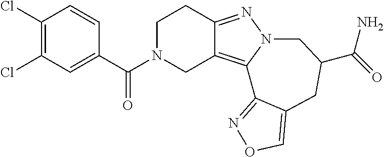 [SMILES: NC(=O)C1Cc2conc2-c2c3c(nn2C1)CCN(C(=O)c1ccc(Cl)c(Cl)c1)C3]

28

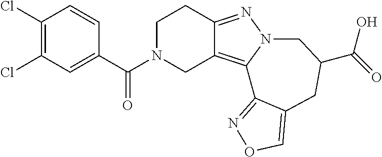 [SMILES: O=C(O)C1Cc2conc2-c2c3c(nn2C1)CCN(C(=O)c1ccc(Cl)c(Cl)c1)C3]

29

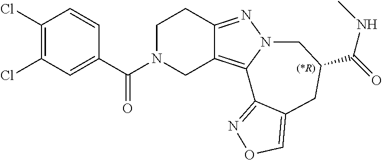 [SMILES: CNC(=O)[C@@H]1Cc2conc2-c2c3c(nn2C1)CCN(C(=O)c1ccc(Cl)c(Cl)c1)C3]

30

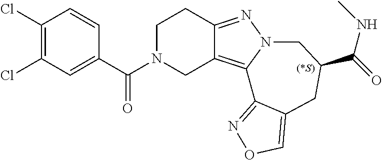 [SMILES: CNC(=O)[C@H]1Cc2conc2-c2c3c(nn2C1)CCN(C(=O)c1ccc(Cl)c(Cl)c1)C3]

31

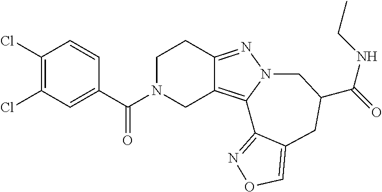 [SMILES: CCNC(=O)C1Cc2conc2-c2c3c(nn2C1)CCN(C(=O)c1ccc(Cl)c(Cl)c1)C3]

32

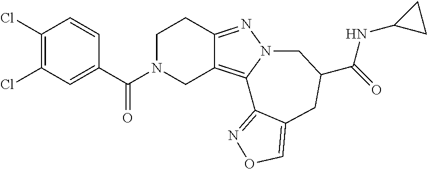 [SMILES: O=C(NC1CC1)C1Cc2conc2-c2c3c(nn2C1)CCN(C(=O)c1ccc(Cl)c(Cl)c1)C3]

33

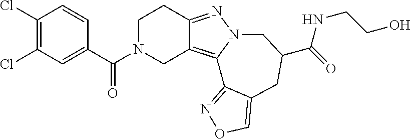 [SMILES: O=C(NCCO)C1Cc2conc2-c2c3c(nn2C1)CCN(C(=O)c1ccc(Cl)c(Cl)c1)C3]

34

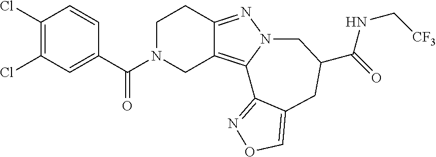 [SMILES: O=C(NCC(F)(F)F)C1Cc2conc2-c2c3c(nn2C1)CCN(C(=O)c1ccc(Cl)c(Cl)c1)C3]

35

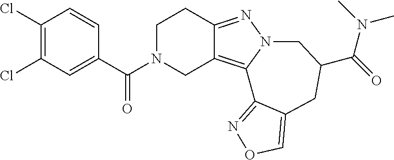 [SMILES: CN(C)C(=O)C1Cc2conc2-c2c3c(nn2C1)CCN(C(=O)c1ccc(Cl)c(Cl)c1)C3]

36

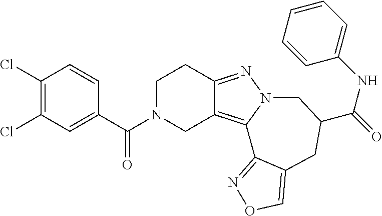 [SMILES: O=C(Nc1ccccc1)C1Cc2conc2-c2c3c(nn2C1)CCN(C(=O)c1ccc(Cl)c(Cl)c1)C3]

37

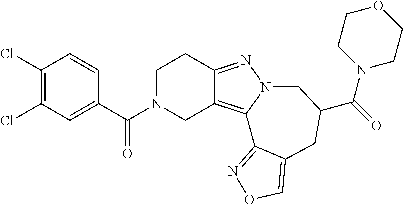 [SMILES: O=C(c1ccc(Cl)c(Cl)c1)N1CCc2nn3c(c2C1)-c1nocc1CC(C(=O)N1CCOCC1)C3]

38

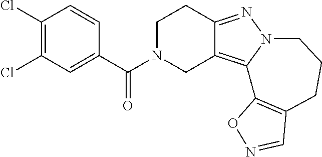 [SMILES: O=C(c1ccc(Cl)c(Cl)c1)N1CCc2nn3c(c2C1)-c1oncc1CCC3]

39

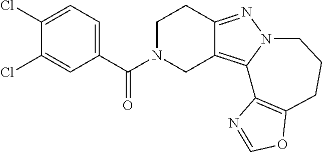 [SMILES: O=C(c1ccc(Cl)c(Cl)c1)N1CCc2nn3c(c2C1)-c1ncoc1CCC3]

40

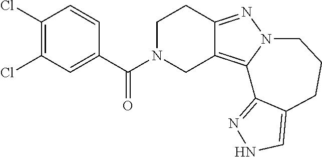 [SMILES: O=C(c1ccc(Cl)c(Cl)c1)N1CCc2nn3c(c2C1)-c1n[nH]cc1CCC3]

41

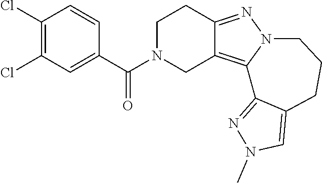 [SMILES: Cn1cc2c(n1)-c1c3c(nn1CCC2)CCN(C(=O)c1ccc(Cl)c(Cl)c1)C3]

42

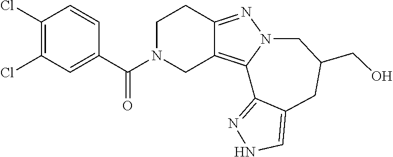 [SMILES: O=C(c1ccc(Cl)c(Cl)c1)N1CCc2nn3c(c2C1)-c1n[nH]cc1CC(CO)C3]

43

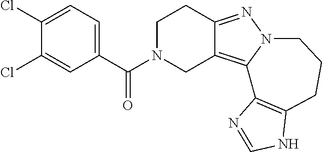 [SMILES: O=C(c1ccc(Cl)c(Cl)c1)N1CCc2nn3c(c2C1)-c1nc[nH]c1CCC3]

44

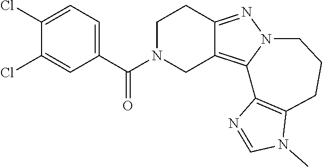 [SMILES: Cn1cnc2c1CCCn1nc3c(c1-2)CN(C(=O)c1ccc(Cl)c(Cl)c1)CC3]

45

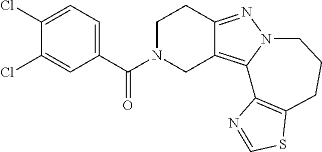 [SMILES: O=C(c1ccc(Cl)c(Cl)c1)N1CCc2nn3c(c2C1)-c1ncsc1CCC3]

46

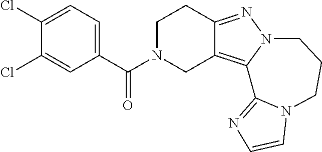 [SMILES: O=C(c1ccc(Cl)c(Cl)c1)N1CCc2nn3c(c2C1)-c1nccn1CCC3]

47

Also disclosed herein are pharmaceutical compositions comprising

(A) at least one compound of Formula (Ia):

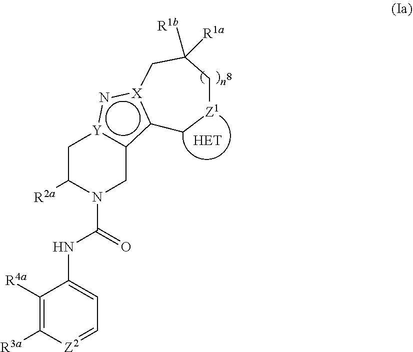

wherein
R^{1b} is selected from the group consisting of: hydrogen, C₁₋₄alkyl, hydroxy, hydroxymethyl, (2,2-difluoroethoxy)methyl, OC₁₋₄alkyl, and fluoro;
R^{1a} is hydrogen or taken together with R^{1b} to form methylenyl;
n^{a} is an integer that is 0, 1, or 2;
R^{2a} is selected from the group consisting of: hydrogen and C₁₋₆alkyl;
R^{3a} is selected from the group consisting of: Cl, CN, and C₁₋₄haloalkyl;
R^{4a} is H, or F;
HET is a 5- or 6-membered heteroaryl, optionally independently substituted with one to two substituents selected from C₁₋₄alkyl, bromo, chloro, fluoro, and hydroxy(C₁₋₄)alkyl;
X and Y are each independently selected from: N or C, such that only one of X and Y is N in any instance;
Z¹ is N or C; and
Z² is N or CF;

and pharmaceutically acceptable salts, stereoisomers, isotopic variants, N-oxides or solvates of compounds of Formula (Ia); and

(B) at least one pharmaceutically acceptable excipient.

An embodiment of the present disclosure is a pharmaceutical composition comprising at least one pharmaceutically acceptable excipient and at least one compound of Formula Ia selected from the group consisting of:

N-(3-Cyano-4-fluorophenyl)-5-methylene-5,6,9,10-tetrahydro-4H-isoxazolo[3,4-
c]pyrido[4′,3′: 3,4]pyrazolo[1,5-a]azepine-11(12H)-carboxamide;
N-(4-Fluoro-3-(trifluoromethyl)phenyl)-5-methylene-5,6,9,10-tetrahydro-4H-
isoxazolo[3,4-c]pyrido[4′,3′: 3,4]pyrazolo[1,5-a]azepine-11(12H)-carboxamide;
N-(3-Cyano-4-fluorophenyl)-5-(hydroxymethyl)-5,6,9,10-tetrahydro-4H-
isoxazolo[3,4-c]pyrido[4′,3′: 3,4]pyrazolo[1,5-a]azepine-11(12H)-carboxamide;
N-(4-Fluoro-3-(trifluoromethyl)phenyl)-5-(hydroxymethyl)-5,6,9,10-tetrahydro-4H-
isoxazolo[3,4-c]pyrido[4′,3′: 3,4]pyrazolo[1,5-a]azepine-11(12H)-carboxamide;
(5S*)-N-(3-Cyano-4-fluorophenyl)-5-((2,2-difluoroethoxy)methyl)-5,6,9,10-
tetrahydro-4H-isoxazolo[3,4-c]pyrido[4′,3′: 3,4]pyrazolo[1,5-a]azepine-11(12H)-
carboxamide;
(5S*)-5-((2,2-Difluoroethoxy)melhyl)-N-(4-fluoro-3-(trifluoromethyl)phenyl)-
5,6,9,10-tetrahydro-4H-isoxazolo[3,4-c]pyrido[4′,3′: 3,4]pyrazolo[1,5-a]azepine-
11(12H)-carboxamide;
(5R*)-N-(3-Cyano-4-fluorophenyl)-5-((2,2-difluoroethoxy)methyl)-5,6,9,10-
tetrahydro-4H-isoxazolo[3,4-c]pyrido(4′,3′: 3,4]pyrazolo[1,5-a]azepine-11(12H)-
carboxamide;
(5R*)-5-((2,2-Difluoroethoxy)methyl)-N-(4-fluoro-3-(trifluoromethyl)phenyl)-
5,6,9,10-tetrahydro-4H-isoxazolo[3,4-c]pyrido[4′,3′: 3,4]pyrazolo[1,5-a]azepine-
11(12H)-carboxamide;
N-(3-Cyano-4-fluorophenyl)-5-methylene-5,6,9,10-tetrahydro-4H-isoxazolo[5,4-
c]pyrido[4′,3′: 3,4]pyrazolo[1,5-a]azepine-11(12H)-carboxamide;
N-(4-Fluoro-3-(trifluoromethyl)phenyl)-5-methylene-5,6,9,10-tetrahydro-4H-
isoxazolo[5,4-c]pyrido]4′3′: 3,4)pyrazolo[1,5-a]azepine-11(12H)-carboxamide;
N-(3-Cyano-4-fluorophenyl)-5-hydroxy-5,6,9,10-tetrahydro-4H-
isoxazolo[3,4-c]pyrido[4′,3′: 3,4]pyrazolo[1,5-a]azepine-11(12H)-carboxamide;
N-(4-Fluoro-3-(trifluoromethyl)phenyl)-5-methyl-5,6,9,10-tetrahydro-4H-
isoxazolo[3,4-c]pyrido[4′,3′: 3,4]pyrazolo[1,5-a]azepine-11(12H)-carboxamide;
N-(3-Cyano-4-fluorophenyl)-5-methyl-5,6,9,10-tetrahydro-4H-isoxazolo[3,4-
c]pyrido[4′,3′: 3,4]pyrazolo[1,5-a]azepine-11(12H)-carboxamide;
(10R)-N-(3-Cyano-4-fluorophenyl)-10-methyl-5,6,9,10-tetrahydro-4H-isoxazolo[5,4-
c]pyrido[4′,3′: 3,4]pyrazolo[1,5-a]azepine-11(12H)-carboxamide;
(10R)-N-(4-Fluoro-3-(trifluoromethyl)phenyl)-10-methyl-5,6,9,10-tetrahydro-4H-
isoxazolo[5,4-c]pyrido[4′3′: 3,4]pyrazolo[1,5-a]azepine-11(12H)-carboxamide;
(11R)-N-(3-Cyano-4-fluorophenyl)-11-methyl-6,7,10,11-tetrahydro-5H-pyrido[2,3-
c]pyrido[4′3′: 3,4]pyrazolo[1,5-a]azepine-12(13H)-carboxamide;
(11R)-N-(4-Fluoro-3-(trifluoromethyl)phenyl)-11-methyl-6,7,10,11-tetrahydro-5H-
pyrido[2,3-c]pyrido[4′,3′: 3,4]pyrazolo[1,5-a|azepine-12(13H)-carboxamide;
(10R)-N-(3-Cyano-4-fluorophenyl)-10-methyl-5,6,9,10-tethydro-4H-isoxazolo[3,4-
c]pyrido[4′,3′: 3,4]pyrazolo[1,5-a]azepine-11(12H)-carboxamide;
(10R)-N-(4-Fluoro-trifluoromethyl))-10-methyl-5,6,9,10-tetrahydro-4H-
isoxazolo[3,4-c]pyrido[4′,3′: 3,4]pyrazolo[1,5-a]azepine-11(12H)-carboxamide;
N-(3-Chloro-4-flurophenyl)-3-methyl-6,7,10,11-tetrahydro-5H-pyrido[4′,3′: 3,4]-pyrazolo[1,5-
a][1,2,4]triazolo[3,4-c][1,4]diazepine-12(13H)-carboxamide;
N(3-Chloro-4-fluorophenly)-3-methyl-6,7,10,11-tetrahydro-5H-pyrido[4′,3′: 3,4]-pyrazolo[1,5-
a][1,2,4]triazolo[3,4-c][1,4]diazepine-12(13H)-carboxamide;
(11R)-N-(3-Chloro-4-fluorophenyl)-11-methyl-6,7,10,11-tetrahydro-5H-
pyrido[4′,3′: 3,4]pyrazolo[1,5-a][1,2,4]triazolo[3,4-c][1,4]diazepine-12(13H)-
carboxamide;
(11R)-N-(3-Chloro-4-fluorophenyl)-11-methyl-6,7,10,11-tetrahydro-5H-
pyrido[4′3′: 3,4]pyrazolo[1,5-a][1,2,4]triazolo[3,4-c][1,4]diazepine-12(13H)-
carboxamide;
N-(3-Cyano-4-fluorophenyl)-6,7,10,11-tetrahydro-5H-pyridazino[3,4-c]pyrido-
[4′,3′: 3,4]pyrazolo[1,5-a]azepine-12(13H)-carboxamide;
N-(3-Chloro-4-fluorophenyl)-4,5,6,9,10,12-hexahydropyrazolo[3,4-
c]pyrido[4′3′: 3,4]pyrazolo[1,5-a]azepine-11(2H)-carboxamide;
N-(3-Cyano-4-fluorophenyl)-4,5,6,9,10,12-hexahydropyrazolo[3,4-
c]pyrido[4′,3′: 3,4]pyrazolo[1,5-alazepine-11(2H)-carboxamide;
N-(3-Cyano-4-fluorophenyl)-6,7,10,11-tetrahydro-5H-pyrido[2,3-
c]pyrido[4′,3′: 3,4]pyrazolo[1,5-a]azepine-12(13H)-carboxamide;
N-(4Fluoro-3-(trifluoromethyl)phenyl)-6,7,10,11-tetrahydro-5H-pyrido[2,3-
c]pyrido[4′,3′: 3,4]pyrazolo[1,5-a]azepine-12(13H)-carboxamide;
N-(3-Chloro-4-fluorophenyl)-2-methyl-4,5,6,9,10,12-hexahydropyrazolo[3,4-
c]pyrido[4′,3′: 3,4]pyrazolo[1,5-a]azepine-11(2H)-carboxamide;
N-(3-Chloro-4-fluorophenyl)-1-methyl-4,5,6,9,10,12-hexahydropyrazolo[3,4-
c]pyrido[4′,3′: 3,4]pyrazolo[1,5-a]azepine-11(1H)-carboxamide;
N-(3-Chloro-4-fluorophenyl)-5,6,9,10-tetrahydro-4H-isoxazolo[3,4-
c]pyrido[4′3′: 3,4]pyrazolo[1,5-a]azepine-11(12H)-carboxamide;
N-(3Chloro-4-fluorophenyl)-5,6,9,10-tetrahydro-4H-isoxazolo[3,4-
c]pyrido[4′3′: 3,4]pyrazolo[1,5-a]azepine-11(12H)-carboxamide;
N-(3-Cyano-4-fluorophenyl)-5,6,9,10-tetrahydro-4H-isoxazolo[5″,4″: 3′,4′]-
cyclohepta[1′,2′: 3,4]pyrazolo[1,5-a]pyrazine-11(12H)-carboxamide;
N-(3-Cyano-fluorophenyl)-5,6,9,10-tetrahyro-4H-isoxazolo[5″,4″: 3′4′]-
cyclohepta[1′,2′: 3,4]pyrazolo[1,5-a]pyrazine-11(12H)-carboxamide;
N-(3-Cyano-4-fluorophenyl)-5,6,9,10-tetrahydro-4H-isoxazolo[3″,4″: 3′4′]-
cyclohepta[1′,2′: 3,4]pyrazolo[1,5-a]pyrazine-11(12H)-carboxamide; and
N-(4-Fluoro-3-(trifluoromethyl)phenyl)-5,6,9,10-tetrahydro-4H-isoxazolo-
[3″,4″: 3′,4,]cyclohepta[1′,2′: 3,4]pyrazolo[1,5-a]pyrazine-11(12H)-carboxamide;

as well as any pharmaceutically acceptable salt, N-oxide or solvate of such compound, or any pharmaceutically acceptable prodrugs of such compound, or any pharmaceutically active metabolite of such compound.

In embodiments, the pharmaceutical composition may also comprise at least one additional active or therapeutic agent. Additional active therapeutic agents may include, for example, an anti-HBV agent such as an HBV polymerase inhibitor, interferon, viral entry inhibitor, viral maturation inhibitor, capsid assembly modulator, reverse transcriptase inhibitor, immunomodulatory agent such as a TLR-agonist, or any other agents that affect the HBV life cycle and/or the consequences of HBV infection. The active agents of the present disclosure are used, alone or in combination with one or more additional active agents, to formulate pharmaceutical compositions of the present disclosure.

As used herein, the term “composition” or “pharmaceutical composition” refers to a mixture of at least one compound useful within the present disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the present disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the present disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the present disclosure and are physiologically acceptable to the patient. Supplementary active compounds may also be incorporated into the compositions. The “pharmaceutically acceptable carrier” may further include a pharmaceutically acceptable salt of the compound useful within the present disclosure. Other additional ingredients that may be included in the pharmaceutical compositions used in the practice of the present disclosure are known in the art and described, for example in Remington's Pharmaceutical Sciences (Genaro, Ed., Mack Publishing Co., 1985, Easton, Pa.), which is incorporated herein by reference.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the active agents may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds of the present disclosure can be provided in the form of tablets or capsules, or as a solution, emulsion, or suspension. To prepare the oral compositions, the compounds may be formulated to yield a dosage of, e.g., from about 0.05 to about 100 mg/kg daily, or from about 0.05 to about 35 mg/kg daily, or from about 0.1 to about 10 mg/kg daily. For example, a total daily dosage of about 5 mg to 5 g daily may be accomplished by dosing once, twice, three, or four times per day.

Oral tablets may include a compound according to the present disclosure mixed with pharmaceutically acceptable excipients such as inert diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract or may be coated with an enteric coating.

Capsules for oral administration include hard and soft gelatin capsules. To prepare hard gelatin capsules, compounds of the present disclosure may be mixed with a solid, semi-solid, or liquid diluent. Soft gelatin capsules may be prepared by mixing the compound of the present disclosure with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this present disclosure may also be administered by non-oral routes. For example, the compositions may be formulated for rectal administration as a suppository. For parenteral use, including intravenous, intramuscular, intraperitoneal, or subcutaneous routes, the compounds of the present disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering the compounds of the present disclosure may utilize a patch formulation to affect transdermal delivery.

The invention also relates to a process for the preparation of a pharmaceutical composition according to the invention, comprising combining an effective amount of the compound of formula (I) as disclosed herein, in intimate admixture with a pharmaceutically acceptable carrier.

Compounds of the present disclosure may alternatively be administered in methods of this present disclosure by inhalation, via the nasal or oral routes, e.g., in a spray formulation also containing a suitable carrier.

Methods of Use

Provided herein are compounds, e.g., the compounds of formula (I), formula (Ia), or pharmaceutically acceptable salts thereof, which are notably useful in the treatment or prevention of HBV infection or of an HBV-associated (or HBV-induced) condition or disease in a subject in need thereof.

Without being bound to any particular mechanism of action, these compounds are believed to modulate or disrupt HBV capsid assembly and other HBV core protein (HBc) functions necessary for HBV replication or the generation of infectious particles and/or may disrupt HBV capsid assembly leading to empty capsids with greatly reduced infectivity or replication capacity, in other words, the compounds provided herein may act as Capsid Assembly Modulators or core protein allosteric modulators (CpAMs).

The compounds provided herein have potent antiviral activity, and are believed to exhibit favorable metabolic properties, tissue distribution, safety and pharmaceutical profiles, and to be suitable for use in humans. Disclosed compounds may modulate (e.g., accelerate, delay, inhibit, disrupt or reduce) normal viral capsid assembly or disassembly, bind capsid or alter metabolism of cellular polyproteins and precursors. The modulation may occur when the capsid protein is mature, or during viral infectivity. Disclosed compounds can be used in methods of modulating the activity or properties of HBV cccDNA, or the generation or release of HBV RNA particles from within an infected cell.

A compound of the application may accelerate the kinetics of HBV capsid assembly, thereby preventing or competing with the encapsidation of the Pol-pgRNA complex and thus blocking the reverse transcription of the pgRNA.

A compound of the application can be assessed e.g., by evaluating the capacity of the compound to induce or to not induce speckling of the Hepatitis B virus core protein (HBc). HBc is a small protein of about 21 kDa, which forms the icosahedral capsid. HBc has been described e.g., in Diab et al. 2018 (Antiviral Research 149 (2018) 211-220).

Capsid assembly modulators may induce the formation of morphologically intact capsids or the formation of pleomorphic non-capsid structures. Pleomorphic non-capsid structures can be visualized in stable HBV-replicating cell lines by immunofluorescence staining against the HBV core protein and appear as “core speckling” in the nucleus and cytoplasm.

The term “HBc speckling” thus refers to the capacity of inducing the formation of such pleomorphic noncapsid structures.

In an aspect, the application relates more particularly to a compound (as herein described), which does not induce speckling of HBc.

In another aspect, the application relates more particularly to a compound (as herein described), which induces speckling of HBc.

The capacity to induce or to not induce HBc speckling can be assessed by any means which the person of ordinary skill in the art finds appropriate, e.g., by:
contacting a compound of the application with HBV-infected cells (e.g., cells from a (stable) HBV-infected cell line or HBV infected cells which have been previously collected from an HBV patient);
optionally fixing and permeabilizing the cells, or optionally lysing the cells; and
determining whether contacting of these cells with the compound of the application induces or does not induce HBc speckling in these cells.

Determining whether contacting of these cells with the compound of the application induces or does not induce HBc speckling can e.g., involve immunofluorescence staining against HBc, more particularly immunofluorescence staining against HBc with an anti-HBc antibody. Examples of method to determine whether a compound of the application has or not the capacity to induce HBc speckling comprise the method described in the examples below, and the immunofluorescence assay described in Corcuera et al. 2018 (Antiviral Research (2018), doi/10.1016/j.antiviral.2018.07.011, “Novel non-heteroarylpyrimidine (HAP) capsid assembly modifiers have a different mode of action from HAPs in vitro”; cf § 2.8 of Corcuera et al. 2018). FIG. 5 of Corcuera et al. 2018 illustrates HBV core morphology when a test compound induces HBc speckling (cf. the HAP-treated cells of FIG. 5) and when a test compound does not induce HBc speckling (cf. in FIG. 5, those cells which are treated with a CAM other than HAP).

Complementarily, confirmation that a compound is inducing the formation of pleiomorphic non-capsid structures or not can be obtained by implementing a cell-free biochemical assay using recombinant HBV core dimers (i.e., not using HBV-infected cells but using recombinant HBV core dimers) and using analytical size exclusion chromatography and electron microscopy analysis: cf. e.g., § 2.4-2.5 and FIGS. 2-3 of Corcuera et al. 2018; cf. e.g., Materials and Methods, as well as FIG. 2 of Berke et al. 2017 (Antimicrobial Agents and Chemotherapy August 2017 volume 61 Issue 8 e00560-17 “Capsid Assembly Modulators have a dual mechanism of action in primary human hepatocytes infected with Hepatitis B virus”); cf. e.g., the experimental section and FIG. 4 of Huber et al 2018 (ACS Infect Dis. 2018 Dec. 24. doi: 10.1021/acsinfecdis.8b00235; “Novel Hepatitis B Virus Capsid-Targeting Antiviral that Aggregates Core Particles and Inhibits Nuclear Entry of Viral Cores”).

The disclosed compounds are useful in the prevention or treatment of an HBV infection or of an HBV-induced disease in mammal in need thereof, more particularly in a human in need thereof.

In a non-limiting aspect, these compounds may (i) modulate or disrupt HBV assembly and other HBV core protein functions necessary for HBV replication or the generation of infectious particles. (ii) inhibit the production of infectious virus particles or infection, or (iii) interact with HBV capsid to effect defective viral particles with reduced infectivity or replication capacity acting as capsid assembly modulators. In particular, and without being bound to any particular mechanism of action, it is believed that the disclosed compounds are useful in HBV treatment by disrupting, accelerating, reducing, delaying and/or inhibiting normal viral capsid assembly and/or disassembly of immature or mature particles, thereby inducing aberrant capsid morphology leading to antiviral effects such as disruption of virion assembly and/or disassembly, virion maturation, virus egress and/or infection of target cells. The disclosed compounds may act as a disruptor of capsid assembly interacting with mature or immature viral capsid to perturb the stability of the capsid, thus affecting its assembly and/or disassembly. The disclosed compounds may perturb protein folding and/or salt bridges required for stability, function and/or normal morphology of the viral capsid, thereby disrupting and/or accelerating capsid assembly and/or disassembly. The disclosed compounds may bind capsid and alter metabolism of cellular polyproteins and precursors, leading to abnormal accumulation of protein monomers and/or oligomers and/or abnormal particles, which causes cellular toxicity and death of infected cells. The disclosed compounds may cause failure of the formation of capsids of optimal stability, affecting efficient uncoating and/or disassembly of viruses (e.g., during infectivity). The disclosed compounds may disrupt and/or accelerate capsid assembly and/or disassembly when the capsid protein is immature. The disclosed compounds may disrupt and/or accelerate capsid assembly and/or disassembly when the capsid protein is mature. The disclosed compounds may disrupt and/or accelerate capsid assembly and/or disassembly during viral infectivity which may further attenuate HBV viral infectivity and/or reduce viral load. The disruption, acceleration, inhibition, delay and/or reduction of capsid assembly and/or disassembly by the disclosed compounds may eradicate the virus from the host organism. Eradication of HBV from a subject by the disclosed compounds advantageously obviates the need for chronic long-term therapy and/or reduces the duration of long-term therapy.

An additional embodiment of the present disclosure is a method of treating a subject suffering from an HBV infection, comprising administering to a subject in need of such treatment an effective amount of at least one compound of Formula (I).

In another aspect, provided herein is a method of reducing the viral load associated with an HBV infection in an individual in need thereof, comprising administering to the individual a therapeutically effective amount of a compound of Formula (I), or a pharmaceutically acceptable salt thereof.

In another aspect, provided herein is a method of reducing reoccurrence of an HBV infection in an individual in need thereof, comprising administering to the individual a therapeutically effective amount of a compound of Formula (I), or a pharmaceutically acceptable salt thereof.

In another aspect, provided herein is a method of inhibiting or reducing the formation or presence of HBV DNA-containing particles or HBV RNA-containing particles in an individual in need thereof, comprising administering to the individual a therapeutically effective amount of a compound of Formula (I), or a pharmaceutically acceptable salt thereof.

In another aspect, provided herein is a method of reducing an adverse physiological impact of an HBV infection in an individual in need thereof, comprising administering to the individual a therapeutically effective amount of a compound of Formula (I), or a pharmaceutically acceptable salt thereof.

In another aspect, provided herein is a method of inducing remission of hepatic injury from an HBV infection in an individual in need thereof, comprising administering to the individual a therapeutically effective amount of a compound of Formula (I), or a pharmaceutically acceptable salt thereof.

In another aspect, provided herein is a method of reducing the physiological impact of long-term antiviral therapy for HBV infection in an individual in need thereof, comprising administering to the individual a therapeutically effective amount of a compound of Formula (I), or a pharmaceutically acceptable salt thereof.

In another aspect, provided herein is a method of prophylactically treating an HBV infection in an individual in need thereof, wherein the individual is afflicted with a latent HBV infection, comprising administering to the individual a therapeutically effective amount of a compound of Formula (I), or a pharmaceutically acceptable salt thereof.

An additional embodiment of the present disclosure is a method of treating a subject suffering from an HBV infection, comprising administering to a subject in need of such treatment an effective amount of at least one compound of Formula (Ia).

In another aspect, provided herein is a method of reducing the viral load associated with an HBV infection in an individual in need thereof, comprising administering to the individual a therapeutically effective amount of a compound of Formula (Ia), or a pharmaceutically acceptable salt thereof.

In another aspect, provided herein is a method of reducing reoccurrence of an HBV infection in an individual in need thereof, comprising administering to the individual a therapeutically effective amount of a compound of Formula (Ia), or a pharmaceutically acceptable salt thereof.

In another aspect, provided herein is a method of inhibiting or reducing the formation or presence of HBV DNA-containing particles or HBV RNA-containing particles in an individual in need thereof, comprising administering to the individual a therapeutically effective amount of a compound of Formula (Ia), or a pharmaceutically acceptable salt thereof.

In another aspect, provided herein is a method of reducing an adverse physiological impact of an HBV infection in an individual in need thereof, comprising administering to the individual a therapeutically effective amount of a compound of Formula (Ia), or a pharmaceutically acceptable salt thereof.

In another aspect, provided herein is a method of inducing remission of hepatic injury from an HBV infection in an individual in need thereof, comprising administering to the individual a therapeutically effective amount of a compound of Formula (Ia), or a pharmaceutically acceptable salt thereof.

In another aspect, provided herein is a method of reducing the physiological impact of long-term antiviral therapy for HBV infection in an individual in need thereof, comprising administering to the individual a therapeutically effective amount of a compound of Formula (Ia), or a pharmaceutically acceptable salt thereof.

In another aspect, provided herein is a method of prophylactically treating an HBV infection in an individual in need thereof, wherein the individual is afflicted with a latent HBV infection, comprising administering to the individual a therapeutically effective amount of a compound of Formula (ta), or a pharmaceutically acceptable salt thereof.

In embodiments, the disclosed compounds are suitable for monotherapy. In embodiments, the disclosed compounds are effective against natural or native HBV strains. In embodiments, the disclosed compounds are effective against HBV strains resistant to currently known drugs.

In another embodiment, the compounds provided herein can be used in methods of modulating (e.g., inhibiting or disrupting) the activity, stability, function, and viral replication properties of HBV cccDNA.

In yet another embodiment, the compounds of the present disclosure can be used in methods of diminishing or preventing the formation of HBV cccDNA.

In another embodiment, the compounds provided herein can be used in methods of modulating (e.g., inhibiting or disrupting) the activity of HBV cccDNA.

In yet another embodiment, the compounds of the present disclosure can be used in methods of diminishing the formation of HBV cccDNA.

In another embodiment, the disclosed compounds can be used in methods of modulating, inhibiting, or disrupting the generation or release of HBV RNA particles from within the infected cell.

In a further embodiment, the total burden (or concentration) of HBV RNA particles is modulated. In a preferred embodiment, the total burden of HBV RNA is diminished.

In another embodiment, the methods provided herein reduce the viral load in the individual to a greater extent or at a faster rate compared to the administering of a compound selected from the group consisting of an HBV polymerase inhibitor, interferon, viral entry inhibitor, viral maturation inhibitor, distinct capsid assembly modulator, antiviral compounds of distinct or unknown mechanism, and any combination thereof.

In another embodiment, the methods provided herein cause a lower incidence of viral mutation and/or viral resistance than the administering of a compound selected from the group consisting of an HBV polymerase inhibitor, interferon, viral entry inhibitor, viral maturation inhibitor, distinct capsid assembly modulator, antiviral compounds of distinct or unknown mechanism, and combination thereof.

In another embodiment, the methods provided herein further comprise administering to the individual at least one HBV vaccine, a nucleoside HBV inhibitor, an interferon or any combination thereof.

In an aspect, provided herein is a method of treating an HBV infection in an individual in need thereof, comprising reducing the HBV viral load by administering to the individual a therapeutically effective amount of a compound of Formula (I), or a pharmaceutically acceptable salt thereof, alone or in combination with a reverse transcriptase inhibitor; and further administering to the individual a therapeutically effective amount of HBV vaccine.

An additional embodiment of the present disclosure is a method of treating a subject suffering from an HBV infection, comprising administering to a subject in need of such treatment an effective amount of at least one compound of Formula (I).

In another aspect, provided herein is a method of reducing the viral load associated with an HBV infection in an individual in need thereof, comprising administering to the individual a therapeutically effective amount of a compound of Formula (I), or a pharmaceutically acceptable salt thereof.

In another aspect, provided herein is a method of reducing reoccurrence of an HBV infection in an individual in need thereof, comprising administering to the individual a therapeutically effective amount of a compound of Formula (I), or a pharmaceutically acceptable salt thereof.

In another aspect, provided herein is a method of inhibiting or reducing the formation or presence of HBV DNA-containing particles or HBV RNA-containing particles in an individual in need thereof, comprising administering to the individual a therapeutically effective amount of a compound of Formula (I), or a pharmaceutically acceptable salt thereof.

In another aspect, provided herein is a method of reducing an adverse physiological impact of an HBV infection in an individual in need thereof, comprising administering to the individual a therapeutically effective amount of a compound of Formula (I), or a pharmaceutically acceptable salt thereof.

In another aspect, provided herein is a method of inducing remission of hepatic injury from an HBV infection in an individual in need thereof, comprising administering to the individual a therapeutically effective amount of a compound of Formula (I), or a pharmaceutically acceptable salt thereof.

In another aspect, provided herein is a method of reducing the physiological impact of long-term antiviral therapy for HBV infection in an individual in need thereof, comprising administering to the individual a therapeutically effective amount of a compound of Formula (I), or a pharmaceutically acceptable salt thereof.

In another aspect, provided herein is a method of prophylactically treating an HBV infection in an individual in need thereof, wherein the individual is afflicted with a latent HBV infection, comprising administering to the individual a therapeutically effective amount of a compound of Formula (I), or a pharmaceutically acceptable salt thereof.

In an embodiment, the methods provided herein further comprise monitoring the HBV viral load of the subject, wherein the method is carried out for a period of time such that the HBV virus is undetectable.

The application also relates to a compound of formula (I) or a pharmaceutical composition comprising said compound of formula (I), as disclosed herein, for use as a medicament.

In an aspect, provided herein is a method of treating an HBV infection in an individual in need thereof, comprising reducing the HBV viral load by administering to the individual a therapeutically effective amount of a compound of Formula (Ia), or a pharmaceutically acceptable salt thereof, alone or in combination with a reverse transcriptase inhibitor, and further administering to the individual a therapeutically effective amount of HBV vaccine.

An additional embodiment of the present disclosure is a method of treating a subject suffering from an HBV infection, comprising administering to a subject in need of such treatment an effective amount of at least one compound of Formula (Ia).

In another aspect, provided herein is a method of reducing the viral load associated with an HBV infection in an individual in need thereof, comprising administering to the individual a therapeutically effective amount of a compound of Formula (a), or a pharmaceutically acceptable salt thereof.

In another aspect, provided herein is a method of reducing reoccurrence of an HBV infection in an individual in need thereof, comprising administering to the individual a therapeutically effective amount of a compound of Formula (Ia), or a pharmaceutically acceptable salt thereof.

In another aspect, provided herein is a method of inhibiting or reducing the formation or presence of HBV DNA-containing particles or HBV RNA-containing particles in an individual in need thereof, comprising administering to the individual a therapeutically effective amount of a compound of Formula (a), or a pharmaceutically acceptable salt thereof.

In another aspect, provided herein is a method of reducing an adverse physiological impact of an HBV infection in an individual in need thereof, comprising administering to the individual a therapeutically effective amount of a compound of Formula (Ia), or a pharmaceutically acceptable salt thereof.

In another aspect, provided herein is a method of inducing remission of hepatic injury from an HBV infection in an individual in need thereof, comprising administering to the individual a therapeutically effective amount of a compound of Formula (Ia), or a pharmaceutically acceptable salt thereof.

In another aspect, provided herein is a method of reducing the physiological impact of long-term antiviral therapy for HBV infection in an individual in need thereof, comprising administering to the individual a therapeutically effective amount of a compound of Formula (Ia), or a pharmaceutically acceptable salt thereof.

In another aspect, provided herein is a method of prophylactically treating an HBV infection in an individual in need thereof, wherein the individual is afflicted with a latent HBV infection, comprising administering to the individual a therapeutically effective amount of a compound of Formula (Ia), or a pharmaceutically acceptable salt thereof.

In an embodiment, the methods provided herein further comprise monitoring the HBV viral load of the subject, wherein the method is carried out for a period of time such that the HBV virus is undetectable.

The application also relates to such a compound or pharmaceutically acceptable salt, or to such a pharmaceutical composition, for use in the prevention or treatment of an HBV infection or of an HBV-induced disease in mammal in need thereof.

The application also relates to such a compound or pharmaceutically acceptable salt, or to such a pharmaceutical composition, for use in the prevention, the prevention of aggravation, the amelioration or the treatment of chronic Hepatitis B.

The application relates to such a compound or pharmaceutically acceptable salt, or to such a pharmaceutical composition, for use in the prevention, the prevention of aggravation, the amelioration or the treatment of a HBV-induced disease or condition.

HBV-induced or related disease or condition includes progressive liver fibrosis, inflammation and necrosis leading to cirrhosis, end-stage liver disease, and hepatocellular carcinoma Additionally, HBV acts as a helper virus to hepatitis delta virus (HDV), and it is estimated that more than 15 million people may be HBV/HDV co-infected worldwide, with an increased risk of rapid progression to cirrhosis and increased hepatic decompensation, than patients suffering from HBV alone (Hughes, S A et al. Lancet 2011, 378, 73-85). HDV, infects therefore subjects suffering from HBV infection. In a particular embodiment, the compounds of the invention may be used in the treatment and/or prophylaxis of HBV/HDV co-infection, or diseases associated with HBV/HDV co infection. Therefore, in a particular embodiment, the HBV infection is in particular HBV/HDV co-infection, and the mammal, in particular the human, may be HBV/HDV co-infected, or be at risk of HBV/HDV co infection.

Thus, the application also relates to such a compound or pharmaceutically acceptable salt, or to such a pharmaceutical composition, for any of the above-mentioned uses, more particularly for use in the prevention, the prevention of aggravation, the amelioration, or the treatment of one or more of the following items:
the prevention of chronic hepatis infection, more particularly chronic hepatis B infection (ie, preventing that the hepatitis (B) infection becomes chronic);
the amelioration or treatment of a hepatitis-associated or hepatitis-induced (chronic) disease or condition, more particularly of a hepatitis B-associated or hepatitis B-induced (chronic) disease or condition;
the prevention of the aggravation of a hepatitis-associated or hepatitis-induced (chronic) disease or condition, more particularly of a hepatitis B-associated or hepatitis B-induced (chronic) disease or condition;
the amelioration (regression, or absence of progression) of the stage of liver fibrosis, or of the extent of liver damage, induced by a (chronic) hepatitis infection, more particularly by a (chronic) hepatitis B infection;
the amelioration (reduction) of the fibrosis progression rate of a (chronic) hepatitis infection, more particularly the prevention of cirrhosis in a subject having a (chronic) hepatitis infection, more particularly by a (chronic) hepatitis B infection (e.g., preventing that the subject reaches the cirrhotic stage of fibrosis).

### Combinations

Provided herein are combinations of one or more of the disclosed compounds with at least one additional therapeutic agent. In embodiments, the methods provided herein can further comprise administering to the individual at least one additional therapeutic agent. In embodiments, the disclosed compounds are suitable for use in combination therapy. The compounds of the present disclosure may be useful in combination with one or more additional compounds useful for treating HBV infection. These additional compounds may comprise compounds of the present disclosure or compounds known to treat, prevent, or reduce the symptoms or effects of HBV infection.

In an exemplary embodiment, additional active ingredients are those that are known or discovered to be effective in the treatment of conditions or disorders involved in HBV infection, such as another HBV capsid assembly modulator or a compound active against another target associated with the particular condition or disorder involved in HBV infection, or the HBV infection itself. The combination may serve to increase efficacy (e.g., by including in the combination a compound potentiating the potency or effectiveness of an active agent according to the present disclosure), decrease one or more side effects, or decrease the required dose of the active agent according to the present disclosure. In a further embodiment, the methods provided herein allow for administering of the at least one additional therapeutic agent at a lower dose or frequency as compared to the administering of the at least one additional therapeutic agent alone that is required to achieve similar results in prophylactically treating an HBV infection in an individual in need thereof.

Such compounds include but are not limited to HBV combination drugs, HBV vaccines, HBV DNA polymerase inhibitors, immunomodulatory agents, toll-like receptor (TLR) modulators, interferon alpha receptor ligands, hyaluronidase inhibitors, hepatitis b surface antigen (HBsAg) inhibitors, cytotoxic T-lymphocyte-associated protein 4 (ipi4) inhibitors, cyclophilin inhibitors, HBV viral entry inhibitors, antisense oligonucleotide targeting viral mRNA, short interfering RNAs (siRNA) and ddRNAi endonuclease modulators, ribonucleotide reductase inhibitors, HBV E antigen inhibitors, covalently closed circular DNA (cccDNA) inhibitors, famesoid X receptor agonists, HBV antibodies, CCR2 chemokine antagonists, thymosin agonists, cytokines, nucleoprotein modulators, retinoic acid-inducible gene 1 simulators, NOD2 stimulators, phosphatidylinositol 3-kinase (PI3K) inhibitors, indoleamine-2,3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and any other agent that affects the HBV life cycle and/or affect the consequences of HBV infection or combinations thereof.

In embodiments, the compounds of the present disclosure may be used in combination with an HBV polymerase inhibitor, immunomodulatory agents, interferon such as pegylated interferon, viral entry inhibitor, viral maturation inhibitor, capsid assembly modulator, reverse transcriptase inhibitor, a cyclophilin/TNF inhibitor, immunomodulatory agent such as a TLR-agonist, an HBV vaccine, and any other agent that affects the HBV life cycle and/or affect the consequences of HBV infection or combinations thereof.

In particular, the compounds of the present disclosure may be used in combination with one or more agents (or a salt thereof) selected from the group consisting of

HBV reverse transcriptase inhibitors, and DNA and RNA polymerase inhibitors, including but not limited to: lamivudine (3TC, Zeffix, Heptovir, Epivir, and Epivir-HBV), entecavir (Baraclude, Entavir), adefovir dipivoxil (Hepsara, Preveon, bis-POM PMEA), tenofovir disoproxil fumarate (Viread, TDF or PMPA);

interferons, including but not limited to interferon alpha (IPN-α), interferon beta (IFN-β), interferon lambda (IFN-λ), and interferon gamma (IFN-γ);

viral entry inhibitors;

viral maturation inhibitors;

literature-described capsid assembly modulators, such as, but not limited to BAY 41-4109;

reverse transcriptase inhibitor:

an immunomodulatory agent such as a TLR-agonist; and

agents of distinct or unknown mechanism, such as but not limited to AT-61 ((E)-N-(1-chloro-3-oxo-1-phenyl-3-(piperidin-1-yl)prop-1-en-2-yl)benzamide), AT-130 ((E)-N-(1-bromo-1-(2-methoxyphenyl)-3-oxo-3-(piperidin-1-yl)prop-1-en-2-yl)-4-nitrobenzamide), and similar analogs.

In embodiments, the additional therapeutic agent is an interferon. The term “interferon” or “IFN” refers to any member the family of highly homologous species-specific proteins that inhibit viral replication and cellular proliferation and modulate immune response. Human interferons are grouped into three classes; Type 1, which include interferon-alpha (IFN-α), interferon-beta (IFN-β), and interferon-omega (IFN-ω), Type II, which includes interferon-gamma (IFN-γ), and Type III, which includes interferon-lambda (IFN-λ). Recombinant forms of interferons that have been developed and are commercially available are encompassed by the term “interferon” as used herein. Subtypes of interferons, such as chemically modified or mutated interferons, are also encompassed by the term “interferon” as used herein. Chemically modified interferons include pegylated interferons and glycosylated interferons. Examples of interferons also include, but are not limited to, interferon-alpha-2a, interferon-alpha-2b, interferon-alpha-n1, interferon-beta-1α, interferon-beta-1b, interferon-lamda-1, interferon-lamda-2, and interferon-lamda-3. Examples of pegylated interferons include pegylated interferon-alpha-2a and pegylated interferon alpha-2b.

Accordingly, in one embodiment, the compounds of Formula I, can be administered in combination with an interferon selected from the group consisting of interferon alpha (IFN-α), interferon beta (IFN-β), interferon lambda (IFN-λ), and interferon gamma (IFN-γ). In one specific embodiment, the interferon is interferon-alpha-2a, interferon-alpha-2b, or interferon-alpha-n1. In another specific embodiment, the interferon-alpha-2a or interferon-alpha-2b is pegylated. In a preferred embodiment, the interferon-alpha-2a is pegylated interferon-alpha-2a (PEGASYS).

In another embodiment, the additional therapeutic agent is selected from immune modulator or immune stimulator therapies, which includes biological agents belonging to the interferon class.

Further, the additional therapeutic agent may be an agent that disrupts the function of other essential viral protein(s) or host proteins required for HBV replication or persistence.

In another embodiment, the additional therapeutic agent is an antiviral agent that blocks viral entry or maturation or targets the HBV polymerase such as nucleoside or nucleotide or non-nucleos(t)ide polymerase inhibitors. In a further embodiment of the combination therapy, the reverse transcriptase inhibitor and/or DNA and/or RNA polymerase inhibitor is Zidovudine, Didanosine. Zalcitabine, ddA. Stavudine, Lamivudine. Abacavir. Emtricitabine. Entecavir, Apricitabine, Atevirapine, ribavirin, acyclovir, famciclovir, valacyclovir, ganciclovir, valganciclovir, Tenofovir. Adefovir, PMPA, cidofovir, Efavirenz, Nevirapine, Delavirdine, or Etravirine.

In an embodiment, the additional therapeutic agent is an immunomodulatory agent that induces a natural, limited immune response leading to induction of immune responses against unrelated viruses. In other words, the immunomodulatory agent can affect maturation of antigen presenting cells, proliferation of T-cells and cytokine release (e.g., IL-12, IL-18, IFN-alpha, -beta, and -gamma and TNF-alpha among others).

In a further embodiment, the additional therapeutic agent is a TLR modulator or a TLR agonist, such as a TLR-7 agonist or TLR-9 agonist. In further embodiment of the combination therapy, the TLR-7 agonist is selected from the group consisting of SM360320 (9-benzyl-8-hydroxy-2-(2-methoxy-ethoxy)adenine) and AZD 8848 (methyl [3-({[3-(6-amino-2-butoxy-8-oxo-7,8-dihydro-9H-purin-9-yl)propyl][3-(4-morpholinyl)propyl]amino}methyl)phenyl]acetate).

In any of the methods provided herein, the method may further comprise administering to the individual at least one HBV vaccine, a nucleoside HBV inhibitor, an interferon or any combination thereof. In an embodiment, the HBV vaccine is at least one of RECOMBIVAX HB, ENGERIX-B, ELOVAC B, GENEVAC-B, or SHANVAC B.

In another aspect, provided herein is method of treating an HBV infection in an individual in need thereof, comprising reducing the HBV viral load by administering to the individual a therapeutically effective amount of a compound of the present disclosure alone or in combination with a reverse transcriptase inhibitor; and further administering to the individual a therapeutically effective amount of HBV vaccine. The reverse transcriptase inhibitor may be one of Zidovudine, Didanosine, Zalcitabine, ddA, Stavudine. Lamivudine, Abacavir, Emtricitabine, Entecavir, Apricitabine. Atevirapine, ribavirin, acyclovir, famciclovir, valacyclovir, ganciclovir, valganciclovir, Tenofovir, Adefovir, PMPA, cidofovir, Efavirenz, Nevirapine. Delavirdine, or Etravirine.

For any combination therapy described herein, synergistic effect may be calculated, for example, using suitable methods such as the Sigmoid-Eₘₐₓ equation (Holford & Scheiner, 1981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

Thus, the application also relates to a product comprising a first compound and a second compound as a combined preparation for simultaneous, separate or sequential use in the prevention or treatment of an HBV infection or of an HBV-induced disease in mammal in need thereof, wherein said first compound is different from said second compound, wherein said first compound is the compound or pharmaceutically acceptable salt as herein described, or the pharmaceutical composition of the application, and wherein said second compound is another HBV inhibitor. For example, a second compound is another HBV inhibitor which is selected from the group consisting HBV combination drugs, HBV DNA polymerase inhibitors, immunomodulators, toll-like (TLR) receptor modulators, interferon alpha receptor ligands, hyaluronidase inhibitors, hepatitis b surface antigen (HbsAg) inhibitors, cytotoxic T-lymphocyte-associated protein 4 (ipi4) inhibitors, cyclohilin inhibitors, HBV viral entry inhibitors, antisense oligonucleotide targeting viral mRNA, short interfering RNAs (siRNA) and ddRNAi endonuclease modulators, ribonucleotide reductase inhibitors. HBV E antigen inhibitors, covalently closed circular DNA (cccDNA) inhibitors, famsoid X receptor agonists. HBV antibodies, CCR2 chemokine antagonists, thymosin agonists, cytokines, nucleoprotein modulators, retinoic acid-inducible gene 1 stimulators, NOD2 stimulators, phosphatidylinositol 3-kinase (PI3K) inhibitors, indole amine 2,3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and other HBV drugs.

### Methods

The application relates to a method for the preparation of a compound of Formula (I) as described herein.

In embodiments, the method comprises at least one step from among steps a), b), c), d), e), f), g), h), i), j), k), l), m), n), o), p), q), r) and s):

a) reacting a compound of Formula (II),

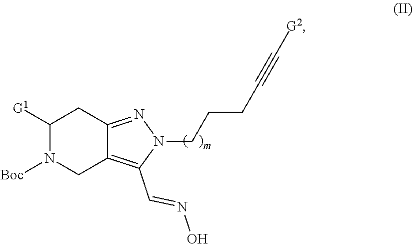

with NaOCl to form a compound of Formula (III),

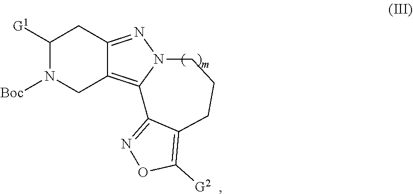

wherein

m is an integer of 0 or 1:

G¹ is H or CH₃;

G² is H, C₁₋₄alkyl, CF or phenyl;

with the proviso that when m is 1, G¹ and G² are not both H;
b) reacting a compound of Formula (III),

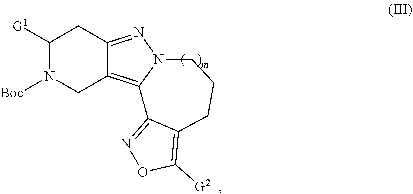

with a strong acid, such as hydrochloric acid (HCl), or TFA to form a compound of formula (IV),

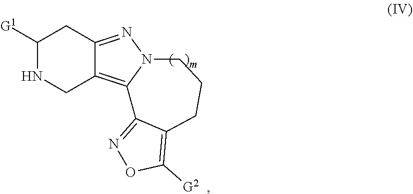

wherein

m is an integer of 0 or 1;

G¹ is H or CH₂;

G² is H, C₁₋₄alkyl, CF₃ or phenyl;

c) reacting a compound of Formula (IV),

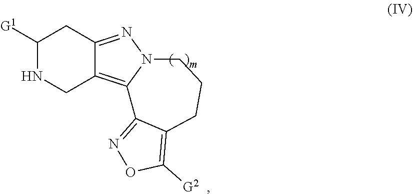

with a compound of formula (V),

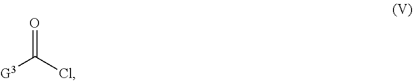

in the presence of non-nucleophilic base, such as triethylamine (Et₃N) or sodium carbonate (Na₂CO₃), to form a compound of formula (VI),

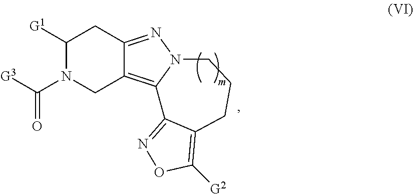

wherein

m is an integer of 0 or 1;

G¹ is H or CH₃:

G2 is H, C₁₋₄alkyl, CF₃ or phenyl;

G³ is phenyl substituted with one or more substituents selected from the group consisting of Cl, F, CF₃, CF₂H, CN, and C₁₋₄alkyl; more particularly, G³ is 3,4-dichlorophenyl;

d) reacting of compound of formula (VII),

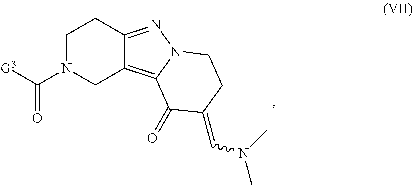

with a compound of formula (VIII),

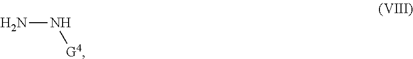

to form a compound of Formula (IX),

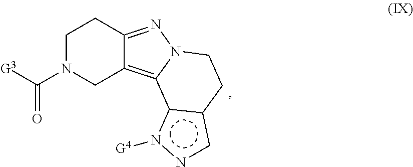

wherein

[symbol] represents a single or a double bond;

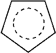

is an aromatic ring;

G³ is phenyl substituted with one or more substituents selected from the group consisting of Cl, F, CF₃, CF₂H, CN, and C₁₋₄alkyl; more particularly, G³ is 3,4-dichlorophenyl;

G⁴ is H or CH₃;

e) reacting a compound of Formula (X),

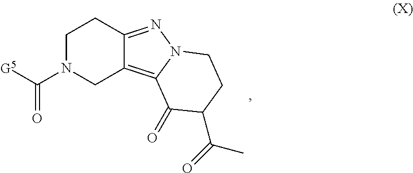

with hydrazine, to form a compound of Formula (XI),

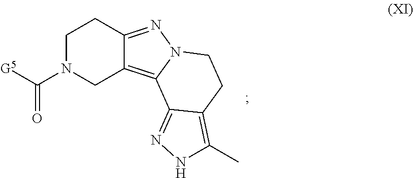

wherein G⁵ is phenyl substituted with one or more substituents selected from the group consisting of Cl, F, CF₃, CF₂H, CN, and C₁₋₄alkyl; more particularly, G³ is 3,4-dichlorophenyl;

f) reacting a compound of Formula (XXV),

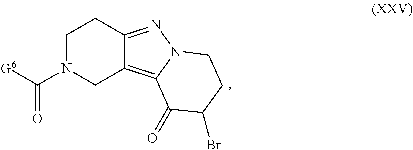

with thioacetamide, to form a compound of Formula (XXVI),

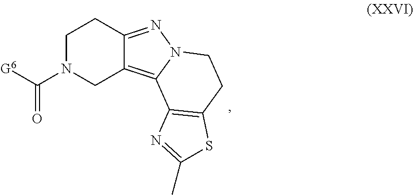

wherein G⁶ is phenyl substituted with one or more substituents selected from the group consisting of Cl, F, CF₃, CF₂H, CN, and C₁₋₄alkyl;

g) reacting a compound of Formula (XII),

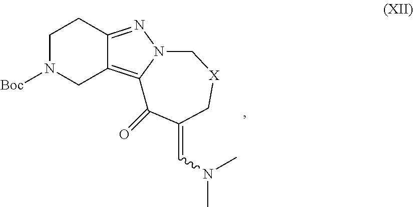

with a compound of Formula (XIII),

H₂N-G⁷ (XIII),

to form a compound of Formula (XIV),

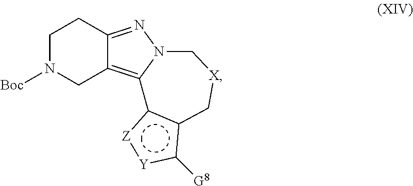

wherein

[symbol] represents a single or a double bond:

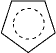

is an aromatic ring:

X is CH₂ or C═CH₂;

G⁷ is OH, NH₂ or NH(CH₃);

G⁸ is H or NH₂;

with the proviso that when G⁷ is NH₂ or NH(CH₃), then G⁸ is H; or when G⁷ is OH, then G⁸ is H or NH₂;

Y is O, NH, N or N(CH₃);

Z is N or O;

h) reacting a compound of Formula (XV).

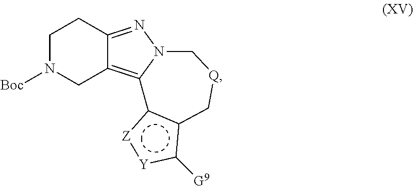

with a strong acid, such as hydrochloric acid (HCl) or TFA (trifluoroacetic acid), to form a compound of Formula (XVI),

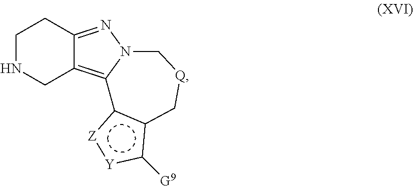

wherein

[symbol] represents a single or a double bond;

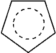

is an aromatic ring:

Q is C═CH₂ or CG¹⁰G¹¹;

G9 is H or NH₂;

G¹⁰ and G¹¹ are independently selected from H, OH, CONHMe, CH₂OH and CONH₂;

Y is O, N, NH or N(CH₃);

Z is N or O;

in embodiments, when G⁶ is NH₂, then Q is C═CH₂, Y is O, and Z is N;

i) reacting a compound of Formula (XVI),

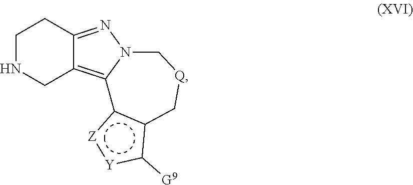

with a compound of Formula (XVII),

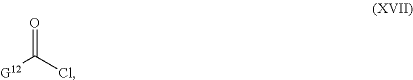

in the presence of non-nucleophilic base, such as triethylamine (Et₃N) or sodium carbonate (Na₂CO₃), to form a compound of Formula (XVIII),

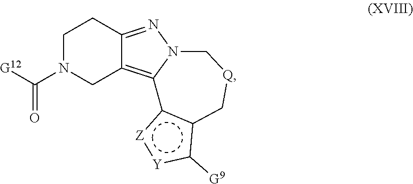

wherein

[symbol] represents a single or a double bond;

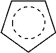

is an aromatic ring;

Q is C═CH₂ or CG¹⁰G¹¹;

G⁹ is H or NH₂;

G¹⁰ and G¹¹ are independently selected from H, OH, CONHMe, CH₂OH and CONH₂;

G¹² is phenyl substituted with one or more substituents selected from the group consisting of Cl, F, CF₃, CF₂H, CN, and C₁₋₄alkyl; more particularly, G¹² is 3,4-dichlorophenyl;

Y is O, N, NH or N(CH₃);

Z is N or O;

in embodiments, when G⁶ is NH₂, then Q is C═CH₂, Y is O, and Z is N;

j) reacting a compound of Formula (XIX),

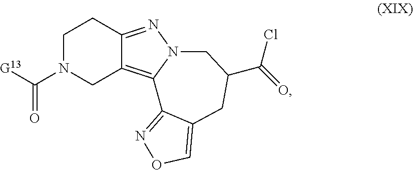

with a compound of Formula (XX),

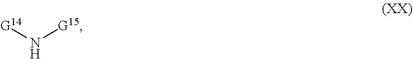

to form a compound of Formula (XXI),

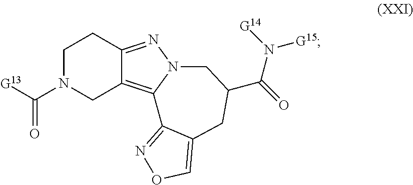

wherein

G¹³ is phenyl substituted with one or more substituents selected from the group consisting of Cl, F, CF₃, CF₂H, CN, and C₁₋₄alkyl; more particularly, G² is 3,4-dichlorophenyl;

G¹⁴ and G¹⁵ are independently selected from H, C₁₋₄alkyl, cyclopropyl, CHCH₂OH, CH₂CF₃ and phenyl; more particularly, one of G¹⁴ and G¹⁵ is H; more particularly, when none of G¹⁴ and G¹⁵ is H, then G¹⁴ is CH₃ and G¹⁵ is CH₃;

or G¹⁴ and G¹⁵ are connected together to form a morpholine ring:

k) reacting a compound of Formula (XXVII),

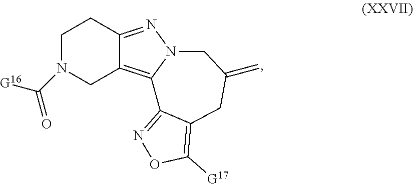

with potassium osmate (K₂OsO₄), in the presence of 4-Methylmorpholine N-oxide (NMO), to form a compound of Formula (XXVIII),

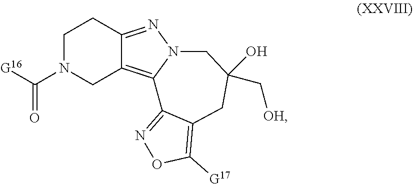

wherein
G¹⁷ is H or NH₂;

G¹⁶ is O-tert-butyl or phenyl substituted with one or more substituents selected from the group consisting of Cl, F, CF₃, CF₂H, CN, and C₁₋₄alkyl; more particularly, G¹⁶ is O^{t}Bu or 3,4-dichlorophenyl;

l) reacting a compound of Formula (XXIX),

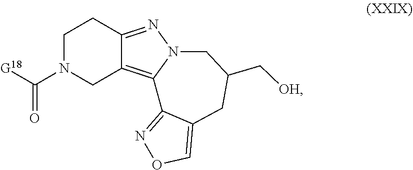

with an oxidizing agent, such as tetrapropylammonium perruthenate (TPAP) in the presence of 4-Methylmorpholine N-oxide (NMO), to form a compound of Formula (XXX);

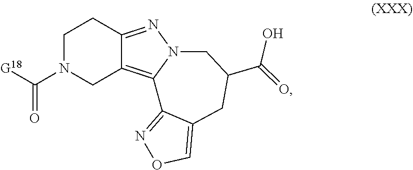

wherein G¹⁸ is O-tert-butyl or phenyl substituted with one or more substituents selected from the group consisting of Cl, F, CF₃, CF₂H, CN, and C₁₋₄alkyl; more particularly, G¹⁸ is O^{t}Bu or 3,4-dichlorophenyl;

m) reacting a compound of Formula (XXXI).

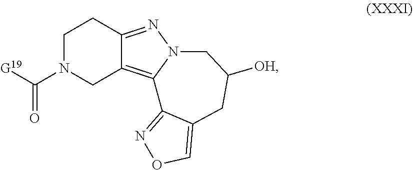

with a fluorinating reagent, such as (diethylamine)sulfur trifluoride (DAST), to form a compound of Formula (XXXII).

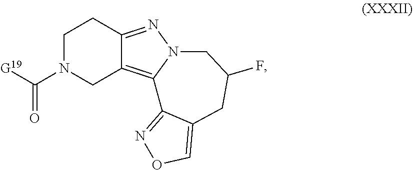

wherein G¹⁹ is phenyl substituted with one or more substituents selected from the group consisting of Cl, F, CF₃, CF₂H, CN, and C₁₋₄alkyl; more particularly, G¹⁹ is 3,4-dichlorophenyl;

n) reacting a compound of Formula (XXXIII),

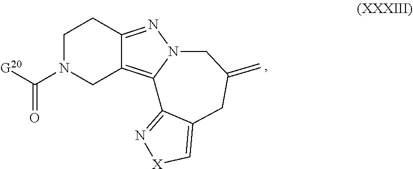

with hydrogen peroxide, in the presence of 9-BBN and sodium hydroxide, to form a compound of Formula (XXXIV),

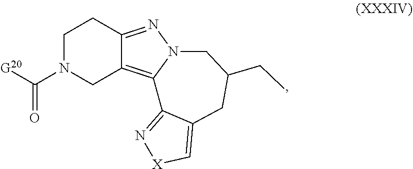

wherein

G²⁰ is O-tert-butyl or phenyl substituted with one or more substituents selected from the group consisting of Cl, F, CF₃, CF₂H, CN, and C₁₋₄alkyl; more particularly, G²⁰ is O^{t}Bu or 3,4-dichlorophenyl:

X is NH or O:

o) reacting a compound of Formula (XXXV),

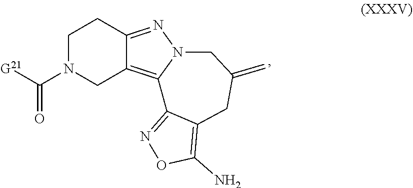

with a methylating agent, in the presence of a non-nucleophilic base, to form a compound of Formula (XXXVI),

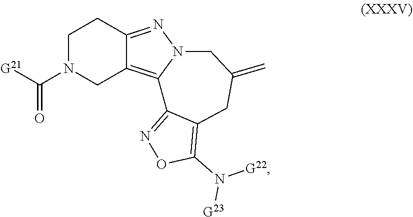

wherein

G²¹ is O-tert-butyl or phenyl substituted with one or more substituents selected from the group consisting of Cl, F, CF₃, CF₂H, CN, and C₁₋₄alkyl; more particularly, G²¹ is 3,4-dichlorophenyl;

G²² and G²³ are independently selected from H and CH₃, with the proviso that at least one of G²² and G²³ is CH₃;

in embodiments, the methylating agent is Mel and the base is NaH; in embodiments, the methylating agent is paraformaldehyde, and the base is NaOMe, then NaBH₄:

p) reacting a compound of Formula (XXXVII),

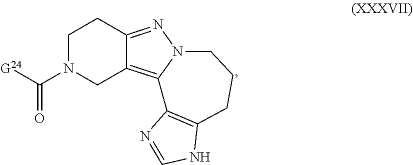

with a methylating agent, such as methyl iodide, in the presence of a non-nucleophilic base, such as sodium hydride, to form a compound of Formula (XXXV III),

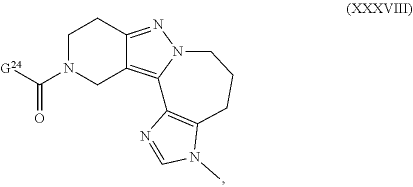

wherein G²⁴ is O-tert-butyl or phenyl substituted with one or more substituents selected from the group consisting of Cl, F, CF₃, CF₂H, CN, and C₁₋₄alkyl; more particularly, G²⁴ is 3,4-dichlorophenyl;

q) reacting a compound of Formula (XXXIX),

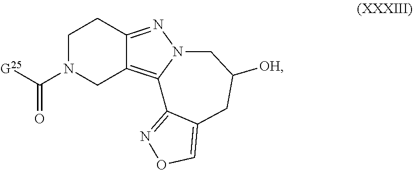

with a methylating agent, such as methyl iodide, in the presence of a non-nucleophilic base, such as sodium hydride, to form a compound of Formula (XL),

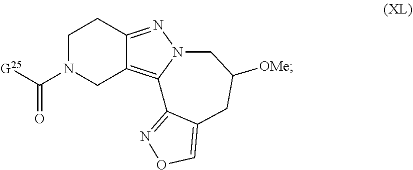

wherein G²⁵ is O-tert-butyl or phenyl substituted with one or more substituents selected from the group consisting of Cl, F, CF₃, CF₂H, CN, and C₁₋₄alkyl; more particularly, G²⁵ is 3,4-dichlorophenyl;

r) reacting a compound of Formula (XXII),

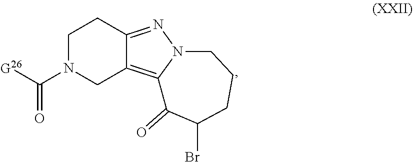

with a compound of Formula (XXIII),

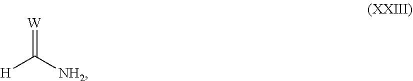

to form a compound of Formula (XXIV),

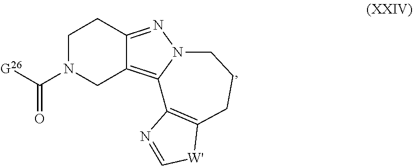

wherein

G²⁶ is phenyl substituted with one or more substituents selected from the group consisting of Cl, F, CF, CF₂H, CN, and C₁₋₄alkyl; more particularly, G²⁶ is 3,4-dichlorophenyl;

W is O or S;

W′ is O, NH, S;

s) reacting a compound of Formula (XLI),

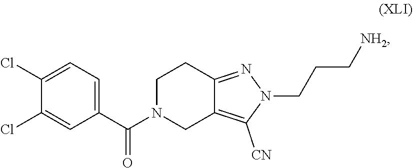 [SMILES: N#Cc1c2c(nn1CCCN)CCN(C(=O)c1ccc(Cl)c(Cl)c1)C2]

with magnesium ethoxide and chloroacetaldehyde, to form a compound of Formula (XLII),

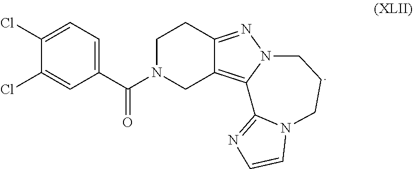 [SMILES: O=C(c1ccc(Cl)c(Cl)c1)N1CCc2nn3c(c2C1)-c1nccn1CCC3]

In embodiments, the process may comprise steps a), b), and c).

In embodiments, the process may comprise steps g), h) and i).

In embodiments, the process may comprise steps g), h), i) and may further comprise step k).

In embodiments, the process may comprise steps g), h), i) and k).

In embodiments, the process may comprise steps g), h), i), k) and further comprise step q).

In embodiments, the process may comprise steps g), h), i), k) and further comprise step m).

In embodiments, the process may comprise steps g), h), i) and further comprise step o).

In embodiments, the process may comprise steps g), h), i) and further comprise step n).

In embodiments, the process may comprise steps g), h), i) and further comprise step l) and n).

In embodiments, the process may comprise steps g), h), i), l), n) and further comprise step j).

In embodiments, the process may comprise steps r) and p).

### Definitions

Listed below are definitions of various terms used to describe this present disclosure. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the applicable art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e. to at least one) of the grammatical object of the article. By way of example, “an element” means one element or more than one element. Furthermore, use of the term “including” as well as other forms, such as “include,” “includes,” and “included,” is not limiting.

As used in the specification and in the claims, the term “comprising” can include the embodiments “consisting of” and “consisting essentially of.” The terms “comprise(s).” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically acceptable carriers, and excludes other compounds.

All ranges disclosed herein are inclusive of the recited endpoint and independently combinable (for example, the range of “from 50 mg to 3M) mg” is inclusive of the endpoints, 50 mg and 300 mg, and all the intermediate values). The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “substantially,” cannot be limited to the precise value specified, in some cases. In at least some instances, the approximating language can correspond to the precision of an instrument for measuring the value.

The term “alkyl” refers to a straight- or branched-chain alkyl group having from 1 to 12 carbon atoms in the chain. Examples of alkyl groups include methyl (Me, which also may be structurally depicted by the symbol, “/”), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. The term C₁₋₄alkyl as used here refers to a straight- or branched-chain alkyl group having from 1 to 4 carbon atoms in the chain. The term C₁₋₆alkyl as used here refers to a straight- or branched-chain alkyl group having from 1 to 6 carbon atoms in the chain.

The term “cycloalkyl” refers to a saturated or partially saturated, monocyclic, fused polycyclic, or spiro polycyclic carbocycle having from 3 to 12 ring atoms per carbocycle. Illustrative examples of cycloalkyl groups include the following entities, in the form of properly bonded moieties:

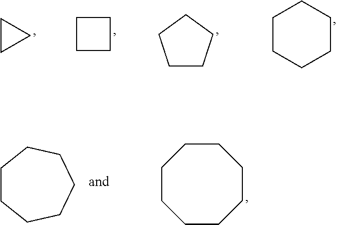

A monocyclic, bicyclic or tricyclic aromatic carbocycle represents an aromatic ring system consisting of 1, 2 or 3 rings, said ring system being composed of only carbon atoms; the term aromatic is well known to a person skilled in the art and designates cyclically conjugated systems of 4n+2 electrons, that is with 6, 10, 14 etc. π-electrons (rule of Hückel).

Particular examples of monocyclic, bicyclic or tricyclic aromatic carbocycles are phenyl, naphthalenyl, anthracenyl.

The term “phenyl” represents the following moiety:

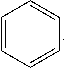

The term “heteroaryl” refers to an aromatic monocyclic or bicyclic aromatic ring system having 5 to 10 ring members and which contains carbon atoms and from 1 to 4 heteroatoms independently selected from the group consisting of N, O, and S. Included within the term heteroaryl are aromatic rings of 5 or 6 members wherein the ring consists of carbon atoms and has at least one heteroatom member. Suitable heteroatoms include nitrogen, oxygen, and sulfur. In the case of 5 membered rings, the heteroaryl ring preferably contains one member of nitrogen, oxygen or sulfur and, in addition, up to 3 additional nitrogens. In the case of 6 membered rings, the heteroaryl ring preferably contains from 1 to 3 nitrogen atoms. For the case wherein the 6 membered ring has 3 nitrogens, at most 2 nitrogen atoms are adjacent. Examples of heteroaryl groups include furyl, thienyl, pyrrolyl, oxazolyl, thiazolyl, imidazolyl, pyrazolyl, oxazolyl, thiazolyl, oxadiazolyl, triazolyl, thiadiazolyl, pyridinyl, pyridazinyl, pyrimidinyl, pyrazinyl, indolyl, isoindolyl, benzofuryl, benzothienyl, indazolyl, benzimidazolyl, benzothiazolyl, benzoxazolyl, benzisoxazolyl, benzothiadiazolyl, benzotriazolyl, quinolinyl, isoquinolinyl and quinazolinyl. Unless otherwise noted, the heteroaryl is attached to its pendant group at any heteroatom or carbon atom that results in a stable structure.

Those skilled in the art will recognize that the species of heteroaryl groups listed or illustrated above are not exhaustive, and that additional species within the scope of these defined terms may also be selected.

The term “cyano” refers to the group —CN.

The terms “halo” or “halogen” represent chloro, fluoro, bromo or iodo.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In cases where a specified moiety or group is not expressly noted as being optionally substituted or substituted with any specified substituent, it is understood that such a moiety or group is intended to be unsubstituted.

The terms “para”, “meta”, and “ortho” have the meanings as understood in the art. Thus, for example, a fully substituted phenyl group has substituents at both “ortho” (o) positions adjacent to the point of attachment of the phenyl ring, both “meta” (m) positions, and the one “para” (p) position across from the point of attachment. To further clarify the position of substituents on the phenyl ring, the 2 different ortho positions will be designated as ortho and ortho′ and the 2 different meta positions as meta and meta′ as illustrated below.

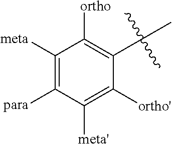

When referring to substituents on a pyridyl group, the terms “para”, “meta”, and “ortho” refer to the placement of a substituent relative to the point of attachment of the pyridyl ring. For example, the structure below is described as 3-pyridyl with the X¹ substituent in the ortho position, the X² substituent in the meta position, and X³ substituent in the para position:

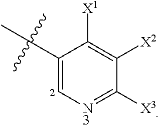

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

The terms “buffered” solution or “buffer” solution are used herein interchangeably according to their standard meaning. Buffered solutions are used to control the pH of a medium, and their choice, use, and function is known to those of ordinary skill in the art. See, for example, G. D. Considine, ed., Van Nostrand's Encyclopedia of Chemistry, p. 261, 5^{th} ed. (2005), describing, inter alia, buffer solutions and how the concentrations of the buffer constituents relate to the pH of the buffer. For example, a buffered solution is obtained by adding MgSO₄ and NaHCO₃ to a solution in a 10:1 w/w ratio to maintain the pH of the solution at about 7.5.

Any formula given herein is intended to represent compounds having structures depicted by the structural formula as well as certain variations or forms. In particular, compounds of any formula given herein may have asymmetric centers and therefore exist in different enantiomeric forms. All optical isomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and rans isomers), as tautomers, or as atropisomers.

It is also to be understood that compounds that have the same molecular formula but differ in the nature or sequence of bonding of their atoms or the arrangement of their atoms in space are termed “isomers.”

Stereoisomers that are not mirror images of one another are termed “diastereomers” and those that are non-superimposable mirror images of each other are termed “enantiomers.” When a compound has an asymmetric center, for example, it is bonded to four different groups, and a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either an individual enantiomer or as a mixture thereof. A mixture containing equal proportions of the enantiomers is called a “racemic mixture.”

“Tautomers” refer to compounds that are interchangeable forms of a particular compound structure, and that vary in the displacement of hydrogen atoms and electrons. Thus, two structures may be in equilibrium through the movement of x electrons and an atom (usually H). For example, enols and ketones are tautomers because they are rapidly interconverted by treatment with either acid or base. Another example of tautomerism is the aci-and nitro-forms of phenyl nitromethane, that are likewise formed by treatment with acid or base.

Tautomeric forms may be relevant to the attainment of the optimal chemical reactivity and biological activity of a compound of interest.

The compounds of this present disclosure may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

Certain examples contain chemical structures that are depicted as an absolute enantiomer but are intended to indicate enantiopure material that is of unknown configuration. In these cases (R*) or (S*) or (*R) or (*S) is used in the name to indicate that the absolute stereochemistry of the corresponding stereocenter is unknown. Thus, a compound designated as (R*) or (*R) refers to an enantiopure compound with an absolute configuration of either (R) or (S). In cases where the absolute stereochemistry has been confirmed, the structures are named using (R) and (S), wherein the absolute configuration is specified according to the Cahn-Ingold-Prelog system.

The symbols [symbol] and [symbol] are used as meaning the same spatial arrangement in chemical structures shown herein. Analogously, the symbols [symbol] and [symbol] are used as meaning the same spatial arrangement in chemical structures shown herein.

Additionally, any formula given herein is intended to refer also to hydrates, solvates, and polymorphs of such compounds, and mixtures thereof, even if such forms are not listed explicitly. Certain compounds of Formula (I), or pharmaceutically acceptable salts of compounds of Formula (I), may be obtained as solvates. Solvates include those formed from the interaction or complexation of compounds of the present disclosure with one or more solvents, either in solution or as a solid or crystalline form. In some embodiments, the solvent is water and the solvates are hydrates. In addition, certain crystalline forms of compounds of Formula (I), or pharmaceutically acceptable salts of compounds of Formula (I) may be obtained as co-crystals. In certain embodiments of the present disclosure, compounds of Formula (I) were obtained in a crystalline form. In other embodiments, crystalline forms of compounds of Formula (I) were cubic in nature. In other embodiments, pharmaceutically acceptable salts of compounds of Formula (I) were obtained in a crystalline form. In still other embodiments, compounds of Formula (I) were obtained in one of several polymorphic forms, as a mixture of crystalline forms, as a polymorphic form, or as an amorphous form. In other embodiments, compounds of Formula (I) convert in solution between one or more crystalline forms and/or polymorphic forms.

Reference to a compound herein stands for a reference to any one of: (a) the actually recited form of such compound, and (b) any of the forms of such compound in the medium in which the compound is being considered when named. For example, reference herein to a compound such as R—COOH, encompasses reference to any one of, for example, R—COOH₍ₛ₎, R—COOH₍ₛₒₗ₎, and R—COO⁻₍ₛₒₗ₎. In this example, R—COOH, refers to the solid compound, as it could be for example in a tablet or some other solid pharmaceutical composition or preparation; R—COOH₍ₛₒₗ₎ refers to the undissociated form of the compound in a solvent; and R—COO⁻₍ₛₒₗ₎ refers to the dissociated form of the compound in a solvent, such as the dissociated form of the compound in an aqueous environment, whether such dissociated form derives from R—COOH, from a salt thereof, or from any other entity that yields R—COO⁻ upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In still another example, an expression such as “reacting an entity with a compound of formula R—COOH” refers to the reacting of (a) such entity in the chemically relevant form, or forms, of such entity that exists, or exist, in the medium in which such reacting takes place, with (b) the chemically relevant form, or forms, of the compound R—COOH that exists, or exist, in the medium in which such reacting takes place. In this regard, if such entity is for example in an aqueous environment, it is understood that the compound R—COOH is in such same medium, and therefore the entity is being exposed to species such as R—COOH_{(aq)} and/or R—COO⁻_{(aq)}, where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation, and deprotonation. No further examples in this regard are provided herein because these interactions and transformations in a given medium are known by any one of ordinary skill in the art.

In another example, a zwitterionic compound is encompassed herein by referring to a compound that is known to form a zwitterion, even if it is not explicitly named in its zwitterionic form. Terms such as zwitterion, zwitterions, and their synonyms zwitterionic compound(s) are standard IUPAC-endorsed names that are well known and part of standard sets of defined scientific names. In this regard, the name zwitterion is assigned the name identification CHEBI:27369 by the Chemical Entities of Biological Interest (ChEBI) dictionary of molecular entities. As generally well known, a zwitterion or zwitterionic compound is a neutral compound that has formal unit charges of opposite sign. Sometimes these compounds are referred to by the term “inner salts”. Other sources refer to these compounds as “dipolar ions”, although the latter term is regarded by still other sources as a misnomer. As a specific example, aminoethanoic acid (the amino acid glycine) has the formula H₂NCH₂COOH, and it exists in some media (in this case in neutral media) in the form of the zwitterion ⁺H₃NCH₂COO⁻. Zwitterions, zwitterionic compounds, inner salts and dipolar ions in the known and well established meanings of these terms are within the scope of this present disclosure, as would in any case be so appreciated by those of ordinary skill in the art. Because there is no need to name each and every embodiment that would be recognized by those of ordinary skill in the art, no structures of the zwitterionic compounds that are associated with the compounds of this present disclosure are given explicitly herein. They are, however, part of the embodiments of this present disclosure. No further examples in this regard are provided herein because the interactions and transformations in a given medium that lead to the various forms of a given compound are known by any one of ordinary skill in the art.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the present disclosure include isotopes of hydrogen, carbon, nitrogen, oxygen, phosphorus, sulfur, fluorine, chlorine, and iodine such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, ¹²⁵I, respectively. Such isotopically labeled compounds are useful in metabolic studies (preferably with ¹⁴C), reaction kinetic studies (with, for example deuterium (i.e., D or ²H); or tritium (i.e., T or ³H)), detection or imaging techniques such as positron emission tomography (PET) or single-photon emission computed tomography (SPECT) including drug or substrate tissue distribution assays, or in radioactive treatment of patients. In particular, an 18F or 11C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this present disclosure and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

According to the foregoing interpretive considerations on assignments and nomenclature, it is understood that explicit reference herein to a set implies, where chemically meaningful and unless indicated otherwise, independent reference to embodiments of such set, and reference to each and every one of the possible embodiments of subsets of the set referred to explicitly.

By way of a first example on substituent terminology, if substituent S¹ₑₓₐₘₚₗₑ is one of S₁ and S₂, and substituent S²ₑₓₐₘₚₗₑ is one of S₃ and S₄, then these assignments refer to embodiments of this present disclosure given according to the choices S¹ₑₓₐₘₚₗₑ is S₁ and S²ₑₓₐₘₚₗₑ is S₃; S¹ₑₓₐₘₚₗₑ is S₁ and S²ₑₓₐₘₚₗₑ is S₄; S¹ₑₓₐₘₚₗₑ is S₂ and S²ₑₓₐₘₚₗₑ is S₃; S¹ₑₓₐₘₚₗₑ is S₂ and S²ₑₓₐₘₚₗₑ is S₄; and equivalents of each one of such choices. The shorter terminology “S¹ₑₓₐₘₚₗₑ is one of S₁ and S₂, and S²ₑₓₐₘₚₗₑ is one of S₃ and S₄” is accordingly used herein for the sake of brevity, but not by way of limitation. The foregoing first example on substituent terminology, which is stated in generic terms, is meant to illustrate the various substituent assignments described herein. The foregoing convention given herein for substituents extends, when applicable, to members such as R¹, R², R³, R⁴, R⁵, G¹, G², G³, G⁴, G⁵, G⁶, G⁷, G⁸, G⁹, G¹⁰, G¹¹, n, L, R, T, Q, W, X, Y, and Z and any other generic substituent symbol used herein.

Furthermore, when more than one assignment is given for any member or substituent, embodiments of this present disclosure comprise the various groupings that can be made from the listed assignments, taken independently, and equivalents thereof. By way of a second example on substituent terminology, if it is herein described that substituent Sₑₓₐₘₚₗₑ is one of S₁, S₂, and S₃, this listing refers to embodiments of this present disclosure for which Sₑₓₐₘₚₗₑ is S₁; Sₑₓₐₘₚₗₑ is S₂; Sₑₓₐₘₚₗₑ is S₃; Sₑₓₐₘₚₗₑ is one of S₁ and S₂; Sₑₓₐₘₚₗₑ is one of S₁ and S₃; Sₑₓₐₘₚₗₑ is one of S₂ and S₃; Sₑₓₐₘₚₗₑ is one of S₁, S₂ and S₃; and Sₑₓₐₘₚₗₑ is any equivalent of each one of these choices. The shorter terminology “Sₑₓₐₘₚₗₑ is one of S₁, S₂, and S₃” is accordingly used herein for the sake of brevity, but not by way of limitation. The foregoing second example on substituent terminology, which is stated in generic terms, is meant to illustrate the various substituent assignments described herein. The foregoing convention given herein for substituents extends, when applicable, to members such as R¹, R², R³, R⁴, R⁵, G¹, G², G³, G⁴, G⁵, G⁶, G⁷, G⁸, G⁹, G¹⁰, G¹¹, n, L, R, T, Q, W, X, Y, and Z and any other generic substituent symbol used herein.

The nomenclature “Cᵢ₋ⱼ” with j>1, when applied herein to a class of substituents, is meant to refer to embodiments of this present disclosure for which each and every one of the number of carbon members, from i to j including i and j, is independently realized. By way of example, the term C₁₋₄ refers independently to embodiments that have one carbon member (C₁), embodiments that have two carbon members (C₂), embodiments that have three carbon members (C₃), and embodiments that have four carbon members (C₄).

The term Cₙ₋ₘalkyl refers to an aliphatic chain, whether straight or branched, with a total number N of carbon members in the chain that satisfies n≤N≤m, with m>n. Any disubstituent referred to herein is meant to encompass the various attachment possibilities when more than one of such possibilities are allowed. For example, reference to disubstituent -A-B-, where A≠B, refers herein to such disubstituent with A attached to a first substituted member and B attached to a second substituted member, and it also refers to such disubstituent with A attached to the second substituted member and B attached to the first substituted member.

The present disclosure includes also pharmaceutically acceptable salts of the compounds of Formula (I), preferably of those described above and of the specific compounds exemplified herein, and methods of treatment using such salts.

The term “pharmaceutically acceptable” means approved or approvable by a regulatory agency of Federal or a state government or the corresponding agency in countries other than the United States, or that is listed in the U. S. Pharmacopoeia or other generally recognized pharmacopoeia for use in animals, and more particularly, in humans.

A “pharmaceutically acceptable salt” is intended to mean a salt of a free acid or base of compounds represented by Formula (I) and Formula (Ia) that are non-toxic, biologically tolerable, or otherwise biologically suitable for administration to the subject. It should possess the desired pharmacological activity of the parent compound. See, generally, G. S. Paulekuhn, et al., “Trends in Active Pharmaceutical Ingredient Salt Selection based on Analysis of the Orange Book Database”, J. Med. Chem., 2007, 50:6665-72, S. M. Berge, et al., “Pharmaceutical Salts”, J Pharm Sci., 1977, 66:1-19, and Handbook of Pharmaceutical Salts, Properties, Selection, and Use. Stahl and Wermuth, Eds., Wiley-VCH and VHCA. Zurich, 2002. Examples of pharmaceutically acceptable salts are those that are pharmacologically effective and suitable for contact with the tissues of patients without undue toxicity, irritation, or allergic response. A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt.

The present disclosure also relates to pharmaceutically acceptable prodrugs of the compounds of Formula (I) and Formula (Ia), and treatment methods employing such pharmaceutically acceptable prodrugs. The term “prodrug” means a precursor of a designated compound that, following administration to a subject, yields the compound m vivo via a chemical or physiological process such as solvolysis or enzymatic cleavage, or under physiological conditions (e.g., a prodrug on being brought to physiological pH is converted to the compound of Formula (I) or Formula (Ia)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

The present disclosure also relates to pharmaceutically active metabolites of the compounds of Formula (I) and Formula (Ia), which may also be used in the methods of the present disclosure. A “pharmaceutically active metabolite” means a pharmacologically active product of metabolism in the body of a compound of Formula (I) or salt thereof or a compound of Formula (Ia) or salt thereof. Prodrugs and active metabolites of a compound may be determined using routine techniques known or available in the art. See, e.g., Bertolini, et al., J Med Chem, 1997, 40, 2011-2016; Shan, et al., J Pharm Sci. 1997, 86 (7), 765-767; Bagshawe, Drug Dev Res. 1995, 34, 220-230; Bodor, Adv Drug Res. 1984, 13, 224-331; Bundgaard, Design of Prodrugs (Elsevier Press, 1985); and Larsen, Design and Application of Prodrugs. Drug Design and Development (Krogsgaard-Larsen, et al., eds., Harwood Academic Publishers. 1991).

As used herein, the term “composition” or “pharmaceutical composition” refers to a mixture of at least one compound provided herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound provided herein within or to the patient such that it can perform its intended function. Typically, such constructs are carried or transported from one organ, or portion of the body, to another organ, or portion of the body. Each carrier must be “acceptable” in the sense of being compatible with the other ingredients of the formulation, including the compound provided herein, and not injurious to the patient. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein. “pharmaceutically acceptable carrier” also includes any and all coatings, antibacterial and antifungal agents, and absorption delaying agents, and the like that are compatible with the activity of the compound provided herein, and are physiologically acceptable to the patient. Supplementary active compounds can also be incorporated into the compositions. The “pharmaceutically acceptable carrier” can further include a pharmaceutically acceptable salt of the compound provided herein. Other additional ingredients that can be included in the pharmaceutical compositions provided herein are known in the art and described, for example in Remington's Pharmaceutical Sciences (Genaro, Ed., Mack Publishing Co., 1985, Easton, Pa.), which is incorporated herein by reference.

The term “stabilizer,” as used herein, refers to polymers capable of chemically inhibiting or preventing degradation of a compound of Formula I. Stabilizers are added to formulations of compounds to improve chemical and physical stability of the compound.

The term “tablet,” as used herein, denotes an orally administrable, single-dose, solid dosage form that can be produced by compressing a drug substance or a pharmaceutically acceptable salt thereof, with suitable excipients (e.g., fillers, disintegrants, lubricants, glidants, and/or surfactants) by conventional tableting processes. The tablet can be produced using conventional granulation methods, for example, wet or dry granulation, with optional comminution of the granules with subsequent compression and optional coating. The tablet can also be produced by spray-drying.

As used herein, the term “capsule” refers to a solid dosage form in which the drug is enclosed within either a hard or soft soluble container or “shell.” The container or shell can be formed from gelatin, starch and/or other suitable substances.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

The term “combination.” “therapeutic combination,” “pharmaceutical combination,” or “combination product” as used herein refer to a non-fixed combination or a kit of parts for the combined administration where two or more therapeutic agents can be administered independently, at the same time or separately within time intervals, especially where these time intervals allow that the combination partners show a cooperative. e.g., synergistic, effect.

The term “modulators” include both inhibitors and activators, where “inhibitors” refer to compounds that decrease, prevent, inactivate, desensitize, or down-regulate HBV assembly and other HBV core protein functions necessary for HBV replication or the generation of infectious particles.

As used herein, the term “capsid assembly modulator” refers to a compound that disrupts or accelerates or inhibits or hinders or delays or reduces or modifies normal capsid assembly (e.g., during maturation) or normal capsid disassembly (e.g., during infectivity) or perturbs capsid stability, thereby inducing aberrant capsid morphology and function. In one embodiment, a capsid assembly modulator accelerates capsid assembly or disassembly, thereby inducing aberrant capsid morphology. In another embodiment, a capsid assembly modulator interacts (e.g. binds at an active site, binds at an allosteric site, modifies and/or hinders folding and the like) with the major capsid assembly protein (CA), thereby disrupting capsid assembly or disassembly. In yet another embodiment, a capsid assembly modulator causes a perturbation in structure or function of CA (e.g., ability of CA to assemble, disassemble, bind to a substrate, fold into a suitable conformation, or the like), which attenuates viral infectivity and/or is lethal to the virus.

As used herein, the term “treatment” or “treating,” is defined as the application or administration of a therapeutic agent, i.e., a compound of the present disclosure (alone or in combination with another pharmaceutical agent), to a patient, or application or administration of a therapeutic agent to an isolated tissue or cell line from a patient (e.g., for diagnosis or ex vivo applications), who has an HBV infection, a symptom of HBV infection or the potential to develop an HBV infection, with the purpose to cure, heal, alleviate, relieve, alter, remedy, ameliorate, improve or affect the HBV infection, the symptoms of HBV infection or the potential to develop an HBV infection. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual” or “subject” refers to a human or a non-human mammal. Non-human mammals include, for example, livestock and pets, such as ovine, bovine, porcine, canine, feline and murine mammals. Preferably, the patient, subject or individual is human.

In treatment methods according to the present disclosure, an effective amount of a pharmaceutical agent according to the present disclosure is administered to a subject suffering from or diagnosed as having such a disease, disorder, or condition. An “effective amount” means an amount or dose sufficient to generally bring about the desired therapeutic or prophylactic benefit in patients in need of such treatment for the designated disease, disorder, or condition. Effective amounts or doses of the compounds of the present disclosure may be ascertained by routine methods such as modeling, dose escalation studies or clinical trials, and by taking into consideration routine factors, e.g., the mode or route of administration or drug delivery, the pharmacokinetics of the compound, the severity and course of the disease, disorder, or condition, the subject's previous or ongoing therapy, the subject's health status and response to drugs, and the judgment of the treating physician. An example of a dose is in the range of from about 0.001 to about 200 mg of compound per kg of subject's body weight per day, preferably about 0.05 to 100 mg/kg/day, or about 1 to 35 mg/kg/day, in single or divided dosage units (e.g., BID, TID, QID). For a 70-kg human, an illustrative range for a suitable dosage amount is from about 0.05 to about 7 g/day, or about 0.2 to about 2.5 g/day.

An example of a dose of a compound is from about 1 mg to about 2,500 mg. In some embodiments, a dose of a compound of the present disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., another drug for HBV treatment) as described herein is less than about 1,000 mg, or less than about 800 mg, or less than about 60 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

Once improvement of the patient's disease, disorder, or condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

HBV infections that may be treated according to the disclosed methods include HBV genotype A, B. C, and/or D infections. However, in an embodiment, the methods disclosed may treat any HBV genotype (“pan-genotypic treatment”). HBV genotyping may be performed using methods known in the art, for example, INNO-LIPA® HBV Genotyping, Innogenetics N.V., Ghent, Belgium).

In an attempt to help the reader of the present application, the description has been separated in various paragraphs or sections. These separations should not be considered as disconnecting the substance of a paragraph or section from the substance of another paragraph or section. To the contrary, the present description encompasses all the combinations of the various sections, paragraphs and sentences that can be contemplated.

Each of the relevant disclosures of all references cited herein is specifically incorporated by reference. The following examples are offered by way of illustration, and not by way of limitation.

### Examples

Exemplary compounds useful in methods of the present disclosure will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

Compounds of Formula (I) and Formula (Ia) may be converted to their corresponding salts using methods known to one of ordinary skill in the art. For example, an amine of Formula (I) is treated with trifluoroacetic acid. HCl, or citric acid in a solvent such as EtO, CH₂Cl₂, THF, MeOH, chloroform, or isopropanol to provide the corresponding salt form. Alternately, trifluoroacetic acid or formic acid salts are obtained as a result of reverse phase HPLC purification conditions. Crystalline forms of pharmaceutically acceptable salts of compounds of Formula (I) and Formula (ta) may be obtained in crystalline form by recrystallization from polar solvents (including mixtures of polar solvents and aqueous mixtures of polar solvents) or from non-polar solvents (including mixtures of non-polar solvents).

Where the compounds according to this present disclosure have at least one chiral center, they may accordingly exist as enantiomers. Where the compounds possess two or more chiral centers, they may additionally exist as diastereomers. It is to be understood that all such isomers and mixtures thereof are encompassed within the scope of the present disclosure.

Compounds represented as “stereomeric mixture” (means a mixture of two or more stereoisomers and includes enantiomers, diastereomers and combinations thereof) are separated by SFC resolution.

Compounds may be obtained as single forms, such as single enantiomers, by form-specific synthesis, or by resolution. Compounds may alternately be obtained as mixtures of various forms, such as racemic (1:1) or non-racemic (not 1:1) mixtures. Where racemic and non-racemic mixtures of enantiomers are obtained, single enantiomers may be isolated using conventional separation methods known to one of ordinary skill in the art, such as chiral chromatography, recrystallization, diastereomeric salt formation, derivatization into diastereomeric adducts, biotransformation, or enzymatic transformation. Where regioisomeric or diastereomeric mixtures are obtained, as applicable, single isomers may be separated using conventional methods such as chromatography or crystallization.

### 1. General Information

### Chemical Names

Chemical names were generated using the chemistry software: ACD/ChemSketch.

### LCMS Methods

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rt) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). All results were obtained with experimental uncertainties that are commonly associated with the method used. Hereinafter, “SQD” means Single Quadrupole Detector, “MSD” Mass Selective Detector, “RT” room temperature, “BEH” bridged ethylsiloxane/silica hybrid, “DAD” Diode Array Detector, “HSS” High Strength silica., “Q-Tof” Quadrupole Time-of-flight mass spectrometers. “CLND”, ChemiLuminescent Nitrogen Detector, “ELSD” Evaporative Light Scanning Detector.

### LCMS Method

(Flow expressed in mL/min; column temperature (T) in ° C.; Run time in minutes).

| Method | | | | | Flow | Run |
|---|---|---|---|---|---|---|
| code | Instrument | Column | Mobile phase | Gradient | Col T | time |
| A | Waters: | Waters: | A: 10 mM | From 95% A | 0.8 | 2 |
| | Acquity ® | BEH C18 | CH₃COONH₄ | to 5% A in 1.3 | 55 | |
| | UPLC ® - | (1.7 μm, | in 95% H₂O + | min, held for | | |
| | DAD and | 2.1*50 mm) | 5% CH₃CN | 0.7 min. | | |
| | SQD | | B: CH₃CN | | | |
| B | Waters: | Waters: | A: 10 mM | From 100% A | 0.6 | 3.5 |
| | Acquity ® | BEH | CH₃COONH₄ | to 5% A in | 55 | |
| | UPLC ® - | (1.8 μm, | in 95% H₂O + | 2.10 min, to | | |
| | DAD and | 2.1*100 mm) | 5% CH₃CN | 0% A in 0.90 | | |
| | SQD | | B: CH₃CN | min, to 5% A | | |
| | | | | in 0.5 min | | |
| C | Agilent | Agilent: | A: 0.1% TFA | From 90% A | 0.65 | 6 |
| | Infinity | Infinity Lab | in water | to 10% A in | 55 | |
| | 1260 HPLC | Poroshell | B: 0.1% TFA | 4.5 min, to 0% | | |
| | interfaced | 120 Bonus- | in MeCN | A in 0.25 min, | | |
| | with | RP (2.7 μm, | | held for 1.25 | | |
| | Agilent 6120 | 2.1 × 50 mm) | | min | | |
| | Quadrupole MS | | | | | |
| D | Agilent | Agilent | A: 0.1% TFA | From 90% A | 0.65 | 6 |
| | Infinity | ZORBAX | in water | to 10% A in | 55 | |
| | 1260 HPLC | StableBond | B: 0.1% TFA | 4.5 min, to 0% | | |
| | interfaced | C18 (1.8 μm, | in MeCN | A in 0.25 min, | | |
| | with | 2.1 × 50 mm) | | held for 1.25 | | |
| | Agilent 6120 | | | min | | |
| | Quadrupole MS | | | | | |
| E | Thermoscientific | Agilent: | A: HCO₂H | 98% A for 2 | 1 | 18.4 |
| | Ultimate | Poroshell | 0.1% in water | min, to 0% A | 30 | |
| | 3000 DAD | EC-C18 | B: HCO₂H | in 10 min, held | | |
| | and | (4 μm, 4.6 × | 0.05% in | for 3.4 min, | | |
| | Brucker | 100 mm) | CH₃CN | back to 98% A | | |
| | HCT ultra | | | in 1.3 min, | | |
| | | | | held for 1.7 | | |
| | | | | min | | |
| F | Thermoscientific | Agilent | A: HCOOH | 50% A for 2 | 1 | 18.4 |
| | Ultimate | Poroshell | 0.1% in waler | min, to 0% A | 30 | |
| | 3000 DAD | EC-C18 | B: HCO₂H | in 10 min, held | | |
| | and | (4 μm, 4.6 × | 0.05% in | for 3.4 min, | | |
| | Brucker | 100 mm) | CH₃CN | back to 50% A | | |
| | HCT ultra | | | in 1.3 min, | | |
| | | | | held for 1.7 | | |
| | | | | min | | |
| G | Thermoscientific | Chiral | A: water | 50% A for 2 | 1 | 31 |
| | Ultimate | technologie: | B: CH₃CN | min, to 0% A | 30 | |
| | 3000 DAD | Chiralpak IC | | in 15 min, held | | |
| | and | (5 μm, 20 × | | for 4 min, back | | |
| | Brucker | 250 mm) | | to 50% A in 2 | | |
| | HCT ultra | | | min, held for 8 | | |
| | | | | min | | |

### SFC Methods

The SFC measurement was performed using an Analytical Supercritical fluid chromatography (SFC) system composed by a binary pump for delivering carbon dioxide (CO₂) and modifier, an autosampler, a column oven, a diode array detector equipped with a high-pressure flow cell standing up to 400 bars. If configured with a Mass Spectrometer (MS) the flow from the column was brought to the (MS). It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Analytical SFC-MS Methods (Flow expressed in mL/min; column temperature (T) in ° C.; Run time in minutes, Back-pressure (BPR) in bars.

### SFC Methods:

| | | | | | Run |
|---|---|---|---|---|---|
| Method | | Mobile | | Flow | time |
| code | Column | phase | Gradient | Col T | BPR |
| SFC_A | Daicel | A: CO₂ | 10%-50% | 2.5 | 9.5 |
| | Chiralpak ® | B: | B in 6 min, | 40 | 130 |
| | ID3 column | iPrOH + 0.2% | hold 3.5 min | | |
| | (3.0 μm, | iPrNH₂ | | | |
| | 150 × 4.6 mm) | | | | |

### NMR Analysis

¹H NMR spectra were recorded on a) a Bruker DRX 500 MHz spectrometer or b) a Bruker Avance 400 MHz spectrometer or c) a Bruker Avance III 400 MHz spectrometer or d) a Bruker Avance 600 MHz spectrometer or e) a Bruker DRX 400 MHz spectrometer or f) a Bruker Avance NEO 400 MHz spectrometer.

NMR spectra were recorded at ambient temperature unless otherwise stated. Data are reported as follow: chemical shift in parts per million (ppm) relative to TMS (δ=0 ppm) on the scale, integration, multiplicity (s=singlet, d=doublet, t=triplet, q=quartet, quin=quintet, sext sextet, sept=septet, m=multiplet, b=broad, or a combination of these), coupling constant(s) J in Hertz (Hz).

### Mass Spectra

Mass spectra were obtained on a Shimadzu LCMS-2020-MSD or Agilent 1200/G⁶¹¹⁰A MSD using electrospray ionization (ESI) in positive mode unless otherwise indicated.

### 2. Abbreviations

| | |
|---|---|
| 9-BBN | 9-Borabicyclo[3.3.1]nonane |
| aq. | Aqueous |
| atm | atmosphere |
| Boc | Tert-butylcarbonyl |
| Boc₂O | Di-tert-butyl dicarbonate |
| BODIPY | Boron-dipyrromethene |
| BuLi | n-butyllithium |
| CA | Capsid Assembly |
| DAST | (Diethylamino)sulfur trifluoride |
| DBU | 1,8-Diazabicyclo[5.4.0]undec-7-ene |
| DCE | Dichloroethane |
| DCM | Dichloromethane |
| DDQ | 2,3-Dicbloro-5,6-dicyano-1,4-benzoquinone |
| DMAP | 4-(Dimethylamino)pyridine |
| DIEA | Diisopropylethyl amine |
| DME | 1,2-Dimethoxyethane |
| DMF | N,N-Dimethylformamide |
| DMF-DMA | N,N-Dimethylformamide dimethyl acetal |
| DNA | Deoxyribonucleic acid |
| DMSO | Dimethyl sulfoxide |
| Et₃N | Triethylamine |
| Et₂O/Ether | Diethyl ether |
| EtOAc/EA | Ethyl acetate |
| EtOH | Ethanol |
| h | Hour |
| HOAc | Acetic acid |
| HMDS | hexamethyldisilazane |
| HMPA | hexamethylphosphoramide |
| HPLC | High Performance Liquid Chromatography |
| i-PrMgCl | Isopropylmagnesium chloride |
| i-PrOH/IPA | Isopropyl alcohol |
| KOtBu | Potassium tert-butoxide |
| LAH | Lithium aluminum hydride |
| LCMS | Liquid Chromatography Mass Spectrometry |
| LDA | Lithium diisopropylamide |
| LHMDS/LiHMDS | Lithium bis(trimethylsilyl)amide |
| MeCN/ACN | Acetonitrile |
| MeI | Methyl iodide |
| MeOH | Methanol |
| min | Minute |
| MsCl | Methanesulfonyl chloride |
| NaHMDS | Sodium bis(trimethylsilyl)amide |
| NaOAc | Sodium acetate |
| NIS | N-iodosuccinimide |
| NMO | 4-Methylmorpholine N-oxide |
| NMR | Nuclear Magnetic Resonance |
| o/n | Overnight |
| o/WE | Over weekend |
| PCC | Pyridinium chlorochromate |
| PE | Petroleum ether |
| Py | Pyridine |
| rt | Room temperature |
| sat. | Saturated |
| TBAF | Tetrabutylammonium fluoride |
| TBDPS | Tert-butyldiphenylsilyl |
| TDAM | Tris(dimethylmino)methane |
| TEA | triethylamine |
| t-BuOK | Potassium tert-butoxide |
| TFA | Trifluoroacetic acid |
| THF | Tetrahydrofuran |
| TMEDA | N,N,N′,N′-Tetramethylethylenediamine |
| TPAP | Tetrapropylammonium perruthenate |
| Δ | Heating under reflux |

### 3. Synthesis of Compounds

### 3.1. Synthesis of the 6-Membered Ring Compounds

### 3.1.1. Synthesis of Key Intermediates

### 3.1.1.1. Synthesis of Intermediates I1-I4, I6

### Intermediate I1

### 5-tert-Butyl 3-ethyl 2-(but-3-yn-1-yl)-2,4,6,7-tetrahydro-5H-pyrazolo[4,3-c]pyridine-3,5-dicarboxylate

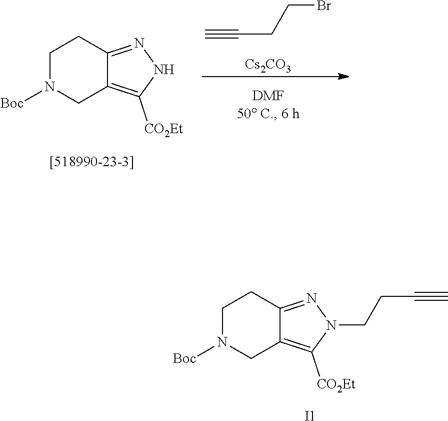

The reaction was performed under anhydrous condition under Ar atmosphere.

To a solution of 5-tert-butyl 3-ethyl 4,5,6,7-tetrahydro-2H-indazole-3,5-dicarboxylate (1.50 g, 5.08 mmol) in DMF (30 mL) were added Cs₂CO₃ (1.65 g, 5.08 mmol) and 4-bromobutyne (477 μL, 5.08 mmol). The reaction mixture was stirred at 50° C. for 1 h. Additional amounts of Cs₂CO₃ (1.65 g, 5.08 mmol) and 4-bromobutyne (477 μL, 5.08 mmol) were added and the reaction mixture was stirred at 50° C. for another hour. The procedure was repeated until completion of the reaction (6 equivalents of Cs₂CO₃ and 4-bromobutyne were added). The reaction mixture was diluted with H₂O (60 mL) and extracted with EtOAc (3×60 mL). The combined organic layers were washed with brine (3×60 mL), dried (Na₂SO₄), filtered and concentrated under reduced pressure to dryness. The crude mixture was purified by flash column chromatography (C-18, mobile phase: MeCN/H₂O, gradient form: 1:9 to 1:1) to afford intermediate I1 (897 mg, 51%) as a yellow oil.

### Intermediate I2

### 5-tert-Butyl 3-ethyl 2-(pent-3-yn-1-yl)-2,4,6,7-tetrahydro-5H-pyrazolo[4,3-c]pyridine-3,5-dicarboxylate

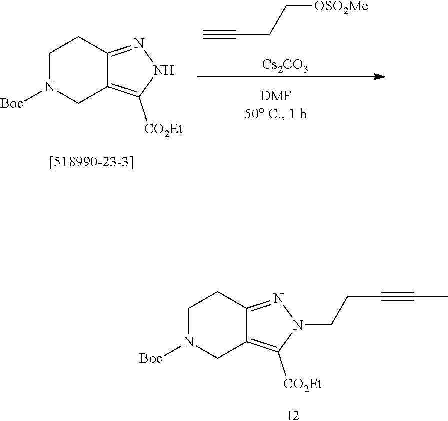

The reaction was performed under anhydrous conditions and under Ar atmosphere.

To a solution of 5-tert-butyl 3-ethyl 4,5,6,7-tetrahydro-2H-indazole-3,5-dicarboxylate (2.00 g, 6.78 mmol) in DMF (40 mL) was added Cs₂CO₃ (4.41 g, 13.5 mmol) and methanesulfonic acid pent-3-ynyl ester (2.20 g, 13.5 mmol). The reaction mixture was stirred at 50° C. for 1 h. diluted with H₂O (100 mL) and extracted with EtOAc (3×100 mL). The combined organic layers were washed brine (3×100 mL), dried (Na₂SO₄), filtered and concentrated under reduced pressure to dryness. The crude mixture was purified by flash column chromatography (C-18, mobile phase: MeCN/H₂O, gradient from: 1:9 to 1:1) to afford intermediate I2 (1.30 g, 53%) as a light yellow oil.

### Intermediate I3

### 5-tert-Butyl 3-ethyl 2-(hex-3-yn-1-yl)-2,4,6,7-tetrahydro-5H-pyrazolo[4,3-c]pyridine-3,5-dicarboxylate

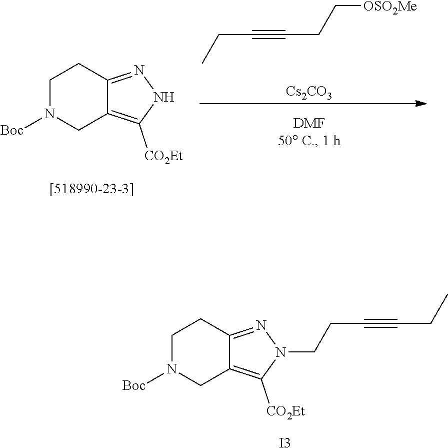

Intermediate I3 (1.18 g, 46%) was prepared in an analogous manner to that described for intermediate I2.

### Intermediate I4

### 5-tert-Butyl 3-ethyl 2-(4-phenylbut-3-yn-1-yl)-2,4,6,7-tetrahydro-5H-pyrazolo[4,3-c]pyridine-3,5-dicarboxylate

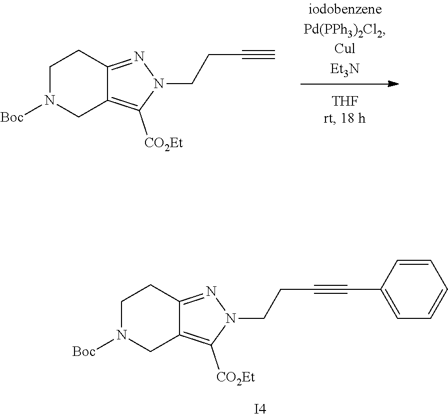

The reaction was performed under anhydrous conditions and under Ar atmosphere.

To a solution of intermediate I1 (500 mg, 1.44 mmol) in THF (6 mL) were added iodobenzene (242 μL, 2.16 mmol) and Et₃N (602 μL, 4.32 mmol). The mixture was degassed with Ar. Pd(PPh₃)₂Cl₂ (50.5 mg, 0.072 mmol) and CuI (27.4 mg, 0.14 mmol) were added and the reaction mixture was stirred at room temperature for 18 h. The reaction mixture was combined with another fraction (0.14 mmol), diluted with EtOAc (80 mL), washed with HCl (1N, aq.) and brine, dried (Na₂SO₄), filtered and concentrated under reduced pressure. The crude mixture was purified by flash column chromatography (silica, mobile phase: cyclohexane-EtOAc, gradient from 100:0 to 80:20) to afford intermediate I4 (475 mg, 70%) as a yellow oil.

### Intermediate I6

### 5-tert-Butyl 3-ethyl (6R)-2-(but-3-yn-1-yl)-6-methyl-2,4,6,7-tetrahydro-5H-pyrazolo[4,3-c]-pyridine-3,5-dicarboxylate

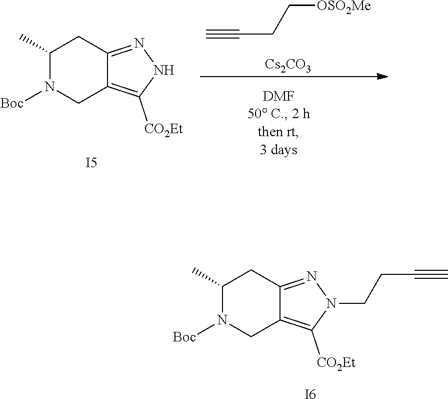

The reaction was performed under anhydrous conditions and under Ar atmosphere.

To a solution of 5-tert-butyl 3-ethyl ((6R)-6-methyl-4,5,6,7-tetrahydro-2H-indazole-3,5-dicarboxylate 15 (1.50 g, 4.85 mmol) and methanesulfonic acid but-3-ynyl ester (1.93 g, 9.70 mmol) in DMF (30 mL) was added Cs₂CO₃ (3.16 g, 9.70 mmol). The reaction mixture was stirred at 50° C. for 2 h then at room temperature for 3 days. Additional quantity of methanesulfonic acid but-3-ynyl ester (0.96 g, 4.85 mmol) and Cs₂CO₃ (1.58 g, 4.85 mmol) was added and the reaction mixture was stirred at 50° C. for another hour. The reaction mixture was diluted with H₂O (70 mL) and extracted with EtOAc (3×50 mL). The combined organic layers were washed with brine (3×50 mL), dried (Na₂SO₄), filtered and concentrated under reduced pressure to dryness. The crude mixture was purified by flash column chromatography (C-18, mobile phase: MeCN/H₂O, gradient from 35:65 to 56:44) to afford intermediate I6 (930 mg, 89%) as a yellow oil.

### 3.1.1.2. Synthesis of Intermediate I11

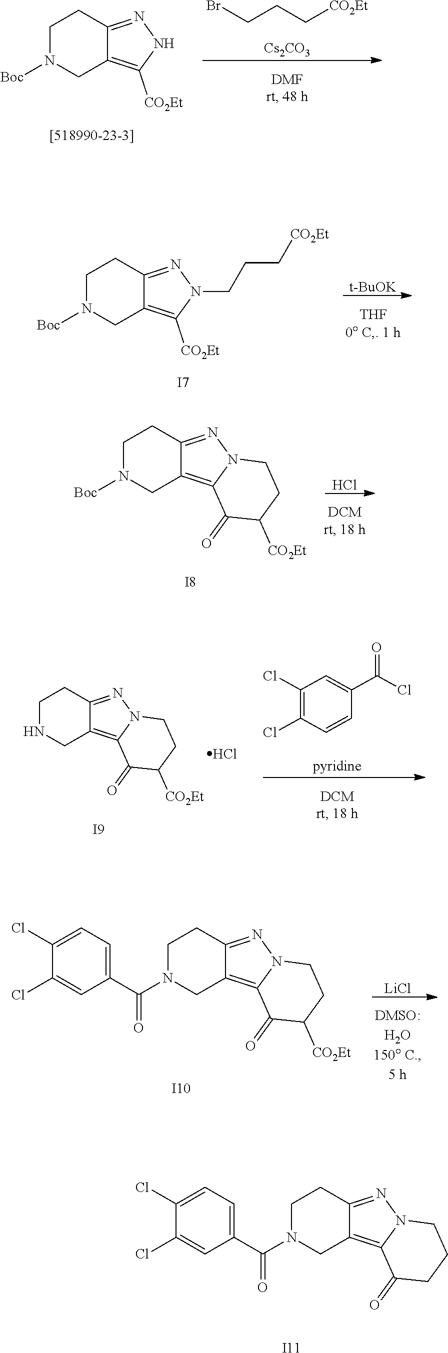

### Intermediate I7

### 5-tert-Butyl 3-ethyl 2-(4-ethoxy-4-oxobutyl)-2,4,6,7-tetrahydro-5H-pyrazolo[4,3-c]pyridine-3,5-dicarboxylate

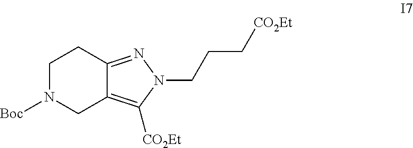 [SMILES: CCOC(=O)CCCn1nc2c(c1C(=O)OCC)CN(C(=O)OC(C)(C)C)CC2]

The reaction was performed under anhydrous conditions and under Ar atmosphere.

To a suspension of Cs₂CO₃ (12.9 g, 39.7 mmol) in DMF (100 mL) were successively added 5-(tert-butyl) 3-ethyl 2,4,6,7-tetrahydro-5H-pyrazolo[4,3-c]pyridine-3,5-dicarboxylate (10.2 g, 33.1 mmol) and ethyl 4-bromobutyrate (5.21 mL, 36.4 mmol). The reaction mixture was stirred at room temperature for 48 h and poured into water (150 mL) and extracted with EtOAc (2×150 mL). The combined organic extracts were washed with brine (3×150 mL), dried (Na₂SO₄), filtered and concentrated under reduce pressure to give intermediate I7 as a yellow oil (15 g, 90% purity, 66/33 mixture of regioisomers) which was used as such in the next step.

### Intermediate I8

### 2-tert-Butyl 9-ethyl 10-oxo-3,4,7,8,9,10-hexahydropyrazolo[1,5-a:4,3-c′]dipyridine-2,9(1H)-dicarboxylate

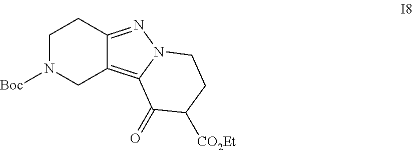 [SMILES: CCOC(=O)C1CCn2nc3c(c2C1=O)CN(C(=O)OC(C)(C)C)CC3]

The reaction was performed under anhydrous conditions and under Ar atmosphere.

To a solution of t-BuOK (7.42 g, 66.2 mmol) in THF (150 mL) at 0° C. was added dropwise a solution of intermediate I7 in THF (150 mL). The reaction mixture was stirred at 0° C. for 1 h. The reaction was quenched with water (200 mL) and acidified with HCl (1N, 150 mL). The layers were separated and the aqueous phase was extracted with EtOAc (2×150 mL). The combined organic layers were dried (Na₂SO₄), filtered and concentrated under reduced pressure. The crude mixture was purified by flash column chromatography (silica, mobile phase: cyclohexane/EtOAc, gradient from 70:30 to 30:70) to give intermediate I8 (8.62 g, 93% purity, 67% over 2 steps) as a colorless gum.

### Intermediate I9

### Ethyl 10-oxo-1,2,3,4,7,8,9,10-octahydropyrazolo[1,5-a:4,3-c′]dipyridine-9-carboxylate Hydrochloride

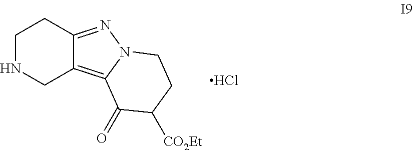 [SMILES: CCOC(=O)C1CCn2nc3c(c2C1=O)CNCC3 ; Cl]

The reaction was performed under anhydrous conditions and under Ar atmosphere.

HCl (4N in 1,4-dioxane, 55.4 mL, 222 mmol) was added at room temperature to a solution of intermediate I8 (8.05 g, 22.2 mmol) in DCM (50 mL). The reaction mixture was stirred for 18 h and diluted with Et₂O (200 ml). The mixture was filtered and the rinsed with Et₂O (100 mL). The solid was dried under vacuum to give intermediate I9 as a white solid which was used as such in the next step.

### Intermediate I10

### Ethyl 2-(3,4-dichlorobenzoyl)-10-oxo-1,2,3,4,7,8,9,10-octahydropyrazolo[1,5-a:4,3-c′]-dipyridine-9-carboxylate

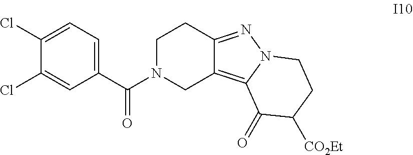 [SMILES: CCOC(=O)C1CCn2nc3c(c2C1=O)CN(C(=O)c1ccc(Cl)c(Cl)c1)CC3]

The reaction was performed under anhydrous conditions and under Ar atmosphere.

To a solution of intermediate I9 in DCM (100 mL) at 0° C. was added pyridine (5.38 mL, 66.5 mmol) followed by a solution of 3,4-dichlorobenzoyl chloride (5.10 g, 24.4 mmol) in DCM (50 mL) dropwise. The reaction mixture was warmed to room temperature and stirred for 18 h. The reaction mixture was diluted with DCM (150 mL) and washed with HCl (1M, aq., 2×150 mL), and brine (150 mL). The organic layer was dried (Na₂SO₄), filtered and concentrated under reduced pressure. The crude mixture was purified by flash column chromatography (silica, mobile phase: DCM/EtOAc, gradient from 100:0 to 70:30) to give intermediate I10 (8.72 g, 90% over 2 steps) as an off-white foam.

### Intermediate I11

### 2-(3,4-Dichlorobenzoyl)-1,2,3,4,8,9-hexahydropyrazolo[1,5-a:4,3-c′]dipyridin-10(7H)-one

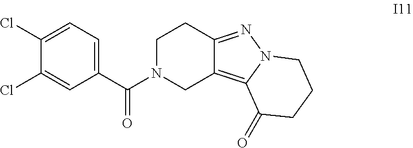 [SMILES: O=C1CCCn2nc3c(c21)CN(C(=O)c1ccc(Cl)c(Cl)c1)CC3]

To a solution of intermediate I10 (1.00 g, 2.3 mmol) in DMSO (18 mL) were added H₂O (2 mL) and LiCl (126 mg, 2.98 mmol). The reaction mixture was stirred at 150° C. for 5 h, cooled to room temperature and diluted with H₂O (100 mL). The solution was stirred for another 30 min. The precipitated was collected by filtration and dried under vacuum overnight at 50° C. to afford intermediate I11 (776 mg, 93%) as a white solid.

### 3.1.2. Synthesis of Isoxazole Derivative Compounds

### 3.1.2.1. Synthesis of Compound 1

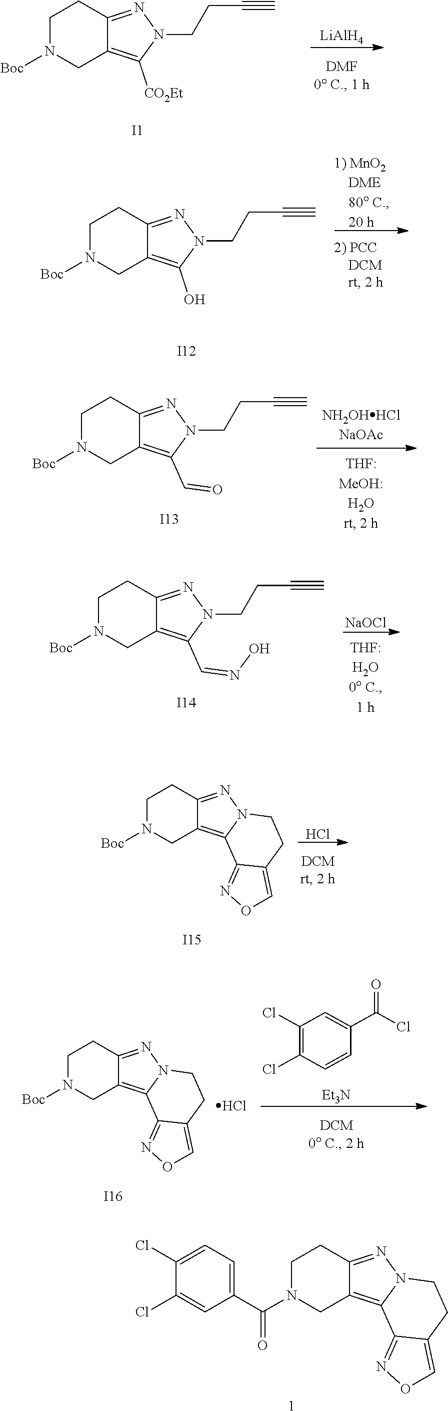

### Intermediate I12

### tert-Butyl 2-(but-3-yn-1-yl)-3-(hydroxymethyl)-2,4,6,7-tetrahydro-5H-pyrazolo[4,3-c]pyridine-5-carboxylate

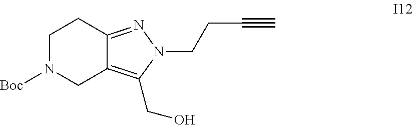 [SMILES: C#CCCn1nc2c(c1CO)CN(C(=O)OC(C)(C)C)CC2]

The reaction was performed under anhydrous conditions and wider Ar atmosphere.

To a solution of intermediate I1 (880) mg, 2.53 mmol) in THF (17 mL) at 0 TC was added LiAlH₄ (192 mg, 5.07 mmol). The reaction mixture was stirred at 0° C. for 1 h. The reaction was quenched with EtOAc (50 mL) and 1120 (5 mL), and a solution of Rochelle's salt (1M, aq., 50 mL) was added. The mixture was stirred for 30 min at room temperature and the layers were separated. The aqueous phase was extracted with EtOAc (50 mL). The combined organic extracts were washed with brine, dried (Na₂SO₄), filtered and concentrated under reduced pressure to afford intermediate I12 (708 mg, 92%) as a light yellow oil.

### Intermediate I13

### Tert-Butyl 2-(but-3-yn-1-yl)-3-formyl-2,4,6,7-tetrahydro-5H-pyrazolo[4,3-c]pyridine-5-carboxylate

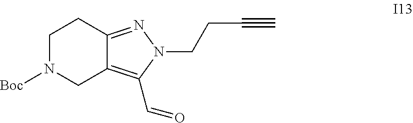 [SMILES: C#CCCn1nc2c(c1C=O)CN(C(=O)OC(C)(C)C)CC2]

The reaction was performed under anhydrous conditions and under Ar atmosphere.

To a suspension of intermediate I12 (705 mg, 2.31 mmol) in DME (30 mL) was added MnO₂ (803 mg, 9.24 mmol). The reaction mixture was stirred at 80° C. for 18 h. Additional quantity of MnO₂ (401 mg, 4.62 mmol) was added and the reaction mixture was stirred for another 2 h at 80° C. The mixture was filtered over a pad of Celite® and the filtrate was concentrated under reduced pressure. The residue was solubilized in DCM (30 mL) and PCC (746 mg, 3.46 mmol) was added. The reaction mixture was stirred at room temperature for 2 h and concentrated under reduced pressure to dryness. The crude mixture was purified by flash column chromatography (silica, mobile phase: cyclohexane/EtOAc, gradient from 100:0 to 60:40) to afford intermediate I13 (282 mg, 40%) as a colorless oil.

### Intermediate I14

### Tert-Butyl 2-(but-3-yn-1-yl)-3-[(hydroxyimino)methyl]-2,4,6,7-tetrahydro-5H-pyrazolo[4,3-c]-pyridine-5-carboxylate

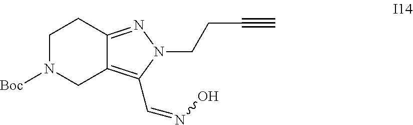 [SMILES: C#CCCn1nc2c(c1C=NO)CN(C(=O)OC(C)(C)C)CC2]

To a solution of intermediate I13 (200 mg, 0.66 mmol) and NaOAc (162 mg, 1.98 mmol) in THF (6.5 mL), MeOH (6.5 mL) and H₂O (13 mL) was added N-hydroxylamine hydrochloride (91.6 mg, 1.32 mmol). The reaction mixture was stirred at room temperature for 2 h and diluted with H₂O (10 mL). The layers were separated and the aqueous phase was extracted with DCM (3×30 mL). The combined organic layers were washed with brine, dried (Na₂SO₄), filtered and concentrated under reduced pressure to afford intermediate I14 (203 mg) which was used as such in the next step.

### Intermediate I15

### Tert-Butyl 4,5,8,9-tetrahydro[1,2]oxazolo[3,4-c]pyrazolo[1,5-a:4,3-c′]dipyridine-10(11H)-carboxylate

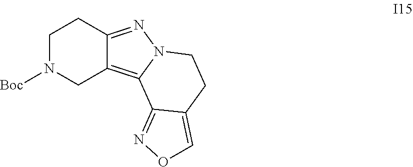 [SMILES: CC(C)(C)OC(=O)N1CCc2nn3c(c2C1)-c1nocc1CC3]

To a solution of intermediate I14 (200 mg, 0.63 mmol) in THF (13 mL) and H₂O (0.8 mL) at 0° C. was added sodium hypochlorite (15% in H₂O, 779 μL, 1.57 mmol). The reaction mixture was stirred at 0° C. for 1 h. The mixture was combined with another fraction (0.24 mmol) and diluted with H₂O (30 mL). The layers were separated and the aqueous phase was extracted with EtOAc (3×30 mL). The combined organic extracts were washed with brine, dried (Na₂SO₄ filtered and concentrated under reduced pressure. The crude mixture was purified by flash column chromatography (silica, mobile phase: cyclohexane/EtOAc, gradient from 100:0 to 20:80) to afford intermediate I15 (116 mg, 42%) as a colorless oil.

### Intermediate I16

### 4,5,8,9,10,11-hexahydro[1,2]oxazolo[3,4-c]pyrazolo[1,5-a:4,3-c′]dipyridine Hydrochloride

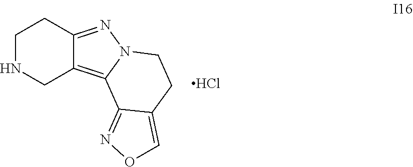 [SMILES: c1onc2c1CCn1nc3c(c1-2)CNCC3 ; Cl]

The reaction was performed under anhydrous conditions and under Ar atmosphere.

To a solution of intermediate I15 (110 mg, 0.35 mmol) in DCM (2 mL) was added HCl (4M in 1,4-dioxane, 1.74 mL, 6.95 mmol). The reaction mixture was stirred at room temperature for 2 h and concentrated under reduced pressure to dryness to afford intermediate I16 (88 mg) which was used as such in the next step.

### Compound 1

### (3,4-Dichlorophenyl)(4,5,8,9-tetrahydro[1,2]oxazolo[3,4-c]pyrazolo[1,5-a:4,3-c′]dipyridin-10(11H)-yl)methanone

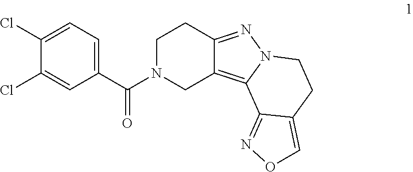 [SMILES: O=C(c1ccc(Cl)c(Cl)c1)N1CCc2nn3c(c2C1)-c1nocc1CC3]

The reaction was performed under Ar atmosphere.

To a solution of crude intermediate I16 in DCM (8 mL) at 0° C. was added Et₃N (144 μL, 1.03 mmol) followed by a solution of 3,4-dichlorobenzoyl chloride (79.3 mg, 0.38 mmol) in DCM (2 mL). The reaction mixture was stirred at 0° C. for 2 h. The reaction mixture was diluted with DCM (30 mL), washed with HCl (1 N, aq., 20 mL), NaHCO₃ (sat., aq., 20 mL) and brine, dried (Na₂SO₄), filtered and concentrated under reduced pressure. The crude mixture was purified by flash column chromatography (silica, mobile phase: DCM/MeOH, gradient form: 100:0 to 95:5) to afford compound 1 (115 mg, 84% over 2 steps) as a white solid.

¹H NMR (40 MHz, DMSO-d₆, 80° C.) δ ppm 8.74 (s, 1H), 7.73-7.64 (m, 2H), 7.46 (dd, J=8.2, 1.8 Hz, 1H), 4.76-4.66 (m, 2H), 4.30 (t, 1=6.7 Hz, 2H), 3.81-3.71 (m, 2H), 3.12 (td, J=6.9, 0.9 Hz, 2H), 2.80 (t, J=5.9 Hz, 2H); LCMS (method E): Rt=9.6 min, m/z calcd. for C₁₈H₁₄Cl₂N₄O₂ 388, m/z found 389 [M+H]⁺.

### 3.1.2.2. Synthesis of Compound 2

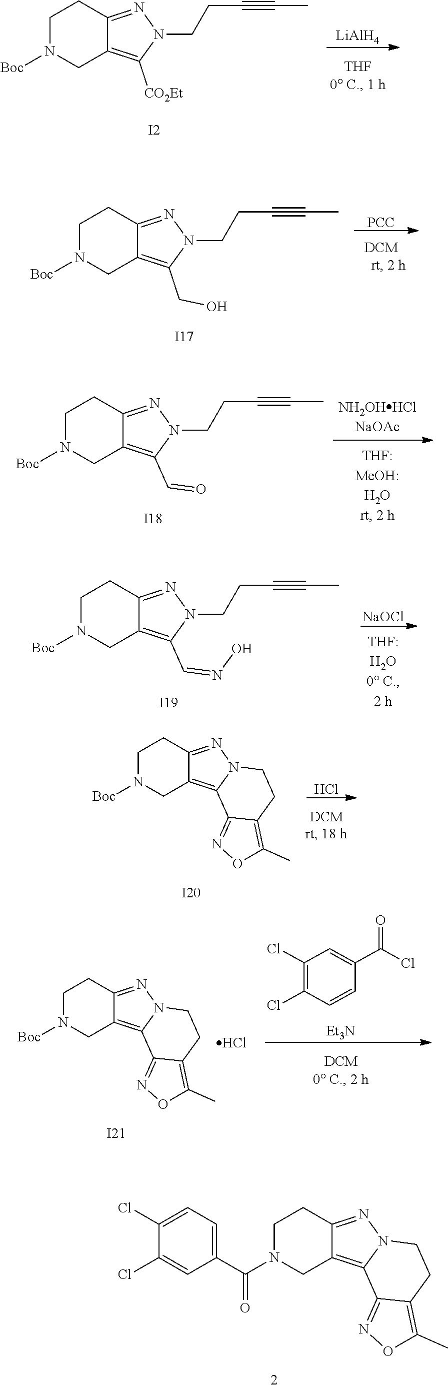

### Intermediate I17

### Tert-Butyl 3-(hydroxymethyl)-2-(pent-3-yn-1-yl)-2,4,6,7-tetrahydro-5H-pyrazolo[4,3-c]pyridine-5-carboxylate

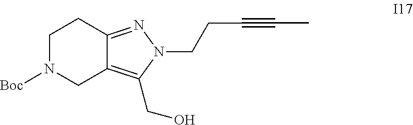 [SMILES: CC#CCCn1nc2c(c1CO)CN(C(=O)OC(C)(C)C)CC2]

The reaction was performed under anhydrous conditions and under Ar atmosphere.

To a solution of intermediate I2 (1.25 g, 3.46 mmol) in THF (30 mL) at 0° C. was added LiAlH₄ (263 mg, 6.92 mmol). The reaction mixture was stirred at 0° C. for 1 h. The reaction was quenched with EtOAc (100 mL) and H₂ (10 mL), and a solution of Rochelle's salt (1M, aq., 100 mL) was added. The mixture was stirred at room temperature for 30 min and the layers were separated. The aqueous phase was extracted with EtOAc (100 mL). The combined organic layers were washed with brine, dried (Na₂SO₄) and concentrated under reduced pressure. The crude mixture was purified by flash column chromatography (silica, mobile phase: cyclohexane/EtOAc, gradient from 100:0 to 20:80) to afford intermediate I17 (991 mg, 90%) as a colorless oil.

### Intermediate I18

### Tert-Butyl 3-formyl-2-(pent-3-yn-1-yl)-2,4,6,7-tetrahydro-5H-pyrazolo[4,3-c]pyridine-5-carboxylate

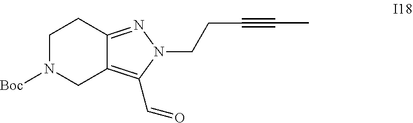 [SMILES: CC#CCCn1nc2c(c1C=O)CN(C(=O)OC(C)(C)C)CC2]

The reaction was performed under anhydrous conditions and under Ar atmosphere.

To a suspension of intermediate I17 (985 mg, 3.08 mmol) in DCM (30 mL) was added PCC (997 mg, 4.62 mmol). The reaction mixture was stirred at room temperature for 2 h and concentrated to dryness. The crude mixture was purified by flash column chromatography (silica, mobile phase: cyclohexane/EtOAc, gradient from 100:0 to 50:50) to afford intermediate I18 (814 mg, 83%) as a colorless oil.

### Intermediate I19

### tert-Butyl 3-[(hydroxyimino)methyl]-2-(pent-3-yn-1-yl)-2,4,6,7-tetrahydro-5H-pyrazolo[4,3-c]-pyridine-5-carboxylate

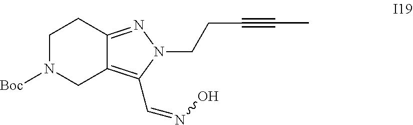 [SMILES: CC#CCCn1nc2c(c1C=NO)CN(C(=O)OC(C)(C)C)CC2]

To a solution of intermediate I18 (400 mg, 1.26 mmol) and NaOAc (310 mg, 3.78 mmol) in THF (13 mL), MeOH (13 mL) and H₂O (26 mL) was added N-hydroxylamine hydrochloride (175 mg, 2.52 mmol). The reaction mixture was stirred at room temperature for 2 h and diluted with H₂O (20 mL). The layers were separated and the aqueous phase was extracted with DCM (3×60 mL). The combined organic layers were washed with brine, dried (Na₂SO₄), filtered and concentrated under reduced pressure to afford intermediate I19 (378 mg, 90%) which was used as such in the next step.

### Intermediate I20

### Tert-Butyl 3-methyl-4,5,8,9-tetrahydro[1,2]oxazolo[3,4-c]pyrazolo[1,5-a:4,3-c′]dipyridine-10(11H)-carboxylate

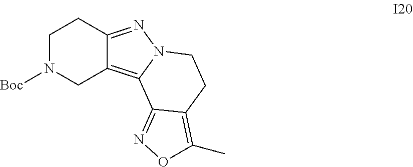 [SMILES: Cc1onc2c1CCn1nc3c(c1-2)CN(C(=O)OC(C)(C)C)CC3]

To a solution of intermediate I19 (370 mg, 1.11 mmol) in THF (20 mL) and H₂O (1.3 ml) at 0° C. was added sodium hypochlorite (15% in H₂O, 1.38 mL, 2.78 mmol). The reaction mixture was stirred at 0° C. for 2 h and diluted with H₂O (60 mL). The layers were separated and the aqueous phase was extracted with EtOAc (3×60 ml). The combined organic layers were washed with brine, dried (Na₂SO₄), filtered and concentrated under reduced pressure. The crude mixture was purified by flash column chromatography (silica, mobile phase: cyclohexane/EtOAc, gradient from 100:0 to 30:70) to afford intermediate I20 (108 mg, 29%) as a colorless oil.

### Intermediate I21

### 3-Methyl-4,5,8,9,10,11-hexahydro[1,2]oxazolo[3,4-c]pyrazolo[1,5-a:4,3-c′]dipyridine Hydrochloride

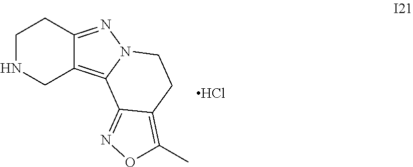 [SMILES: Cc1onc2c1CCn1nc3c(c1-2)CNCC3 ; Cl]

The reaction was performed under anhydrous conditions and under Ar atmosphere.

To a solution of intermediate I20 (100 mg, 0.303 mmol) in DCM (4 mL) was added HCl (4N in 1,4-dioxane, 1.51 mL, 6.04 mmol). The reaction mixture was stirred at room temperature for 18 h, then concentrated to dryness to afford intermediate I21 which was used such as for the next step.

### Compound 2

### 3-Methyl-4,5,8,9,10,11-hexahydro[1,2]oxazolo[3,4-c]pyrazolo[1,5-a:4,3-c]dipyridine

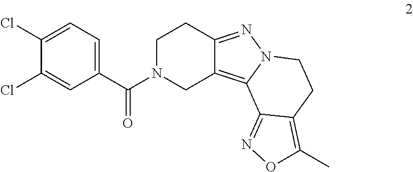 [SMILES: Cc1onc2c1CCn1nc3c(c1-2)CN(C(=O)c1ccc(Cl)c(Cl)c1)CC3]

The reaction was performed under anhydrous conditions and under Ar atmosphere.

To a solution of intermediate I21 in DCM (5 mL) at 0° C. was added Et₃N (125 μL, 0.9 mmol) followed by a solution of 3,4-dichlorobenzoyl chloride (69.1 mg, 0.330 mmol) in DCM (5 mL). The reaction mixture was stirred at 0° C. for 2 h. The reaction mixture was diluted with DCM (30 mL), washed with HCl (1N, aq., 20 mL), NaHCO₃ (sat., aq., 20 mL) and brine, dried (Na₂SO₄), filtered and concentrated under reduced pressure. The crude mixture was purified by flash column chromatography (silica, mobile phase. DCM/MeOH, gradient from 100:0 to 97.3) to afford compound 2 (80 mg, 65% over 2 steps) as a white solid.

¹H NMR (400 MHz, DMSO-d₆, 80° C.) δ ppm 7.73-7.64 (m, 2H), 7.45 (dd, J=8.3, 1.8 Hz, 1H), 4.73-4.68 (m, 2H), 4.28 (t, J=6.8 Hz, 2H), 3.78 (t, J=4.8 Hz, 2H), 3.00 (t, J=6.8 Hz, 2H), 2.79 (t, J=6.0 Hz, 2H), 2.42 (s, 31H); LCMS (method E): Rt=9.9 min, m/z calcd. for C₁₉H₁₆Cl₂N₄O₂ 402, m/z found 403 [M+H]⁺.

### 3.1.2.3. Synthesis of Compound 3

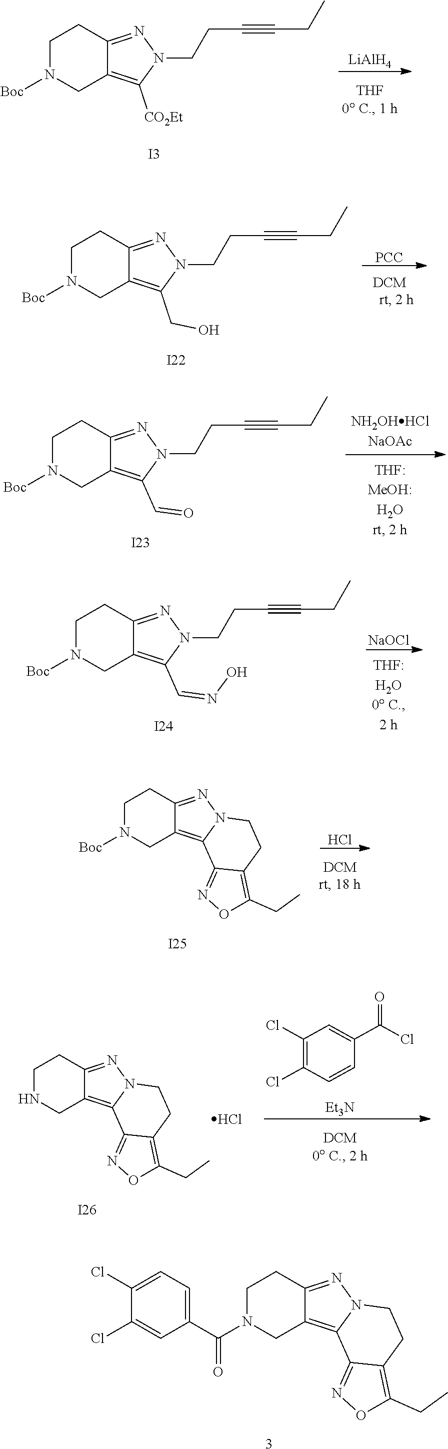

Compound 3 was prepared in an analogous manner to that described for compound 2.

Compound 3, (3,4-dichlorophenyl)(3-ethyl-4,5,8,9-tetrahydro[1,2]oxazolo[3,4-c]pyrazolo[1,5-a:4,3-c′]dipyridin-10(11H)-yl)methanone, was obtained as a white solid.

¹H NMR (400 MHz, DMSO-d₆, 80° C.) δ ppm 7.72 (d, J=8.0 Hz, 1H), 7.70 (d, J=2.0 Hz, 1H), 7.45 (dd, J=8.0, 2.0 Hz, 1H), 4.74-4.66 (m, 2H), 4.28 (t, J=6.9 Hz, 2H), 3.82-3.73 (m, 2H), 3.04 (t, J=6.5 Hz, 2H), 2.87-2.77 (m, 4H), 1.28 (t, J=7.6 Hz, 3H); LCMS (method F): Rt=4.90 min. m/z calcd. for C₂₀H₁₈Cl₂N₄O₂ 416, m/z found 417 [M+H]⁺.

### 3.1.2.4. Synthesis of Compound 4

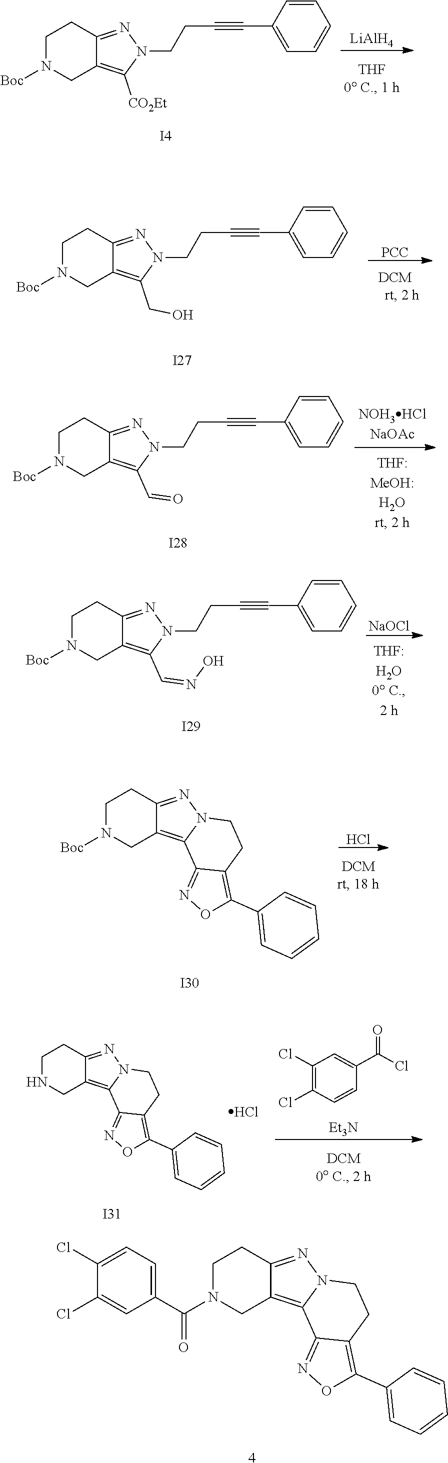

Compound 4 was prepared in an analogous manner to that described for compound 2.

Compound 4, (3,4-Dichlorophenyl)(3-phenyl-4,5,8,9-tetrahydro[1,2]oxazolo[3,4-c]pyrazolo-[1,5-a:4,3-c′]dipyridin-10(11H)-yl)methanone, was obtained as a light yellow solid.

¹H NMR (400 MHz, DMSO-d₆, 80° C.) δ ppm 7.85-7.78 (m, 2H), 7.72 (d, J=8.0 Hz, 1H), 7.72 (d, J=2.0 Hz, 1H), 7.63-7.52 (m, 3H), 7.47 (dd, J=8.0, 2.0 Hz, 1H), 4.78-4.73 (m, 2H), 4.38 (t, J=6.8 Hz, 2H), 3.84-3.75 (m, 2H), 3.36 (t, J=6.8 Hz, 2H), 2.82 (t, J=5.8 Hz, 2H); LCMS (method E): Rt=11.4 min, m/z calcd. for C₂₄H₁₈Cl₂N₄O₂ 464, m/z found 465 [M+H]⁺.

### 3.1.2.5. Synthesis of Compound 5

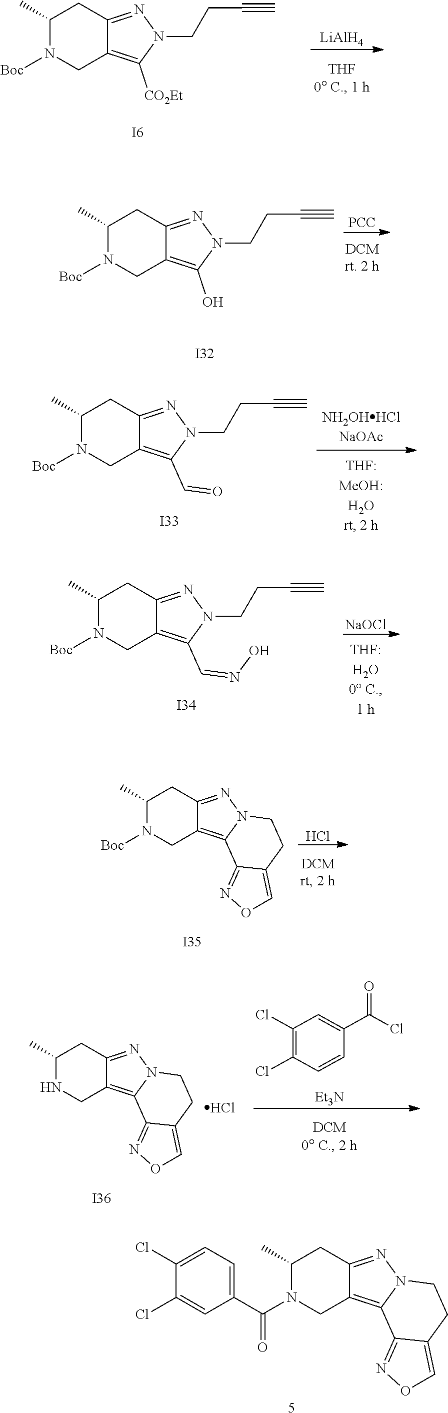

Compound 5 was prepared in an analogous manner to that described for compound 2.

Compound 5, (3,4-Dichlorophenyl)[(9R)-9-methyl-4,5,8,9-tetrahydro[1,2]oxazolo[3,4-c]-pyrazolo[1,5-a:4,3-c′]dipyridin-10(11H)-yl]methanone, was obtained as a white solid.

¹H NMR (400 MHz, DMSO-d₆, 80° C.) δ ppm 8.74 (s, 1H), 7.70 (d, J=8.4 Hz, 1H), 7.69 (d, J=2.0 Hz, 1H), 7.43 (dd, J=8.4, 2.0 Hz, 1H), 5.15-5.02 (m, 1H), 4.72-4.57 (m, 1H), 4.35 (m, 1H), 4.31 (t, J=6.4 Hz, 2H), 3.14-3.10 (m, 2H), 3.00 (dd, J=16.0, 5.6 Hz, 1H), 2.51 (d, J=16.0 Hz, 1H), 1.21 (d, J=6.8 Hz, 3H); LCMS (method E): Rt=9.9 min, m/z calcd. for C₁₉H₁₆Cl₂N₄O₂ 402, m/z found 403 [M+H]⁺.

### 3.1.3. Synthesis of Pyrazole Derivative Compounds

### 3.1.3.1. Synthesis of Compound 6

### (3,4-Dichlorophenyl)(2,4,5,8,9,11-hexahydro-10H-pyrazolo[3,4-c]pyrazolo[1,5-a:4,3-c′]-dipyridin-10-yl)methanone

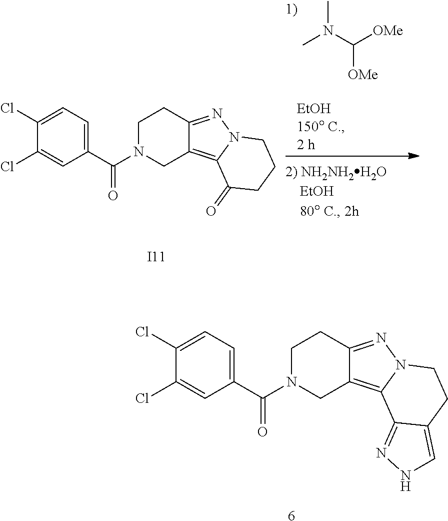

The reaction was performed under anhydrous conditions and under Ar atmosphere.

To a solution of intermediate I11 (210 mg, 0.58 mmol) in EtOH (5 mL) was added N,N-dimethylformamide dimethyl acetate (536 μL, 4.04 mmol). The reaction mixture was stirred at 150° C. for 2 h, concentrated to dryness and co-evaporated with DCM (2×10 mL). The residue was suspended in EtOH (5 mL) and hydrazine monohydrate (559 μL, 11.5 mmol) was added. The reaction mixture was stirred at 80° C. for 2 h and concentrated to dryness. The crude mixture was purified by flash column chromatography (silica, mobile phase: DCM/MeOH, gradient from 100:0 to 90:10). A second purification was performed by flash column chromatography (C-18, mobile phase: MeCN/H₂O, gradient from 10:90 to 60:40) to afford compound 6 (119 mg, 53%) as a white solid.

¹H NMR (400 MHz, DMSO-d₆, 80° C.) δ ppm 12.59 (br.s, 1H), 7.71-7.67 (m, 2H), 7.57 (s, 1H), 7.45 (dd, J=8.2, 2.0 Hz, 1H), 4.76-4.66 (m, 2H), 4.20 (t, J=7.2 Hz, 2H), 3.83-3.71 (m, 2H), 3.02 (t, J=6.7 Hz, 2H), 2.76 (t, J=5.5 Hz 2H); LCMS (method E): Rt=8.6 min, m/z calcd. For C₁₈H₁₅Cl₂N₅O 387, m/z found 388 [M+H]⁺.

### 3.1.3.2. Synthesis of Compound 7

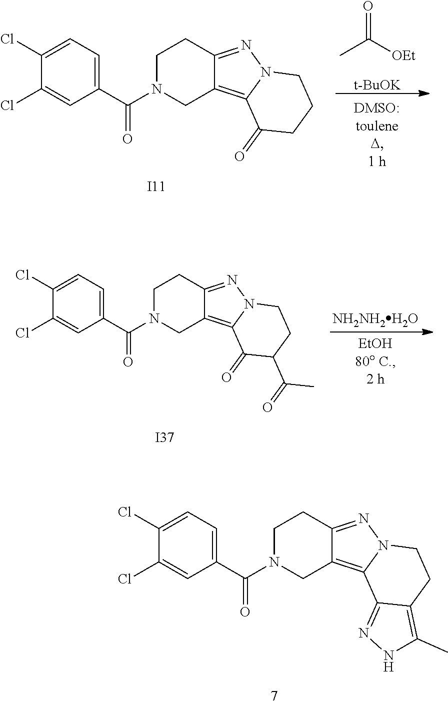

### Intermediate I3

### 9-Acetyl-2-(3,4-dichlorobenzoyl)-1,2,3,4,8,9-hexahydropyrazolo[1,5-a:4,3-c′]dipyridin-10(7H)-one

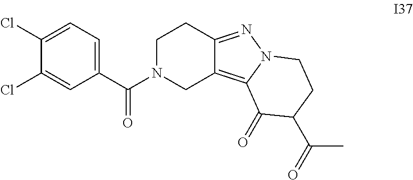 [SMILES: CC(=O)C1CCn2nc3c(c2C1=O)CN(C(=O)c1ccc(Cl)c(Cl)c1)CC3]

The reaction was performed under anhydrous conditions and under Ar atmosphere.

To a solution of intermediate I11 (400 mg, 1.10 mmol) in toluene (4.8 mL) and DMSO (1.1 mL) was added t-BuOK (370 mg, 3.30 mmol) at 0° C. EtOAc (1.40 mL, 14.3 mmol) was added dropwise and the resulting reaction mixture was stirred under reflux for 1 h. The reaction mixture was diluted with EtOAc (10 mL) and NH₄Cl (sat., aq., 100 mL) was added. The layers were separated and the aqueous phase was extracted with EtOAc (2×150 mL). The combined organic layers were washed with water (150 mL) and brine (150 mL), dried (Na₂SO₄), filtered and concentrated under reduced pressure. The crude mixture was purified by flash column chromatography (C-18, mobile phase: H₂O/MeCN, gradient from 80:20 to 60:40). A second purification was performed by flash column chromatography (silica, mobile phase: DCM/MeOH, gradient from 100:0 to 97.5:2.5) to afford intermediate I37 (170 mg, 38%) as a white solid.

### Compound 7

### (3,4-Dichlorophenyl)(3-methyl-2,4,5,8,9,11-hexahydro-10H-pyrazolo[3,4-c]pyrazolo-[1,5-a:4,3-c′]dipyridin-10-yl)methanone

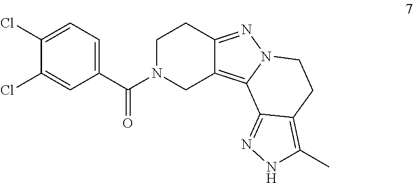 [SMILES: Cc1[nH]nc2c1CCn1nc3c(c1-2)CN(C(=O)c1ccc(Cl)c(Cl)c1)CC3]

The reaction was performed under Ar atmosphere.

Hydrazine monohydrate (401 μL, 8.27 mmol) was added dropwise to a solution of intermediate I37 (168 mg, 0.41 mmol) in EtOH (4 mL) at room temperature. The reaction mixture was stirred at 80° C. for 2 h, concentrated to dryness and co-evaporated with DCM (2×5 mL). The residue was purified by flash column chromatography (C-18, mobile phase: H₂O/MeCN, gradient from 90:10 to 72:28) to give compound 7 (70 mg, 42%) as a pale yellow solid.

¹H NMR (400 MHz, DMSO-d₆, 80° C.) δ ppm 12.31 (br.s, 1H), 7.72-7.67 (m, 2H), 7.44 (dd, J=8.0, 0.8 Hz, 1H), 4.72-4.65 (m, 2H), 4.18 (t, J=6.8 Hz, 2H), 3.81-3.72 (m, 2H), 2.89 (t, J=6.8 Hz, 2H), 2.74 (t, J=5.6 Hz, 2H), 2.22 (s, 3H); LCMS (method E): Rt=8.8 min, m/z calcd. for C₁₉H₁₇Cl₂N₅O 401, m/z found 402 [M+H]⁺.

### 3.1.3.3. Synthesis of Compound 8

### (3,4-Dichlorophenyl)(2-methyl-2,4,5,8,9,11-hexahydro-10H-pyrazolo[3,4-c]pyrazolo-[1,5-a:4,3-c]dipyridin-10-yl)methanone

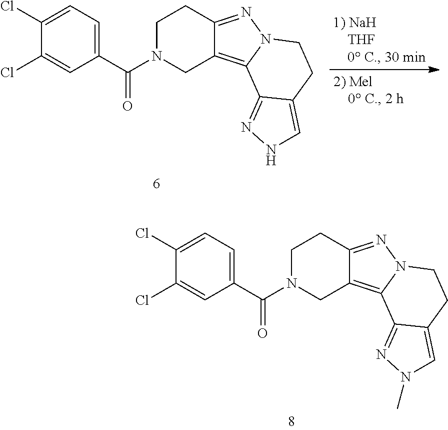 [SMILES: O=C(c1ccc(Cl)c(Cl)c1)N1CCc2nn3c(c2C1)-c1n[nH]cc1CC3 ; Cn1cc2c(n1)-c1c3c(nn1CC2)CCN(C(=O)c1ccc(Cl)c(Cl)c1)C3]

The reaction was performed under anhydrous conditions and under Ar atmosphere.

To a solution of compound 6 (230 mg, 0.41 mmol, 70% purity) in THF (4 mL) was added NaH (60% in mineral oil, 33.3 mg, 0.83 mmol) at 0° C. The reaction mixture was stirred at this temperature for 30 min, then iodomethane (51.9 μL, 0.83 mmol) was added. The reaction mixture was stirred at 0° C. for 2 h, warmed up to room temperature and diluted with water (10 mL). The layers were separated and the aqueous layer was extracted with EtOAc (2×20 mL). The combined organic extracts were dried (Na₂SO₄), filtered and concentrated under vacuum. The crude mixture was combined with another fraction (0.33 mmol) and purified by flash column chromatography (silica, mobile phase: DCM/MeOH, gradient from 100:0 to 90:10). A second purification was performed by preparative HPLC (mobile phase: HO/MeCN, gradient from 50:50 to 0:100). The residue was submitted to another purification by flash column chromatography (silica, mobile phase: DCM/MeOH, gradient from 99:1 to 95:5) to give compound 8 (86.2 mg, 2%).

¹H NMR (400 MHz, DMSO-d₆, 80° C.) δ ppm 7.70 (d, J=8.4 Hz, 1H), 7.69 (d, J=2.0 Hz, 1H), 7.54 (s, 1H), 7.44 (dd, 0.1=8.4, 2.0 Hz, 1H), 4.70 (s, 2H), 4.19 (t, J=7.2 Hz, 2H), 3.86 (s, 3H), 3.77-3.69 (m, 2H), 3.00 (m, 2H), 2.74 (t, J=6.0 Hz, 2H); LCMS (method G): Rt=13.1 min, m/z calcd. for C₁₉H₁₇Cl₂N₅O 401, m/z found 402 [M+H]⁺.

### 3.1.3.4. Synthesis of Compound 9

### (3,4-Dichlorophenyl)(1-methyl-1,4,5,8,9,11-hexahydro-10H-pyrazolo[3,4-c]pyrazolo-[1,5-a:4,3-c′]dipyridin-10-yl)methanone

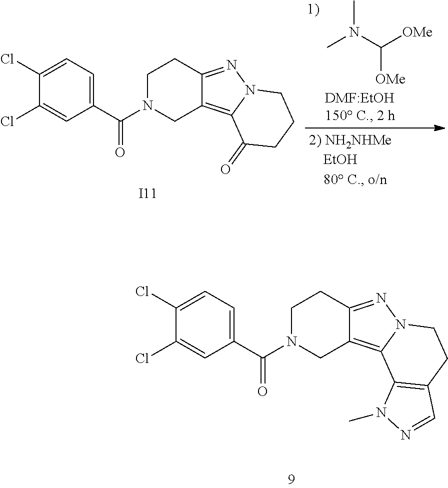

The reaction was performed under anhydrous conditions and under Ar atmosphere.

To a solution of intermediate I11 (400 mg, 1.10 mmol) in DMF (10 mL) was added N,N-dimethylformamide dimethyl acetal (1.02 mL, 7.69 mmol). The reaction mixture was stirred at 150° C. for 2 h, concentrated to dryness and co-evaporated with DCM (2×4 mL). The residue was taken up in EtOH (10 mL) and methylhydrazine (1.16 mL, 22.0 mmol) was added. The reaction mixture was stirred at 80° C. overnight, concentrated under reduced pressure to dryness and co-evaporated with DCM (2×4 mL). The crude mixture was purified by flash column chromatography (silica, mobile phase: DCM/MeOH, gradient from 100:0 to 90:10). A second purification was performed by preparative HPLC (mobile phase: H₂O/MeCN, gradient from 50:50 to 0:100). The residue was triturated in EtAOc, collected by filtration and dried to afford compound 9 (72 mg, 16%) as a white solid.

¹H NMR (400 MHz, DMSO-d₆, 80° C.) δ ppm 7.70 (d, J=8.0 Hz, 1H), 7.69 (d, J=2.0 Hz, 1H), 7.45 (dd, J=8.0, 2.0 Hz, 1H), 7.35 (s, 1H), 4.92 (s, 2H), 4.19 (t, 1=6.8 Hz, 2H), 3.93 (s, 3H), 3.78-3.71 (m, 2H), 2.91 (t, J=7.2 Hz, 2H), 2.76 (1, J=6.0 Hz, 2H); LCMS (method G): Rt=16.9 min, m/z calcd. for C₁₉H₁₇Cl₂N₅O 401, m/z found 402 [M+H]⁺.

### 3.1.4. Synthesis of Thiazole Derivative Compounds

### 3.1.4.1. Synthesis of Compound 10

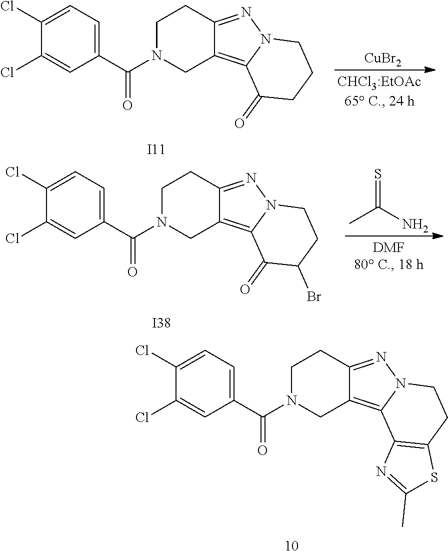

### Intermediate I38

### 9-Bromo-2-(3,4-dichlorobenzoyl)-1,2,3,4,8,9-hexahydropyrazolo[1,5-a:4,3-c′]dipyridin-10(7H)-one

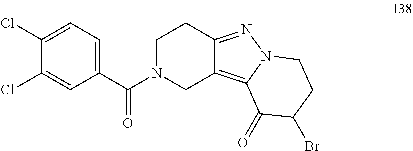 [SMILES: O=C1c2c3c(nn2CCC1Br)CCN(C(=O)c1ccc(Cl)c(Cl)c1)C3]

The reaction was performed under anhydrous conditions and under Ar atmosphere.

To a suspension of CuBr₂ (429 mg, 1.92 mmol) in EtOAc (6 mL) at room temperature was added a solution of intermediate I11 (500 mg, 1.37 mmol) in CHCl₃ (4 mL). The reaction mixture was stirred at 60° C. for 18 h, cooled to room temperature and additional amount of CuBr₂ (61 mg; 0.28 mmol) was added. The reaction mixture was stirred at 65° C. for another 2 h. The addition of CuBr₂ (61 mg, 0.28 mmol) was repeated and the reaction mixture was stirred for 2 h at 65° C. The reaction mixture was slowly added to an EDTA solution and extracted with EtOAc (3×60 mL). The combined organic lavers were washed with brine, dried (Na₂SO₄), filtered, concentrated under reduced pressure to afford intermediate I38 which was used as such in the next step.

### Compound 10

### (3,4-Dichlorophenyl)(2-methyl-4,8,9,11-tetrahydro[1,3]thiazolo[4,5-c]pyrazolo[1,5-a:4,3-c′]-dipyridin-10(5H)-yl)methanone

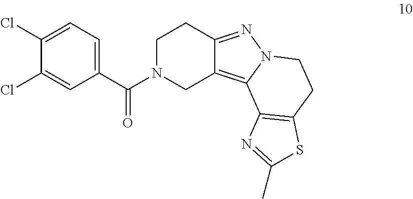 [SMILES: Cc1nc2c(s1)CCn1nc3c(c1-2)CN(C(=O)c1ccc(Cl)c(Cl)c1)CC3]

To a solution of intermediate I38 in DMF (12 mL) was added thioacetamide (81.4 mg, 1.08 mmol). The reaction mixture was stirred at 80° C. for 18 h and diluted with EtOAc and water. The layers were separated and the aqueous phase was extracted with EtOAc (3×60 mL). The combined organic layers were washed with brine, dried (Na₂SO₄), filtered and concentrated under reduced pressure. The crude mixture was purified by flash column chromatography (C-18, mobile phase: H₂O/MeCN, gradient from 85:15 to 0:100). A second purification was performed by flash column chromatography (silica, mobile phase: DCM/MeOH, gradient from 99:1 to 95:5). The residue was co-evaporated with EtOH (3 times), then with a mixture EtOAc/EtOH/DCM (1:1:1) and dried under vacuum at 50° C. to give compound 10 (111 mg, 19% over 2 steps) as a white solid.

¹H NMR (401) MHz, DMSO-d₆, 80° C.) δ ppm 7.72-7.64 (m, 2H), 7.47-7.42 (m, 1H), 4.78 (s, 2H), 4.30 (t, J=7.2 Hz, 2H), 3.78-3.68 (m, 2H), 3.30 (t, J=7.2 Hz, 2H), 2.76 (t, J=6.1 Hz, 2H), 2.68 (s, 3H); LCMS (method E): Rt=10.1 min, m/z calcd. for C₁₉H₁₆Cl₂N₄OS 418, m/z found 419 [M+H]⁺.

### 3.2. Synthesis of the 7-Membered Rings

### 3.2.1. Synthesis of Intermediates

### 3.2.1.1. Synthesis of Intermediate I39

### 5-tert-Butyl 3-ethyl 2-(pent-4-yn-1-yl)-2,4,6,7-tetrahydro-5H-pyrazolo[4,3-c]pyridine-3,5-dicarboxylate

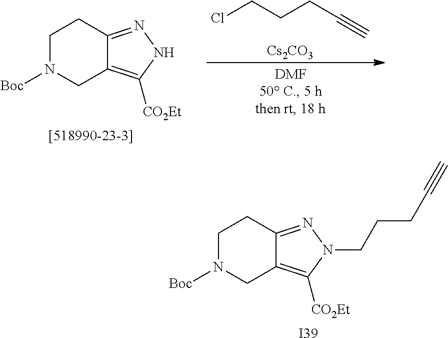

The reaction was performed under anhydrous conditions and under Ar atmosphere.

To a suspension of Cs₂CO₃ (6.62 g, 20.3 mmol) in DMF (60 mL) were successively added 5-tert-butyl 3-ethyl 2,4,6,7-tetrahydro-5H-pyrazolo[4,3-c]pyridine-3,5-dicarboxylate (3.00 g, 10.2 mmol) and 5-chloro-1-pentyne (2.15 mL, 20.3 mmol) at room temperature. The reaction mixture was stirred at 50° C. for 5 h then at room temperature for 18 h. The reaction mixture was poured into water (100 mL) and extracted with EtOAc (3×70 mL). The combined organic extracts were washed with brine (3×100 mL), dried (Na₂SO₄), filtered and concentrated under reduced pressure. The crude mixture was purified by flash column chromatography (C-18, mobile phase: H₂O/MeCN, gradient from 75:25 to 5:95) to give intermediate I39 (2.25 g, 57%, 93% purity) as a pale yellow oil.

### 3.2.1.2. Synthesis of Intermediate I40

### 5-tert-Butyl 3-ethyl 2-(hex-4-yn-1-yl)-2,4,6,7-tetrahydro-5H-pyrazolo[4,3-c]pyridine-3,5-dicarboxylate

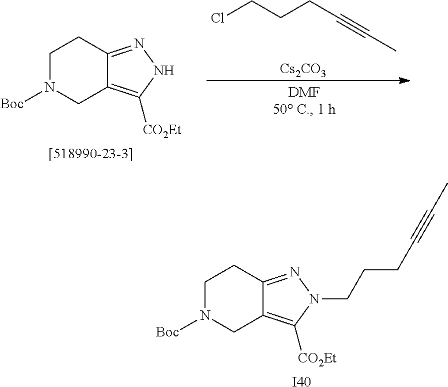

The reaction was performed under anhydrous conditions and under Ar atmosphere.

To a solution of 5-tert-butyl 3-ethyl 2,4,6,7-tetrahydro-5H-pyrazolo[4,3-c]pyridine-3,5-dicarboxylate (4.00 g, 13.5 mmol) in DMF (80 mL) were added Cs₂CO₃ (13.2 g, 40.6 mmol) and 6-chlorohex-2-yne (7.52 g, 40.6 mmol). The reaction mixture was stirred at 50° C. for 1 h and diluted with H₂O (100 mL). The layers were separated and the aqueous phase was extracted with EtOAc (3×100 mL). The combined organic layers were washed with brine (3×100 mL), dried (Na₂SO₄), filtered and concentrated to dryness. The crude mixture was purified by flash column chromatography (C-18, mobile phase: H₂O/MeCN, gradient from: 90:10 to 50:50) to afford intermediate I40 (1.79 g, 35%) as a yellow oil.

### 3.2.1.3. Synthesis of Intermediate I41

### 5-tert-Butyl 3-ethyl 2-(hept-4-yn-1-yl)-2,4,6,7-tetrahydro-5H-pyrazolo[4,3-c]pyridine-3,5-dicarboxylate

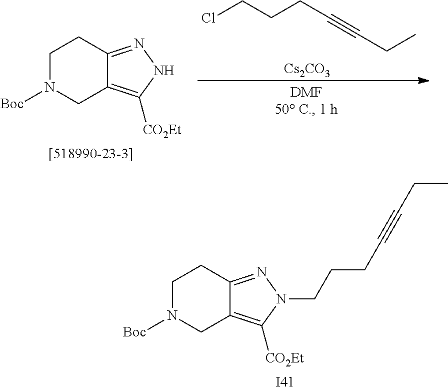

Intermediate I41 (850 mg, 32%) was prepared in an analogous manner to that described for intermediate I40.

### 3.2.1.4. Synthesis of Intermediate I42

### 5-tert-Butyl 3-ethyl (6R)-6-methyl-2-(pent-4-yn-1-yl)-2,4,6,7-tetrahydro-5H-pyrazolo[4,3-c]-pyridine-3,5-dicarboxylate

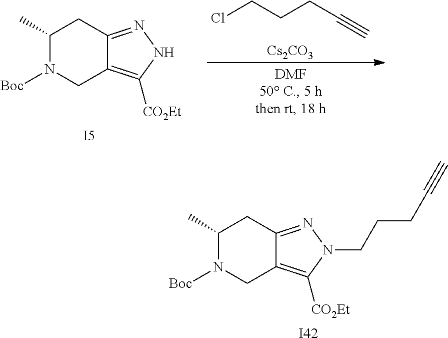

Intermediate I42 (1.17 g, 45%, 94% purity) was prepared in an analogous manner to that described for intermediate I39.

### 3.2.1.5. Synthesis of Intermediate I43

### 5-tert-Butyl 3-ethyl (6R)-2-(hex-4-yn-1-yl)-6-methyl-2,4,6,7-tetrahydro-5H-pyrazolo[4,3-c]-pyridine-3,5-dicarboxylate

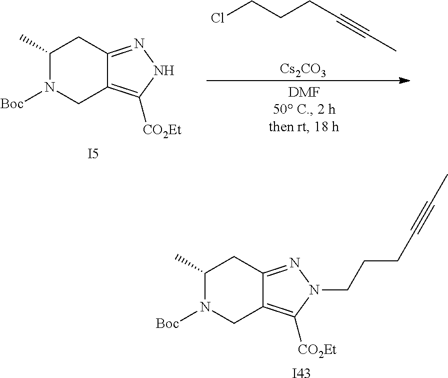

Intermediate I43 (632 mg, 43%, 86% purity) was prepared in an analogous to that described for intermediate I39.

### 3.2.1.6. Synthesis of Intermediate I51

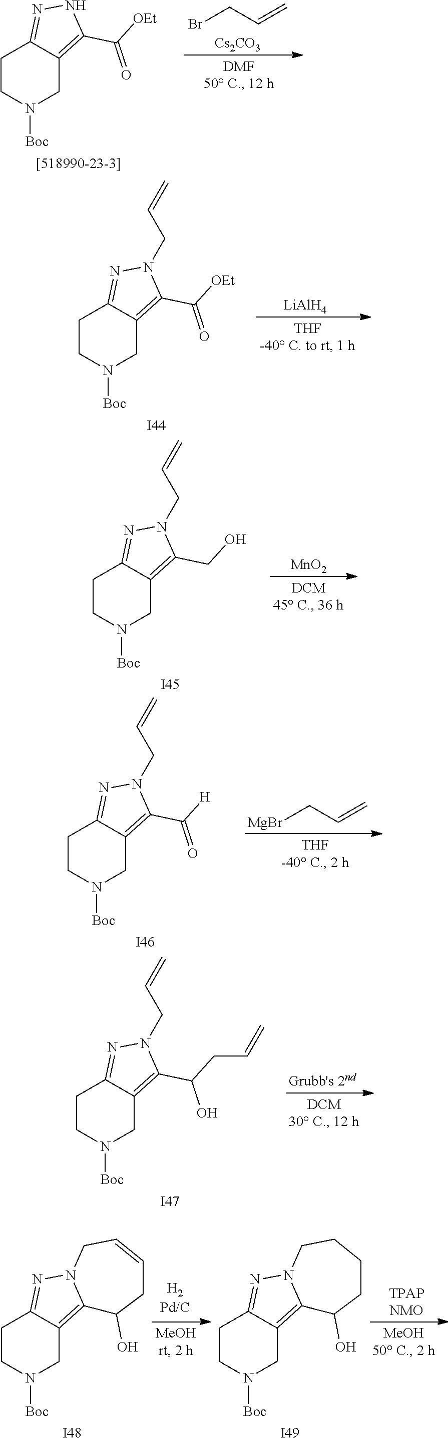

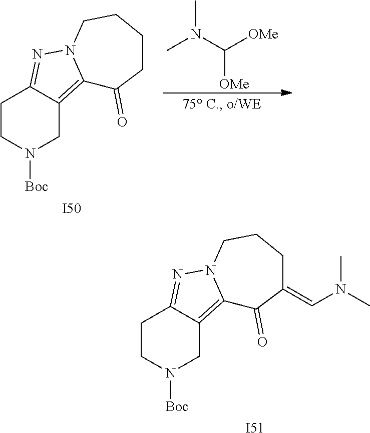

### Intermediate I44

### 5-tert-Butyl 3-ethyl 2-allyl-6,7-dihydro-2H-pyrazolo[4,3-c]pyridine-3,5(4H)-dicarboxylate

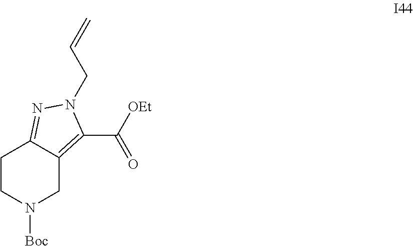 [SMILES: C=CCn1nc2c(c1C(=O)OCC)CN(C(=O)OC(C)(C)C)CC2]

To a mixture of 5-tert-butyl 3-ethyl 6,7-dihydro-2H-pyrazolo[4,3-c]pyridine-3,5(4H)-dicarboxylate (5.00 g, 16.9 mmol) and 3-bromoprop-1-ene (3.07 g, 25.4 mmol) in DMF (50 mL) was added Cs₂CO₃ (13.8 g, 42.3 mmol) in one portion under N₂ atmosphere. The mixture was stirred at 50° C. for 12 h and poured into water (50 mL). The mixture was stirred for 1 min and the aqueous phase was extracted with EtOAc (2×50 mL). The combined organic extracts were washed with brine (2×50 mL), dried (Na₂SO₄), filtered and concentrated under reduced pressure. The crude mixture was purified by flash column chromatography (silica, petroleum ether/EtOAc, gradient from 15:1 to 5:1) to give intermediate I44 (2.70 g, 47%) as a yellow solid.

### Intermediate I45

### tert-Butyl 2-allyl-3-(hydroxymethyl)-6,7-dihydro-2H-pyrazolo[4,3-c]pyridine-5(4H)-carboxylate

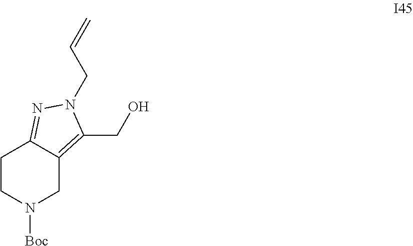 [SMILES: C=CCn1nc2c(c1CO)CN(C(=O)OC(C)(C)C)CC2]

To a mixture of intermediate I44 (1.70 g, 5.07 mmol) in THF (30.00 mL) was added LiAlH₄ (288 mg, 7.60 mmol) in one portion at −40° C. under N2 atmosphere. The reaction mixture was stirred at 20° C. for 1 h and poured into water (10 mL). The mixture was stirred for 1 min and the aqueous phase was extracted with EtOAc (2×30 mL). The combined organic extracts were washed with brine (2×10 mL), dried (Na₂SO₄), filtered and concentrated under reduced pressure. The residue was purified by flash column chromatography (silica. DCM/MeOH, gradient from 50:1 to 20:1) to afford intermediate I45 (1.10 g, 72%) as a yellow solid.

### Intermediate I46

### tert-Butyl 2-allyl-3-formyl-6,7-dihydro-2H-pyrazolo[4,3-c]pyridine-5(4H)-carboxylate

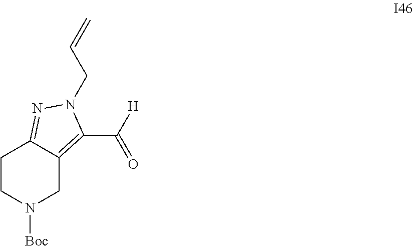 [SMILES: C=CCn1nc2c(c1C=O)CN(C(=O)OC(C)(C)C)CC2]

To a mixture of intermediate I45 (1.10 g, 3.75 mmol) in DCM (10.00 mL) was added MnO₂ (3.26 g, 37.5 mmol) in one portion under N2 atmosphere. The reaction mixture was stirred at 45° C. for 12 h. Additional quantity of MnO₂ (3.26 g, 37.5 mmol) was added and the reaction mixture was stirred at 45° C. for another 24 h. The mixture was filtered and the filtrate was concentrated under reduced pressure. The crude mixture was purified by flash column chromatography (silica, petroleum ether/EtOAc, gradient from 10:1 to 5:1) to afford intermediate I46 (620 mg, 57%) as yellow a solid.

### Intermediate I47

### tert-Butyl 2-allyl-3-(1-hydroxybut-3-en-1-yl)-6,7-dihydro-2H-pyrazolo [4,3-c]pyridine-5(4H)-carboxylate

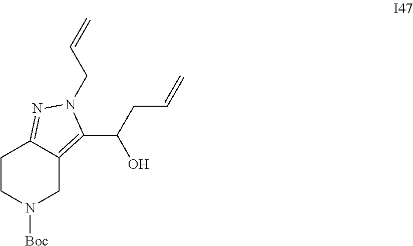 [SMILES: C=CCC(O)c1c2c(nn1CC=C)CCN(C(=O)OC(C)(C)C)C2]

To a mixture of intermediate I46 (800 mg, 2.75 mmol) in THF (5.00 mL) was added allylmagnesium bromide (1M in THF, 8.24 mL, 8.24 mmol) in one portion at −40° C. under N2 atmosphere. The reaction mixture was stirred at −40° C. for 2 h and poured into water (20 mL). The mixture was stirred for 1 min and the aqueous phase was extracted with EtAOc (2×20 ml). The combined organic extracts were washed with brine (2×10 mL), dried (Na₂SO₄), filtered and concentrated under reduced pressure. The crude mixture was purified by flash column chromatography (silica, petroleum ether/EtOAc, gradient from 3:1 to 1:1) to afford intermediate 147 (750 mg, 79%) as a yellow oil.

### Intermediate I48

### tert-Butyl 11-hydroxy-3,4,10,11-tetrahydro-1H-pyrido[4′,3′:3,4] Pyrazolo[1,5-a]azepine-2(7H)-carboxylate

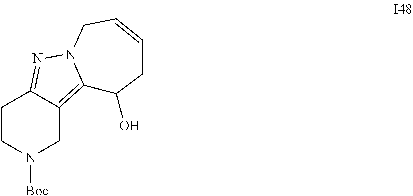 [SMILES: CC(C)(C)OC(=O)N1CCc2nn3c(c2C1)C(O)CC=CC3]

To a mixture of intermediate I47 (750 mg, 2.25 mmol) in DCM (1.20 L) was added Grubbs' 2^{nd}(382 mg, 445 μmol) in one portion under N₂ atmosphere. The reaction mixture was stirred at 30° C. for 12 h and concentrated under reduced pressure. The crude mixture was purified by flash column chromatography (silica, petroleum ether/EtOAc, gradient from 4:1 to 1:1) to afford intermediate 148 (650 mg, 90%) as a yellow solid.

### Intermediate I49

### tert-Butyl 11-hydroxy-1,3,4,7,8,9,10,11-octahydro-2H-pyrido[4′,3′:3,4]pyrazolo[1,5-a]azepine-2-carboxylate

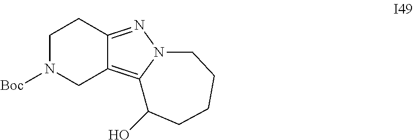 [SMILES: CC(C)(C)OC(=O)N1CCc2nn3c(c2C1)C(O)CCCC3]

Intermediate I48 (2.31 g, 7.56 mmol) was dissolved in MeOH (100 mL). Pd/C (10%, 697 mg, 0.65 mmol) was added and the reaction mixture was stirred under H₂ atmosphere for 2 h. The reaction mixture was filtered and the volatiles were removed under reduced pressure. The residue was purified by flash column chromatography (silica, mobile phase gradient: heptane to EtOAc) to afford intermediate I49 (1.94, 83%) as a white foam.

### Intermediate I50

### tert-Butyl 11-oxo-1,3,4,7,8,9,10,11-octahydro-2H-pyrido[4′,3′:3,4]pyrazolo[1,5-a]azepine-2-carboxylate

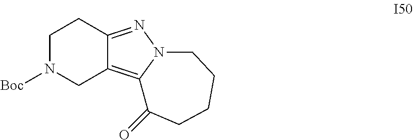 [SMILES: CC(C)(C)OC(=O)N1CCc2nn3c(c2C1)C(=O)CCCC3]

A mixture of intermediate I49 (1.89 g, 6.15 mmol). TPAP (432 mg, 1.23 mmol) and NMO (3.32 g, 24.6 mmol) in MeCN (75 mL) was stirred at 50° C. for 2 h. The volatiles were removed under reduced pressure and the residue was purified by flash column chromatography (silica, mobile phase gradient: heptane to EtOAc) to afford intermediate I50 (1.79 g, 95%).

### Intermediate I51

### tert-Butyl (10E)-10-[(dimethylamino)methylidene]-11-oxo-1,3,4,7,8,9,10,11-octahydro-2H-pyrido[4′,3′:3,4]pyrazolo[1,5-a]azepine-2-carboxylate

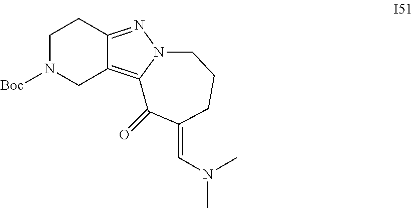 [SMILES: CN(C)/C=C1\CCCn2nc3c(c2C1=O)CN(C(=O)OC(C)(C)C)CC3]

Intermediate I50 (1.79 g, 5.86 mmol) in N,N-dimethylformamide dimethyl acetal (15 mL) was stirred at 75° C. for 72 h. The reaction mixture was diluted with water (20 mL) and the mixture was stirred vigorously for 1 h. The layers were separated and the aqueous phase was extracted with EtOAc (2×20 mL). The combined organic phases were dried (MgSO₄), filtered and evaporated to dryness to afford intermediate I51 (2.07 g, 98%) as a yellow oil.

### 3.2.1.7. Synthesis of Intermediates I62 and I63

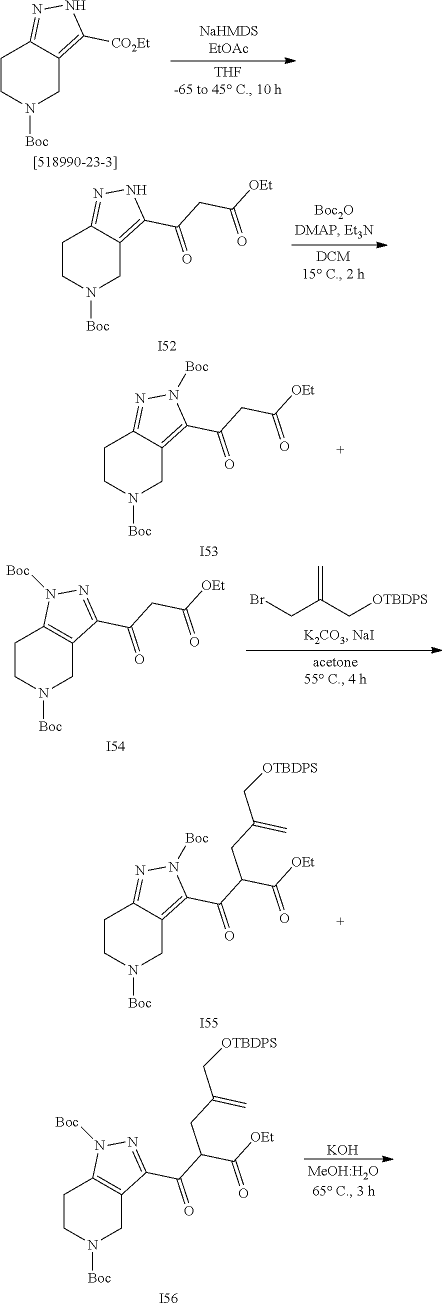

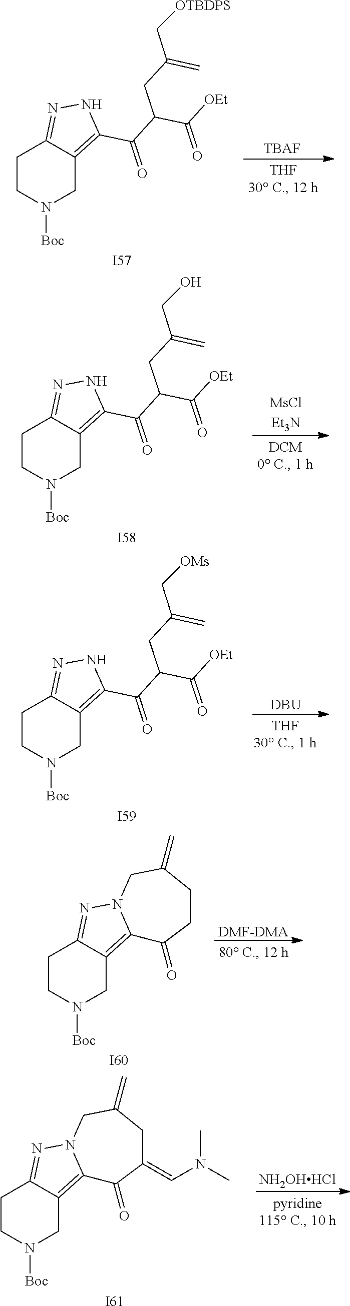

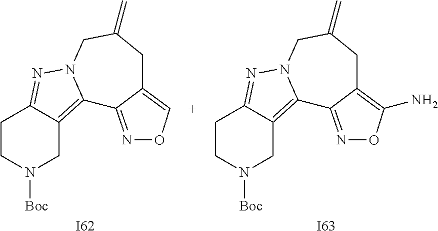 [SMILES: "I62" ; "I63" ; C=C1Cc2conc2-c2c3c(nn2C1)CCN(C(=O)OC(C)(C)C)C3 ; C=C1Cc2c(noc2N)-c2c3c(nn2C1)CCN(C(=O)OC(C)(C)C)C3]

### Intermediate I52

### Tert-Butyl 3-(3-ethoxy-3-oxopropanoyl)-6,7-dihydro-2H-pyrazolo[4,3-c] pyridine-5(4H)-carboxylate

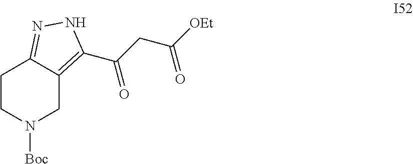 [SMILES: CCOC(=O)CC(=O)c1[nH]nc2c1CN(C(=O)OC(C)(C)C)CC2]

To a solution of ethyl acetate (20.9 g, 237 mmol) in THF (120 mL) was added NaHMDS (1M in THF, 474 mL, 474 mmol) at −65° C. under N2 atmosphere. A solution of 5-tert-butyl 3-ethyl 6,7-dihydro-2H-pyrazolo[4,3-c]pyridine-3,5(4H)-dicarboxylate (28.0 g, 94.8 mmol) in THF (200 mL) was added dropwise into the mixture over 1 h at −65° C. The reaction mixture was stirred at 45° C. for 10 h and quenched with HCl (1N, 1.5 L). The aqueous phase was extracted with EtOAc (1.5 L). The organic phase was dried (Na₂SO₄), filtered and concentrated under reduced pressure. The crude mixture was purified by flash column chromatography (silica, petroleum ether/EtOAc, gradient from 10:1 to 1:1) to give intermediate I52 (28.4 g, 89%) as a yellow solid.

### Intermediates I53 and I54

### Di-tert-butyl 3-(3-ethoxy-3-oxopropanoyl)-6,7-dihydro-2H-pyrazolo[4,3-c]pyridine-2,5(4H)-dicarboxylate and Di-tert-butyl 3-(3-ethoxy-3-oxopropanoyl)-6,7-dihydro-1H-pyrazolo[4,3-c]-pyridine-1,5(4H)-dicarboxylate

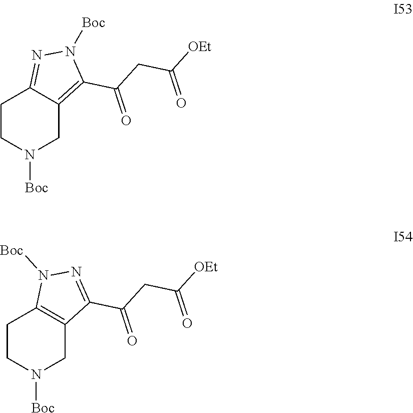 [SMILES: CCOC(=O)CC(=O)c1c2c(nn1C(=O)OC(C)(C)C)CCN(C(=O)OC(C)(C)C)C2 ; CCOC(=O)CC(=O)c1nn(C(=O)OC(C)(C)C)c2c1CN(C(=O)OC(C)(C)C)CC2]

To a mixture of intermediate I52 (18.0 g, 53.4 mmol), Et₃N (16.2 g, 160) mmol) and DMAP (652 mg, 5.34 mmol) in DCM (200 mL) was added Boc₂O (11.6 g, 53.4 mmol). The reaction mixture was stirred at 15° C. for 2 h and poured into HCl (1N, 250 mL). The mixture was extracted with EtOAc (2×200 mL). The combined organic extracts were washed with brine (200 mL), dried (Na₂SO₄), filtered and concentrated under reduced pressure. The residue was purified by flash column chromatography (silica, petroleum ether/EtOAc, gradient from 100:0 to 80:20) to afford a mixture of intermediates 153 and 154 (20 g, 43%) as a colorless oil.

### Intermediates I55 and I56

### Di-tert-butyl3-(4-(((tert-butyldiphenylsilyl)oxy)methyl)-2-(ethoxycarbonyl)pent-4-enoyl)-6,7-dihydro-2H-pyrazolo[4,3-c]pyridine-2,5(4H)-dicarboxylate and Di-tert-butyl3-(4-(((tert-butyl-diphenylsilyl)oxy)methyl)-2-(ethoxycarbonyl)pent-4-enoyl)-6,7-dihydro-1H-pyrazolo[4,3-c]-pyridine-1,5(4H)-dicarboxylate

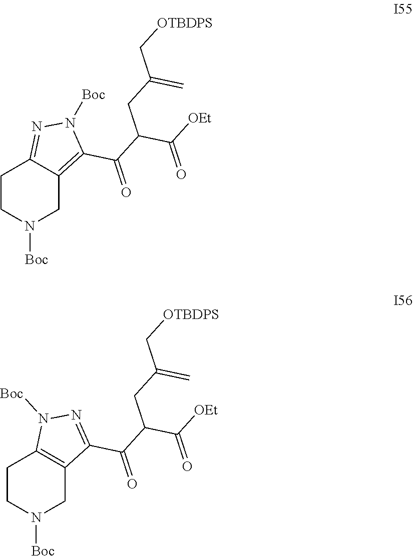

To a mixture of intermediates 153 and 154 (14.0 g, 32.0 mmol) in acetone (150 mL) were added K₂CO₃ (6.64 g, 48.1 mmol), NaI (960 mg, 6.41 mmol) and 2-(bromomethyl)allyloxy-tert-butyl-diphenyl silane (15.0 g, 38.4 mmol). The reaction mixture was stirred at 55° C. for 4 h and poured into HCl (1N, 400 mL) at 0° C. The mixture was extracted with EtOAc (3×300 mL). The combined organic extracts were washed with brine (500 mL), dried (Na₂SO₄), filtered and concentrated under reduced pressure. The crude mixture was purified by flash column chromatography (silica, petroleum ether/EtOAc, gradient from 30:1 to 20:1) to afford a mixture of intermediates 155 and 156 (13.5 g, 53%) as a yellow oil.

### Intermediate I57

### tert-Butyl 3-(4-(((tert-butyldiphenylsilyl)oxy)methyl)pent-4-enoyl)-6,7-dihydro-2H-pyrazolo-[4,3-c]pyridine-5(4H)-carboxylate

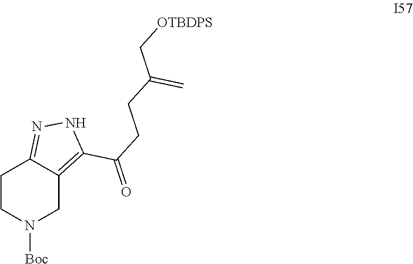

To a mixture of intermediates 155 and 156 (13.5 g, 16.8 mmol) in MeOH (50 mL) was added a solution of KOH (1.89 g, 33.7 mmol) in H₂O (10 mL). The reaction mixture was stirred at 65° C. for 3 h and poured into HCl (1N, 30) mL). The mixture was extracted with EtOAc (3×200 mL). The combined organic extracts were washed with brine (200 mL), dried (Na₂SO₄), filtered and concentrated under reduced pressure. The crude mixture was purified by flash column chromatography (silica, petroleum ether/EtOAc, 3/1) to give intermediate I57 (8.9 g, 92%) as a yellow oil.

### Intermediate I58

### Tert-Butyl 3-(4-(hydroxymethyl)pent-4-enoyl)-6,7-dihydro-2H-pyrazolo[4,3-c]pyridine-5(4H)-carboxylate

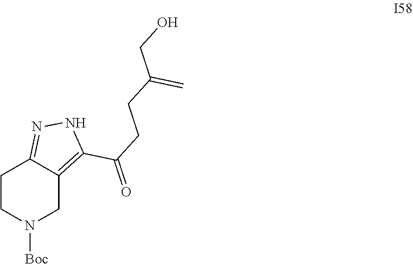 [SMILES: C=C(CO)CCC(=O)c1[nH]nc2c1CN(C(=O)OC(C)(C)C)CC2]

To a solution of intermediate I57 (14.0 g, 22.0 mmol) in THF (50 mL) was added TBAF (1M in THF, 32.9 mL, 32.9 mmol). The reaction mixture was stirred at 30° C. for 12 h and poured into H₂O (100 mL). The aqueous phase was extracted with EtOAc (3×80 mL). The combined organic extracts were washed with brine (100 mL), dried (Na₂SO₄), filtered and concentrated under reduced pressure. The crude mixture was purified by flash column chromatography (silica, petroleum ether/EtOAc, gradient from 2:1 to 1:1) to give intermediate 58 (6.3 g, 84%) as a white solid.

### Intermediate I59

### Tert-Butyl 3-(4-(((methylsulfonyl)oxy)methyl)pent-4-enoyl)-6,7-dihydro-2H-pyrazolo[4,3-c]-pyridine-5(4H)-carboxylate

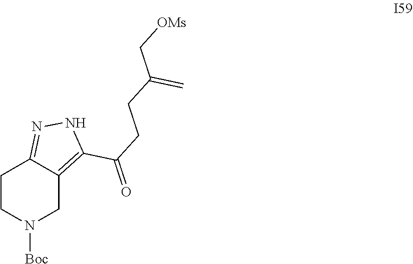 [SMILES: C=C(CCC(=O)c1[nH]nc2c1CN(C(=O)OC(C)(C)C)CC2)COS(C)(=O)=O]

To a mixture of intermediate I58 (6.30 g, 18.4 mmol) and Et₃N (5.59 g, 55.2 mmol) in DCM (30 mL) was added MsCl (4.73 g, 41.3 mmol) at 0° C. under N₂ atmosphere. The reaction mixture was stirred at 0° C. for 1 h and poured into water (60 mL). The aqueous phase was extracted with EtOAc (3×60 mL). The combined organic extracts were washed with brine (60 mL), dried (Na₂SO₄), filtered and concentrated under reduced pressure to afford intermediate I59 which was used as such in the next step.

### Intermediate I60

### Tert-Butyl 8-methylene-11-oxo-3,4,8,9,10,11-hexahydro-1H-pyrido[4′,3′:3,4]pyrazolo[1,5-a]-azepine-2(7H)-carboxylate

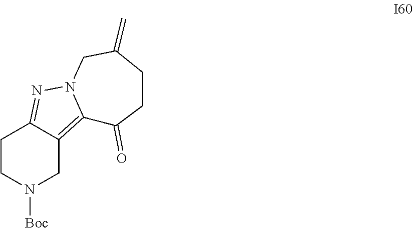 [SMILES: C=C1CCC(=O)c2c3c(nn2C1)CCN(C(=O)OC(C)(C)C)C3]

To a solution of intermediate I59 in THF (60 mL) was added DBU (7.06 g, 46.4 mmol) at 30° C. under N₂ atmosphere. The reaction mixture was stirred at 30° C. for 1 h and poured into water (50 mL). The aqueous phase was extracted with EtOAc (3×50 mL). The combined organic extracts were washed with brine (50 m), dried (Na₂SO₄), filtered and concentrated under reduced pressure. The crude mixture was purified by flash column chromatography (silica, petroleum ether/EtOAc, gradient from 10:1 to 8:1) to afford intermediate I60 (4.2 g, 61% over 2 steps, 85% purity) as colorless oil.

### Intermediate I61

### (Z)-tert-Butyl 10-(dimethylamino)methylene)-8-methylene-1-oxo-3,4,8,9,10,11-hexahydro-1H-pyrido[4′,3′:3,4]pyrazolo[1,5-a]azepine-2(7H)-carboxylate

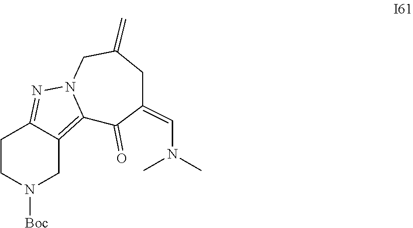 [SMILES: C=C1C/C(=C/N(C)C)C(=O)c2c3c(nn2C1)CCN(C(=O)OC(C)(C)C)C3]

A solution of intermediate I60 (4.20 g, 11.3 mmol) in DMF-DMA (15 mL) was stirred at 80° C. for 12 h and concentrated under reduced pressure. The residue was poured into water (30 mL) and extracted with EtOAc (2×20 mL). The combined organic extracts were washed with brine (2×20 mL), dried (Na₂SO₄), filtered and concentrated under reduced pressure to afford intermediate I61 (4.5 g) which was used as such in the next step.

### Intermediate I62 and I63

### Tert-Butyl 5-methylene-5,6,9,10-tetrahydro-4H-isoxazolo[3,4-c]pyrido[4′,3′:3,4]pyrazolo-[1,5-a]azepine-11(12H)-carboxylate and tert-Butyl 3-amino-5-methylene-5,6,9,10-tetrahydro-4H-isoxazolo[3,4-c]pyrido[4′,3′:3,4]pyrazolo[1,5-a]azepine-11(12H)-carboxylate

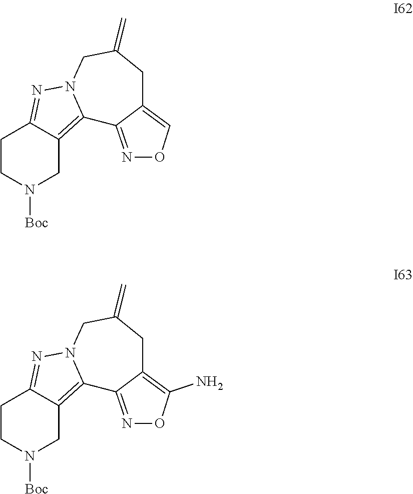 [SMILES: C=C1Cc2conc2-c2c3c(nn2C1)CCN(C(=O)OC(C)(C)C)C3 ; C=C1Cc2c(noc2N)-c2c3c(nn2C1)CCN(C(=O)OC(C)(C)C)C3]

To a solution of intermediate I61 (2.4 g, crude) in pyridine (25 mL) was added hydroxylamine hydrochloride (2.24 g, 32.2 mmol). The reaction mixture was stirred at 115° C. for 10 h and concentrated under reduced pressure. The residue was diluted with H₂O (20 mL) and extracted with EtOAc (3×20 mL). The combined organic extracts were washed with brine (20 mL), dried (Na₂SO₄), filtered and concentrated under reduced pressure. The crude mixture was purified by flash column chromatography (silica, petroleum ether/EtOAc, gradient from 10:1 to 1:1) to afford intermediate I62 (1.4 g, 93% purity) as a white solid and intermediate I63 (0.9 g) as a yellow solid.

### 3.2.1.9. Synthesis of Intermediate I64

### tert-Butyl 5-(hydroxymethyl)-5,6,9,10-tetrahydro-4H-isoxazolo[3,4-c]pyrido[4′,3′:3,4]pyrazolo-[1,5-a]azepine-11(12H)-carboxylate

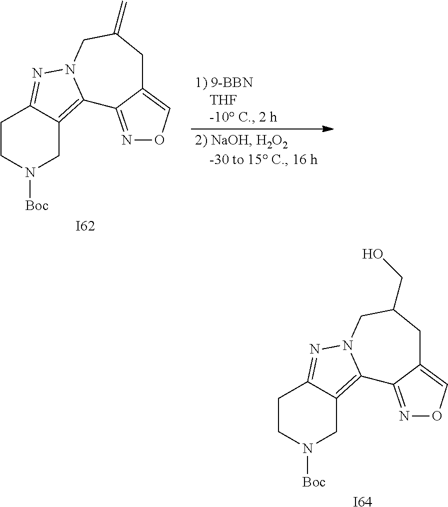 [SMILES: "I62" ; "I64" ; C=C1Cc2conc2-c2c3c(nn2C1)CCN(C(=O)OC(C)(C)C)C3 ; CC(C)(C)OC(=O)N1CCc2nn3c(c2C1)-c1nocc1CC(CO)C3]

To a solution of intermediate I62 (480 mg, 1.40 mmol) in THF (5 mL) was added 9-BBN (0.5 M in THF, 56.1 mL, 23 mmol) at −10° C. The reaction mixture was stirred at −10° C. for 2 h and a solution of NaOH (561 mg, 14.0 mmol) in H₂O (5 mL) was added at −30° C. followed by H₂O₂ (30% purity, 3.18 g, 28.0 mmol). The reaction mixture was stirred at 15° C. for 16 h. The reaction was quenched with NaHSO₃ (sat., aq., 50 mL) and extracted with EtOAc (3×80 mL). The combined organic extracts were dried (Na₂SO₄), filtered and concentrated under reduced pressure. The crude mixture was purified by flash column chromatography (silica, petroleum ether/EtOAc, gradient from 100:0 to 50:50) to afford intermediate I64 (460 mg, 88%) as a white solid.

### 3.2.1.10. Synthesis of Intermediate I67

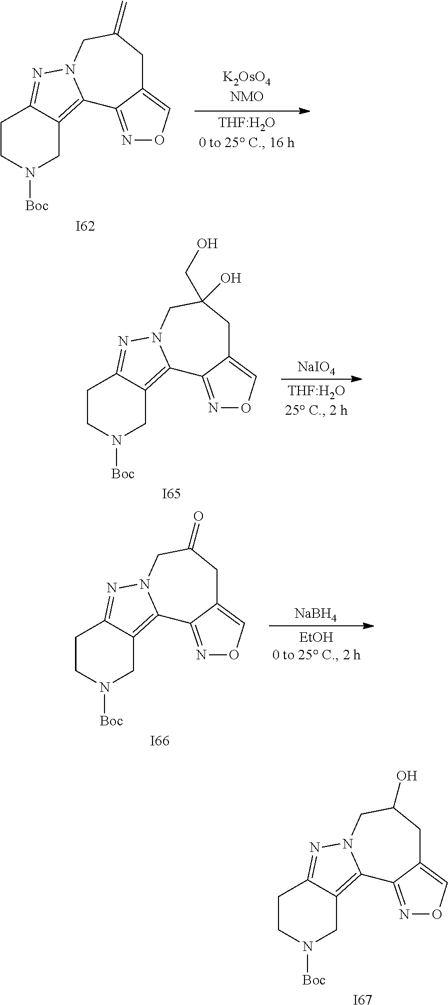

### Intermediate I65

### tert-Butyl 5-hydroxy-5,6,9,10-tetrahydro-4H-isoxazolo[3,4-c]pyrido[4′,3′:3,4]pyrazolo[1,5-a]-azepine-11-(12H)-carboxylate

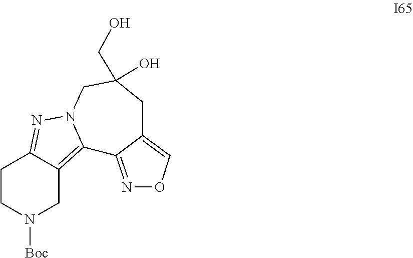 [SMILES: CC(C)(C)OC(=O)N1CCc2nn3c(c2C1)-c1nocc1CC(O)(CO)C3]

To a solution of intermediate I62 (300 mg, 0.88 mmol) in THF (20 mL) and H₂O (10 mL) were added NMO (154 mg, 1.31 mmol) and K₂OsO₄.2H₂O (32.3 mg, 87.6 μmol) at 0° C. The reaction mixture was stirred at room temperature for 16 h. Additional quantity of NMO (154 mg) and K₂OsO₄.2H₂O (50 mg) were added and the reaction mixture was stirred at room temperature for another 16 h. The mixture was diluted with water (20 mL) and extracted with EtOAc (3×20 mL). The combined organic extracts were washed with NaHSO₃ (sat., aq., 3×20 mL), dried (Na₂SO₄), filtered and concentrated under reduced pressure to afford intermediate I65 (334 mg) which was used as such in the next step.

### Intermediate I66

### Tert-Butyl 5-oxo-5,6,9,10-tetrahydro-4H-isoxazolo[3,4-c]pyrido[4′,3′:3,4]pyrazolo[1,5-a]-azepine-11(12H)-carboxylate

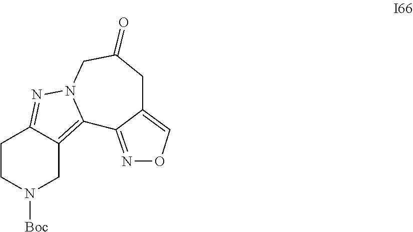 [SMILES: CC(C)(C)OC(=O)N1CCc2nn3c(c2C1)-c1nocc1CC(=O)C3]

To a solution of intermediate I65 in THF (3.3 mL) and H₂O (3.3 mL) was added NaIO₄ (563 mg, 2.63 mmol). The reaction mixture was stirred at room temperature for 2 h and diluted with water (50 mL). The layers were separated and the aqueous phase was extracted with EtOAc (2×40 mL). The combined organic extracts were dried (Na₂SO₄), filtered and concentrated under reduced pressure to afford intermediate I66 (320 mg) which was used as such in the next step.

### Intermediate I67

### tert-Butyl 5-hydroxy-4,5,6,9,10-tetrahydro-4H-isoxazolo[3,4-c]pyrido[4′,3′:3,4]pyrazolo[1,5-a]-azepine-11(12H)-carboxylate

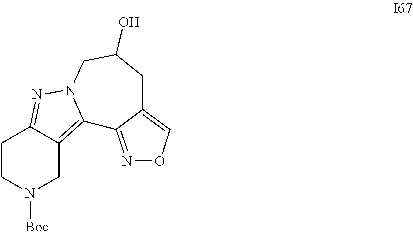 [SMILES: CC(C)(C)OC(=O)N1CCc2nn3c(c2C1)-c1nocc1CC(O)C3]

To a solution of intermediate I66 in EtOH (3 mL) was added NaBH₄ (65.9 mg, 1.74 mmol) at 0° C. The reaction mixture was stirred at room temperature for 2 h and quenched with NH₄Cl (sat., aq., 20 mL). The layers were separated and the aqueous phase was extracted with EtOAc (3×40 mL). The combined organic extracts were dried (Na₂SO₄), filtered and concentrated under reduced pressure to afford intermediate I67 (230 mg) which was used as such in the next step.

### 3.2.1.11. Synthesis of Intermediate I68

### Tert-Butyl 5-methylene-4,5,6,9,10,12-hexahydropyrazolo[3,4-c]pyrido[4′,3′:3,4]pyrazolo[1,5-a]-azepine-11(2H)-carboxylate

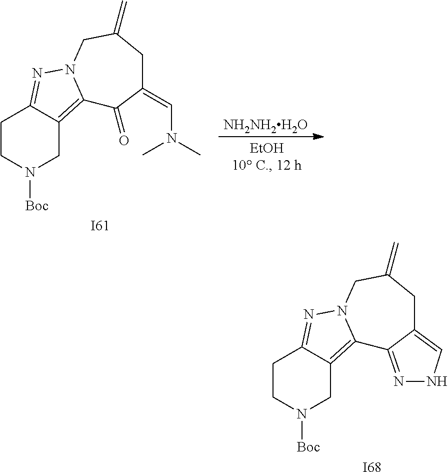

To a solution of intermediate I61 (1.4 g) in EtOH (20 mL) was added hydrazine (376 mg, 7.37 mmol). The reaction mixture was stirred at 10° C. for 12 h and poured into HCl (1N, 40 mL). The mixture was stirred for 1 min and the aqueous phase was extracted with EtAOc (2×40) mL). The combined organic extracts were washed with brine (2×30 mL), dried (Na₂SO₄), filtered and concentrated under reduced pressure. The crude mixture was purified by flash column chromatography (silica, petroleum ether/EtOAc, gradient from 5:1 to 1:1) to give intermediate I68 (1.02 g) as a white solid.

### 3.2.1.11. Synthesis of Intermediate I74

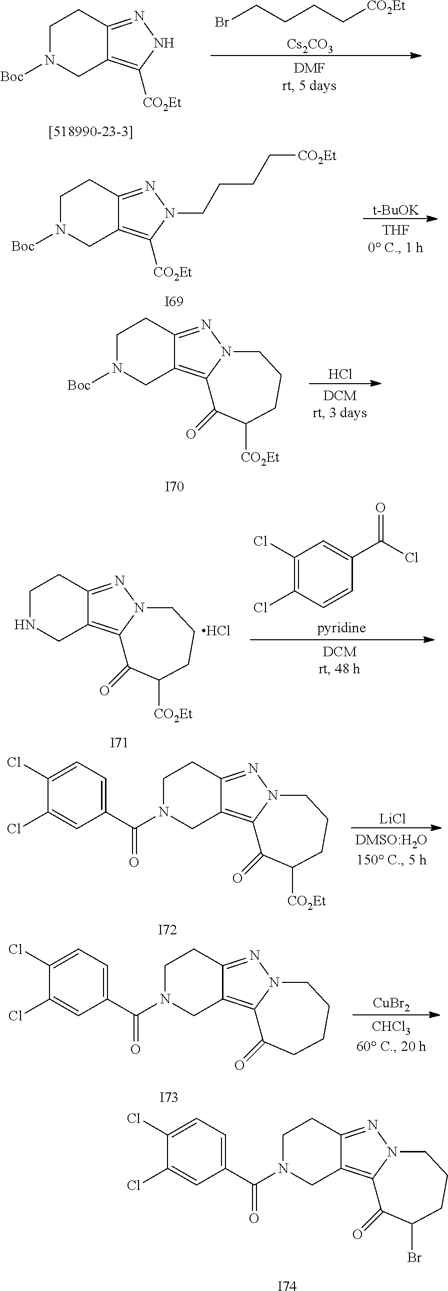

### Intermediate I69

### 5-tert-Butyl 3-ethyl 2-(5-ethoxy-5-oxopentyl)-2,4,6,7-tetrahydro-5H-pyrazolo[4,3-c]pyridine-3,5-dicarboxylate

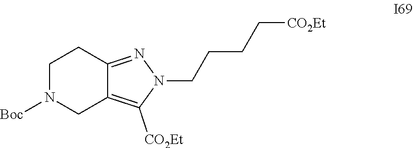 [SMILES: CCOC(=O)CCCCn1nc2c(c1C(=O)OCC)CN(C(=O)OC(C)(C)C)CC2]

The reaction was performed under anhydrous conditions and under Ar atmosphere.

To a suspension of Cs₂CO₃ (16.2 g, 49.6 mmol) in DMF (120 mL) were successively added 5-tert-butyl 3-ethyl 2,4,6,7-tetrahydro-5H-pyrazolo[4,3-c]pyridine-3,5-dicarboxylate (12.2 g, 41.3 mmol) and ethyl 5-bromovalerate (7.19 mL, 45.4 mmol). The reaction mixture was stirred at 110) room temperature for 5 days, poured into water (150 mL) and extracted with EtOAc (2×150 mL). The combined organic extracts were washed with brine (3×150 mL), dried (Na₂SO₄), filtered and concentrated under to dryness to give intermediate I69 as a yellow oil which was engaged in the next step as such.

### Intermediate I70

### 2-tert-Butyl 10-ethyl 11-oxo-1,3,4,7,8,9,10,11-octahydro-2H-pyrido[4′,3′:3,4]pyrazolo[1,5-a]-azepine-2,10-dicarboxylate

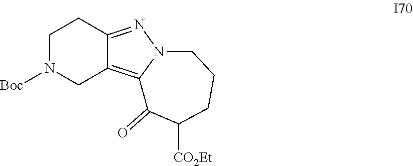 [SMILES: CCOC(=O)C1CCCn2nc3c(c2C1=O)CN(C(=O)OC(C)(C)C)CC3]

The reaction was performed under anhydrous conditions and under Ar atmosphere.

To a solution of t-BuOK (9.26 g, 82.6 mmol) in THF (19) mL) at 0° C. was added dropwise a solution of intermediate I69 in THF (19 mL). The reaction mixture was stirred at 0° C. for 1 h. The reaction was quenched with water (200 mL) and acidified with HCl (1N, aq., 200 mL). The aqueous phase was extracted with EtOAc (2×200 mL). The combined organic layers were dried (Na₂SO₄), filtered and concentrated under reduced pressure. The crude mixture was purified by flash column chromatography (silica, mobile phase: cyclohexane/EtOAc, gradient from 70:30 to 30:70) to give intermediate I70 (9.07 g, 55% over 2 steps) as a colorless gum.

### Intermediate I71

### Ethyl 11-oxo-1,3,4,7,8,9,10,11-octahydro-2H-pyrido[4′,3′:3,4]pyrazolo[1,5-a]azepine-10-carboxylate Hydrochloride

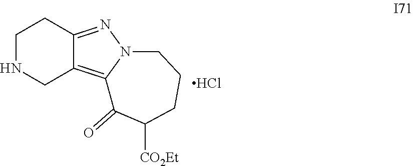 [SMILES: CCOC(=O)C1CCCn2nc3c(c2C1=O)CNCC3 ; Cl]

The reaction was performed under anhydrous conditions and under Ar atmosphere. HCl (4N in 1,4-dioxane, 57.1 mL, 228 mmol) was added to a solution of intermediate I70 (9.07 g, 22.8 mmol) in DCM (50 mL). The reaction mixture was stirred at room temperature for 3 days and diluted with Et₂O (20 mL). The solid was collected by filtration, washed with Et₂O (100 mL) and dried under vacuum to give intermediate I71 as a white solid which was engaged in the next step as such.

### Intermediate I72

### Ethyl 2-(3,4-dichlorobenzoyl)-11-oxo-1,3,4,7,8,9,10,11-octahydro-2H-pyrido[4′,3′:3,4]pyrazolo-[1,5-a]azepine-10-carboxylate

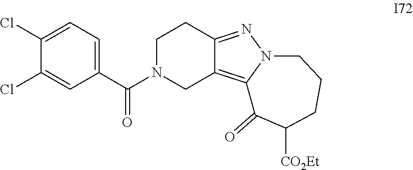 [SMILES: CCOC(=O)C1CCCn2nc3c(c2C1=O)CN(C(=O)c1ccc(Cl)c(Cl)c1)CC3]

The reaction was performed under anhydrous conditions and under Ar atmosphere.

To a suspension of intermediate I71 in DCM (100 mL) at 0° C. was added pyridine (5.47 mL, 67.6 mmol), followed by a solution of 3,4-dichlorobenzoyl chloride (5.19 g, 24.8 mmol) in DCM (50 mL). The reaction mixture was warmed up to room temperature and stirred for 18 h. Additional amount of pyridine (0.82 mL, 22.5 mmol) and 3,4-dichlorobenzoyl chloride (2.36 g, 11.3 mmol) were added. The reaction mixture was stirred at room temperature for an additional 24 h. Pyridine (1.82 mL, 22.5 mmol) and 3,4-dichlorobenzoyl chloride (2.36 g, 11.3 mmol) were added again and the reaction mixture was further stirred for 5 h. The mixture was diluted with DCM (150 mL) and washed with HCl (1M, aq., 2×150 mL) and brine (150 mL). The organic layer was dried (Na₂SO₄), filtered and concentrated under reduced pressure. The crude mixture was purified by flash column chromatography (silica, mobile phase: DCM/MeOH, gradient from 99:1 to 95:5). The residue was taken up in a mixture of DCM and MeOH (9/1; 150 mL) and washed with NaHCO₃ (sat., aq., 150 mL). The layers were separated and the aqueous phase was extracted with a mixture of DCM and MeOH (9:1:100 mL). The combined organic layers were dried (Na₂SO₄), filtered and concentrated under reduced pressure to give intermediate I72 (9.00 g, 89% over 2 steps) as a white foam.

### Intermediate I73

### 2-(3,4-Dichlorobenzoyl)-1,2,3,4,7,8,9,10-octahydro-11H-pyrido[4′,3′:3,4]pyrazolo[1,5-a]azepin-11-one

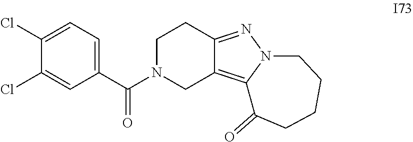 [SMILES: O=C1CCCCn2nc3c(c21)CN(C(=O)c1ccc(Cl)c(Cl)c1)CC3]

To a solution of intermediate I72 (1.00 g, 2.22 mmol) in DMSO (18 mL) was added H₂O (2 mL) and LiCl (122 mg, 2.89 mmol). The reaction mixture was stirred at 150° C. for 5 h. The mixture was cooled to room temperature and poured into water (500 mL). The mixture was stirred for 1 h. The precipitate was collected by filtration and dried under vacuum overnight at 50° C. to afford intermediate I73 (709 mg, 84%) as a white solid.

### Intermediate I74

### 10-Bromo-2-(3,4-dichlorobenzoyl)-1,2,3,4,7,8,9,10-octahydro-1H-pyrido[4′,3′:3,4]pyrazolo-[1,5-a]azepin-1-one

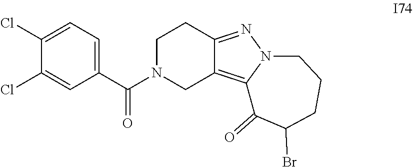 [SMILES: O=C1c2c3c(nn2CCCC1Br)CCN(C(=O)c1ccc(Cl)c(Cl)c1)C3]

The reaction was performed under anhydrous conditions and under Ar atmosphere. To a suspension of CuBr₂ (4.03 g, 18.0 mmol) in CHCl₃ (15 mL) at room temperature was added intermediate I73 (3.79 g, 10.0 mmol). The reaction mixture was stirred at 60° C. for 18 h. Additional quantity of CuBr₂ (1.34 g, 6.01 mmol) was added and the reaction mixture was stirred for another 2 h. The reaction mixture was concentrated under reduced pressure and the crude mixture was purified by flash column chromatography (silica gel, mobile phase: DCM/MeOH, gradient from 100:0 to 97:3) to give two fractions of intermediate I74: fraction A (225 mg, 4%, 75% purity) and fraction B containing impurities. Fraction B was purified by flash column chromatography (C-18, mobile phase: H₂O/MeCN, gradient from 75:25 to 0:100) to give intermediate I74 (2.07 g, 40%, 90% purity) as a green foam.

### 3.2.2. Synthesis of Compounds

### 3.2.2.1. Synthesis of Isoxazole Derivative Compounds

### 3.2.2.1.1. Synthesis of Compound 11

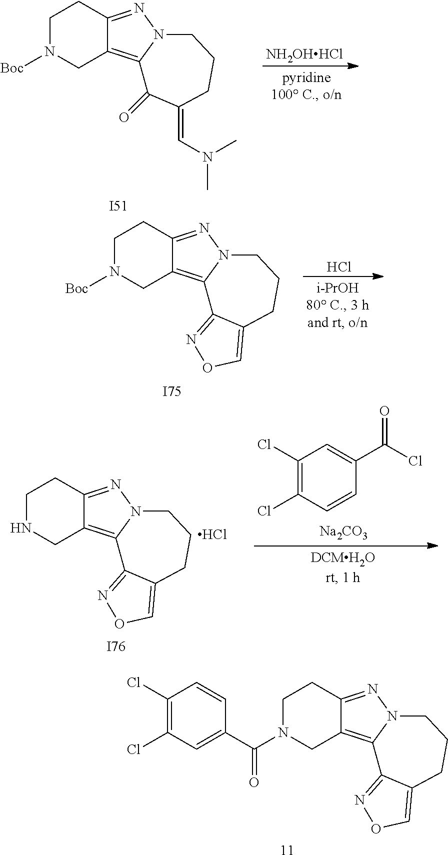

### Intermediate I75

### tert-Butyl 5,6,9,10-tetrahydro-4H-[1,2]oxazolo[3,4-c]pyrido[4′,3′:3,4]pyrazolo[1,5-a]azepine-11(12H)-carboxylate

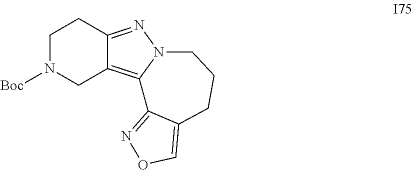 [SMILES: CC(C)(C)OC(=O)N1CCc2nn3c(c2C1)-c1nocc1CCC3]

A mixture of intermediate I51 (100 mg, 0.28 mmol) and N-hydroxylamine hydrochloride (116 mg, 1.66 mmol) in pyridine (5 mL) was stirred at 100° C. overnight. The volatiles were removed under reduced pressure and the crude mixture was purified by flash column chromatography (silica, mobile phase gradient: heptane to EtOAc) to afford intermediate I75 (40 mg, 44%).

### Intermediate I76

### 5,6,9,10,11,12-Hexahydro-4H-[1,2]oxazolo[3,4-c]pyrido[4′,3′:3,4]pyrazolo[1,5-a]azepine Hydrochloride

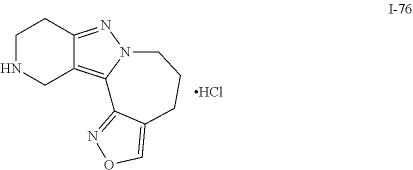 [SMILES: c1onc2c1CCCn1nc3c(c1-2)CNCC3 ; Cl]

HCl (6M in 1-PrOH, 0.75 mL, 4.5 mmol) was added to a solution of intermediate I75 (40 mg, 0.12 mmol) in i-PrOH (5 mL). The reaction mixture was stirred at 80° C. for 1 h, then at room temperature overnight, and at 80° C. for another 2 h. The volatiles were removed under reduced pressure to afford intermediate I76 that was used as such in the next step.

### Compound 11

### (3,4-Dichlorophenyl)(5,6,9,10-(tetrahydro-4H-[1,2]oxazolo[3,4-c]pyrido[4′,3′:3,4]pyrazolo-[1,5-a]azepin-11(12H)-yl)methanone

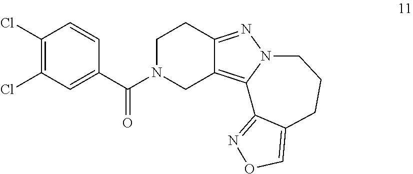 [SMILES: O=C(c1ccc(Cl)c(Cl)c1)N1CCc2nn3c(c2C1)-c1nocc1CCC3]

A mixture of intermediate I76, 3,4-dichlorobenzoyl chloride (27.5 mg, 0.13 mmol) and Na₂CO₃ (25.7 mg, 0.24 mmol) in DCM (5 mL) and water (5 mL) was stirred vigorously at room temperature for 1 h. The organic layer was loaded on a silica cartridge and purified by flash column chromatography (silica, mobile phase gradient: heptane/EtOAc) to afford compound 11 (33.8 mg, 69% over 2 steps).

¹H NMR (400 MHz, DMSO-d₆, 100° C.) δ ppm 8.72 (br s, 1H), 7.65-7.69 (m, 2H), 7.42 (dd. J=8.1, 2.0 Hz, 1H), 4.71 (s, 2H), 4.43-4.49 (m, 2H), 3.68-3.79 (m, 2H), 2.88-2.92 (m, 2H), 2.73 (t, J=5.9 Hz, 2H), 2.07-2.14 (m, 2H); LCMS (method A): Rt=1.02 min, m/z calcd. for C₁₉H₁₆Cl₂N₄O₂ 402, m/z found 4031 [M+H]⁺.

### 3.2.2.1.2. Synthesis of Compound 12

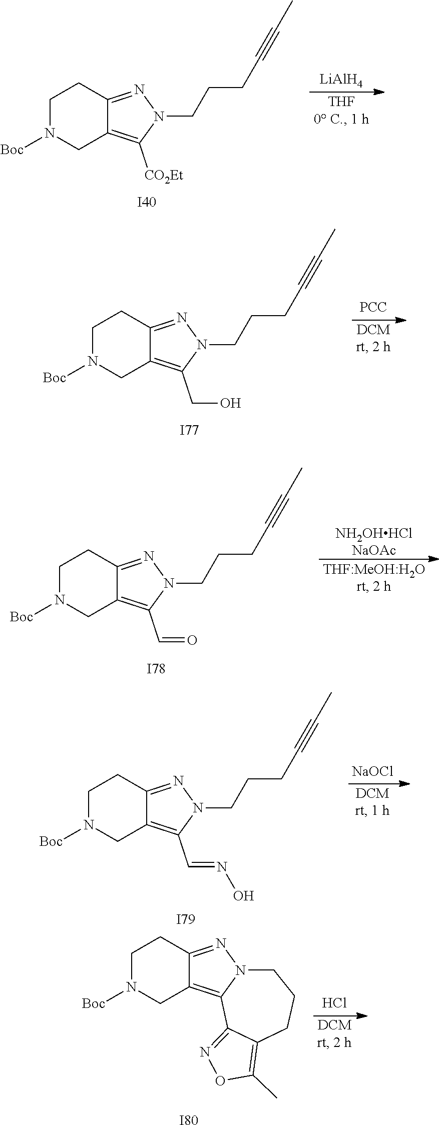

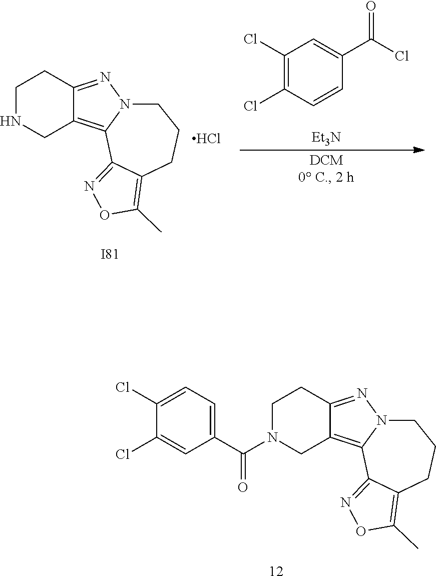

### Intermediate I77

### tert-Butyl 2-(hex-4-yn-1-yl)-3-(hydroxymethyl)-2,4,6,7-tetrahydro-5H-pyrazolo[4,3-c]pyridine-5-carboxylate

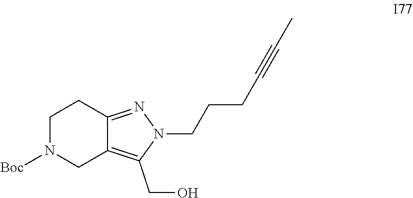 [SMILES: CC#CCCCn1nc2c(c1CO)CN(C(=O)OC(C)(C)C)CC2]

The reaction was performed under anhydrous conditions and under Ar atmosphere.

To a solution of intermediate I40 (885 mg, 2.36 mmol) in THF (20 mL) was added LiAlH₄ (179 mg, 4.71 mmol). The reaction mixture was stirred at 0° C. for 1 h. The reaction was quenched with EtOAc (100 mL) and H₂O (10 mL). A solution of Rochelle's salt (1M, aq., 100 mL) was added and the mixture was stirred for 30 min. The layers were separated and the aqueous layer was extracted with EtOAc (100 mL). The combined organic layers were washed with brine (3×100 mL), dried (Na₂SO₄), filtered and concentrated under to dryness. The crude mixture was purified by flash column chromatography (silica, mobile phase: DCM-MeOH, gradient from 100:0 to 80:20) to give intermediate I77 (614 mg, 78%) as a light yellow oil.

### Intermediate I78

### tert-Butyl 3-formyl-2-(hex-4-yn-1-yl)-2,4,6,7-tetrahydro-5H-pyrazolo[4,3-c]pyridine-5-carboxylate

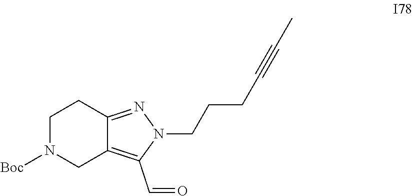 [SMILES: CC#CCCCn1nc2c(c1C=O)CN(C(=O)OC(C)(C)C)CC2]

The reaction was performed under anhydrous conditions and under Ar atmosphere.

To a solution of intermediate I77 (614 mg, 1.84 mmol) in DCM (20 mL) was added PCC (595 mg, 2.76 mmol). The reaction mixture was stirred at room temperature for 2 h and the mixture was concentrated to dryness. The crude mixture was purified by flash column chromatography (silica, mobile phase: cyclohexane/EtOAc, gradient from 100:0 to 60:40) to afford intermediate I78 (512 mg, 84%) as a colorless oil.

### Intermediate I79

### tert-Butyl 2-(hex-4-yn-1-yl)-3-[(hydroxyimino)methyl]-2,4,6,7-tetrahydro-5H-pyrazolo[4,3-c]-pyridine-5-carboxylate

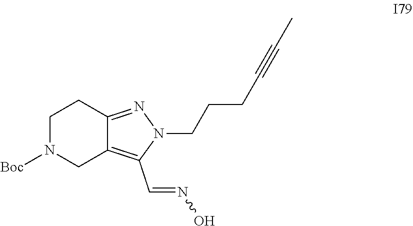 [SMILES: CC#CCCCn1nc2c(c1C=NO)CN(C(=O)OC(C)(C)C)CC2]

To a solution of intermediate I78 (512 mg, 1.55 mmol) and NaOAc (380 mg, 4.64 mmol) in THF (15 mL), MeOH (15 mL) and 120 (30 mL) was added N-hydroxylamine hydrochloride (215 mg, 3.09 mmol). The reaction mixture was stirred at room temperature for 2 h. The mixture was diluted with H₂O (20 mL) and the aqueous phase was extracted with DCM (3×60 mL). The combined organic layers were washed with brine (3×60 mL), dried (Na₂SO₄), filtered and concentrated to dryness to afford intermediate I79 which was used as such in the next step.

### Intermediate I80

### tert-Butyl 3-methyl-5,6,9,10-tetrahydro-4H-[1,2]oxazolo[3,4-c]pyrido[4′,3′:3,4]pyrazolo[1,5-a]-azepine-11(12H)-carboxylate

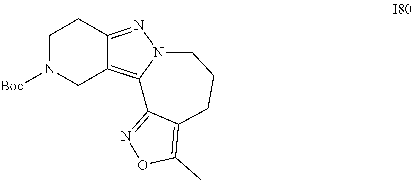 [SMILES: Cc1onc2c1CCCn1nc3c(c1-2)CN(C(=O)OC(C)(C)C)CC3]

To a solution of intermediate I79 in DCM (31.6 mL) at 0° C. was added sodium hypochlorite (14% in H₂O, 1.63 mL, 3.75 mmol). The reaction mixture was stirred at room temperature for 1 h and diluted with MeOH (16 mL), water (50 mL) and DCM (130 mL). The mixture was washed with K₂CO₃ (sat., aq., 50 mL). The layers were separated and the aqueous phase was extracted with DCM (2×50 mL). The combined organic extracts were dried (Na₂SO₄), filtered and concentrated under reduced pressure. The crude mixture was purified by flash column chromatography (silica, mobile phase: cyclohexane/EtOAc gradient from 80:20 to 20:80) to give intermediate I80 (199 mg, 36% over 2 steps, 93% purity).

### Intermediate I81

### 3-Methyl-5,6,9,10,11,12-hexahydro-4H-[1,2]oxazolo[3,4-c]pyrido[4′,3′:3,4]pyrazolo[1,5-a]-azepine Hydrochloride

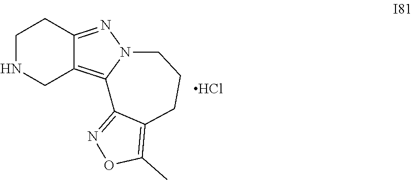 [SMILES: Cc1onc2c1CCCn1nc3c(c1-2)CNCC3 ; Cl]

The reaction was performed under anhydrous conditions and under Ar atmosphere.

To a solution of intermediate I80 (158 mg, 0.46 mmol) in DCM (2 mL) was added HCl (4N in 1,4-dioxane, 2.29 mL, 9.18 mmol). The reaction mixture was stirred at room temperature for 2 h and concentrated to dryness to afford intermediate I81 which was used as such in the next step.

### Compound 12

### (3,4-Dichlorophenyl)(3-methyl-5,6,9,10-tetrahydro-4H-[1,2]oxazolo[3,4-c]pyrido[4′,3′:3,4]-pyrazolo[1,5-a]azepin-11(12H)-yl)methanone

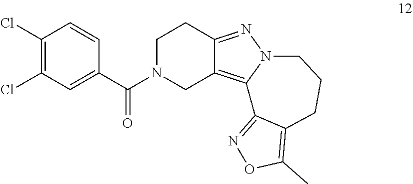 [SMILES: Cc1onc2c1CCCn1nc3c(c1-2)CN(C(=O)c1ccc(Cl)c(Cl)c1)CC3]

The reaction was performed under anhydrous conditions and under Ar atmosphere.

To a solution of intermediate I81 in DCM (5 mL) at 0° C. was added Et₃N (192 μL, 1.38 mmol) and a solution of 3,4-dichlorobenzoyl chloride (125 mg, 0.60 mmol) in DCM (5 mL). The reaction mixture was stirred at 0° C. for 2 h. The reaction mixture was diluted with DCM (30 mL), washed with HCl (1N, aq., 2×20 mL), NaHCO₃ (sat., aq., 2×20 mL) and brine (20 mL), dried (Na₂SO₄), filtered and concentrated under reduced pressure. The crude mixture was purified by flash column chromatography (silica, mobile phase: DCM/MeOH, gradient from 100:0 to 97:3). A second purification was performed by flash column chromatography (silica, mobile phase: DCM/MeOH, gradient from 100:0 to 97:3). The residue was diluted with DCM (20 mL). The solution was washed with NaHCO₃ (sat., aq., 2×10 mL), dried (Na₂SO₄), filtered and concentrated under reduced pressure to dryness. The residue was again purified by flash column chromatography (silica, mobile phase: DCM/MeOH, gradient from 100:0 to 97:3). Another purification was performed via reverse phase column chromatography. The product was co-evaporated with EtOH and dried at 50° C. for 3 days to afford compound 12 (95 mg, 50% over 2 steps) as a white solid.

¹H NMR (400 MHz, DMSO-d₆, 80° C.) δ ppm 7.70 (d, J=8.0 Hz, 1H), 7.68 (d, J=2.0 Hz, 1H), 7.43 (dd, J=8.0, 2.0 Hz, 1H), 4.70 (s, 2H), 4.48-4.41 (M, 2H), 3.79-3.62 (m, 2H), 2.80-2.70 (m, 4H), 2.37 (s, 3H), 2.16-2.05 (m, 2H); LCMS (method E): Rt=10.5 min, m/z calcd. for C₂₄H₁₈Cl₂N₄O₂ 416, m-z found 417 [M+H]⁺.

### 3.2.2.1.3. Synthesis of Compound 13

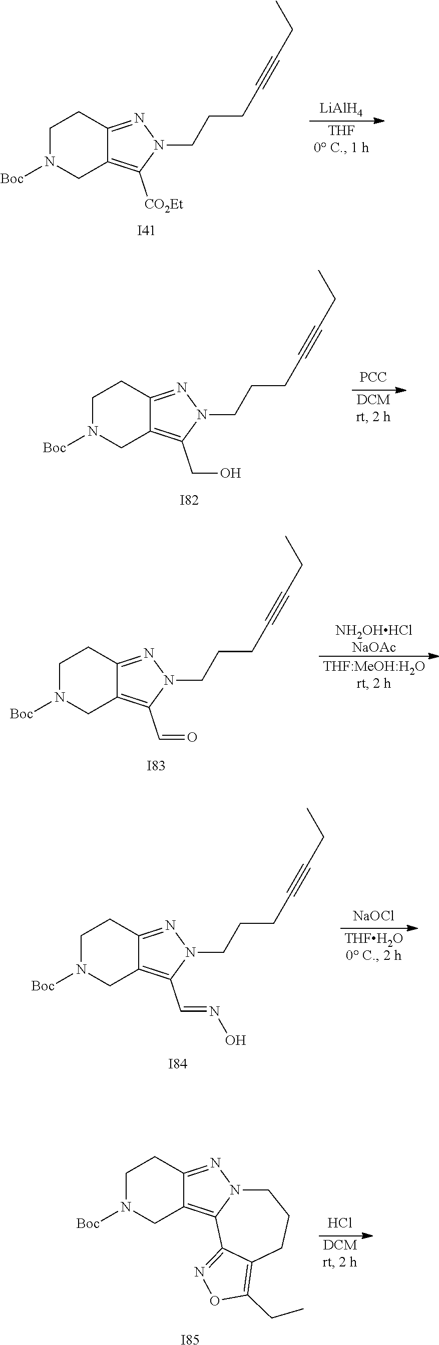

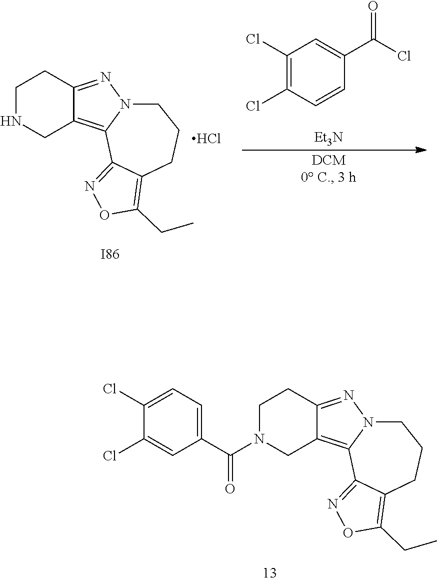

Compound 13 (98 mg) was prepared in an analogous manner to that described for compound 12. Compound 13, (3,4-Dichlorophenyl)(3-ethyl-5,6,9,10-tetrahydro-4H-[1,2]oxazolo[3,4-c]-pyrido[4′,3′:3,4]pyrazolo[1,5-a]azepin-11(12H)-yl)methanone, was obtained as a white solid.

¹H NMR (400 MHz, DMSO-d₆, 80° C.) δ ppm 7.72-7.65 (m, 2H), 7.46-7.39 (m, 1H), 4.71 (s, 2H), 4.48-4.41 (m, 2H), 3.81-3.66 (m, 2H), 2.82-2.70 (m, 6H), 2.16-2.05 (m, 2H), 1.25 (t, J=7.5 Hz, 3H); LCMS (method E): Rt=11.0 min, m/z calcd. for C₂₁H₂₀Cl₂N₄O₂ 430, m/z found 431 [M+H]⁺.

### 3.2.2.1.4. Synthesis of Compound 14

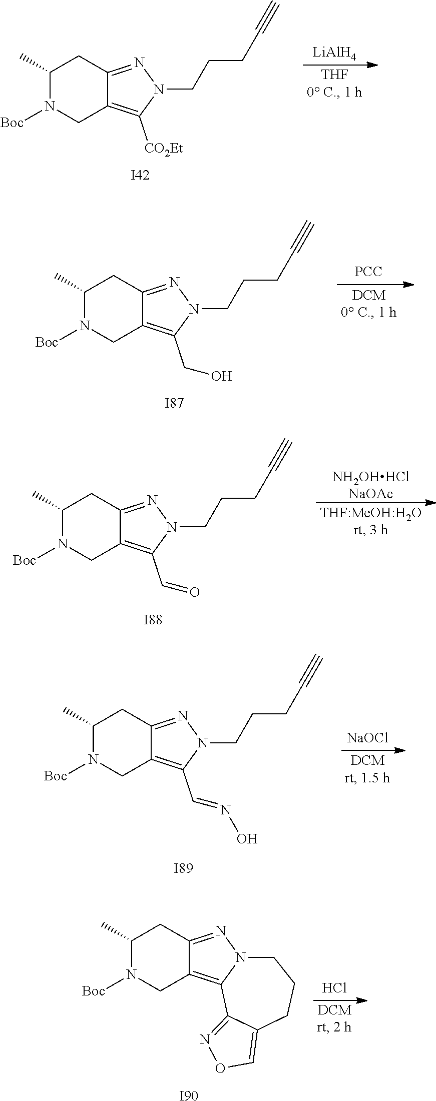

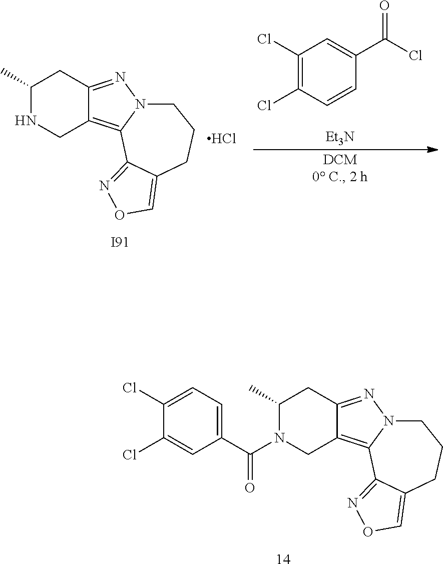

Compound 14 (22 mg) was prepared in an analogous manner to that described for compound 12. Compound 14, (3,4-dichlorophenyl)[(10R)-10-methyl-5,6,9,10-tetrahydro-4H-[1,2]oxazolo-[3,4-c]pyrido[4′,3′:3,4]pyrazolo[1,5-a]azepin-11(12H)-yl]methanone, was obtained as a white solid.

¹H NMR (400 MHz, DMSO-d₆, 80° C.) δ ppm 8.75 (s, 1H), 7.71 (d, J=8.2 Hz, 1H), 7.68 (d, J=1.9 Hz, 1H), 7.43 (dd, J=8.2, 1.9 Hz, 1H), 5.20-5.05 (m, 1H), 4.72-4.55 (m, 1H), 4.52-4.46 (m, 2H), 4.28 (d, J==17.1 Hz, 1H), 2.95-2.90 (m, 2H), 2.60-2.54 (m, 2H), 2.20-2.06 (m, 2H), 1.20 (d, J=6.9 Hz, 3H); LCMS (method E): Rt=10.4 min, m/z calcd. for C₂₀H₁₈Cl₂N₄O₂ 416, m/z found 417 [M+H]⁺.

### 3.2.2.1.5. Synthesis of Compound 15

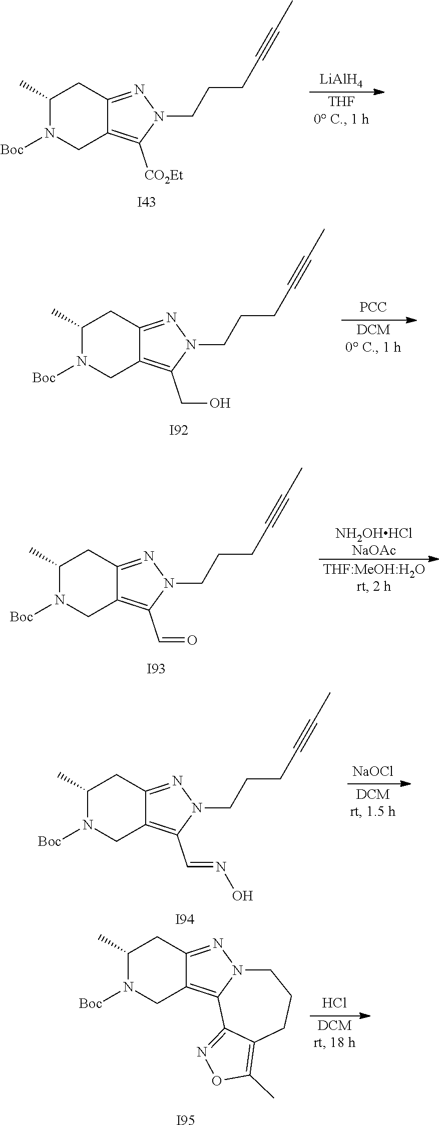

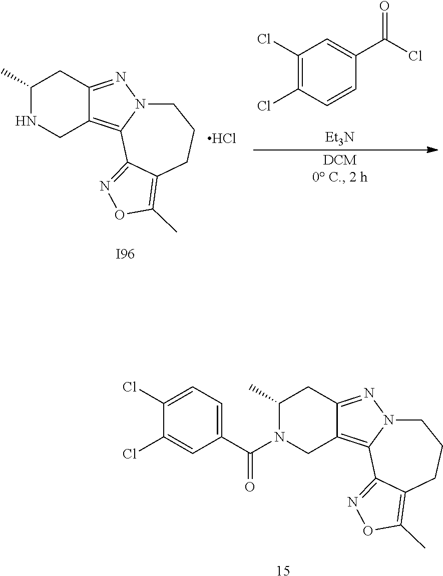

Compound 15 (60 mg) was prepared in an analogous manner to that described for compound 12. Compound 15, (3,4-dichlorophenyl)[(10R)-3,10-dimethyl-5,6,9,10-tetrahydro-4H-[1,2]oxazolo-[3,4-c]pyrido[4′,3′:3,4]pyrazolo[1,5-a]azepin-11(12H)-yl]methanone, was obtained as a white solid.

¹H NMR (400 MHz, DMSO-d₆ 80° C.) δ ppm 7.70 (d, J=8.0 Hz, 1H), 7.67 (d, J=1.8 Hz, 1H), 7.42 (dd, J=8.0, 1.8 Hz, 1H), 5.19-5.01 (m, 1H), 4.72-4.56 (m, 1H), 4.47 (t, J=5.3 Hz, 2H), 4.26 (d, J=17.0 Hz, 1H), 2.95-2.92 (m, 1H), 2.79 (t, J=6.1 Hz, 2H), 2.56 (d, J=17.0 Hz, 1H), 2.38 (s, 31H), 2.22-2.05 (m, 2H), 1.20 (d, J=6.9 Hz, 3H); LCMS (method E): Rt=10.8 min, m/z calcd. for C₂₁H₂₀Cl₂N₄O₂ 430, m/z found 431 [M+H]⁺.

### 3.2.2.1.6. Synthesis of Compound 16

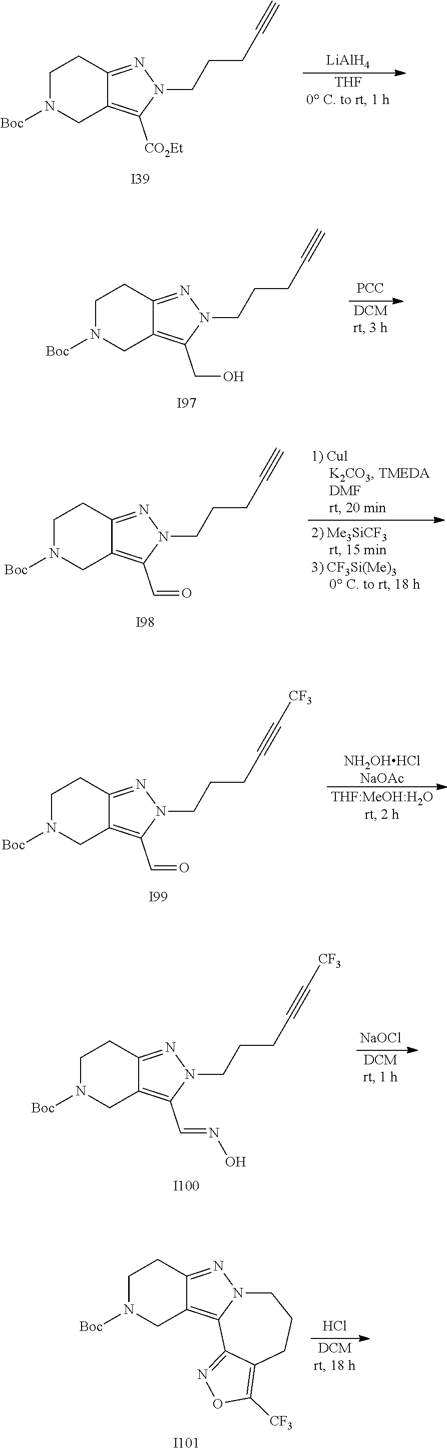

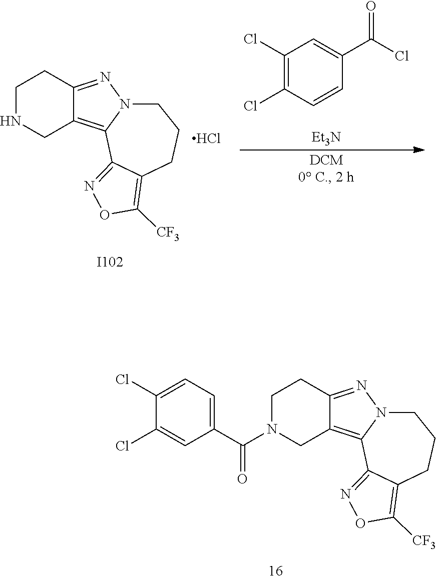

### Intermediate I97

### Tert-Butyl 3-(hydroxymethyl)-2-(pent-4-yn-1-yl)-2,4,6,7-tetrahydro-5H-pyrazolo[4,3-c]pyridine-5-carboxylate

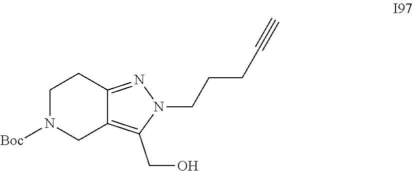 [SMILES: C#CCCCn1nc2c(c1CO)CN(C(=O)OC(C)(C)C)CC2]

### Intermediate I98

### Tert-Butyl 3-formyl-2-(pent-4-yn-1-yl)-2,4,6,7-tetrahydro-5H-pyrazolo[4,3-c]pyridine-3-carboxylate

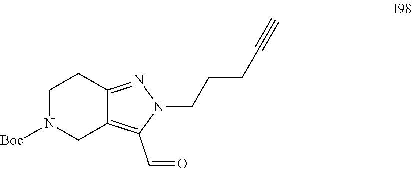 [SMILES: C#CCCCn1nc2c(c1C=O)CN(C(=O)OC(C)(C)C)CC2]

Intermediate I98 (757 mg, 72%, 90% purity) was prepared in an analogous manner to that described for intermediate I78.

### Intermediate I99

### Tert-Butyl 3-formyl-2-(6,6,6-trifluorohex-4-yn-1-yl)-2,4,6,7-tetrahydro-5H-pyrazolo[4,3-c]-pyridine-5-carboxylate

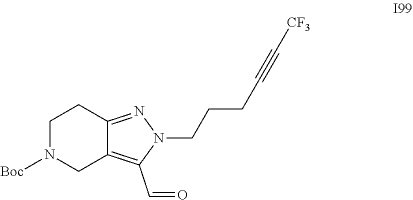 [SMILES: CC(C)(C)OC(=O)N1CCc2nn(CCCC#CC(F)(F)F)c(C=O)c2C1]

The reaction was performed under anhydrous conditions.

A mixture of CuI (567 mg, 2.98 mmol), K₂CO₃ (823 mg, 5.96 mmol) and TMEDA (446 μL, 2.98 mmol) in DMF (12 mL) was vigorously stirred at room temperature for 20 min. Trimethyl(trifluoromethyl)silane (587 μL, 3.97 mmol) was added and the mixture was stirred at room temperature for 15 min. The mixture was cooled to 0° C. and a solution of intermediate I98 (630 mg, 1.99 mmol) and trimethyl(trifluoromethyl)silane (587 μL, 3.97 mmol) in DMF (12 mL) at 0° C. was added. The reaction mixture was stirred at 0° C. for 30 min, and at room temperature for 18 h. The mixture was diluted with water (50 mL). The layers were separated and the aqueous phase was extracted with EtOAc (3×40 mL). The combined organic extracts were washed with brine (3×30 mL), dried (Na₂SO₄), filtered and concentrated under reduced pressure. The crude mixture was purified by flash column chromatography (silica, mobile phase: cyclohexane/EtOAc, gradient from 100:0 to 60:40) to give intermediate I99 (188 mg, 24%) as a colorless oil.

### Intermediate I100

### Tert-Butyl 3-[(hydroxyimino)methyl]-2-(6,6,6-trifluorohex-4-yn-1-yl)-2,4,6,7-tetrahydro-5f-pyrazolo[4,3-c]pyridine-5-carboxylate

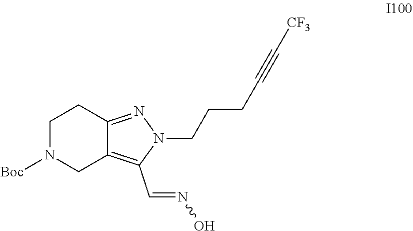 [SMILES: CC(C)(C)OC(=O)N1CCc2nn(CCCC#CC(F)(F)F)c(C=NO)c2C1]

Intermediate I100 (202 mg) was prepared in an analogous manner to that described for intermediate I79.

### Intermediate I101

### Tert-Butyl 3-(trifluoromethyl)-5,6,9,10-tetrahydro-4H-[1,2]oxazolo[3,4-c]pyrido[4′,3′:3,4]-pyrazolo[1,5-a]azepine-11(12H)-carboxylate

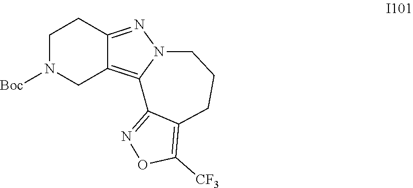 [SMILES: CC(C)(C)OC(=O)N1CCc2nn3c(c2C1)-c1noc(C(F)(F)F)c1CCC3]

Intermediate I101 (78 mg, 39% over 2 steps, 95% purity) was prepared in an analogous manner to that described for intermediate I80.

### Intermediate I102

### 3-(Trifluoromethyl)-5,6,9,10,11,12-hexahydro-4H-[1,2]oxazolo[3,4-c]pyrido[4′,3′:3,4]pyrazolo-[1,5-a]azepine Hydrochloride

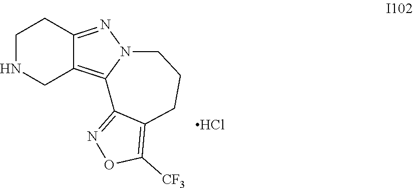 [SMILES: FC(F)(F)c1onc2c1CCCn1nc3c(c1-2)CNCC3 ; Cl]

Intermediate I102 was prepared in an analogous manner to that described for intermediate I81.

### Compound 16

### (3,4-Dichlorophenyl)[3-(trifluoromethyl)-5,6,9,10-tetrahydro-4H-[1,2]oxazolo[3,4-c]pyrido[4′,3′:3,4]pyrazolo[1,5-a]azepin-11(12H)-yl]methanone

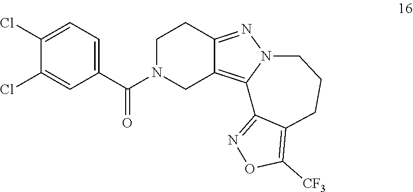 [SMILES: O=C(c1ccc(Cl)c(Cl)c1)N1CCc2nn3c(c2C1)-c1noc(C(F)(F)F)c1CCC3]

Compound 16 (76 mg, 87% over 2 steps) was prepared in an analogous manner to that described for compound 12.

¹H NMR (400 MHz, DMSO-d₆, 80° C.) δ ppm 7.70 (d, J=8.4 Hz, 1H), 7.69 (d, J=2.0 Hz, 1H), 7.45 (dd, J=8.4, 2.0 Hz, 1H), 4.77-4.73 (m, 2H), 4.55-4.50 (m, 2H), 3.80-3.72 (m, 2H), 3.07-3.02 (m, 2H), 2.77 (t, J=6.0 Hz, 2H), 2.23-2.17 (m, 2H); LCMS (method E): Rt=11.5 min, m/z calcd. for C₂₀H₁₅Cl₂F₃N₄O₂ 470, m/z found 471 [M+H]⁺.

### 3.2.2.1.8. Synthesis of Compound 17

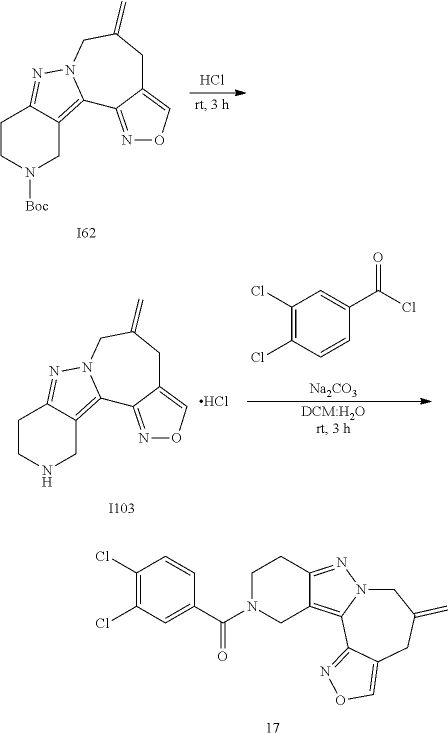

### Intermediate I103

### 5-Methylidene-5,6,9,10,11,12-hexahydro-4H-[1,2]oxazolo[3,4-c]pyrido[4′,3′:3,4]pyrazolo-[1,5-a]azepine Hydrochloride

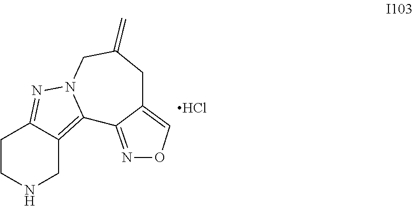 [SMILES: C=C1Cc2conc2-c2c3c(nn2C1)CCNC3 ; Cl]

A mixture of intermediate I62 (1.02 g, 2.98 mmol) in HCl (4N in 1,4-dioxane, 8.0 mL, 32.0 mmol) was stirred at room temperature for 3 h and concentrated under reduced pressure to afford intermediate I103 which was used as such in the next step.

### Compound 17

### (3,4-Dichlorophenyl)(5-methylidene-5,6,9,10-tetrahydro-4H-[1.2]oxazolo[3,4-c]pyrido-[4′,3′:3,4]pyrazolo[1,5-a]azepin-11(12H)-yl)methanone

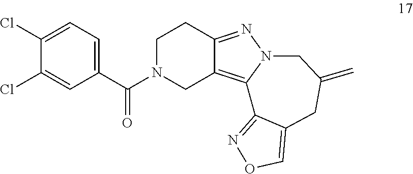 [SMILES: C=C1Cc2conc2-c2c3c(nn2C1)CCN(C(=O)c1ccc(Cl)c(Cl)c1)C3]

To a solution of intermediate I103 in DCM (6 mL) and water (6 mL) were added 3,4-dichlorobenzoyl chloride (749 mg, 3.58 mmol) and Na₂CO₃ (631 mg, 5.96 mmol). The reaction mixture was stirred at room temperature for 3 h. The layers were separated and the aqueous phase was extracted with DCM. The combined organic extracts were dried (Na₂SO₄), filtered and adsorbed onto silica. The crude mixture was purified by flash column chromatography (silica, mobile phase gradient: 50-80% heptane/EtOAc) to afford compound 17 (916 mg, 74% over 2 steps) as a white foamy solid.

¹H NMR (400 MHz, CDCl₃) δ ppm 8.40-8.21 (m, 1H), 7.58 (d, J=2.0 Hz, 1H), 7.56-7.44 (m, 1H), 7.30 (dd, J=8.2, 2.0 Hz, 1H), 5.36 (s, 1H), 5.28 (s, 1H), 5.00-4.89 (m, 2H), 4.68 (s, 1H), 4.04 (s, 1H), 3.76-3.53 (m, 3H), 3.00-2.74 (m, 3H); LCMS (method C): Rt=3.03 min, m/z calcd. for C₂₀H₁₆Cl₂N₄O₂ 414, m/z found 415 [M+H]⁺.

### 3.2.2.1.9. Synthesis of Compound 18

### (3,4-Dichlorophenyl)[5-hydroxy-5-(hydroxymethyl)-5,6,9,10-tetrahydro-4H-[1,2]oxazolo-[3,4-c]pyrido[4′,3′:3,4]pyrazolo[1,5-a]azepin-11(12H)-yl]methanone

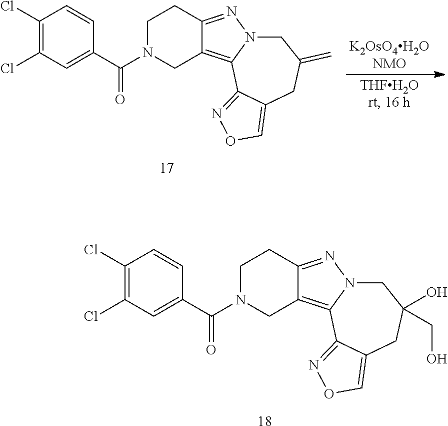 [SMILES: C=C1Cc2conc2-c2c3c(nn2C1)CCN(C(=O)c1ccc(Cl)c(Cl)c1)C3 ; "K2OsO4·H2ONMO" ; "THF·H2Ort, 16 h" ; "17" ; O=C(c1ccc(Cl)c(Cl)c1)N1CCc2nn3c(c2C1)-c1nocc1CC(O)(CO)C3 ; "18"]

A mixture of compound 17 (250 mg, 0.6 (0 mmol), K₂OsO₄.2H₂O (22.2 mg, 60.2 μmol) and NMO (106 mg, 0.90 mmol) in THF (1.4 mL) and water (0.7 mL) was stirred at room temperature for 16 h. The mixture was diluted with H₂O (15 mL) and extracted with EtOAc (3×15 mL). The combined organic extracts were washed with brine, dried (Na₂SO₄), filtered and concentrated under reduced pressure.

A fraction of the crude mixture (50 mg) was purified by flash column chromatography (silica, mobile phase gradient: 0-10% MeOH/EtOAc) to give compound 18 (21 mg) as a white solid.

¹H NMR (400 MHz, DMSO-d₆) δ 8.85 (d, J=33.7 Hz, 1H), 7.87-7.67 (m, 2H), 7.48 (dd, J=8.2, 1.9 Hz, 1H), 5.10 (s, 1H), 5.00-4.89 (m, 1H), 4.78 (s, 1H), 4.67-4.22 (m, 3H), 3.93 (s, 1H), 3.58 (s, 1H), 3.40 (overlaps with solvent), 3.00-2.62 (m, 4H); LCMS (method C): Rt=2.27 min, m/z calcd. for C₂₀H₁₈Cl₂N₄O₄ 448, m/z found 449 [M+H]⁺.

### 3.2.2.1.10. Synthesis of Compound 19

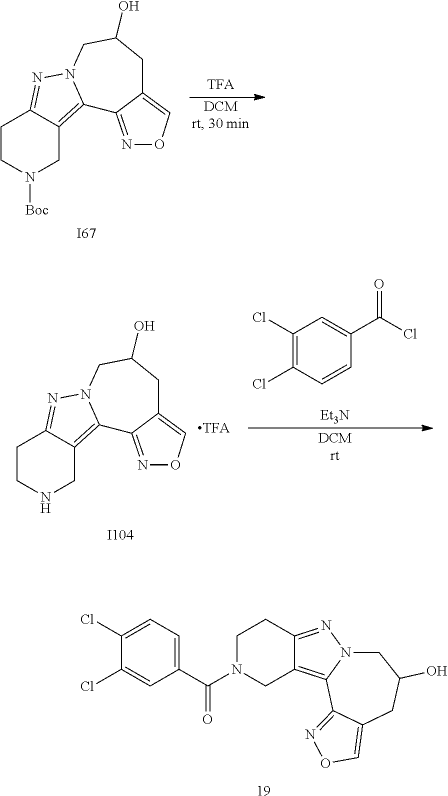

### Intermediate I104

### 5,6,9,10,11,12-Hexahydro-4H-isoxazolo[3,4-c]pyrido[4′,3′:3,4]pyrazolo[1,5-a]azepin-5-ol.TFA

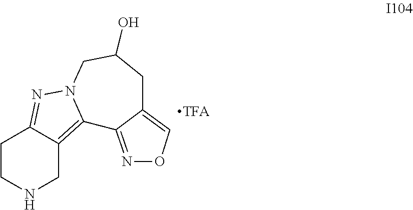 [SMILES: OC1Cc2conc2-c2c3c(nn2C1)CCNC3 ; "·TFA"]

To a mixture of intermediate I67 (120 mg) in DCM (5 mL) was added TFA (1.54 g, 13.5 mmol). The reaction mixture was stirred at 25° C. for 30 min and concentrated under reduced pressure to afford intermediate I104 which was used as such in the next step.

### Compound 19

### (3,4-Dichlorophenyl)(5-hydroxy-5,6,9,10-tetrahydro-4H-isoxazolo[3,4-c]pyrido[4′,3′:3,4]pyrazolo[1,5-a]azepin-11(12H)-yl)methanone

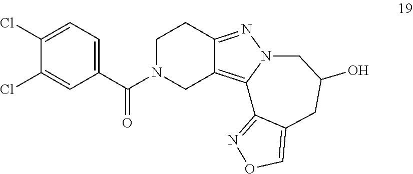 [SMILES: O=C(c1ccc(Cl)c(Cl)c1)N1CCc2nn3c(c2C1)-c1nocc1CC(O)C3]

Compound 19 was prepared in an analogous manner analogous to that described for compound 23.

¹H NMR (400 MHz, DMSO-d₆) δ ppm 9.00-8.75 (m, 1H), 7.77-7.70 (m, 2H), 7.47 (br d, J=8.3 Hz, 1H), 5.50-5.30 (m, 1H), 4.78 (br s, 1H), 4.66-4.39 (m, 3H), 4.25 (br s, 1H), 4.03-3.83 (m, 1H), 3.57 (br s, 1H), 3.05-2.89 (m, 2H), 2.83-2.62 (m, 2H).

### 3.2.2.1.11. Synthesis of Compound 20

### (3,4-Dichlorophenyl)(5-methoxy-5,6,9,10-tetrahydro-4H-isoxazolo[3,4-c]pyrido[4′,3′:3,4]-pyrazolo[1,5-a]azepin-11(12H)-yl)methanone

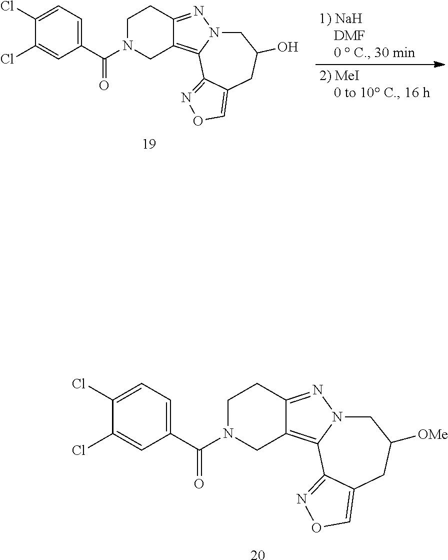

A solution of compound 19 (12.0 mg, 28.6 μmol) in DMF (1 mL) was added NaH (60% dispersion in mineral oil, 2.29 mg, 57.2 μmol) at 0° C. under N₂ atmosphere. The reaction mixture was stirred at this temperature for 30 min and Mel (8.13 mg, 57.2 μmol) was added. The reaction mixture was stirred at 10° C. for 16 h under N₂ atmosphere and poured into water (10 mL). The aqueous phase was extracted with EtOAc (2×5 mL). The combined organic extracts were washed with brine (10 mL), dried (Na₂SO₄), filtered and concentrated under reduced pressure. The crude mixture combined with another fraction (10 mg scale) and purified by reverse phase HPLC (Gilson GX-281 semi-prep-HPLC with Phenomenex Synergi C18 (10 μm, 150×25 mm), or Boston Green ODS C18 (5 μm, 150×30 mm), and mobile phase of 5-99% MeCN in water (with 0.225% FA) over 10 min and then hold at 100% MeCN for 2 min. at a flow rate of 25 mL/min) to give compound 20 (9 mg, 69%, 95% purity) as white solid.

MS (ESI): m/z calcd. for C₂₀H₁₈Cl₂N₄O₃ 432.1; m/z found 433.1 [M+H]⁺; ¹H NMR (400 MHz, CDCl₃) δ ppm 8.37-8.27 (m, 1H), 7.60 (s, 1H), 7.51 (d, J=9.6 Hz, 1H), 7.34-7.32 (m, 1H), 4.79-4.70 (m, 2H), 4.51-4.47 (m, 1H), 3.96-3.92 (m, 2H), 3.78-3.57 (m, 1H), 3.41 (s, 3H), 3.22-3.16 (m, 1H), 3.12-2.65 (m, 4H).

### 3.2.2.1.12. Synthesis of Compound 21

### (3,4-Dichlorophenyl)(5-fluoro-5,6,9,10-tetrahydro-4H-[1,2]oxazolo[3,4-c]pyrido[4′,3′:3,4]-pyrazolo[1,5-a]azepin-11(12H)-yl)methanone

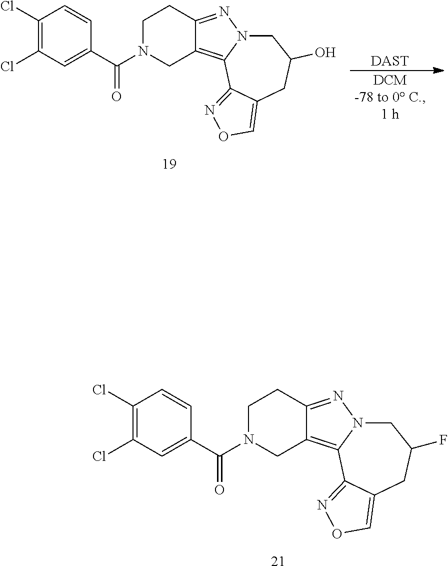

To a solution of compound 19 (42.8 mg, 0.10 mmol) in DCM (2.1 mL) at −78° C. was added DAST (18.8 μL, 0.15 mmol). The reaction mixture was warmed to 0° C. and stirred for 1 h. The reaction was quenched with NaHCO₃ (sat., aq.). The layers were separated and the aqueous phase was extracted with DCM (3 times). The combined organic extracts were washed with brine, dried (Na₂SO₄), filtered and concentrated under reduced pressure. The crude mixture was purified by preparative TLC (80% EtOAc/heptane) to afford compound 21 (7.5 mg, 17%) as a white solid.

¹H NMR (400 MHz, MeOD) δ ppm 8.65 (d, J=34.4 Hz, 1H), 7.78-7.59 (m, 2H), 7.43 (s, 1H), 5.47-5.20 (m, 1H), 4.90 (overlap with water peak), 4.80-4.49 (m, 2H), 4.23-3.90 (m, 1H), 3.71 (s, 1H), 3.30 (overlap with solvent peak), 2.94-2.77 (m, 2H); LCMS (method D): Rt=3.07 min, m/r calcd. for C₁₉H₁₅Cl₂FN₄O₂ 420, m/z found 421 [M+H]⁺.

### 3.2.2.1.13. Synthesis of Compound 22

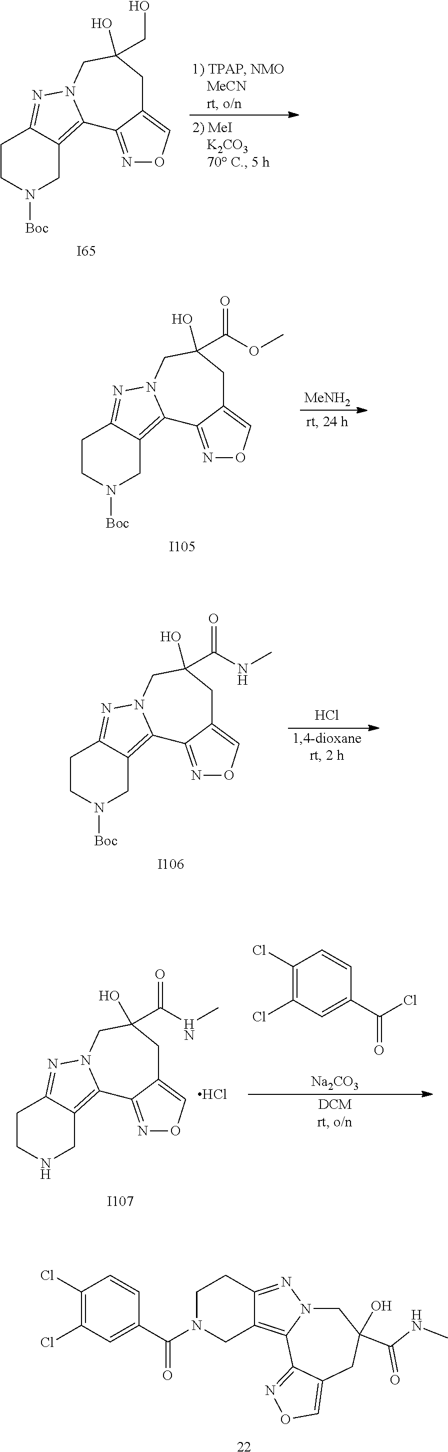

### Intermediate I105

### 11-tert-Butyl 5-methyl 5-hydroxy-5,6,9,10-tetrahydro-4f-[1,2]oxazolo[3,4-c]pyrido[4′,3′:3,4]-pyrazolo[1,5-a]azepine-5,11(12H)-dicarboxylate

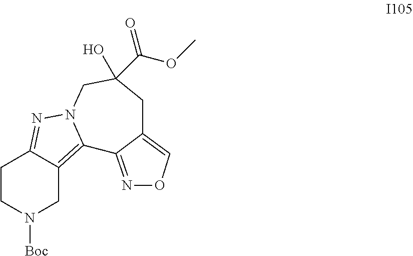 [SMILES: COC(=O)C1(O)Cc2conc2-c2c3c(nn2C1)CCN(C(=O)OC(C)(C)C)C3]

To a solution of intermediate I65 (375 mg, 1.00 mmol) in MeCN (3.5 mL) was added TPAP (35.0 mg, 0.10 mmol) and NMO (1.17 g, 9.96 mmol). The reaction mixture was stirred at room temperature overnight. An additional 0.1 equiv of TPAP (35.0 mg, 0.10 mmol) was added and stirring was continued for 2 h. iodomethane (620 μL, 9.% mmol) and K₂CO₃ (275 mg, 1.99 mmol) were added to the mixture. The reaction mixture was stirred at 70° C. for 5 h and diluted with EtOAc and HCl. The layers were separated and the aqueous phase was extracted with EtOAc (3 times). The combined organic extracts were washed with brine, dried (Na₂SO₄), filtered and concentrated under reduced pressure. The crude mixture was adsorbed onto silica and purified by flash column chromatography (silica, mobile phase gradient: 60-100% EtOAc/heptane) to afford intermediate I105 (89 mg, 22%) as a solid.

### Intermediate I106

### tert-Butyl 5-hydroxy-5-(methylcarbamoyl)-5,6,9,10-tetrahydro-4H-[1,2]oxazolo[3,4-c]pyrido-[4′,3′:3,4]pyrazolo[1,5-a]azepine-11(12H)-carboxylate

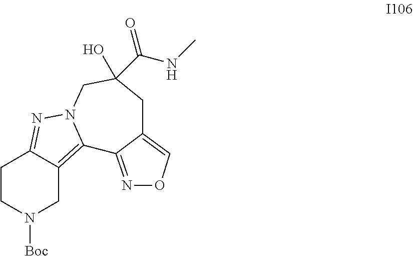 [SMILES: CNC(=O)C1(O)Cc2conc2-c2c3c(nn2C1)CCN(C(=O)OC(C)(C)C)C3]

Intermediate I105 (89.0 mg, 0.22 mmol) was dissolved in a solution of methylamine (2M in THF, 2.20 mL, 4.40 mmol) and the reaction mixture was stirred at room temperature for 24 h. The mixture was concentrated under reduced pressure to afford intermediate I106 which was used as such in the next step.

### Intermediate I107

### 5-Hydroxy-N-methyl-5,6,9,10,11,12-hexahydro-4H-[1,2]oxazolo[3,4-c]pyrido[4′,3′:3,4]-pyrazolo[1,5-a]azepine-5-carboxamide Hydrochloride

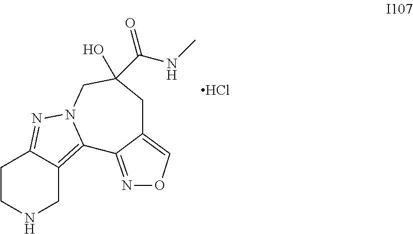 [SMILES: CNC(=O)C1(O)Cc2conc2-c2c3c(nn2C1)CCNC3 ; Cl]

To a solution of intermediate I106 in 1,4-dioxane (2 mL) was added HCl (4N in 1,4-dioxane, 1 mL, 4 mmol). The reaction mixture was stirred at room temperature for 2 h and concentrated under reduced pressure to afford intermediate I107 which was used as such in the next step.

### Compound 22

### 11-(3,4-Dichlorobenzoyl)-S-hydroxy-N-methyl-5,6,9,10,11,12-hexahydro-4H-[1,2]oxazolo-[3,4-c]pyrido[4′,3′:3,4]pyrazolo[1,5-a]azepine-5-carboxamide

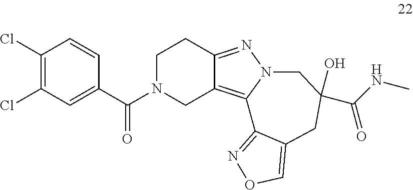 [SMILES: CNC(=O)C1(O)Cc2conc2-c2c3c(nn2C1)CCN(C(=O)c1ccc(Cl)c(Cl)c1)C3]

To a solution of intermediate I107 in DCM (1 mL) and water (1 mL) were added 3,4-dichlorobenzoyl chloride (69.1 mg, 0.33 mmol) and Na₂CO₃ (46.6 mg, 0.44 mmol). The reaction mixture was stirred at room temperature overnight. The volatiles were removed under reduced pressure and the aqueous phase was extracted with EtOAc (twice). The combined organic extracts were washed with brine, dried (Na₂SO₄), filtered and concentrated under reduced pressure. The crude mixture was adsorbed onto silica and purified by flash column chromatography (silica, mobile phase gradient: 0-10% MeOH/EtOAc). The residue was washed EtOAc and MeOH to give compound 22 (44 mg, 42% over 3 steps).

¹H NMR (400 MHz, DMSO-d₆) δ ppm 8.99-8.76 (m, 1H), 8.09 (s, 1H), 7.85-7.67 (m, 2H), 7.48 (dd, J=8.2, 2.0 Hz, 1H), 6.10 (d, J=17.4 Hz, 1H), 4.89-4.36 (m, 4H), 3.93 (s, 1H), 3.59 (s, 1H), 3.33-3.18 (m, 1H), 3.13-2.89 (m, 1H), 2.85-2.69 (m, 2H), 2.65 (s, 3H). LCMS (method C): Rt=2.70 min, m/z calcd. for C₂₁H₁₉ClN₃O₄ 475, m/z found 476 [M+H]⁺.

### 3.2.2.1.14. Synthesis of Compound 23

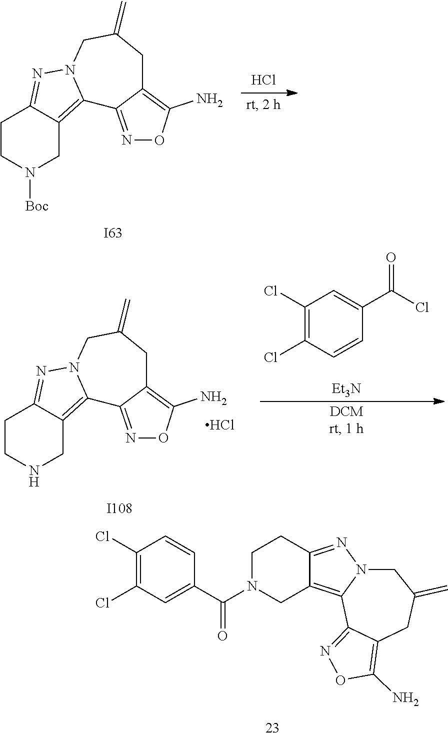

### Intermediate I108

### 5-Methylidene-5,6,9,10,11,12-hexahydro-4H-[1,2]oxazolo[3,4-c)pyrido[4′,3′:3,4]pyrazolo-1,5-a]azepin-3-amine Hydrochloride

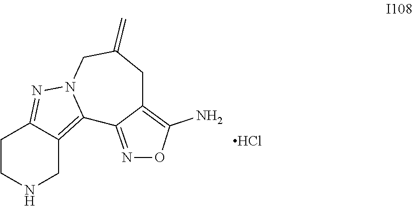 [SMILES: C=C1Cc2c(noc2N)-c2c3c(nn2C1)CCNC3 ; Cl]

To a solution of intermediate I63 (300 mg, 0.84 mmol) was added HCl (4M in 1,4-dioxane, 6 mL, 24.0 mmol). The reaction mixture was stirred at room temperature for 2 h and concentrated under reduced pressure to afford intermediate I108 which was used as such in the next step.

### Compound 23

### (3-Amino-5-methylidene-5,6,9,10-tetrahydro-4H-[1,2]oxazolo[3,4-c]pyrido[4′,3′:3,4]pyrazolo-[1,5-a]azepin-11(12H)-yl)(3,4-dichlorophenyl)methanone

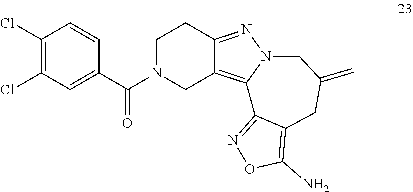 [SMILES: C=C1Cc2c(noc2N)-c2c3c(nn2C1)CCN(C(=O)c1ccc(Cl)c(Cl)c1)C3]

To a mixture of intermediate I108 and 3,4-dichlorobenzoyl chloride (158 mg, 0.76 mmol) in DCM (6.17 mL) was added Et₃N (1.00 mL, 7.19 mmol). The reaction mixture was stirred at room temperature for 1 h and concentrated under reduced pressure. The crude mixture was adsorbed onto silica and purified by flash column chromatography (silica, mobile phase gradient: 70-100% EtOAc/heptane) to afford compound 23 (157 mg, 51% over 2 steps).

¹H NMR (400 MHz, acetone-d₆) δ ppm 7.61-7.78 (m, 2H), 7.39-7.56 (m, 1H), 6.22 (m, 2H), 5.18-5.34 (m, 2H), 4.77-5.00 (m, 3H), 4.56-4.70 (m, 1H), 3.92-4.07 (m, 1H), 3.63-3.78 (m, 1H), 3.36-3.52 (m, 2H), 2.78 (m, 2H); LCMS (method C): Rt=2.85 min, m/z calcd. for C₂₀H₁₇Cl₂N₅O₄ 429, m/z found 430[M+H]⁺.

### 3.2.2.1.15. Synthesis of Compound 24

### [3-Amino-5-hydroxy-5-(hydroxymethyl)-5,6,9,10-tetrahydro-4H-[1,2]oxazolo[3,4-c]pyrido-[4′,3′:3,4]pyrazolo[1,5-a]azepin-11(12H)-yl](3,4-dichlorophenyl)methanone

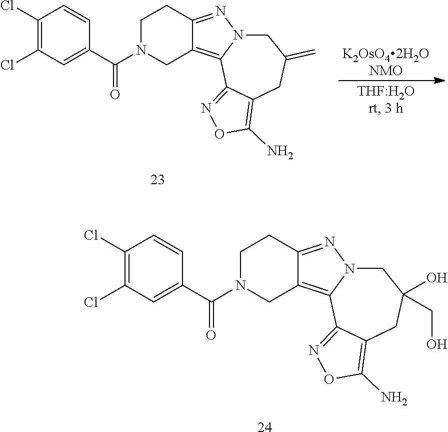 [SMILES: C=C1Cc2c(noc2N)-c2c3c(nn2C1)CCN(C(=O)c1ccc(Cl)c(Cl)c1)C3 ; "K2OsO4·2H2ONMO" ; "THF:H2Ort, 3 h" ; "23" ; Nc1onc2c1CC(O)(CO)Cn1nc3c(c1-2)CN(C(=O)c1ccc(Cl)c(Cl)c1)CC3 ; "24"]

A mixture of compound 23 (56.0 mg 0.13 mmol), K₂OsO₄.2H₂O (4.80 mg, 13.0 μmol) and NMO (22.9 mg, 0.20 mmol) in THF (0.3 mL) and water (0.15 mL) was stirred for at room temperature for 3 h. The mixture was diluted with H₂O (15 mL) and extracted with EtOAc (3×15 mL). The combined organic extracts were washed with brine, dried (Na₂SO₄), filtered and concentrated under reduced pressure. The crude mixture was adsorbed onto silica and purified by flash column chromatography (silica, mobile phase gradient: 0-10% MeOH/EtOAc). The residue was dissolved in MeCN and water and lyophilized to obtain compound 24 (24.8 mg, 41%) as a white solid.

¹H NMR (400 MHz, DMSO-d₆) δ ppm 7.82-7.69 (m, 2H), 7.53-7.42 (m, 1H), 6.67 (d, J=26.7 Hz, 2H), 5.04-4.94 (m, 1H), 4.85-4.19 (m, 5H), 3.91 (s, 1H), 3.55 (s, 1H), 2.86-2.54 (m, 5H, overlapping with solvent); LCMS (method C): Rt=2.31 min, m/z calcd. for C₂₀H₁₉Cl₂N₅O₄ 463, m/z found 464 [M+H]⁺.

### 3.2.2.1.16. Synthesis of Compound 25

### (3,4-dichlorophenyl)[3-(methylamino)-5-methylidene-5,6,9,10-tetrahydro-4H-[1,2]oxazolo-[3,4-c]pyrido[4′,3′:3,4]pyrazolo[1,5-a]azepin-11(12H)-yl]methanone

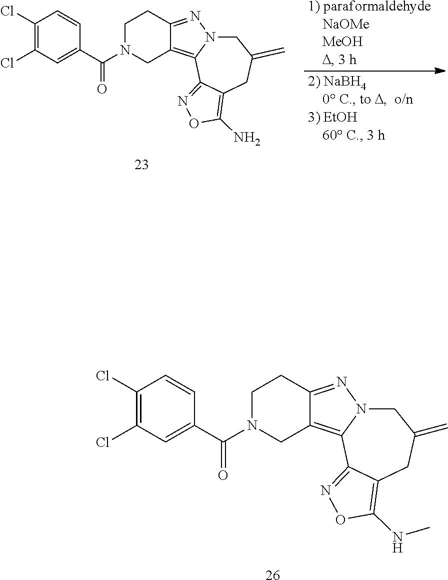

To a mixture of compound 23 (30.0 mg, 69.7 μmol) and paraformaldehyde (30.0 mg) in MeOH (1 mL) was added NaOMe (15.1 mg, 0.28 mmol). The reaction mixture was stirred under refluxed for 3 h, then cooled to 0° C. NaBH₄ (10.5 mg, 0.28 mmol) was added and the reaction mixture was stirred under reflux overnight. The solvent was evaporated, EtOH was added and the stirring was continued at 60° C. for 3 h. The mixture was cooled to room temperature and the reaction was quenched with NH₄Cl (sat., aq.). The mixture was diluted with water and EtOAc. The aqueous phase was extracted with EtOAc (3 times). The combined organic extracts were dried (Na₂SO₄), filtered and concentrated under reduced pressure. The residue was dissolved in DMSO, loaded onto a reverse phase column and purified by HPLC (mobile phase gradient: 10-100% MeCN/water with 0.1% TFA) to afford compound 25 (7 mg).

LCMS (method C): Rt=2.91 min, m/z calcd. for C₂₁H₁₉Cl₂N₅O₂ 444.3, m/z found 444.1 [M+H]⁺.

### 3.2.2.1.17. Synthesis of Compound 26 (3,4-Dichlorophenyl)[3-(dimethylamino)-5-methylidene-5,6,9,10-tetrahydro-4H-[1,2]oxazolo-[3,4-c]pyrido[4′,3′:3,4]pyrazolo[1,5-a]azepin-11(12H)-yl]methanone

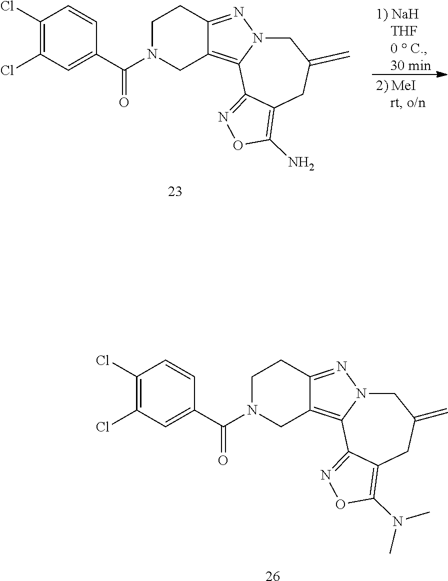

To a solution of compound 23 (25.0 mg, 58.1 μmol) in THF (1.05 mL) at 0° C. was added NaH (95% purity, 2.20 mg, 87.2 μmol). The reaction mixture was stirred at 0° C. for 30 min and iodomethane (4.00 μL, 63.9 μmol) was added. The reaction mixture was warmed to room temperature and stirred overnight. Additional amount of NaH (232 μmol) and iodomethane (0.58 mmol) were added and the reaction mixture was stirred for another 5 h at room temperature. The reaction was quenched with NH₄Cl (sat., aq.) and diluted with water. The layers were separated and the aqueous phase was extracted with EtOAc (twice). The combined organic extracts were washed with brine, dried (Na₂SO₄), filtered and concentrated under reduced pressure. The crude mixture was purified by preparative TLC (mobile phase: 80% EtOAc/heptane) to afford compound 26 (13 mg, 49%) as a white solid.

¹H NMR (400 MHz, MeOD) δ ppm 7.57-7.74 (m, 2H), 7.33-7.46 (m, 1H), 5.19-5.33 (m, 2H), 4.79-4.86 (m, 2H), 4.52-4.70 (m, 2H), 3.96-4.12 (m, 1H), 3.53-3.76 (m, 3H), 2.99-3.17 (m, 6H), 2.73-2.92 (m, 2H); LCMS (method C): Rt=3.49 min, m/z calcd. for C₂₂H₂₁Cl₂N₅O₂ 457, m/z found 458 [M+H]⁺.

### 3.2.2.1.18. Synthesis of Compound 27

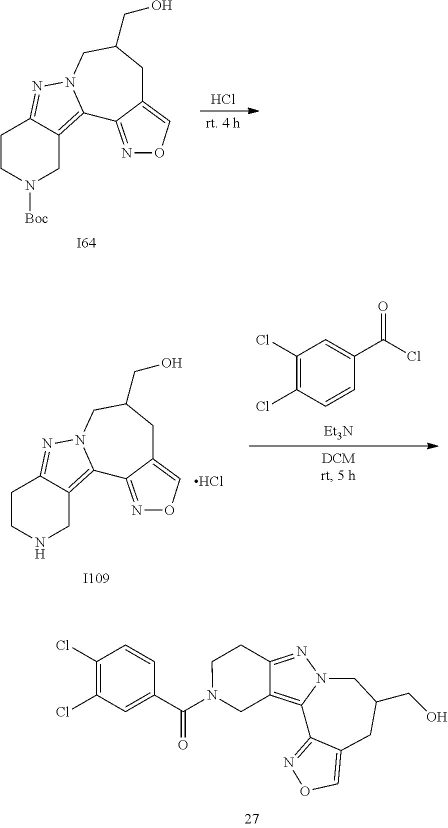

### Intermediate I109

### (5,6,9,10,11,12-Hexahydro-4H-[1,2]oxazolo[3,4-c]pyrido[4′,3′:3,4]pyrazolo[1,5-a]azepin-5-yl)-methanol Hydrochloride

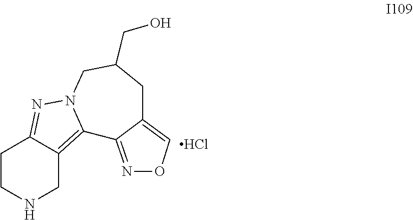 [SMILES: OCC1Cc2conc2-c2c3c(nn2C1)CCNC3 ; Cl]

A mixture of intermediate I64 (2.00 g, 5.55 mmol) and HCl (4M in 1,4-dioxane, 20 mL, 80.0 mmol) was stirred at room temperature for 4 h. The mixture was concentrated under reduced pressure to afford intermediate I109 which was used as such in the next step.

### Compound 27

### (3,4-Dichlorophenyl)[5-(hydroxydiethyl)-5,6,9,10-tetrahydro-4H-[1,2]oxazolo[3,4-c]pyrido-[4′,3′:3,4]pyrazolo[1,5-a]azepin-11(12H)-yl]methanone

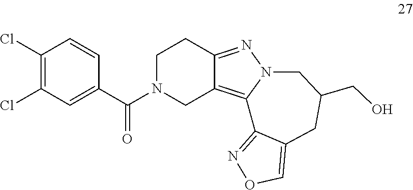 [SMILES: O=C(c1ccc(Cl)c(Cl)c1)N1CCc2nn3c(c2C1)-c1nocc1CC(CO)C3]

To a suspension of intermediate I109 in DCM (20 mL) were added 3,4-dichlorobenzoyl chloride (1.28 g, 6.10 mmol) and Et₃N (7.71 mL, 55.5 mmol). The reaction mixture was stirred at room temperature for 5 h and concentrated under reduced pressure. The crude mixture was adsorbed onto silica and purified by flash column chromatography (silica, mobile phase gradient: 40-100% EtOAc:heptane) to afford compound 27 (1.57 g, 65% over 2 steps).

LCMS (method C): Rt=2.39 min, m/z calcd. for C₂₀H₁₈Cl₂N₄O 432, m/z found 433 [M+H]⁺; ¹H NMR (400 MHz, DMSO-d₆) δ ppm 8.90-8.82 (m, 1H), 7.76-7.73 (m, 21), 7.48 (dd, J=1.51, 8.16 Hz, 1H), 4.95 (br s, 1H), 4.79 (s, 1H), 4.65-4.40 (m, 2H), 4.35-4.10 (m, 1H), 3.93 (br s, 1H), 3.59 (br s, 1H), 3.45-3.30 (m, 2H), 3.05-2.90 (m, 1H), 2.74-2.54 (m, 3H), 2.14 (br s, 1H).

### 3.2.2.1.19. Synthesis of Compound 28

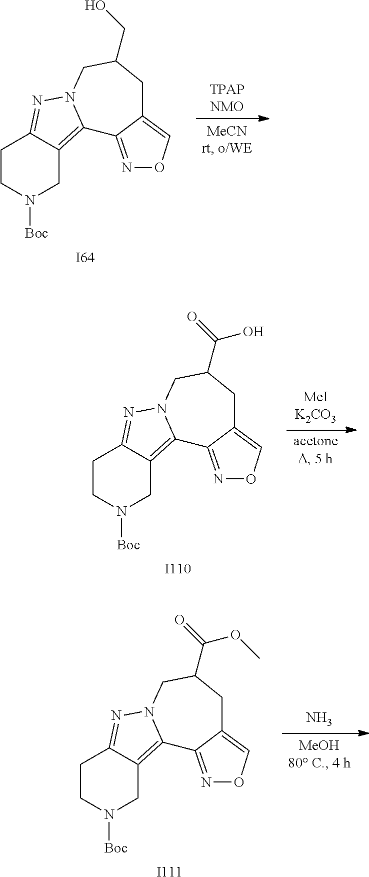

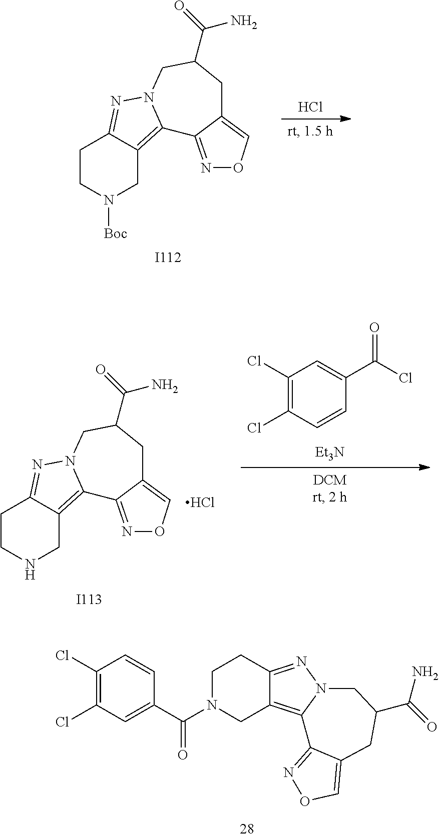

### Intermediate I110

### 11-(tert-Butoxycarbonyl)-5,6,9,10,11,12-hexahydro-4H-[1,2]oxazolo[3,4-c]pyrido[4′,3′:3,4]-pyrazolo[1,5-a]azepine-5-carboxylic Acid

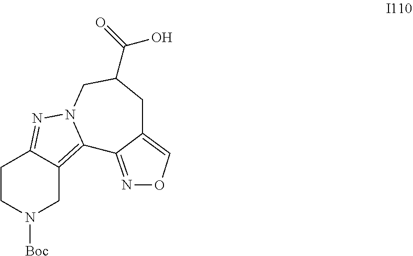 [SMILES: CC(C)(C)OC(=O)N1CCc2nn3c(c2C1)-c1nocc1CC(C(=O)O)C3]

A mixture of intermediate I64 (750 mg, 2.08 mmol). TPAP (73.1 mg, 0.21 mmol) and NMO (2.44 g, 20.8 mmol) in MeCN (15 mL) was stirred at room temperature for 72 h. The reaction mixture was diluted with EtOAc, water and HC (1N, aq.). The layers were separated and the aqueous phase was extracted with EtOAc (3 times). The combined organic extracts were washed with brine, dried (Na₂SO₄), filtered and concentrated under reduced pressure to afford intermediate I110 which was used as such in the next step.

### Intermediate I111

### 11-tert-Butyl 5-methyl 5,6,9,10-tetrahydro-4H-[1,2]oxazolo[3,4-]pyrido[4′,3′:3,4]pyrazolo-[1,5-a]azepine-5,11(12H)-dicarboxylate

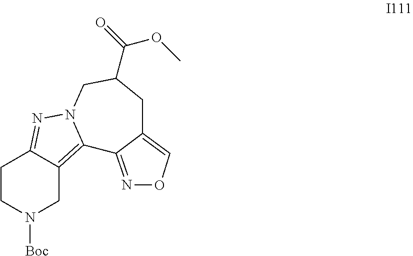 [SMILES: COC(=O)C1Cc2conc2-c2c3c(nn2C1)CCN(C(=O)OC(C)(C)C)C3]

To a mixture of crude intermediate I110 and K₂CO₃ (406 mg, 2.94 mmol) in acetone (11 mL) was added Mel (457 μL, 7.35 mmol). The reaction mixture was stirred under reflux for 5 h. The mixture was filtered and the filtrate was concentrated under reduced pressure. The crude mixture was adsorbed onto silica and purified by flash column chromatography (silica, mobile phase gradient: 40-80% EtOAc/heptane) to afford intermediate I111 (320 mg, 40% over 2 steps).

### Intermediate I112

### tert-Butyl 5-carbamoyl-5,6,9,10-tetrahydro-4H-[1,2]oxazolo[3,4-c]pyrido[4′,3′:3,4]pyrazolo-[1,5-a]azepine-11(12H)-carboxylate

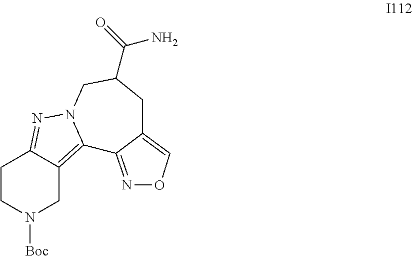 [SMILES: CC(C)(C)OC(=O)N1CCc2nn3c(c2C1)-c1nocc1CC(C(N)=O)C3]

To a solution of intermediate I111 (130 mg, 0.34 mmol) in MeOH (1 mL) was added NH₃ (20% in H₂O, 1.00 mL, 14.8 mmol). The reaction mixture was stirred at room temperature overnight, then at 80° C. for 4 h. The mixture was concentrated under reduced pressure. The mixture was combined with another fraction (0.21 mmol). The residue was dissolved in EtOAc and the solution was washed with NaHCO₃ (aq.) (3 times). The organic phase was dried (Na₂SO₄), filtered and concentrated under reduced pressure to afford intermediate I112.

### Intermediate I113

### 5,6,9,10,11,12-Hexahydro-4H-[1,2]oxazolo[3,4-c]pyrido[4′,3′:3,4]pyrazolo[1,5-a]azepine-5-carboxamide Hydrochloride

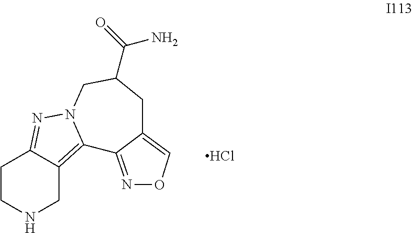 [SMILES: NC(=O)C1Cc2conc2-c2c3c(nn2C1)CCNC3 ; Cl]

A mixture of intermediate I112 and HCl (4N in 1,4-dioxane, 1.5 mL, 6.00 mmol) was stirred at room temperature for 1.5 h and the mixture was concentrated under reduced pressure to afford intermediate I113 which was used as such in the next step.

### Compound 28

### 11-(3,4-Dichlorobenzoyl)-5,6,9,10,11,12-hexahydro-4H-[1,2]oxazolo[3,4-c]pyrido[4′,3′:3,4]-pyrazolo[1,5-a]azepine-5-carboxamide

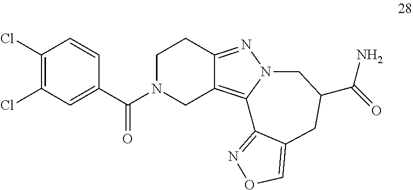 [SMILES: NC(=O)C1Cc2conc2-c2c3c(nn2C1)CCN(C(=O)c1ccc(Cl)c(Cl)c1)C3]

To a suspension of intermediate I113 and 3,4-dichlorobenzoyl chloride (35.3 mg, 0.17 mmol) in DCM (2.0 mL) was added Et₃N (0.11 mL, 0.77 mmol). The reaction mixture was stirred at room temperature for 2 h. filtered and concentrated under reduced pressure. The crude mixture was purified by recrystallization from EtOH to afford compound 28 (30.7 mg, 45% over 3 steps). LCMS (method C): Rt=2.15 min, m/z calcd. for C₂₀H₁₇Cl₂N₅O₃ 445, m/z found 446 [M+H]⁺; ¹H NMR (DMSO-d₆, 75° C.) δ ppm 8.81 (s, 1H), 7.66-7.75 (m, 2H), 7.39-7.50 (m, 1H), 4.58-4.79 (m, 3H), 4.44-4.55 (m, 1H), 3.63-3.85 (m, 2H), 2.92-3.23 (m, overlaps with solvent peak), 2.70-2.79 (m, 2H).

### 3.2.2.1.20. Synthesis of Compound 29

### 11-(3,4-Dichlorobenzoyl)-5,6,9,10,11,12-hexahydro-4H-[1,2]oxazolo[3,4-c]pyrido[4′,3′:3,4]-pyrazolo[1,5-a]azepine-5-carboxylic Acid

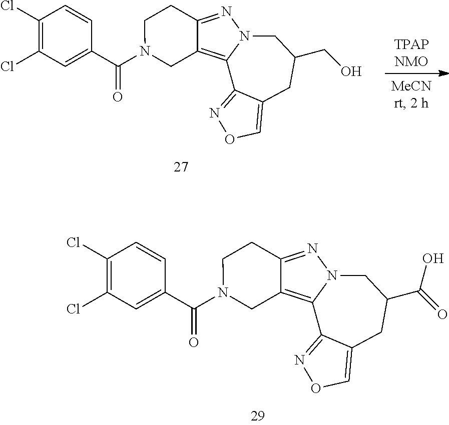

To a solution of compound 27 (200 mg) in MeCN (10 mL) were added TPAP (40.6 mg, 115 μmol) and NMO (270 mg, 2.31 μmol). The reaction mixture was stirred at room temperature for 2 h and quenched with HCl (1N, 25 mL). The mixture was diluted with water (20 mL) and extracted with EtOAc (3×30 mL). The combined organic extracts were dried (Na₂SO₄), filtered and concentrated under reduced pressure. The crude mixture was purified by reverse phase HPLC (Gilson GX-281 semi-prep-HPLC with Phenomenex Synergi C18 (10 μm, 150×25 mm), or Boston Green ODS C18 (5 μm, 150×30 mm), and mobile phase of 5-99% MeCN in water (with 0.225% FA) over 10 min and then hold at 100% MeCN for 2 min. at a flow rate of 25 mL/min) to give compound 29 (12.5 mg) as a white solid.

MS (ESI): m/z calcd. for C₂₀H₁₆Cl₂N₄O₄ 446.1; m/z found, 447.0 [M+H]⁺; ¹H NMR (400 MHz, DMSO-d₆) δ ppm 9.10-8.74 (m, 1H), 7.96-7.66 (m, 2H), 7.47 (br d, J=8.0 Hz, 1H), 5.06-4.41 (m, 4H), 3.91 (br s, 1H), 3.57 (br s, 1H), 3.25-2.94 (m, 3H), 2.73 (br s, 2H).

### 3.2.2.1.21. Synthesis of Compounds 30 and 31

### (5′R)-11-(3,4-Dichlorobenzoyl)-N-methyl-5,6,9,10,11,12-hexahydro-4H-[1,2]oxazolo[3,4-c]-pyrido[4′,3′:3,4]pyrazolo[1,5-a]azepine-5-carboxamide and (5*S)-11-(3,4-Dichlorobenzoyl)-N-methyl-5,6,9,10,11,12-hexahydro-4H-[1,2]oxazolo[3,4-c]pyrido[4′,3′:3,4]pyrazolo[1,5-a]azepine-5-carboxamide

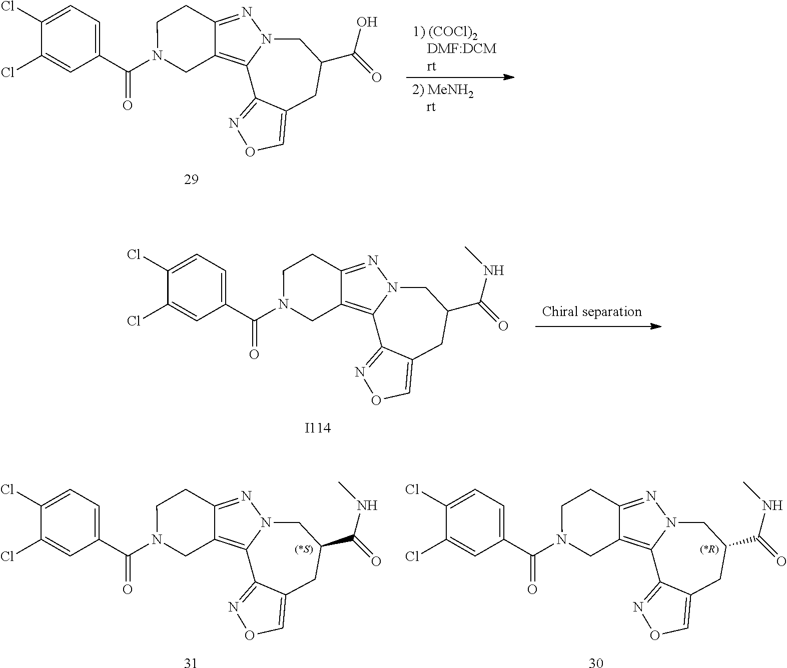

To a suspension of compound 29 (268 mg, 0.60 mmol) in DCM (7.66 mL) were added DMF (76.6 μL) and oxalyl chloride (2M in DCM, 899 μL, 1.80 mmol). The reaction mixture was stirred at room temperature for 1 h. To this orange solution was added methylamine (2M in THF, 1.50 mL, 3.00 mmol). The reaction mixture was stirred at room temperature overnight and concentrated under reduced pressure. The residue was dissolved in EtOAc and the organic layer was washed with NaHCO₃ (aq.), NaOH (1M, aq.) and brine, dried (Na₂SO₄), filtered and concentrated under reduced pressure. The crude mixture was combined with another fraction (0.15 mmol) and purified by flash column chromatography (silica, mobile phase gradient: 0-10% MeOH/EtOAc). The enantiomers were separated via Prep SFC (Stationary phase: Chiralcel Diacel OJ 20×250 mm, mobile phase: CO₂, EtOH+0.4% i-PrNH₂) to afford compound 30 (52 mg, 15%) and compound 31 (56 mg, 16%).

Compound 30: ¹H NMR (400 MHz, DMSO-d₆, 100° C.) δ ppm 8.77 (s, 1H), 7.64-7.77 (m, 3H), 7.42 (dd, J=8.1, 2.0 Hz, 1H), 4.65-4.76 (m, 2H), 4.57-4.64 (m, 1H), 4.41-4.51 (m, 1H), 3.82 (s, 1H), 3.67-3.78 (m, 2H), 3.02-3.14 (m, 2H), 2.70-2.77 (m, 2H), 2.61 (d, J=4.6 Hz, 3H); LCMS (method B): Rt=1.67 min. m/z calcd. for C₂₁H₁₉Cl₂N₅O₃ 459, m/z found 460 [M+H]⁺.

Compound 31: ¹H NMR (400 MHz, DMSO-d₆, 100° C.) δ ppm 8.77 (s, 1H), 7.69-7.75 (m, 2H), 7.64-7.70 (m, 1H), 7.42 (dd, J=8.4, 2.0 Hz, 1H), 4.65-4.76 (m, 2H), 4.58-4.64 (m, 1H), 4.42-4.50 (m, 1H), 3.82 (s, 1H), 3.68-3.79 (m, 2H), 3.07-3.14 (m, 1H), 2.98-3.06 (m, 1H), 2.71-2.77 (m, 2H), 2.61 (d, J=4.6 Hz, 3H); LCMS (method B): Rt=1.68 min, m/z calcd. for C₂₁H₁₉Cl₂N₅O₃ 459, m/z found 460 [M+H]⁺.

### 3.2.2.1.22. Synthesis of Compound 32

### 11-(3,4-Dichlorobenzoyl)-N-ethyl-5,6,9,10,11,12-hexahydro-4H-[1,2]oxazolo[3,4-c]pyrido-[4′,3′:3,4]pyrazolo[1,5-a]azepine-5-carboxamide

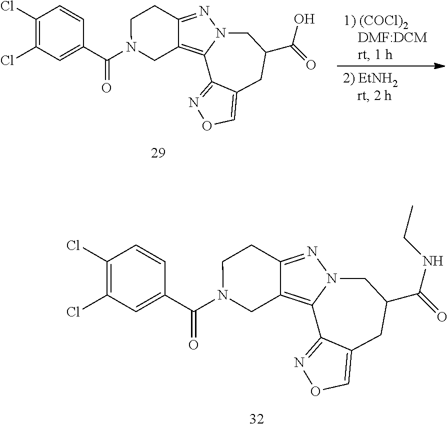

To a suspension of compound 29 (35.0 mg, 78.3 μmol) in DCM (1 mL) were added DMF (10 μL, 0.13 mmol) and oxalyl chloride (2M in DCM, 78.3 μL, 157 μmol). The reaction mixture was stirred at room temperature for 1 h. To this orange solution was added ethylamine (2M in THF, 0.20 mL, 0.40 mmol). The reaction mixture was stirred at room temperature for 2 h and concentrated under reduced pressure. The residue was washed with MeOH to afford compound 32 (23.1 mg, 62%) as a white solid.

¹H NMR (400 MHz, DMSO-d₆) δ 8.90 (d, J=33.9 Hz, 1H), 8.13 (s, 1H), 7.81-7.66 (m, 2H), 7.47 (dd, J=8.5, 1.8 Hz, 1H), 4.78 (s, 1H), 4.67-4.38 (m, 3H), 3.92 (s, 1H), 3.58 (s, 1H), 3.18-2.57 (m, 7H), 1.00 (t, J=6.8 Hz, 3H); LCMS (method C): Rt=2.62 min, m/z calcd. for C₂₂H₂₁Cl₂N₅O₃ 473, m/z found 474 [M+H]⁺.

### 3.2.2.1.23. Synthesis of Compound 33

### N-Cyclopropyl-11-(3,4-dichlorobenzoyl)-5,6,9,10,11,12-hexahydro-4H-[1,2]oxazolo[3,4-c]-pyrido[4′,3′:3,4]pyrazolo[1,5-a]azepine-5-carboxamide

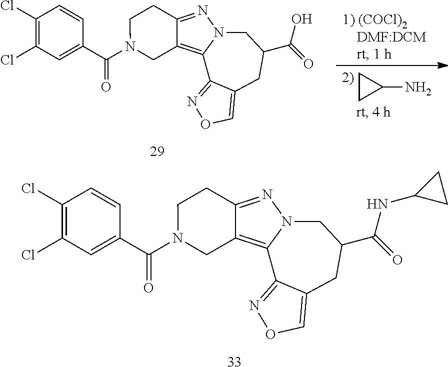

Compound 33 was prepared in an analogous manner to that described for compound 32. However, the product was precipitated out of the solution during stirring. The solid was collected by filtration and washed with MeOH to afford compound 33 (31 mg, 81%) as a white solid.

LCMS (method C): Rt=2.62 min, m/z calcd. for C₂₃H₂₁Cl₂N₅O₃ 485, m/z found 486 [M+H]⁺.

### 3.2.2.1.24. Synthesis of Compound 34

### 11-(3,4-Dichlorobenzoyl)-N-(2-hydroxyethyl)-5,6,9,10,11,12-hexahydro-4H-[1,2]oxazolo-[3,4-c]pyrido[4′,3′:3,4]pyrazolo[1,5-a]azepine-5-carboxamide

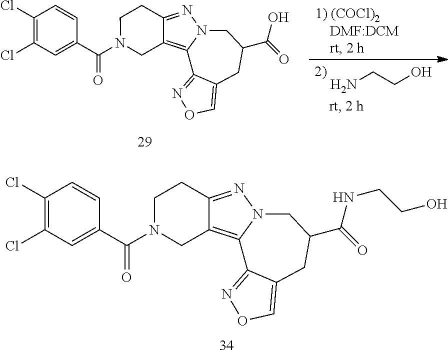

To a suspension of compound 29 (60.0 mg, 0.13 mmol) in DCM (1.00 mL) were added DMF (1.04 μL, 13.4 μmol) and oxalyl chloride (2M in DCM, 0.29 mL, 0.54 mmol). The reaction mixture was stirred at room temperature for 2 h. Ethanolamine (40.5 μL, 0.67 mmol) was added and the reaction mixture was stirred for another 2 h. The mixture was diluted with DCM and water. The layers were separated and the aqueous phase was extracted with DCM (3 times). The combined organic extracts were washed with brine, dried (MgSO₄), filtered and partially concentrated under reduced pressure. The mixture was purified by preparative TLC (100% EtOAc) to afford compound 34 (13.2 mg, 20%) as a pale yellow solid.

¹H NMR (400 MHz, DMSO-d₆) δ8.90 (d, J=33.9 Hz, 1H), 8.19 (s, 1H), 7.80-7.70 (m, 2H), 7.52-7.42 (m, 1H), 4.83-4.41 (m, 5H), 3.92 (s, 1H), 3.58 (s, 1H), 3.20-2.64 (m, 8H); LCMS (method D): Rt=2.38 min, m/z calcd. for C₂₂H₂₁Cl₂N₅O₄ 489, m/z found 490 [M+H]⁺.

### 3.2.2.1.25. Synthesis of Compound 35

### 11-(3,4-Dichlorobenzoyl)-N-(2,2,2-trifluoroethyl)-5,6,9,10,11,12-hexahydro-4H-[1,2]oxazolo-[3,4-c]pyrido[4′,3′:3,4]pyrazolo[1,5-a]azepine-5-carboxamide

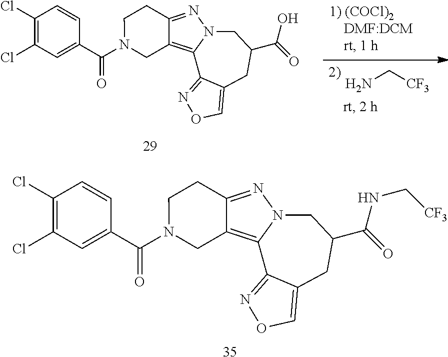

Compound 35 was prepared according to the procedure reported for the synthesis of compound 32.

The mixture was concentrated under reduced pressure and MeOH was added. The mixture was adsorbed onto silica and purified by flash column chromatography (silica gel, mobile phase gradient: 0-10% MeOH/EtOAc) to afford compound 35 (18.4 mg, 52%) as a white solid.

LCMS (method C): Rt=2.88 min. m-z calcd. for C₂₂H₁₈Cl₂F₃N₅O₃ 527, m/z found 528 [M+H]⁺; ¹H NMR (400 MHz, DMSO-d₆) δ 8.77-9.04 (m, 2H), 7.69-7.81 (m, 2H), 7.47 (br d, J=7.3 Hz, 1H), 4.78 (br s, 1H), 4.52-4.66 (m, 3H), 3.84-4.04 (m, 3H), 3.52-3.64 (m, 1H), 2.99-3.15 (m, 3H), 2.68-2.82 (m, 2H).

### 3.2.2.1.26. Synthesis of Compound 36

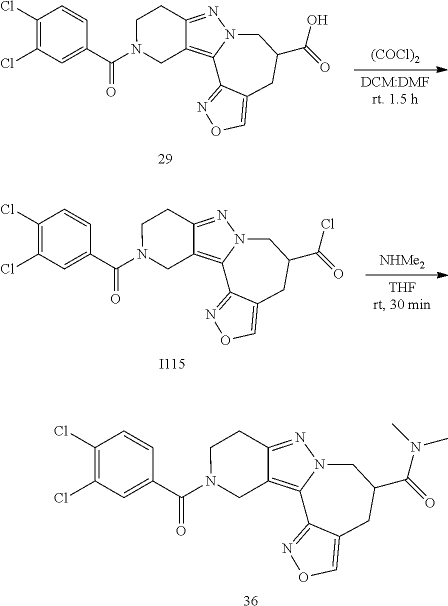

### Intermediate I115

### 11-(3,4-Dichlorobenzoyl)-5,6,9,10,11,12-hexahydro-4H-[1,2]oxazolo[3,4-]pyrido[4′,3′:3,4]-pyrazolo[1,5-a]azepine-5-carbonyl Chloride

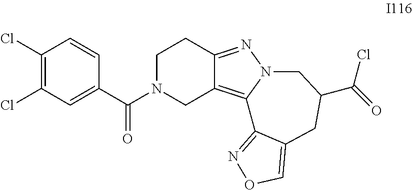 [SMILES: O=C(Cl)C1Cc2conc2-c2c3c(nn2C1)CCN(C(=O)c1ccc(Cl)c(Cl)c1)C3]

To a solution of compound 29 (58.0 mg, 0.13 mmol) in DCM (0.6 mL) and DMF (54 μL) under N2 atmosphere was added oxalyl chloride (2M in DCM, 0.13 mL, 0.26 mmol). The reaction mixture was stirred at room temperature for 1.5 h to afford intermediate I115 and the mixture was split into 3 batches that were used in subsequent reactions.

### Compound 36

### 11-(3,4-Dichlorobenzoyl)-N,N-dimethyl-5,6,9,10,11,12-hexahydro-4H-[1,2]oxazolo[3,4-c]pyrido[4′,3′:3,4]pyrazolo[1,5-a]azepine-5-carboxamide

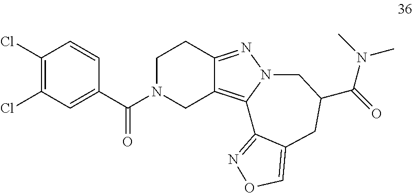 [SMILES: CN(C)C(=O)C1Cc2conc2-c2c3c(nn2C1)CCN(C(=O)c1ccc(Cl)c(Cl)c1)C3]

To a solution of intermediate I115 was added dimethylamine (2M in THF, 0.11 mL, 0.22 mmol) and the reaction mixture was stirred at room temperature for 30 min. The mixture was concentrated under reduced pressure and purified by preparative TLC (mobile phase: 2% MeOH/EtOAc) to afford compound 36 (18.7 mg, 91%).

LCMS (method D): Rt=2.91 min, m/z calcd. for C₂₂H₂₁Cl₂N₅O₃ 473, m/z found 474 [M+H]⁺; ¹H NMR (400 MHz, MeOD) δ ppm 8.52-8.70 (m, 1H), 7.54-7.75 (M, 2H), 7.41 (br d, J=7.8 Hz, 1H), 4.58-4.78 (m, 2H), 4.34-4.52 (m, 1H), 3.93-4.17 (m, 1H), 3.64-3.74 (m, 1H), 3.41-3.57 (m, 1H), 3.00-3.25 (m, 5H), 2.91-2.98 (m, 3H), 2.76-2.88 (m, 3H).

### 3.2.2.1.27. Synthesis of Compound 37

### 11-(3,4-Dichlorobenzoyl)-N-phenyl-5,6,9,10,11,12-hexahydro-4H-[1,2]oxazolo[3,4-c]-pyrido[4′,3′:3,4]pyrazolo[1,5-a]azepine-5-carboxamide

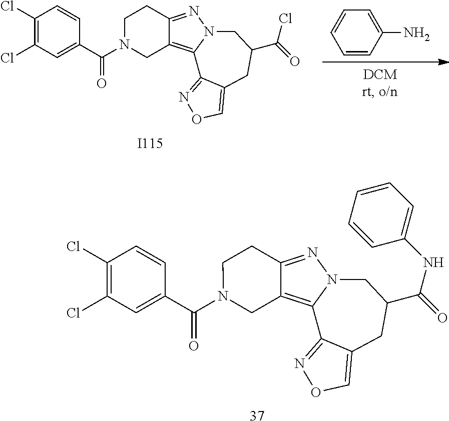

To a solution of intermediate I115 (30.0 mg, 64.4 μmol) in DCM (1 mL) was added aniline (29.3 μL, 0.32 mmol). The reaction mixture was stirred at room temperature overnight and concentrated under reduced pressure. The crude mixture was purified by reverse phase HPLC (Gilson, 100 mm×30 mm, 10-100% ACN/water both containing 0.1% TFA). The residue was washed with DCM and MeOH to afford compound 37 (7.6 mg, 23%) as an off-white solid.

LCMS (method C): Rt=3.30 min, m/z calcd. for C₂₆H₂₁Cl₂N₅O₃ 521, m/z found 522 [M+H]⁺: ¹H NMR (400 MHz, DMSO-d₆) δ ppm 10.13-10.30 (m, 1H), 8.82-9.11 (m, 1H), 7.70-7.82 (m, 2H), 7.41-7.63 (m, 3H), 7.31 (t, J=8.1 Hz, 2H), 7.02-7.09 (m, 1H), 4.51-4.87 (m, 4H), 3.85-3.98 (m, 1H), 3.52-3.66 (m, 1H), 3.05-3.29 (m, 3H), 2.68-2.83 (m, 2H).

### 3.2.2.1.28. Synthesis of Compound 38

### [1-(3,4-Dichlorobenzoyl)-5,6,9,10,11,12-hexahydro-4H-[1,2]oxazolo[3,4-c]pyrido[4′,3′:3,4]-pyrazolo[1,5-a]azepin-5-yl](morpholin-4-yl)methanone

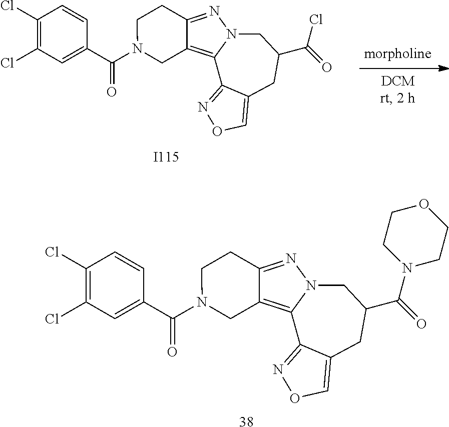 [SMILES: "I115" ; O=C(Cl)C1Cc2conc2-c2c3c(nn2C1)CCN(C(=O)c1ccc(Cl)c(Cl)c1)C3 ; "DCMrt, 2 h" ; "morpholine" ; O=C(c1ccc(Cl)c(Cl)c1)N1CCc2nn3c(c2C1)-c1nocc1CC(C(=O)N1CCOCC1)C3 ; "38"]

To a solution of intermediate I115 was added morpholine (7.49 mg, 86 μmol) and the reaction mixture was stirred at room temperature for 2 h. The mixture was concentrated under reduced pressure and purified by preparative TLC (mobile phase: 2% MeOH/EtOAc) to afford compound 38 (11.3 mg, 50%) as a white solid.

LCMS (method C): Rt=2.72 min, m/z calcd. for C₂₄H₂₃Cl₂N₅O₄ 515, m/z found 516 [M+H]⁺.

### 3.2.2.1.29. Synthesis of Compound 39

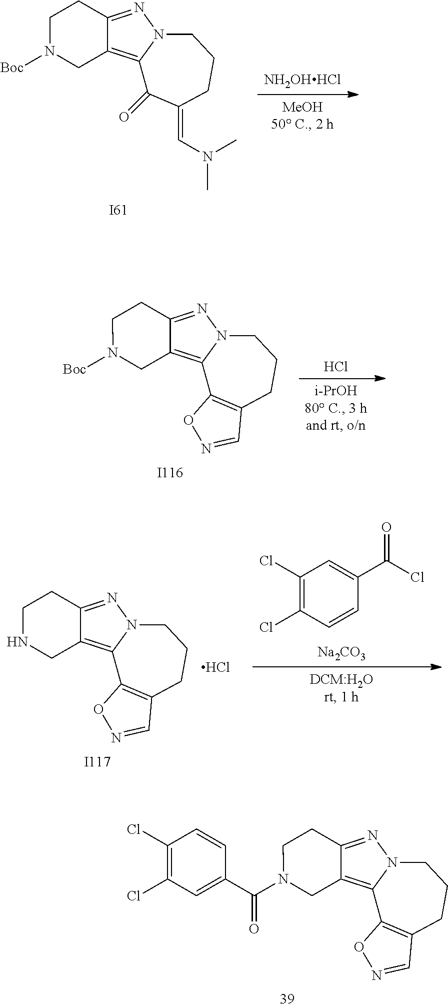

### Intermediate I116

### Tert-Butyl 4,5,6,9,10,12-hexahydro-11H-[1,2]oxazolo[5,4-c]pyrido[4′,3′:3,4]pyrazolo[1,5-a]-azepine-1-carboxylate

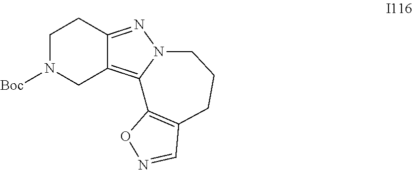 [SMILES: CC(C)(C)OC(=O)N1CCc2nn3c(c2C1)-c1oncc1CCC3]

Hydroxylamine hydrochloride (68 μL, 1.11 mmol) was added to a solution of intermediate I51 (100 mg, 0.28 mmol) in MeOH (5 mL). The reaction mixture was stirred at 50° C. for 2 h. The volatiles were removed under reduced pressure and the residue was purified by flash column chromatography (silica, mobile phase gradient: heptane to EtOAc) to afford intermediate I116 (58 mg, 63%).

### Intermediate I117

### 5,6,9,10,11,12-Hexahydro-4H-[1,2]oxazolo[5,4-c]pyrido[4′,3′:3,4]pyrazolo[1,5-a]azepine Hydrochloride

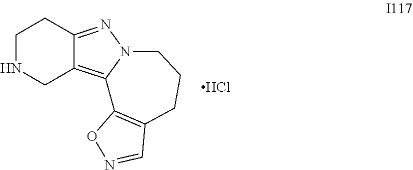 [SMILES: c1noc2c1CCCn1nc3c(c1-2)CNCC3 ; Cl]

HCl (6M in i-PrOH, 0.75 mL, 4.50 mmol) was added to a solution of intermediate I116 (58 mg, 0.18 mmol) in i-PrOH (5 mL). The reaction mixture was stirred at 80° C. for 1 h and at room temperature overnight. The volatiles were removed under reduced pressure to afford intermediate I117 which was used as such in the next step.

### Compound 39

### (3,4-Dichlorophenyl)(4,5,6,9,10,12-hexahydro-11H-[1,2]oxazolo[5,4-c]pyrido[4′,3′:3,4]-pyrazolo[1,5-a]azepin-11-yl)methanone

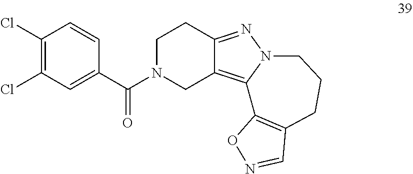 [SMILES: O=C(c1ccc(Cl)c(Cl)c1)N1CCc2nn3c(c2C1)-c1oncc1CCC3]

A mixture of intermediate I117, 3,4-dichlorobenzoyl chloride (39.8 mg, 0.18 mmol) and Na₂CO₃ (37.2 mg, 0.35 mmol) in DCM (5 mL) and water (5 mL) was stirred vigorously at room temperature for 1 h. The organic layer was loaded on a silica cartridge and purified by flash column chromatography (silica, mobile phase gradient: heptane to EtOAc) to afford compound 39 (26.9 mg, 38% over 2 steps).

¹H NMR (400 MHz, DMSO-d₆, 100° C.) δ ppm 8.46 (s, 1H), 7.69 (d, J=4.8 Hz, 1H), 7.68 (d, J=1.1 Hz, 1H), 7.44 (dd, J=8.3, 1.9 Hz, 1H), 4.80 (br s, 2H), 4.38-4.45 (m, 2H), 3.69-3.79 (m, 2H), 2.81 (t, J=6.1 Hz, 2H), 2.73 (t, J=5.8 Hz, 2H), 2.08-2.17 (m, 2H); LCMS (method A): Rt=0.99 min, m/z calcd. for C₁₉H₁₆Cl₂N₄O₂ 402, m/z found 403 [M+H]⁺.

### 3.2.2.2. Synthesis of Oxazole Derivative Compounds

### Synthesis of Compound 40

### (3,4-Dichlorophenyl)(4,5,6,9,10,12-hexahydro-11H-[1,3]oxazolo[4,5-c]pyrido-[4′,3′:3,4]pyrazolo[1,5-a]azepin-11-yl)methanone

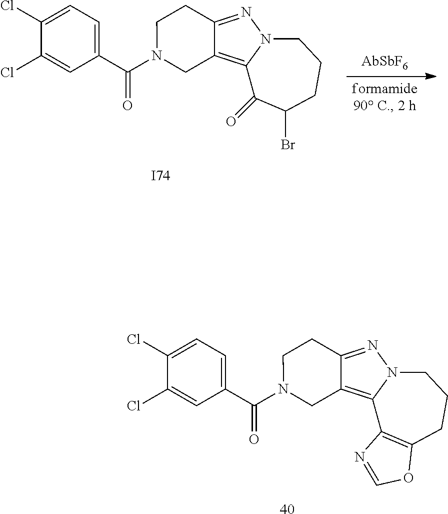 [SMILES: "I74" ; O=C1c2c3c(nn2CCCC1Br)CCN(C(=O)c1ccc(Cl)c(Cl)c1)C3 ; "AbSbF6" ; "formamide90° C., 2 h" ; O=C(c1ccc(Cl)c(Cl)c1)N1CCc2nn3c(c2C1)-c1ncoc1CCC3 ; "40"]

The reaction was performed under anhydrous conditions.

To a solution of intermediate I74 (200 mg, 0.44 mmol) in formamide (2 mL) was added AgSbF₆ (150 mg, 0.44 mmol). The reaction mixture was stirred at 90° C. under microwave irradiation for 2 h. The reaction mixture was diluted with DCM (20 mL), filtered through a pad of Celite® and the filtrate was concentrated under reduced pressure. The crude mixture was purified by flash column chromatography (silica gel, mobile phase: DCM/MeOH, gradient from: 100:0 to 98:2). The product was dried at 50° C. overnight to afford compound 40 (64 mg, 36%) as a white solid.

¹H NMR (400 MHz, DMSO-d₆, 80° C.) δ ppm 8.27 (s, 1H), 7.70 (d, J=8.5 Hz, 1H), 7.67 (d, J=1.9 Hz, 1H), 7.43 (dd, J=8.5, 1.9 Hz, 1H), 4.80 (s, 2H), 4.38-4.30 (m, 2H), 3.78-3.65 (m, 2H), 3.07 (t, J=5.6 Hz, 2H), 2.72 (t, J=5.7 Hz, 2H), 2.25-2.12 (m, 2H); LCMS (method E): Rt=9.8 min, m/z calcd. for C₁₉H₁₆Cl₂N₄O₂ 402, m/z found 403 [M+H]⁺.

### 3.2.2.3. Synthesis of Pyrazole Derivative Compounds

### 3.2.2.3.1. Synthesis of Compound 41

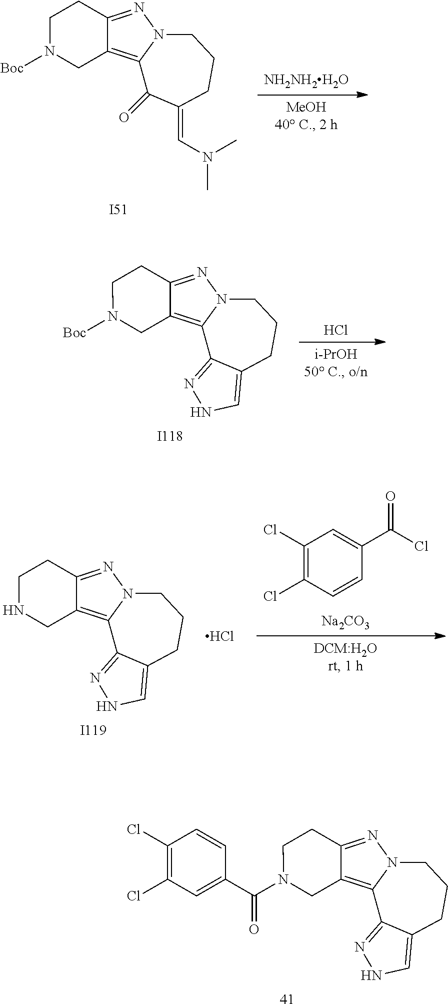

### Intermediate I118

### Tert-Butyl 2,5,6,9,10,12-hexahydropyrazolo[3,4-c]pyrido[4′,3′:3,4]pyrazolo[1,5-a]azepine-11(4H)-carboxylate

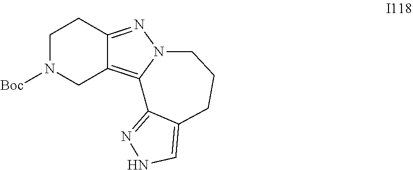 [SMILES: CC(C)(C)OC(=O)N1CCc2nn3c(c2C1)-c1n[nH]cc1CCC3]

Hydrazine monohydrate (50% in H₂O, 34.56 μL, 0.55 mmol) was added to a solution of intermediate I51 (100 mg, 0.28 mmol) in MeOH (5 mL). The reaction mixture was stirred at 40° C. for 2 h. The volatiles were removed under reduced pressure and the residue was purified by flash column chromatography (silica, mobile phase gradient: heptane to EtOAc) to afford intermediate I118 (57 mg, 62%) as a white powder.

### Intermediate I119

### 2,4,5,6,9,10,11,12-Octahydropyrazolo[3,4-c]pyrido[4′,3′:3,4]pyrazolo[1,5-a]azepine Hydrochloride

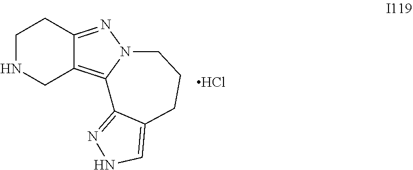 [SMILES: c1[nH]nc2c1CCCn1nc3c(c1-2)CNCC3 ; Cl]

HCl (6M in f-PrOH, 288 μL, 1.73 mmol) was added to a solution of intermediate I118 (57.0 mg, 0.17 mmol) in i-PrOH (5 mL). The reaction mixture was stirred overnight at 50° C. The volatiles were removed under reduced pressure to afford intermediate I119 which was used as such in the next step.

### Compound 41

### (3,4-Dichlorophenyl)(2,5,6,9,10,12-hexahydropyrazolo[3,4-c]pyrido[4′,3′,3,4]pyrazolo[1,5-a]-azepin-11(4H)-yl)methanone

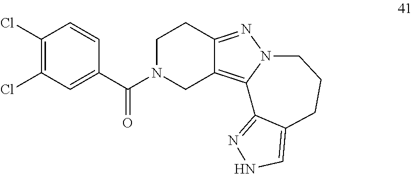 [SMILES: O=C(c1ccc(Cl)c(Cl)c1)N1CCc2nn3c(c2C1)-c1n[nH]cc1CCC3]

A mixture of intermediate I119, 3,4-dichlorobenzoyl chloride (39.2 mg, 0.18 mmol) and Na₂CO₃ (36.7 mg, 0.35 mmol) in DCM (5 mL) and water (5 mL) was stirred vigorously at room temperature for 1 h. The mixture was loaded on a silica cartridge and the mixture was purified by flash column chromatography (silica, mobile phase gradient: heptane to EtOAc). The residue was purified via preparative HPLC (stationary phase: RP XBridge Prep C18 OBD-10 μm, 30×150 mm, mobile phase: NH₄HCO₃ (0.25% in water)/MeCN) to afford compound 41 (31.7 mg, 46% over 2 steps).

¹H NMR (400 MHz, DMSO-d₆, 100° C.) δ ppm 12.39-12.93 (m, 1H), 7.62-7.68 (n, 2H), 7.52 (s, 1H), 7.40 (dd, J=8.3, 1.8 Hz, 1H), 4.73 (s, 2H), 4.33-4.39 (m, 2H), 3.64-3.80 (m, 2H), 2.92 (br s, 1H), 2.84-2.89 (m, 2H), 2.69 (t, J=5.9 Hz, 2H); LCMS (method A): Rt=0.88 min, m/z calcd. for C₁₉H₁₇Cl₂N₅O 401, m/z found 402 [M+H]⁺.

### 3.2.2.3.2. Synthesis of Compound 42

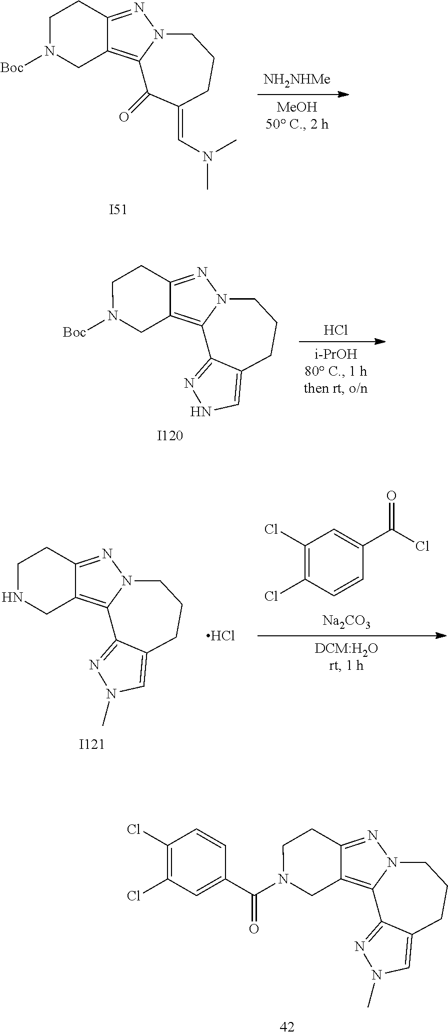

### Intermediate I120

### Tert-Butyl 2-methyl-2,5,6,9,10,12-hexahydropyrazolo[3,4-c]pyrido[4′,3′:3,4]pyrazolo[1,5-a]-azepine-11(4H)-carboxylate

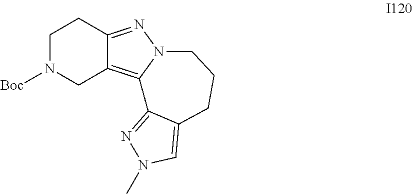 [SMILES: Cn1cc2c(n1)-c1c3c(nn1CCC2)CCN(C(=O)OC(C)(C)C)C3]

Methylhydrazine (29.8 μL, 0.56 mmol) was added to a solution of intermediate I51 (1M) mg, 0.28 mmol) in MeOH (5 mL). The reaction mixture was stirred at 50° C. for 2 h. The volatiles were removed under reduced pressure and the residue was purified by flash column chromatography (silica gel, mobile phase gradient: heptane to EtOAc) to afford intermediate I120 (50 mg, 52%) as a white powder.

### Intermediate I121

### 2-Methyl-2,4,5,6,9,10,11,12-octahydropyrazolo[3,4-c]pyrido[4′,3′:3,4]pyrazolo[1,5-a]azepine Hydrochloride

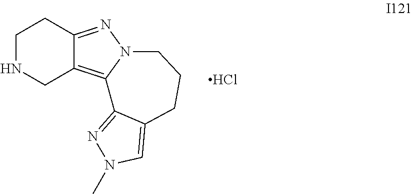 [SMILES: Cn1cc2c(n1)-c1c3c(nn1CCC2)CCNC3 ; Cl]

HCl (6M in i-PrOH, 500 μL, 3.00 mmol) was added to a solution of intermediate I120 (50 mg, 0.15 mmol) in i-PrOH (10 mL). The reaction mixture was stirred at 80° C. for 1 h and at room temperature overnight. The volatiles were removed under reduced pressure to afford intermediate I121 which was used as such in the next step.

### Compound 42

### (3,4-Dichlorophenyl)(2-methyl-2,5,6,9,10,12-hexahydropyrazolo[3,4-c]pyrido[4′,3′:3,4]-pyrazolo[1,5-a]azepin-11(4H)-yl)methanone

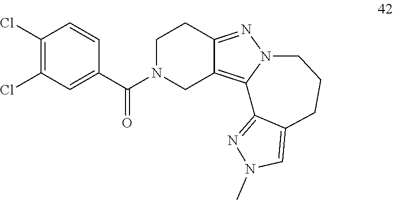 [SMILES: Cn1cc2c(n1)-c1c3c(nn1CCC2)CCN(C(=O)c1ccc(Cl)c(Cl)c1)C3]

A mixture of intermediate I121, 3,4-dichlorobenzoyl chloride (33.0 mg, 0.15 mmol) and Na₂CO₃ (30.9 mg, 0.29 mmol) in DCM (5 mL) and water (5 mL) was stirred vigorously at room temperature for 1 h. The mixture was loaded on a silica cartridge and the mixture was purified by flash column chromatography (silica, mobile phase gradient: heptane/EtOAc). The residue was purified via preparative HPLC (stationary phase: RP XBridge Prep C18 OBD-10 μm, 30×15 (mm, mobile phase: NH₄HCO₃ (0.25% in water)/MeCN) to afford compound 42 (32.1 mg, 53% over 2 steps).

¹H NMR (400 MHz, DMSO-d₆, 100° C.) δ ppm 7.64-7.69 (m, 2H), 7.49 (s, 1H), 7.41 (dd, J=8.1, 2.0 Hz, 1H), 4.72 (br s, 2H), 4.31-4.37 (m, 2H), 3.78 (s, 3H), 3.65-3.75 (m, 2H), 2.79-2.85 (m, 2H), 2.68 (t, J=5.9 Hz, 2H), 2.02-2.09 (m, 2H); LCMS (method A): Rt=1.00 min, m/z calcd. for C₂₀H₁₉Cl₂N₅O 415, m/z found 416 [M+H]⁺.

### 3.2.2.3.3. Synthesis of Compound 43

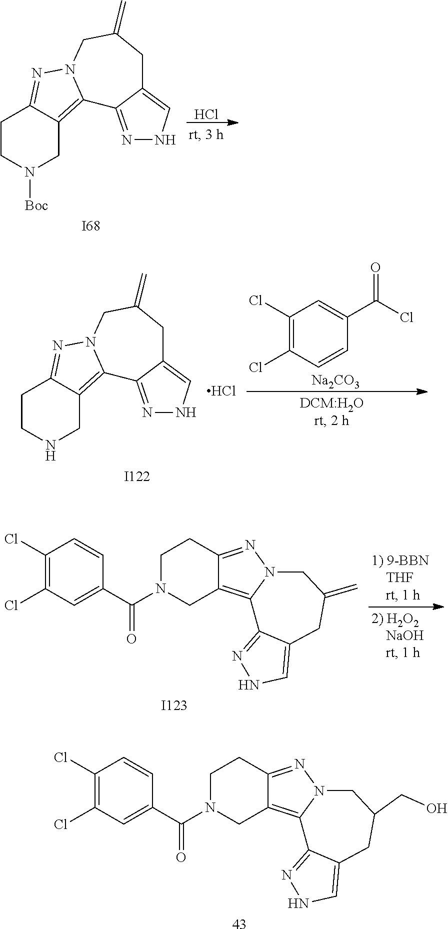

### Intermediate I122

### 5-Methylidene-2,4,5,6,9,10,11,12-octahydropyrazolo[3,4-c]pyrido[4′,3′:3,4]pyrazolo[1,5-a]-azepine Hydrochloride

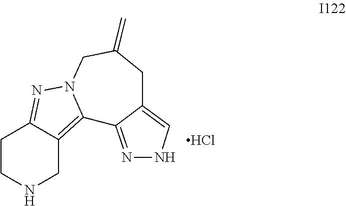 [SMILES: C=C1Cc2c[nH]nc2-c2c3c(nn2C1)CCNC3 ; Cl]

A solution of intermediate I68 (812 mg, 2.38 mmol) in HCl (4M in 1,4-dioxane, 6.0 mL, 24.0 mmol) was stirred at room temperature for 3 h and the mixture was concentrated under reduced pressure to afford intermediate I122 which was used as such in the next step.

### Intermediate I123

### (3,4-Dichlorophenyl)(5-methylidene-2,5,6,9,10,12-hexahydropyrazolo[3,4-c]pyrido[4′,3′:3,4]pyrazolo[1,5-a]azepin-11(4H)-yl)methanone

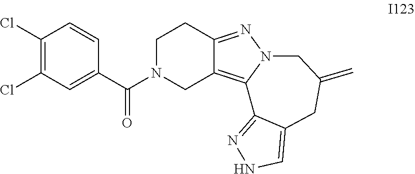 [SMILES: C=C1Cc2c[nH]nc2-c2c3c(nn2C1)CCN(C(=O)c1ccc(Cl)c(Cl)c1)C3]

To a mixture of intermediate I122, 3,4-dichlorobenzoyl chloride (257 mg, 1.23 mmol) were added DCM (34 mL) and H₂O (34 mL). Na₂CO₃ (247 mg, 2.33 mmol) was added and the reaction mixture was stirred vigorously at room temperature for 2 h. The layers were separated and the aqueous phase was extracted with DCM. The combined organic extracts were washed with brine, dried (MgSO₄), filtered and concentrated under reduced pressure. MeOH was added to the residue. The solution was filtered and concentrated under reduced pressure to afford intermediate I123 which was used as such in the next step.

### Compound 43

### (3,4-Dichlorophenyl)[5-(hydroxymethyl)-2,5,6,9,10,12-hexahydropyrazolo[3,4-c]pyrido-[4′,3′:3,4]pyrazolo[1,5-a]azepin-11(41)-yl]methanone

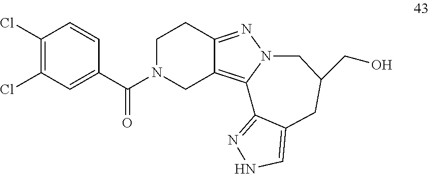 [SMILES: O=C(c1ccc(Cl)c(Cl)c1)N1CCc2nn3c(c2C1)-c1n[nH]cc1CC(CO)C3]

Intermediate I123 was dissolved in THF (0.7 mL), 9-BBN (0.5M in THF, 0.60 mL, 0.30 mmol) was added and the mixture was stirred at room temperature for 1 h. NaOH (1M, aq., 0.1 mL, 0.1 mmol) and H₂O₂ (0.1 mL) were added and the reaction mixture was stirred for another 1 h. The mixture was diluted with water and extracted with EtOAc. The combined organic extracts were concentrated under reduced pressure. The crude mixture was purified by preparative TLC (100% EtOAc) to afford compound 43 (4.0 mg, 15% over 3 steps).

LCMS (method D): Rt=2.38 min, m-z calcd. for C₂₀H₁₉Cl₂N₅O₂ 431, m/z found 432 [M+H]⁺; ¹H NMR (400 Hz, MeOD) δ ppm 7.54-7.70 (m, 1H), 7.34-7.50 (m, 1H), 7.32-7.73 (m, 2H), 4.91-4.98 (m, 1H), 4.50-4.81 (m, 2H), 4.12-4.27 (m, 1H), 3.93-4.10 (m, 1H), 3.41-3.77 (m, 3H), 2.61-3.08 (m, 4H), 2.15-2.34 (m, 1H).

### 3.2.2.4. Synthesis of Imidazole Derivative Compounds

### 3.2.2.4.1. Synthesis of Compound 44

### (3,4-Dichlorophenyl)(4,5,6,9,10,12-hexahydroimidazo[4,5-c]pyrido[4′,3′:3,4]pyrazolo[1,5-a]azepin-11(3H)-yl)methanone

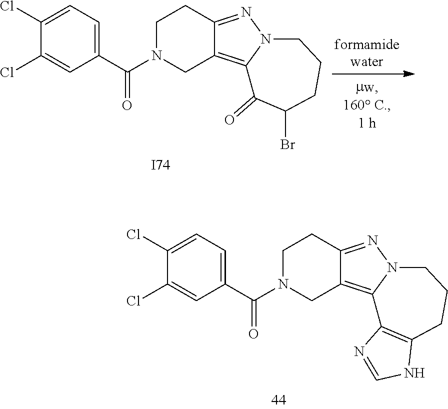 [SMILES: "I74" ; O=C1c2c3c(nn2CCCC1Br)CCN(C(=O)c1ccc(Cl)c(Cl)c1)C3 ; O=C(c1ccc(Cl)c(Cl)c1)N1CCc2nn3c(c2C1)-c1nc[nH]c1CCC3]

The reaction was performed under Ar atmosphere.

To a solution of intermediate I74 (800 mg, 1.75 mmol) in formamide (8.37 mL, 210 mmol) was added H₂O (0.88 mL, 49.0 mmol). The reaction mixture was stirred at 160° C. under microwave irradiations for 1 h and diluted with DCM (10 mL) and water (3 mL). The layers were separated and the aqueous phase was extracted with DCM. The combined organic layers were dried (Na₂SO₄), filtered, and concentrated under reduced pressure. The crude mixture was purified by reverse flash column chromatography (C-18, mobile phase: H₂O/MeCN, gradient from 95:5 to 50:50) to give two fractions of compound 44: fraction A (200 mg, 90% purity, 26%) and fraction B (158 mg, 92% purity, 21%). Fraction A was purified by flash column chromatography (silica, mobile phase: DCM/MeOH, gradient from 100:0 to 98:2) to afford compound 44 (130 mg, 18%).

¹H NMR (400 MHz, DMSO-d₆, 80° C.) δ ppm 11.65 (s, 1H), 7.69 (d, J=8.4 Hz, 1H), 7.67 (d, J=1.6 Hz, 1H), 7.58 (s, 1H), 7.43 (d, J=2.0 Hz, 1H), 4.81 (s, 2H), 4.33-4.19 (m, 2H), 3.79-3.64 (m, 2H), 2.95 (t, J=6.0 Hz, 2H), 2.69 (t, J=5.6 Hz, 2H), 2.15-2.08 (m, 2H); LCMS (method E): Rt 7.6 min, m/z calcd. for C₁₉H₁₇Cl₂N₅O 401, m/z found 402 [M+H]⁺.

### 3.2.2.4.2. Synthesis of Compound 45

### (3,4-Dichlorophenyl)(3-methyl-4,5,6,9,10,12-hexahydroimidazo[4,5-c]pyrido[4′,3′:3,4]pyrazolo-[1,5-a]azepin-11(3H)-yl)methanone

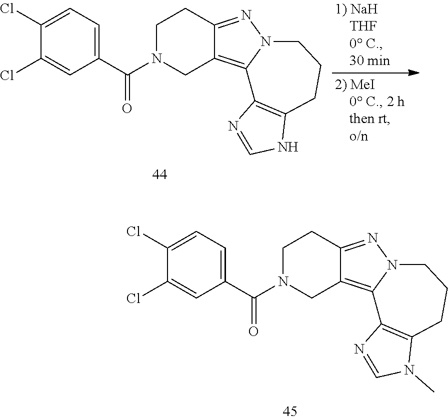 [SMILES: O=C(c1ccc(Cl)c(Cl)c1)N1CCc2nn3c(c2C1)-c1nc[nH]c1CCC3 ; Cn1cnc2c1CCCn1nc3c(c1-2)CN(C(=O)c1ccc(Cl)c(Cl)c1)CC3]

The reaction was performed under anhydrous conditions and under Ar atmosphere.

To a solution of compound 44 (138 mg, 0.31 mmol, 91% purity) in THF (4 mL) was added NaH (60% in mineral oil, 25.1 mg, 0.63 mmol) at 0° C. The mixture was stirred at this temperature for 30 min. Iodomethane (39.0 μL 0.63 mmol) was added and the reaction mixture was stirred at 0° C. for 2 h and at room temperature overnight. The mixture was combined with another fraction (0.13 mmol). The mixture was diluted with water (10 mL). The layers were separated and the aqueous phase was extracted with EtOAc (2×30 mL). The combined organic layers were dried (Na₂SO₄), filtered and concentrated under reduced pressure. The crude mixture was purified by flash column chromatography (silica, mobile phase: DCM/MeOH, gradient from 100:0 to 97:3) to afford compound 45 (110 mg, 69%) as a white solid.

¹H NMR (400 MHz, DMSO-d₆, 80° C.) δ ppm 7.68 (d, J=8.0 Hz, 1H), 7.66 (d, J=2.0 Hz, 1H), 7.59 (s, 1H), 7.42 (dd, J=8.0, 2.0 Hz, 1H), 4.79 (s, 2H), 4.32-4.25 (m, 2H), 3.78-3.65 (m, 2H), 3.56 (s, 3H), 2.89 (t, J=6.4 Hz, 2H), 2.68 (t, J=6.0 Hz, 2H), 2.19-2.11 (m, 2H); LCMS (method E): Rt=8.1 min, m/z calcd. for C₂₀H₁₉Cl₂N₅O 415, m/z found 416 [M+H]⁺.

### 3.2.2.4. Synthesis of Thiazole Derivative Compounds

### Synthesis of Compound 46

### (3,4-Dichlorophenyl)(4,5,6,9,10,12-hexahydro-11H-pyrido[4′,3′:3,4]pyrazolo[1,5-a][1,3]-thiazolo[4,5-c]azepin-11-yl)methanone

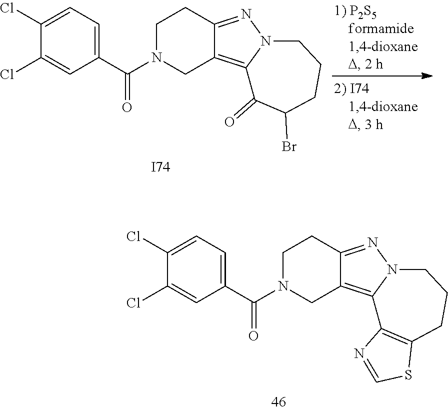 [SMILES: "I74" ; O=C1c2c3c(nn2CCCC1Br)CCN(C(=O)c1ccc(Cl)c(Cl)c1)C3 ; O=C(c1ccc(Cl)c(Cl)c1)N1CCc2nn3c(c2C1)-c1ncsc1CCC3]

In a solution of phosphorus pentasulfide (340 mg, 0.77 mmol) in 1,4-dioxane (2 mL) was added formamide (349 μL, 8.75 mmol) at room temperature. The reaction mixture was stirred under reflux for 2 h and cooled to room temperature. The solid was filtered off and the filtrate was added to a solution of intermediate I74 (200 mg, 0.437 mmol) in 1,4-dioxane (1 mL). The reaction mixture was stirred under reflux for 3 h. The mixture was diluted with DCM (15 mL) and filtered through a pad of Celite®. The filtrate was concentrated under vacuum. The crude mixture was purified by flash column chromatography (silica, mobile phase: DCM/MeOH, gradient from: 100:0 to 97:3). The product was dried at 50° C. under vacuum overnight to give compound 46 (140 mg, 76%) as a white solid.

¹H NMR (400 MHz, DMSO-d₆, 80° C.) δ ppm 8.97 (s, 1H), 7.69 (d, J=8.2 Hz, 1H), 7.67 (d, J=1.7 Hz, 1H), 7.43 (dd, J=8.2, 1.7 Hz, 1H), 4.83 (s, 2H), 4.45-4.33 (m, 2H), 3.78-3.66 (m, 2H), 3.24 (t, J=5.7 Hz, 2H), 2.73 (t, J=5.7 Hz, 2H), 2.25-2.18 (m, 2H); LCMS (method E): Rt=10.3 min, m/z calcd. for C₁₉H₁₆Cl₂N₄OS 418, m/z found 419 [M+H]⁺.

### 3.2.2.5. Synthesis of Compounds 47-50

### Synthesis of Intermediate I26

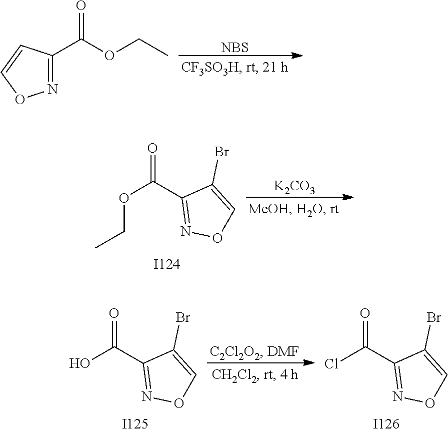

To a mixture of ethyl isoxazole-3-carboxylate [3209-70-9] (14.7 g, 104 mmol) and NBS [128-08-5] (55.7 g, 313 mmol) at 0° C., trifluoromethanesulfonic acid 11493-13-61 (175 mL, 1.98 mol) was added dropwise. The mixture was stirred at 0° C. for 30 min, warmed to room temperature and stirred for 21 h. The reaction mixture was quenched at 0° C. with saturated NaHCO₃ aqueous solution (500 mL) and neutralized with solid Na₂CO₃. The mixture was diluted with EtOAc (250 mL) and Et₂O (250 mL). The layers were separated, and the aqueous layer was extracted with Et₂O (4×250 mL). The combined organic layers were dried over Na₂SO₄, filtered and concentrated to dryness. The residue was purified by column chromatography (cyclohexane/EtOAc from 100:0 to 80:20) to afford I124 (10.3 g, 45%) as a white solid.

A solution of K₂CO₃ [584-08-7] (9.42 g, 68.2 mmol) in H₂O (82 mL) was added to a solution of I124 (10 g, 45.5 mmol) in MeOH (165 ml) at 0° C. The reaction was warmed to room temperature and stirred until the starting material was consumed. The reaction crude was concentrated, H₂O and EtOAc were added. The layers were separated, the aqueous layer was extracted with EtOAc (2×40 mi), acidified with HCl 3M (pH˜2) and extracted with EtOAc (3×9) ml). The organic layer was dried over Na₂SO₄ and concentrated to afford I125 (8.6 g, 99%) as a white solid.

The reaction was performed in anhydrous conditions under argon atmosphere.

To a solution of 1125 (5.00 g, 26.0 mmol) in CH₂Cl₂ (50 mL) were added oxalyl chloride [79-37-8] (6.6 mL, 78.1 mmol) and DMF (0.202 mL, 2.61 mmol). The mixture was stirred at room temperature for 4 h. The reaction mixture was concentrated to dryness and co-evaporated with DCM (3×20 mL) to afford I126 as a yellow oil. The crude was used as such in the next step without any further purification.

### Synthesis of Compounds 47-50

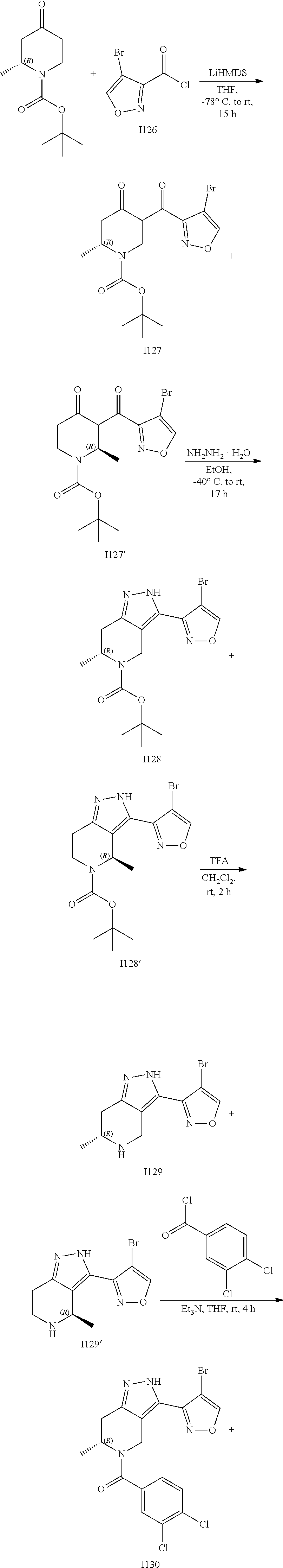

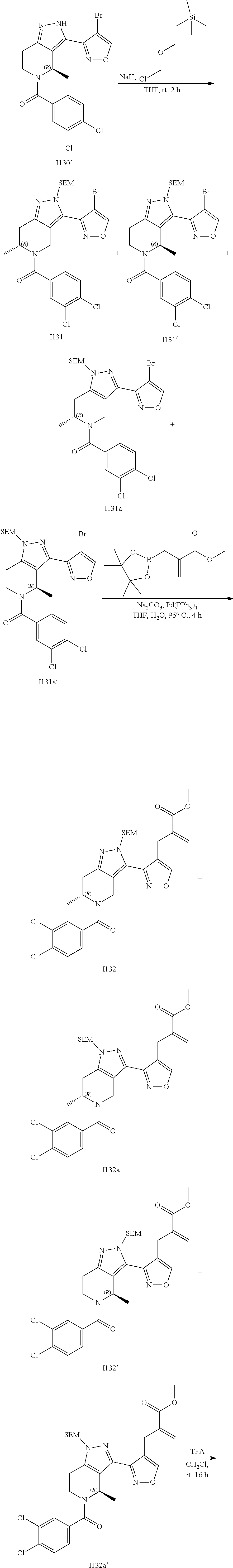

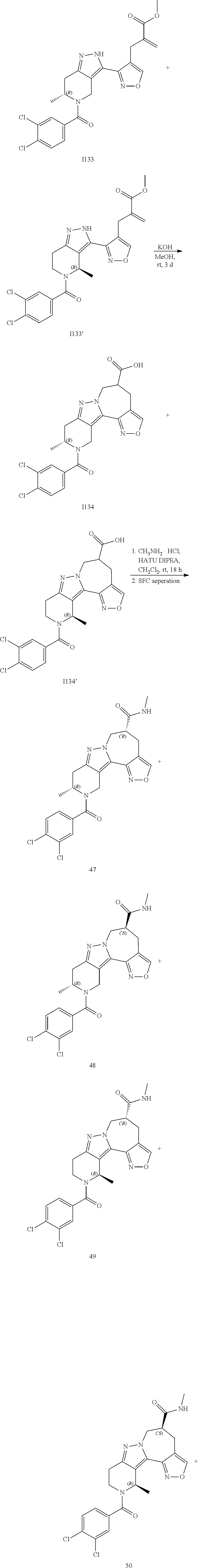

### Intermediates I127 and I127′

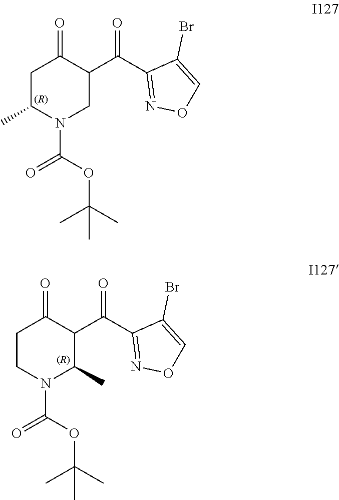

The reaction was performed in anhydrous conditions under argon atmosphere.

To a solution of (2R)-2-methyl-4-oxo-piperidine-1-carboxylic acid tert-butyl ester [790667-43-5] (5.6 g, 26.0 mmol) in THF (50 mL) at −78° C., LiHMDS [4039-32-1] (39 mL, 39 mmol, 1M in THF) was added dropwise and stirred at −78° C. for 30 min. Then the mixture vs added via cannula to a solution of I126 (6 g, 28.7 mmol) in THF (50 mL) at −78° C. and slowly warmed to ambient temperature and stirred for 15 h. The reaction mixture was quenched with a saturated aqueous solution of NH₄Cl (120 mL), and the aqueous layer was extracted with EtOAc (3×120 mL). The combined organic layers were washed with brine (100 mL), dried over Na₂SO₄, filtered and concentrated. The crude was purified by column chromatography (cyclohexane/EtOAc from 100:0 to 0:100) to yield a mixture of I127 and I127′ as an orange oil.

### Intermediates I128 and I128′

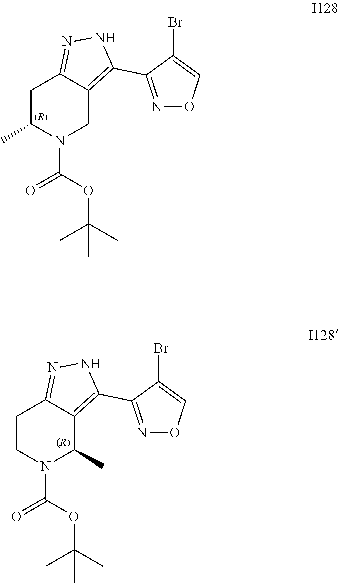

To a solution of I127 and I127′ (6.19 g, 16.0 mmol) in EtOH (74 mL) at −40° C., hydrazine monohydrate [7803-57-8] (4.0) g, 79.9 mmol) was added and stirred at room temperature for 17 h. The reaction mixture was concentrated, then saturated NaHCO₃ aqueous solution (100 mL) was added and extracted with EtOAc (3×80 mL). The combined organic layers were washed with brine (100 mL), dried over Na₂SO₄, filtered and concentrated. The residue was purified by column chromatography (cyclohexane/EtOAc from 100:0 to 50:50) to afford a mixture of I128 and I128′ as a white solid.

### Intermediates I129 and I129′

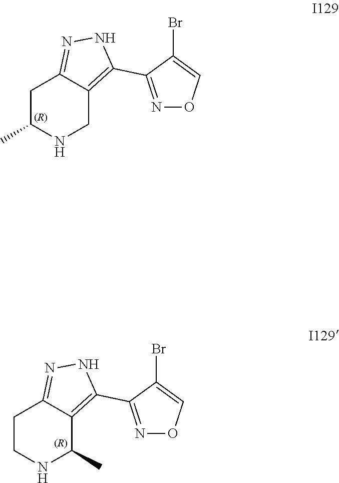

TFA [76-05-1] (1.0 mL, 13.07 mmol) was added to a solution of I128 and I128′ (100 mg, 0.261 mmol) in CH₂Cl₂ (1 mL) and stirred at room temperature for 2 h. The reaction mixture was basified with a saturated NaHCO₃ aqueous solution, diluted with HO (5 mL) and extracted with EtOAc (3×5 mL). The combined organic layers were dried over Na₂SO₄, filtered and concentrated to yield a mixture of I129 and I129′ as a white solid (used as such in the next step).

### Intermediates I130 and I130′

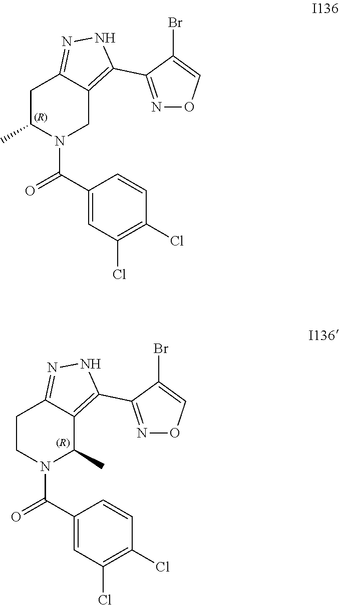

To a solution of a mixture of I129 and I129′ (2.9 g, 10.24 mmol) in THF (80 mL), Et₃N [12144-8] (4.3 mL, 30.7 mmol) and 3,4-dichlorobenzoyl chloride [3024-72-4] (2.6 g, 12.3 mmol) were added at 0° C. The mixture was stirred at room temperature for 4 h. Aqueous NH₄Cl saturated solution (5 mL) was added and the aqueous layer was extracted with EtOAc (3×5 mL). The combined organic layers were washed with brine (10 mL), dried over Na₂SO₄, filtered and concentrated. The residue was purified by flash chromatography (DCM/MeOH from 100/0 to 99/1) to afford a mixture of I130 and I130′ (4.17 g, 83%) as a white solid.

### Intermediates I131, I131′, I131a and I131a′

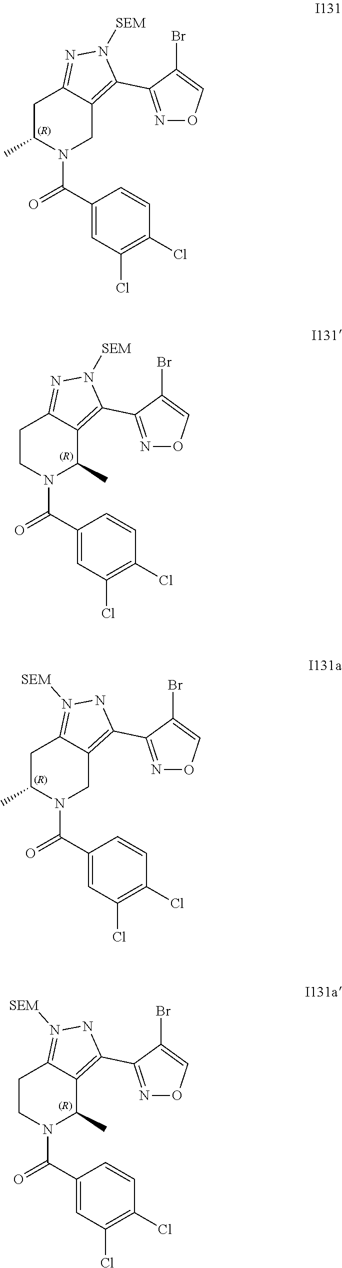

The reaction was performed under Argon atmosphere.

To a solution of I130 and I130′ (1.03 g, 2.26 mmol) in THF (20 mL), NaH [7646-69-7] (135 mg, 3.39 mmol, 60%) was added at 0° C. After stirring 15 min, 2-(trimethylsilyl)ethoxymethyl chloride [76513-69-4] (0.480 mL, 2.71 mmol) was added. The resulting mixture was stirred at room temperature for 2 h before a saturated NH₄Cl aqueous solution (20 mL) was added. The aqueous layer was extracted with EtOAc (3×15 mL). The combined organic layers were washed with brine (20 mL), dried over Na₂SO₄, filtered and concentrated. The residue was purified by column chromatography (cyclohexane/EtOAc from 100/0 to 80/20) to yield a mixture of isomers I131, I131′, I131a and I131a′ (961 mg, 72%) as a white solid.

### Intermediates I132, I132a, I132′ and I132a′

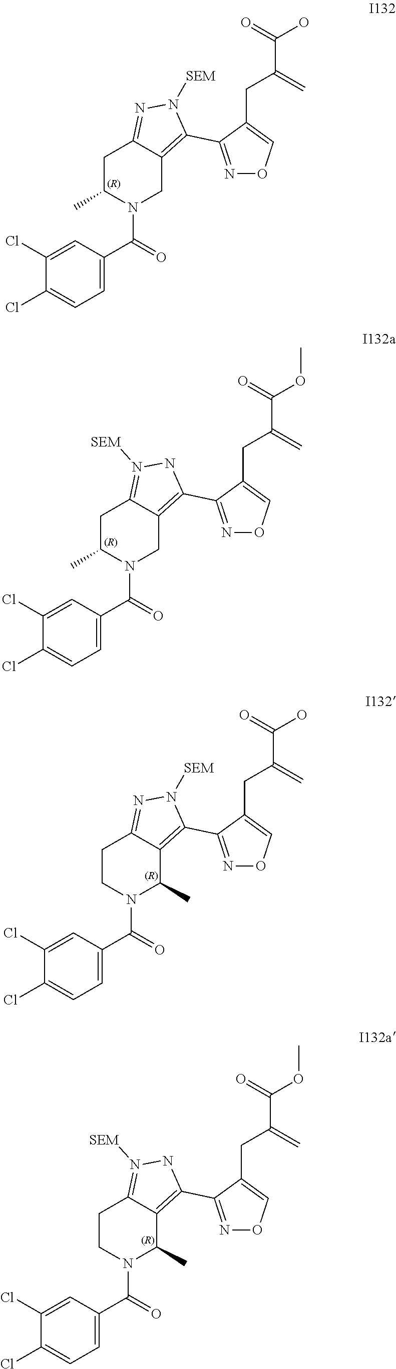

The reaction was performed under argon atmosphere.

A mixture of I131, I131′, I1319 and I131a′ (823 mg, 1.40 mmol), boronic ester [153989-28-7] (635 mg, 2.81 mmol) and Na₂CO₃ [497-19-8] (446 mg, 4.21 mmol) in THF (11 mL) and H₂O (2.5 mL) was degassed by bubbling argon for 10 min. Then, Pd(PPh₃)₄ [1421-01-3] (162 mg, 0.140 mmol) was added and purged with argon before stirred in a sealed tube at 95° C. for 2 h. Boronic ester [153989-28-7] (635 mg, 2.81 mmol), Na₂CO₃ [497-19-8] (446 mg, 4.21 mmol) and Pd(PPh₃)₄ [4221-01-3] (162 mg, 0.140 mmol) were added and stirred at 95° C. for 2 h. Boronic ester [153989-28-7] (317 mg, 1.40 mmol). Na₂CO₃ [497-19-8] (223 mg, 2.11 mmol) and Pd(PPh₃)₄ [4221-01-3] (0.0811 g, 0.0702 mmol) were added and stirred at 95° C. for additionally 2 h. The crude was diluted with water (50 mL) and the aqueous layer was extracted with EtOAc (3×50 mL). The combined organic layers were dried over Na₂SO₄, filtered and concentrated. The crude was purified by flash chromatography (cyclohexane/EtOAc from 100:0 to 70:30) to afford a mixture of I132, I132a, I132′ and I132a′ as a yellowish oil (used as such in the next step).

### Intermediates I133 and I133′

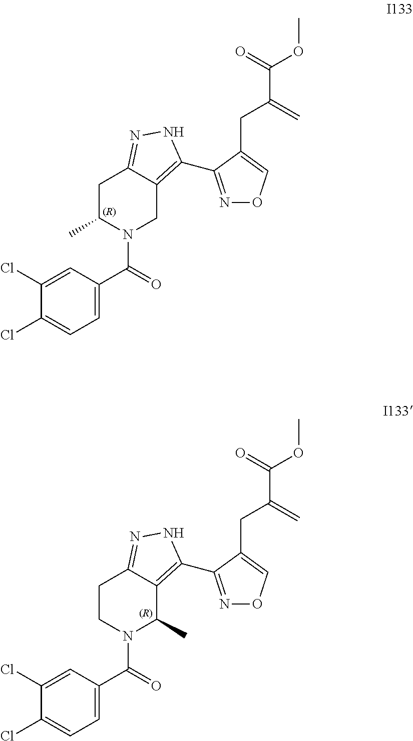

To a solution of a mixture of I132, I132a, I132′ and I132a′ (490 mg, 0.81 mmol) in CH₂Cl₂ (1.6 mL), TFA [76-05-1] (1.6 mL, 20.2 mmol) was added. The mixture was stirred at room temperature for 2 h. TFA [76-05-1] (1.6 mL, 20.2 mmol) was added and stirred for 16 h. The reaction mixture was concentrated to dryness and co-evaporated with EtOH (3×8 mL) to yield a mixture of I133 and I133′ as an orange oil. The product was used as such in the next step without any further purification.

### Intermediate I134 and I134′

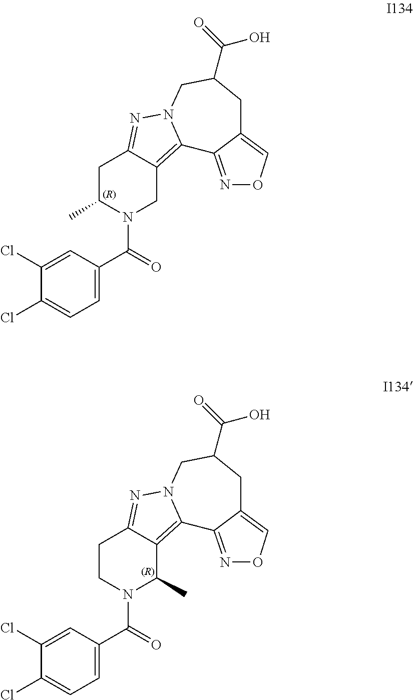

To a solution of a mixture of I133 and I133′ (826 mg, 0.806 mmol) in MeOH (18 mL), KOH [1310-58-3] (266 mg, 4.03 mmol) was added. The mixture was stirred at room temperature for 16 h. KOH [1310-58-3] (133 mg, 2.02 mmol) was added and stirred at room temperature for 3 days. The reaction mixture was acidified with HCl (1 M) aqueous solution (until pH˜2.6 mL), diluted with water (20 mL) and then extracted with EtOAc (3×30 mL). The combined organic layers were dried over Na₂SO₄, filtered and concentrated. The residue was purified by reverse phase flash chromatography (water/MeCN from 80:20 to 0:100) to afford a mixture of I134 and I134′ as a white solid (used as such in the next step).

### Compound 47-50

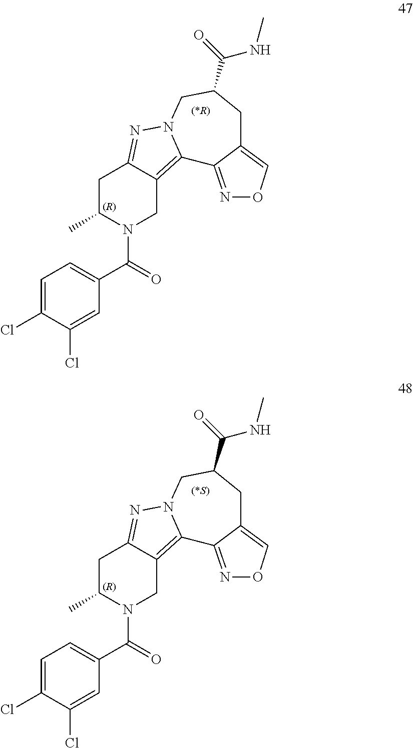

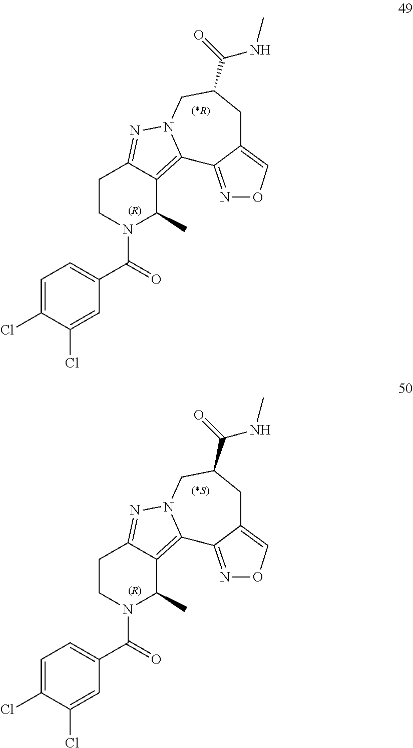

The reaction was performed in anhydrous conditions under argon atmosphere.

To a solution of a mixture of 1134 and 1134′ (840 mg, 0.80 mmol) and DIPEA [7087-68-5] (0.419 mL, 2.40 mmol) in CH₂Cl₂ (13 mL) at 0° C. were added methylamine hydrochloride [593-51-1] (81 mg, 1.20 mmol) and HATU [148893-10-1] (457 mg, 1.20 mmol). The mixture was warmed to room temperature and stirred for 18 h. The reaction mixture was quenched with a saturated NH₄Cl aqueous solution (40 mL) and extracted with DCM (3×30 mL). The combined organic layers were dried over Na₂SO₄, filtered and concentrated. The residue was purified by column chromatography (DCM/MeOH from 100:0 to 95:5) and reverse phase flash chromatography (water/MeCN from 80:20 to 0:100), followed by co-evaporation with EOH (3×10 mL). The white solid was then purified by Prep SFC (Stationary phase: Chiralpak Daicel ID 20×250 mm, Mobile phase: CO₂, ⁱPrOH+0.4ⁱPrNH₂) to yield compound 47 (22 mg), compound 48 (28 mg), compound 49 (22 mg) and compound 50 (33 mg) as white solids.

### Final Compound 47

### (5R,10R)-11-(3,4-dichlorobenzoyl)-N,10-dimethyl-5,6,9,10,11,12-hexahydro-4H-isoxazolo[3,4-c]pyrido[4′,3:3,4]pyrazolo[1,5-a]azepine-5-carboxamide

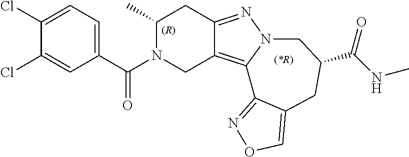

¹H NMR (400 MHz, DMSO-d₄) δ ppm 1.15 (d, J=6.82 Hz 3H) 2.54 (d, J=15.85 Hz, 1H) 2.61 (d, J=4.62 Hz, 3H) 2.91-2.97 (m, 3H) 3.00-3.14 (m, 2H) 4.23 (br d, J=17.39 Hz, 1H) 4.40-4.52 (m, 1H) 4.60-4.66 (m, 1H) 5.02-5.22 (m, 1H) 7.41 (dd, J=8.14, 1.98 Hz, 1H) 7.65-7.78 (m, 3H) 8.77 (s, 1H)

SFC (Method: SFC_A): Rt: 8.52 min. 100.0) %, m/z for C₂₂H₂₁Cl₂N₅O₃ 473.10, found 533 [M+iPrNH2]⁺.

LCMS (Method: B): Rt=1.75 min, m/z calcd. for C₂₂H₂₁Cl₂N₅O₃ 473, m/z found 474 [M+H]⁺

### Final Compound 48

### (5S,10R)-11-(3,4-dichlorobenzoyl)-N,10-dimethyl-5,6,9,10,11,12-hexahydro-4H-isoxazolo[3,4-c]pyrido[4′,3′:3,4]pyrazolo[1,5-a]azepine-5-carboxamide

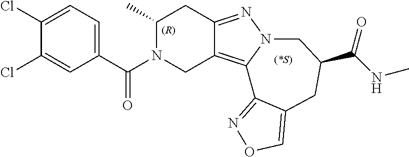

¹H NMR (400 MHz, DMSO-d₆) δ ppm 1.18 (d, J=:6.82 Hz, 3H) 2.56 (d, J=15.85 Hz, 1H) 2.60-2.63 (m, 3H) 2.89-2.97 (m, 3H) 2.98-3.15 (m, 2H) 4.26 (br d, J=17.39 Hz, 1H) 4.43-4.52 (m, 1H) 4.63 (dt, J=14.64, 1.71 Hz, 1H) 4.95-5.21 (m, 1H) 7.40 (dd, J=8.14, 1.98 Hz, 1H) 7.62-7.76 (m, 3H) 8.78 (br s, 1H)

SFC (Method: SFC_A): Rt: 7.24 min, 100.00%, m/z for C₂₂H₂₁Cl₂N₃O₃ 473.10. found 533 [M+iPrNH2]⁺.

LCMS (Method: B): Rt=1.76 min, m/z calcd. for C₂₂H₂₁Cl₂N₅O₃ 473, m/z found 474 [M+H]⁺

### Final Compound 49

### (5R,12R)-11-(3,4-dichlorobenzoyl)-N,12-dimethyl-5,6,9,10,11,12-hexahydro-4H-isoxazolo[3,4-c]pyrido[4′,3′:3,4]pyrazolo[1,5-a]azepine-5-carboxamide

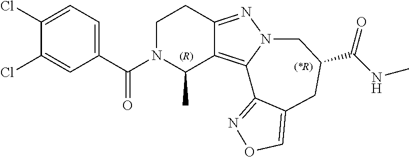

¹H NMR (400 MHz, DMSO-dt) δ ppm 1.51 (d, J=6.60 Hz, 3H) 2.62 (d, J=4.62 Hz, 3H) 2.68-2.74 (m, 2H) 2.87-2.98 (m, 4H) 2.99-3.13 (m, 2H) 4.39-4.48 (m, 1H) 4.57-4.65 (m, 1H) 7.36 (dd, J=8.25, 1.87 Hz, 1H) 7.59-7.76 (m, 3H) 8.79 (s, 1H)

SFC (Method: SFC_A): Rt: 7.05 min, 100.00%, m/z for C₂₂H₂₁Cl₂N₅O₃ 473.10. found 533 [M+iPrNH2]⁺.

LCMS (Method: B): Rt=1.77 min, m/z calcd. for C₂₂H₂₁Cl₂N₅O₃ 473, m/z found 474 [M+H]⁺

### Final Compound 50

### (5S,12R)-11-(3,4-dichlorobenzoyl)-N,12-dimethyl-5,6,9,10,11,12-hexahydro-4H-isoxazolo[3,4-c]pyrido[4′,3′:3,4]pyrazolo[1,5-a]azepine-5-carboxamide

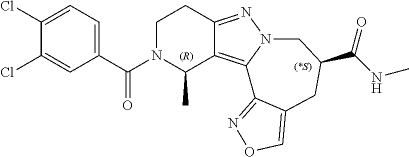

¹H NMR (400 MHz, DMSO-d₆) δ ppm 1.40-1.46 (m, 3H) 2.61 (d, J=4.62 Hz, 3H) 2.66-2.83 (m, 2H) 2.91-3.13 (m, 6H) 4.34-4.49 (m, 1H) 4.54-4.62 (m, 1H) 7.38 (dd, J=8.14, 1.98 Hz, 1H) 7.62-7.75 (m, 3H) 8.79 (s, 1H)

SFC (Method: SFC_A): Rt: 7.75 min, 100.00%, m/z calc. for C₂₂H₂₁Cl₂N₅O₃ 473.10, found 533 [M+iPrNH2]⁺.

LCMS (Method: B): Rt=1.76 min, m/z calcd. for C₂₂H₂₁Cl₂N₅O₃ 473, m/z found 474 [M+H]⁺

### 3.2.2.6. Synthesis of Compound 51

### (2-amino-4,5,6,9,10,12-hexahydro-11H-pyrido[4′,3′:3,4]pyrazolo[1,5-a]thiazolo[4,5-c]azepin-11-yl)(3,4-dichlorophenyl)methanone

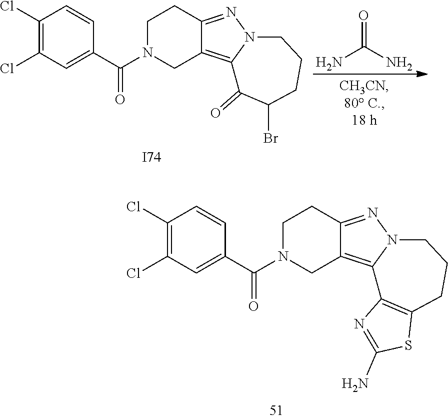

The reaction was performed in anhydrous conditions under argon atmosphere.

To a solution of 174 (510 mg, 1.12 mmol) in ACN (6 mL), thiourea [62-56-6] (84.9 mg, 1.12 mmol) was added. The reaction was stirred at 80° C. for 18 h. H₂O (10 mL) and EtOAc (3×30 mL) were added. The organic layer was separated, washed with brine, dried over Na₂SO₄, filtered, concentrated and purified by column chromatography (DCM/MeOH from 100/0 to 95/5) to yield compound 51 (102 mg, 21%) as a yellow solid.

LCMS (Method: E): Rt: 9.5 min, m/z calcd. for C₁₉H₁₇Cl₂N₅OS 433, m/z found 434 [M+H]⁺

### 3.2.2.7. Synthesis of Compounds 52

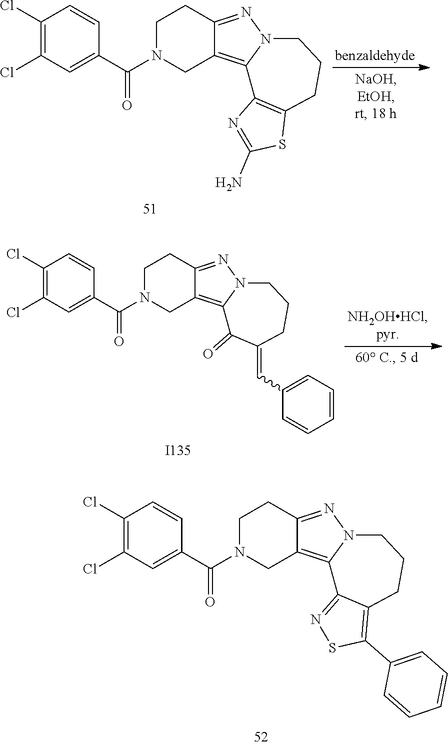 [SMILES: Nc1nc2c(s1)CCCn1nc3c(c1-2)CN(C(=O)c1ccc(Cl)c(Cl)c1)CC3 ; "I135" ; O=C1C(=Cc2ccccc2)CCCn2nc3c(c21)CN(C(=O)c1ccc(Cl)c(Cl)c1)CC3 ; O=C(c1ccc(Cl)c(Cl)c1)N1CCc2nn3c(c2C1)-c1nsc(-c2ccccc2)c1CCC3]

To a solution of NaOH [1310-73-2] (103 mg, 2.56 mmol) in EtOH (9.4 mL), I73 (650 mg, 1.72 mmol) and benzaldehyde [100-52-7] (174 μL, 1.72 mmol) were added. The reaction was stirred 18 hours at room temperature before it was diluted with DCM (40 mL) and water (20 mL). The layers were separated, and the aqueous layer was extracted with DCM (2×150 mL). Combined organic layers were washed with brine, dried over Na₂SO₄, filtered and concentrated to afford I135 (770 mg, 74%) as a white solid.

The reaction was performed in anhydrous condition under argon atmosphere. To a solution of I135 (220 mg, 0.472 mmol) in pyridine (8 mL). N-hydroxylamine hydrochloride [7803-57-8] (164 mg, 2.36 mmol) was added. The reaction was stirred 5 days at 60° C. The reaction mixture was diluted with EtOAc (50 mL), washed with aqueous HCl 1N solution (2×50 mL), brine, dried over Na₂SO₄ and concentrated. The residue was purified by flash chromatography (DCM/MeOH from 10/0 to 95/5). The obtained solid was co-evaporated with EtOAc and EtOH, dried under vacuum at 50° C. to afford compound 52 (28 mg, 12%) as a white solid.

LCMS (Method: E): Rt: 11.6 min, m/z calcd. for C₂₅H₂₀Cl₂N₄O₂ 478, m/z found 479 [M+H]⁺

### 3.2.2.8. Synthesis of Compounds 53

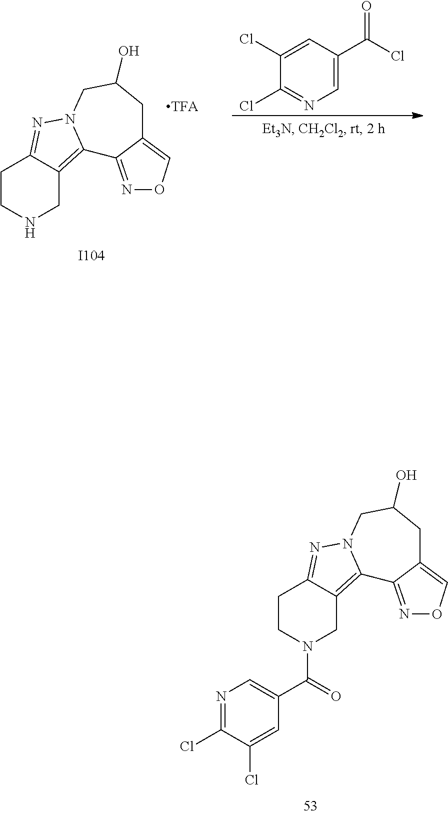 [SMILES: "I104" ; OC1Cc2conc2-c2c3c(nn2C1)CCNC3 ; O=C(Cl)c1cnc(Cl)c(Cl)c1 ; O=C(c1cnc(Cl)c(Cl)c1)N1CCc2nn3c(c2C1)-c1nocc1CC(O)C3]

To a suspension of I104 (20 mg, 0.071 mmol) and 5,6-dichloro-pyridinecarbonyl chloride [54127-29-6] in DCM (0.8 mL), Et₃N [121-44-8] (56 μL, 0.400 mmol, 0.728 g/mL) was added. The reaction was stirred for 2 h at room temperature. The crude mixture was purified by prep TLC (100% EtOAc-run plate 2×) to yield compound 53 (19 mg, 65%) as a white solid.

¹H NMR (500 MHz, DMSO-δ6): δ 8.69-8.89 (m, 1H), 8.40-8.54 (m, 1H), 8.15-8.28 (m, 1H), 5.19 (brs, 1H), 4.60-4.86 (m, 2H), 4.40-4.56 (m, 2H), 4.24 (br s, 1H), 3.57-3.96 (m, 2H), 2.89-3.04 (m, 2H), 2.67-2.82 (m, 2H).

LCMS (Method: E): Rt: 2.38 min, m/z calcd. for C₁₈H₁₅C₂N₅O₃ 419, m/z found 420 [M+H]⁺.

Intermediates and compounds of Formula (Ia) can be prepared by the following methods.

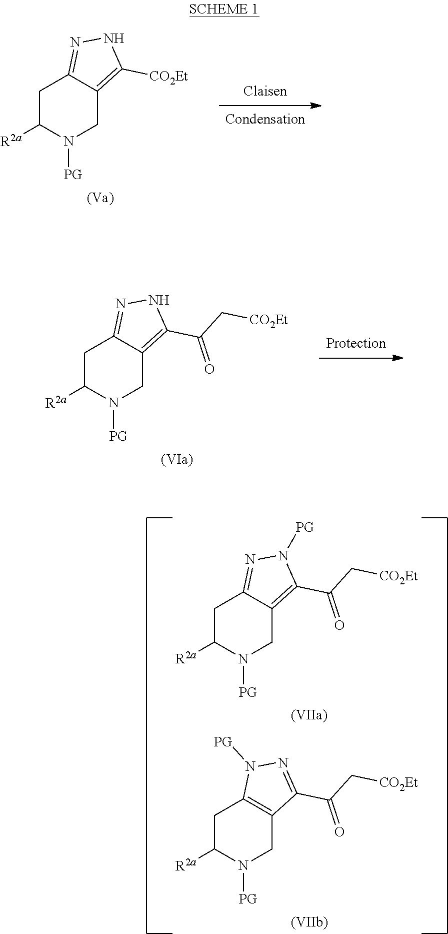

According to SCHEME 1, a compound of formula (Va), where R^{2a} is H or C₁₋₆alkyl, and PG is BOC, undergoes a Claisen-type reaction or acylation with ethyl acetate; in the presence of a suitable base such as sodium hydride, potassium hydride, lithium diisopropylamide (LDA), lithium hexamethyldisilylamide (LHMDS), sodium bis(trimethylsilyl)amide (NaHMDS), potassium butoxide, and the like; preferably sodium bis(trimethylsilyl)amide (NaHMDS); in a suitable solvent such as tetrahydrofuran (THF), dioxane, dimeth-oxyethane, toluene, xylenes, acetonitrile (ACN), dimethysulfoxide, dimethylformamide (DMF), dimethylacetamide (DMA), N-methylpyrrolidone, and the like; preferably THF; at a temperature ranging from −70 to 100° C., preferably −65 to 40° C.; for a period of 2 h to 24 h. A compound of formula (Via) is protected employing established methodologies, such as those described in T. W. Greene and P. G. M. Wuts, “Protective Groups in Organic Synthesis,” 3 ed., John Wiley & Sons, 1999, to provide a mixture of compounds of formula (VIIa) and formula (VIIb), where R^{2a} is H or C₁₋₄alkyl, and PG is BOC.

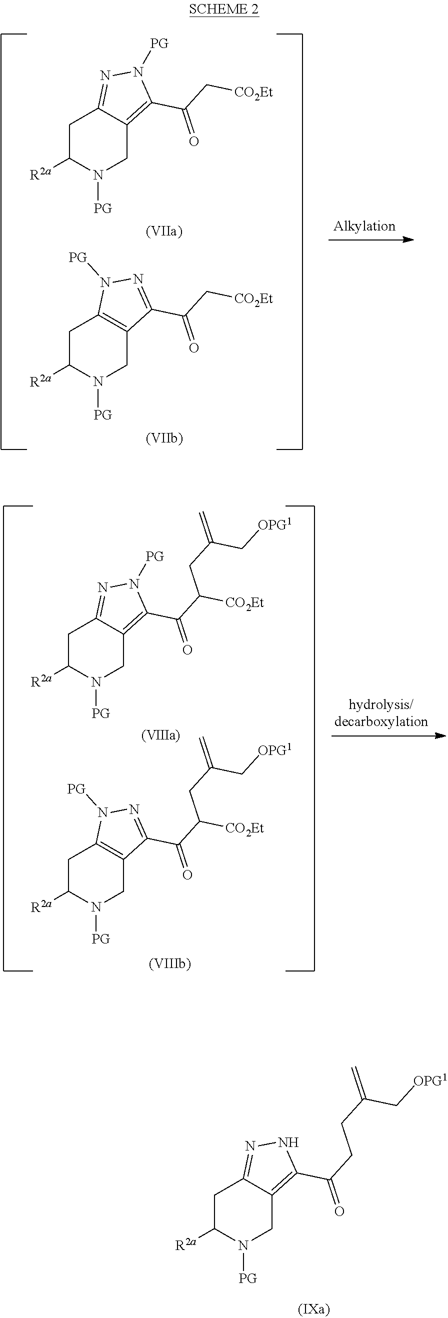

According to SCHEME 2, alkylation of β-ketoester compounds of formula (VIIa) and (VIIb), where R^{2a} is H or C₁₋₆alkyl, and PG is BOC, is achieved employing an alkyl halide such as ((2-(bromomethyl)allyl)oxy)(tert-butyl)diphenylsilane, a base such as K₂CO₃; NaI; in a suitable solvent such as acetone, and the like; to provide a mixture of compounds of formulas (VIIIa) and (VIIIb). Hydrolysis/decarboxylation of a mixture of compounds of formula (VIIIa) and (VIIIb) is achieved using a base such as with potassium hydroxide, and the like; in a suitable solvent such a as MeOH, H₂O, or a mixture thereof to provide a compound of formula (IXa).

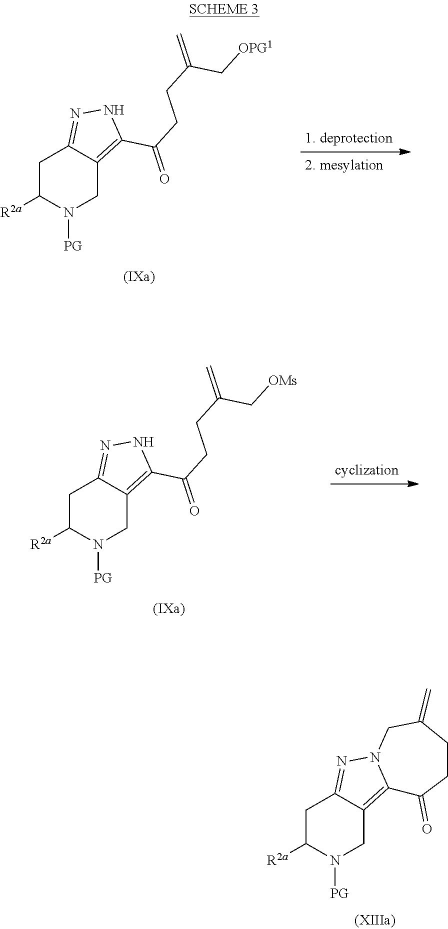

According to SCHEME 3, a compound of formula (IXa), where R^{2a} is H or C₁₋₆alkyl, PG is BOC, PG¹ is TBDSP; is de-silylated with tetra-n-butylammonium fluoride (TBAF), in a suitable solvent such as THF and the like. Subsequent mesylation of the hydroxy employing methanesulfonyl chloride (mesyl chloride), a suitable base such as triethylamine (TEA), in a suitable solvent such as DCM, and the like, provides a compound of formula (XIIa). Intramolecular cyclization employing a base such as DBU, in a suitable solvent such as THF, and the like, provides compounds of formula (XIIa), where n^{a} is 1. Compounds of formula (XIIIa), where n^{a} is 0 or 2 may be prepared in a manner analogous to compounds of formula (XIIIa) where n^{a} is 1.

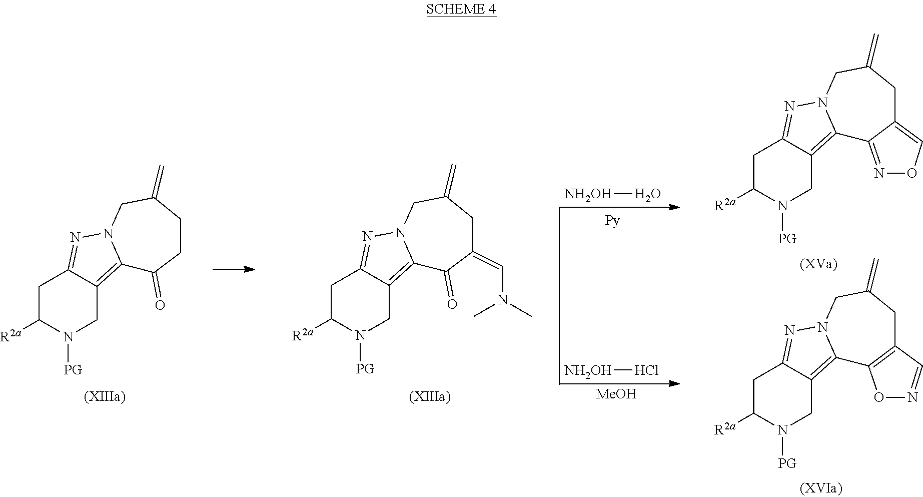

According to SCHEME 4, a compound of formula (XIIIa) is treated with DMA to afford the dimethyl enamine compound of formula (XIVa), which upon treatment with hydroxylamine hydrochloride; in the presence of a tertiary base such as pyridine, and the like, at a temperature of about 70-115° C.; affords a compound of formula (XVa). In a similar fashion, a compound of formula (XIVa) is treated with hydroxylamine hydrochloride, in the presence of methanol, to afford a compound of formula (XVIa).

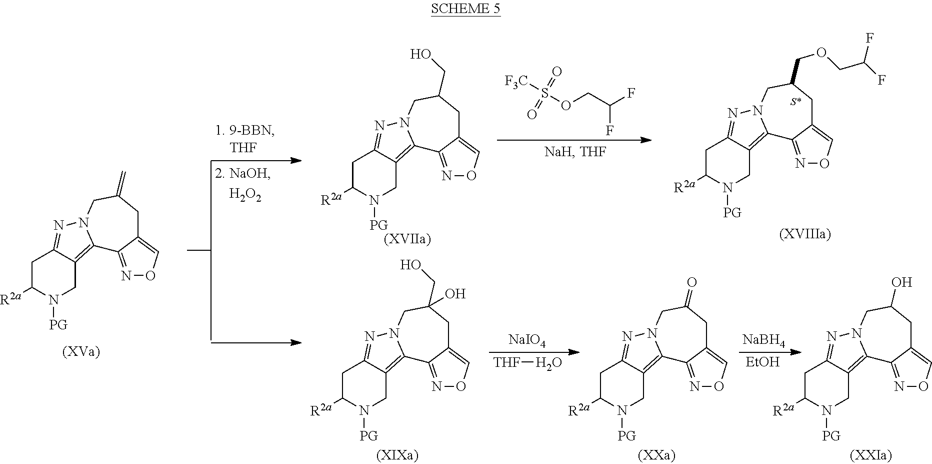

According to SCHEME 5, the alkenyl moiety of a compound of formula (XVa) is regioselectively converted to its corresponding terminal alcohol compound of formula (XVIIa) by the action of 9-borabicyclo[3.3.1]nonane (9-BBN), followed by treatment with hydrogen peroxide, and hydroxide, to afford a compound of formula (XVIIa). Said terminal alcohol is further derivatized using methods well known to one of skill in the art. For example, the alcohol is oxidized to the corresponding aldehyde by the action of a suitable oxidizing agent such as manganese oxide. Alternatively, the alcohol functional group may also be alkylated with a suitable electrophile such as 2,2-difluoroethyl trifluoromethanesulfonate; a suitable base such as NaH, and the like; in a suitable solvent such as THF, and the like; to provide a compound of formula (XVIIIa).

Alternatively, a compound of formula (XVa), where R^{4a} is H or C₁₋₄alkyl, undergoes an osmium-catalyzed dihydroxylation, employing conditions known to one skilled in the art, to provide a compound of formula (XIXa). For example, a compound of formula (XVa), where R^{4a} is H or C₁₋₄alkyl; is reacted with an oxidant such as an osmium-containing compound like OsO₄ (or OsOs₄ can also be prepared in situ by the oxidation of K₂OsO₂(OH)₄ with NMO); an amine oxide co-oxidant such as NMO, and the like; in a suitable solvent such as THF, acetone, H₂O, or a mixture thereof; to provide a compound of formula (XIXa). A compound of formula (XIXa) upon treatment with an oxidizing agent such as sodium periodate and the like; affords a compound of formula (XXa). Reduction of the ketone of formula (XXa) to an alcohol of formula (XXIa) is achieved by reaction of a hydride source such as sodium borohydride; and the like, a suitable solvent such as an alcoholic solvent.

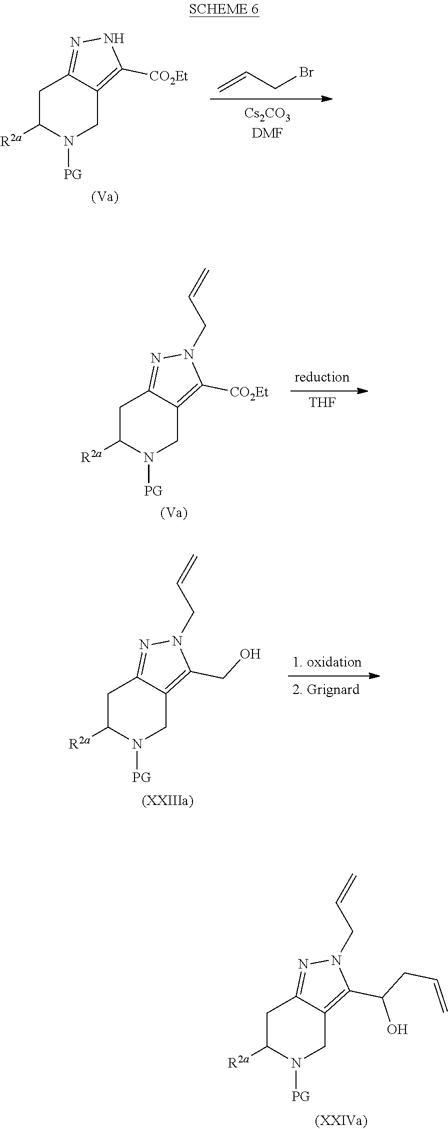

According to SCHEME 6, a commercially available or synthetically accessible alkyl halide, such as 3-bromoprop-1-ene, is reacted with a compound of formula (Va), where R^{2a} is H or C₁₋₆alkyl; an inorganic base such as Cs₂CO₃, potassium carbonate, and the like; in a suitable solvent such as DMF, THF, pyridine, and the like; to provide a compound of formula (XXIIa). The ester functionality of a compound of formula (XXIIa) is reduced by a hydride source such as lithium aluminum hydride, sodium borohydride, or the like: in a suitable solvent such as THF, and the like; at temperatures ranging from −40° C. to 40° C.; to afford an alcohol of formula (XXIIIa). A compound of formula (XXIVa) is prepared in two steps. In a first step, oxidation to the corresponding aldehyde is achieved employing conditions known to one skilled in the art, for example. Swern oxidation conditions ((COCl)₂/DMSO), or TPAP-NMO conditions. In a second step, reaction of the aldehyde intermediate with a Grignard reagent, such as allyl magnesium bromide: in an aprotic solvent, such as THF, and the like; at a temperature ranging from −40° C. to 40° C.; provides a compound of formula (XXIVa), where PG is Boc and R^{2a} is H or C₁₋₆alkyl.

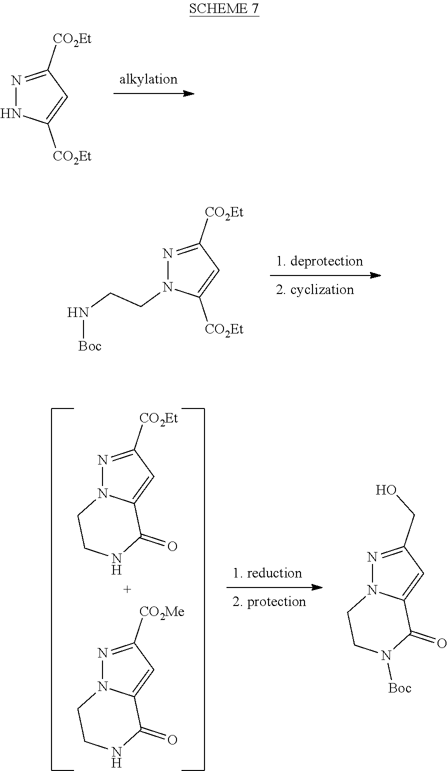

According to SCHEME 7, commercially available or synthetically accessible diethyl 1H-pyrazole-3,5-dicarboxylate is alkylated with tert-butyl N-(2-bromoethyl)carbamate: a base such as Cs₂CO₃, and the like; in a suitable solvent such as DMF, and the like; to provide diethyl 1-(2-((tert-butoxycarbonyl)amino)ethyl)-1H-pyrazole-3,5-dicarboxylate. Diethyl 1-(2-((tert-butoxy-carbonyl)amino)ethyl)-1H-pyrazole-3,5-dicarboxylate is deprotected employing established methodologies, such as those described in T. W Greene and P. G. M. Wuts. “Protective Groups in Organic Synthesis,” 3 ed., John Wiley & Sons, 1999; then subsequently treated under basic conditions to form a mixture of ethyl 4-oxo-4,5,6,7-tetrahydropyrazolo[1,5-a]pyrazine-2-carboxylate and methyl 4-oxo-4,5,6,7-tetrahydropyrazolo[1,5-a]pyrazine-2-carboxylate. A mixture of ethyl 4-oxo-4,5,6,7-tetrahydropyrazolo[1,5-a]pyrazine-2-carboxylate and methyl 4-oxo-4,5,6,7-tetrahydropyrazolo[1,5-a]pyrazine-2-carboxylate is with a hydride source such as LAH, and the like; followed by protection of the amino functionality using conventional methods, such as by treatment with Boc-anhydride, to afford tert-butyl 2-(hydroxymethyl)-6,7-dihydropyrazolo[1,5-a]pyrazine-5(4H)-carboxylate.

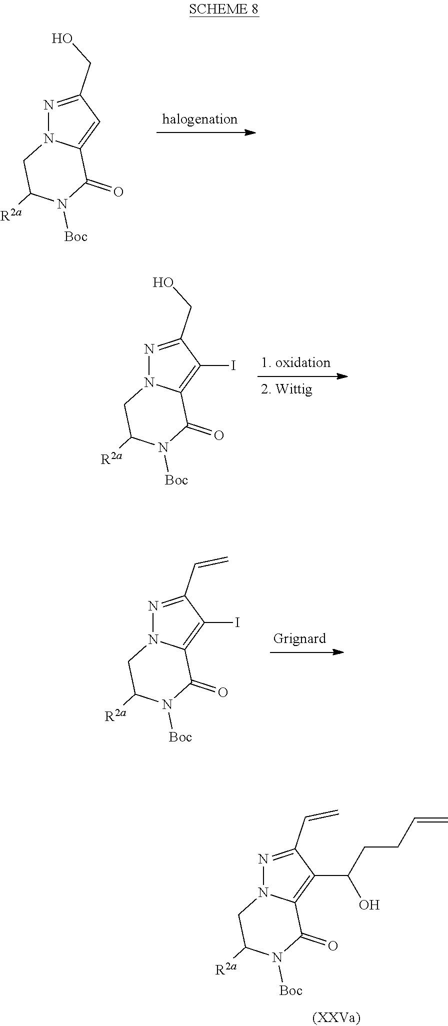

According to SCHEME 8, iodination of tert-butyl 2-(hydroxymethyl)-6,7-dihydropyrazolo[1,5-a]pyrazine-5(4H)-carboxylate is achieved employing a halogenating agent such as N-iodosuccinimide, and the like: in a suitable solvent such as ACN, and the like; at temperatures of about 15° C.; provides tert-butyl 2-(hydroxymethyl)-3-iodo-6,7-dihydropyrazolo[1,5-a]-pyrazine-5(4H)-carboxylate. Subsequent oxidation of tert-butyl 2-(hydroxymethyl)-3-iodo-6,7-dihydropyrazolo[1,5-a]pyrazine-5(4H)-carboxylate is achieved with a suitable oxidizing agent, such as Dess-Martin periodinane (DMP); in a suitable solvent such as dichloromethane, and the like; at temperatures ranging from about 0° C. to about 25° C.; for a period of approximately 0.5 to 4 hours; to provide tert-butyl 2-formyl-3-iodo-6,7-dihydropyrazolo[1,5-a]pyrazine-5(4H)-carboxylate.

tert-Butyl 2-formyl-3-iodo-6,7-dihydropyrazolo[1,5-a]pyrazine-5(4H)-carboxylate is reacted with a Wittig type reagent such as methyltriphenylphosphonium bromide: a base such as NaHMDS, and the like; in an organic solvent such as THF, toluene, and the like; to provide tert-butyl 3-iodo-2-vinyl-6,7-dihydropyrazolo[1,5-a]pyrazine-5(4H)-carboxylate, tert-Butyl 3-iodo-2-vinyl-6,7-dihydropyrazolo[1,5-a]pyrazine-5(4H)-carboxylate is reacted under conventional Grignard reaction conditions with pent-4-enal: in the presence of an organomagnesium halide such as i-PrMgCl, and the like; in a suitable solvent such as THF, and the like; to provide a compound of formula (XXVa), where R^{2a} is H.

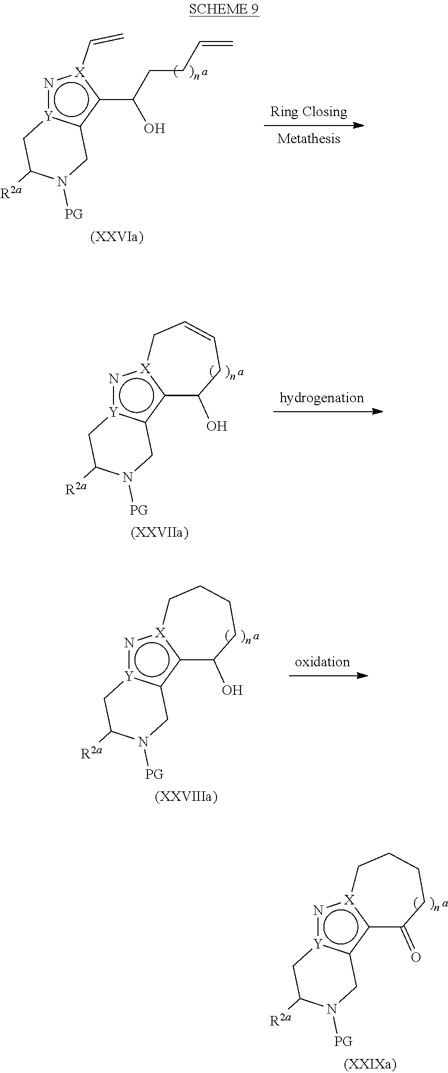

According to SCHEME 9, a compound of formula (XXVIa), which includes compounds of formula (XXIVa) and (XXVa), undergoes a ring closing metathesis reaction employing dichloro[1,3-bis(2,4,6-trimethylphenyl)-2-imidazolidinylidene](2-isopropoxyphenylmethylene)-ruthenium(II) (Hoveyda-Grubbs 11 catalyst); in a solvent such as DCM, and the like; for a period of 16-24 h; to provide a compound of formula (XXVIIa).

A compound of formula (XXVII), where PG is Boc. Y is C and X is N. and R² is H or C₁₋₆ alkyl; is reduced employing hydrogenation conditions, in the presence of a palladium catalyst, including but not limited to, Pd on carbon, Pd(dppf)Cl₂ or Pd(PPh₃)₄; in a suitable solvent or solvent system such as DMF, methanol, dioxane/water, and the like; to provide a compound of formula (XXVIIIa), where PG is Boc, Y is C and X is N, n^{a} is 1, and R^{2a} is H or C₁₋₆alkyl.

Oxidation of a compound of formula (XXVIII) to a compound of formula (XXIXa) is achieved employing conditions known to one skilled in the art. For example, reaction of an alcohol compound of formula (XXVIIIa), with the oxidation catalyst tetrapropylammonium perruthenate (TPAP); and N-methylmorpholine N-oxide (NMO) as the co-oxidant; in a suitable solvent such as ACN, DCM, DMF, and the like; provides a compound of formula (XXIXa), where X is N and Y is C.

In a similar fashion, a compound of formula (XXVIIa), where X is C and Y is N; is first oxizided under TPAP conditions previously described, followed by reduction of the double bond employing hydrogenation conditions previously described to provide a compound of formula (XXIX), where PG is Boc, Y is N and X is C, n^{a} is 1, and R^{2a} is H or C₁₋₆alkyl.

Compounds of formula (XXIXa), where n^{a} is 0 or 2 may be prepared in a manner analogous to compounds of formula (XXIXa) where n^{a} is 1.

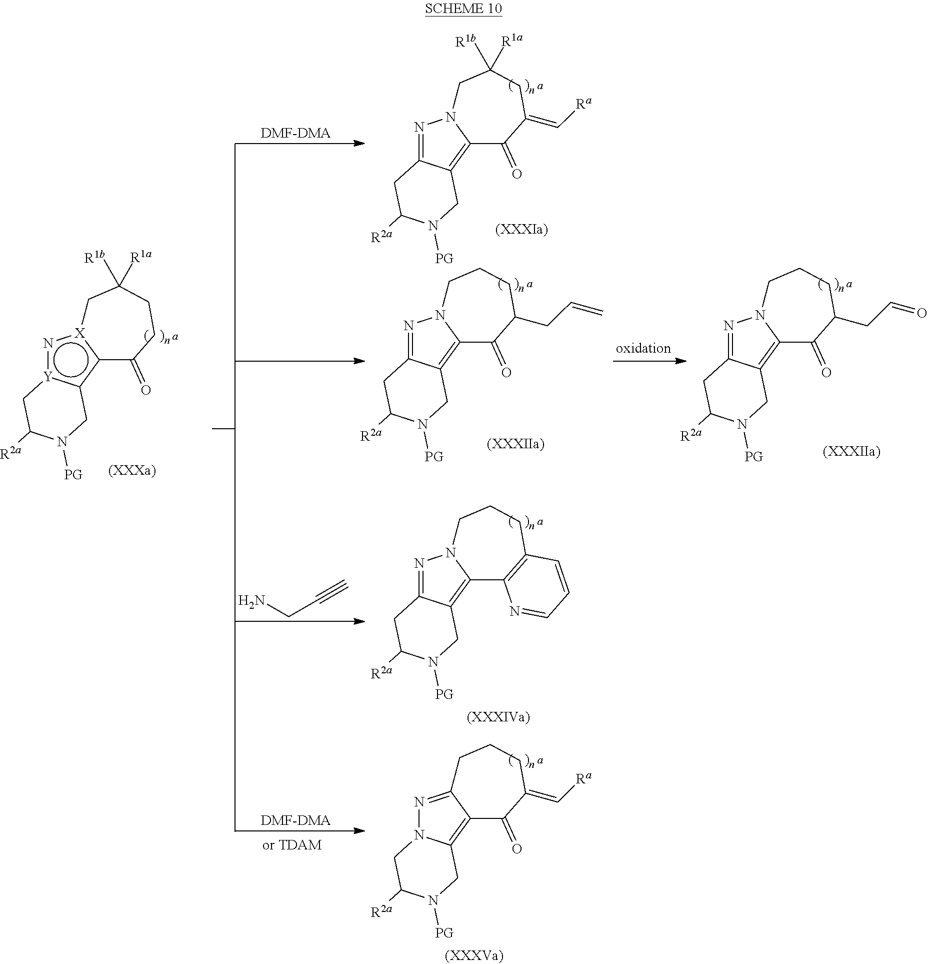

According to SCHEME 10, a ketone compound of formula (XXXa), where X is N, Y is C, R^{1b} and R^{1a} are H, or R^{1b} and R^{1a} come together to form a methylene, R^{2a} is H or C₁₋₆alkyl, and PG is Boc; is condensed with dimethylformamide-dimethyl acetal (DMF-DMA) to afford a compound of formula (XXXIa) where R^{a} is OH or N(CH₃)₂, and n^{a} is 1.

A compound of formula (XXXa), where X is N, Y is C, R^{1b} and R^{1a} are H, R^{2a} is H or C₁₋₆alkyl, n^{a} is 1, and PG is BOC; is alkylated with allyl bromide; in the presence of a strong organometallic base such as LDA; in the presence of HMPA; in an aprotic organic solvent such as THF, and the like; to afford a compound of formula (XXXIIa). Oxidation of a compound of formula (XXXIIa) to an aldehyde compound of formula (XXXIIIa) is achieved under conditions known to one skilled in the art, for example, osmium tetroxide, sodium periodate, Swern oxidation conditions, and the like.

A compound of formula (XXXa), where X is N, Y is C, R^{1b} and R^{1a} are H, R^{2a} is H or C₁₋₆alkyl, n^{a} is 1, and PG is BOC; is reacted under amination/cyclization conditions with propargyl amine; a gold catalyst such as NaAuCl₄.2H₂O, and the like, in a suitable solvent such as EtOH, and the like; to provide a compound of formula (XXXIVa).

A ketone compound of formula (XXXa), where X is C, Y is N, R^{1b} and R^{1a} are H, R^{2a} is H or C₁₋₆alkyl, and PG is Boc; is condensed with dimethylformamide-dimethyl acetal (DMF-DMA) to afford an enaminone compound of formula (XXXVa). In an alternate method, tris(dimethyl-amino)methane (TDAM) is reacted with a compound of formula (XXXa), in a solvent such as toluene, and the like; at temperatures of about 115° C.; for a period of 12-20 h; to provide a compound of formula (XXXVa), where R^{a} is N(CH₃)₂, and n^{a} is 1.

Compounds of formulas (XXXIa), (XXXIIIa), (XXXIVa), and (XXXVa), where n is 0 or 2 may be prepared in a manner analogous to compounds of formulas (XXXIa), (XXXIIIa), (XXXIVa), and (XXXVa), where n^{a} is 1.

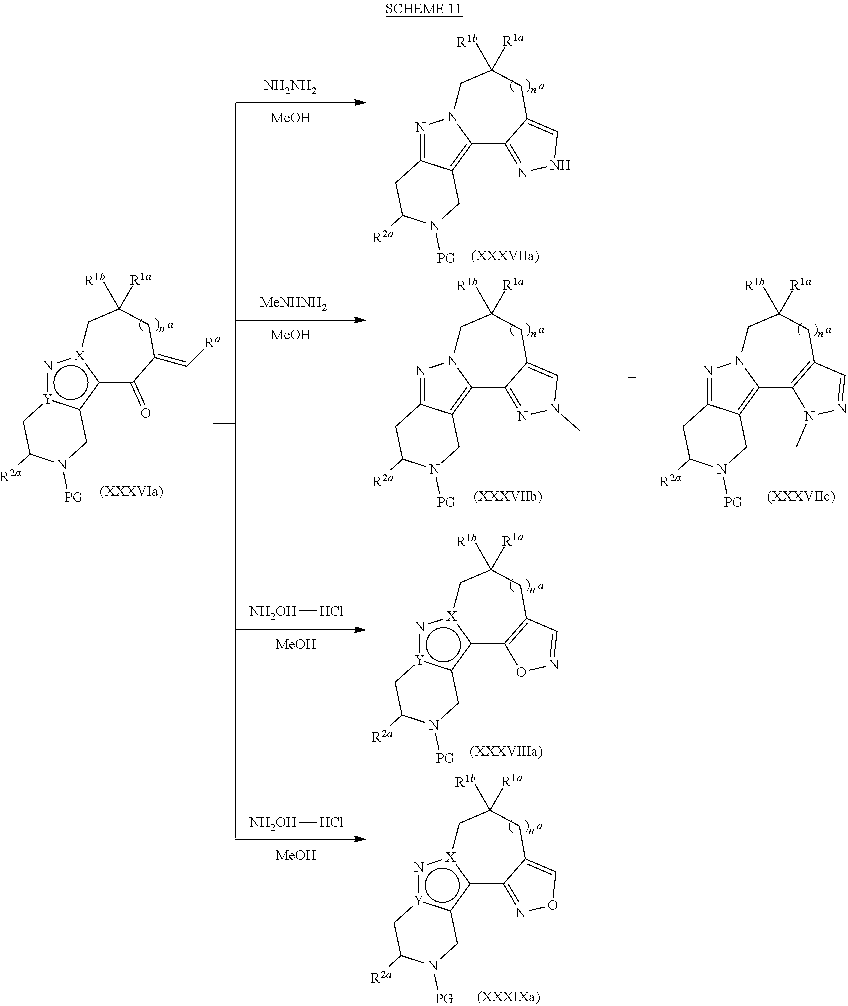

According to SCHEME 11, compounds of formulas (XXXVIIa, XXXVIIb, XXXVIIc), are prepared by reacting a compound of formula (XXXVIa), where X is N, Y is C, n^{a} is 1, and R^{a} is OH; with a hydrazine such as methylhydrazine or hydrazine hydrate; in a suitable solvent such as MeOH, and the like.

A compound of formula (XXXVIa), where R^{a} is OH or N(CH₃)₂; is treated with hydroxylamine hydrochloride; in the presence of a tertiary base such as pyridine, and the like, at temperatures ranging from 70° C. to 115° C.; to afford an isoxazole compound of formula (XXXVIIIa). In a similar fashion, a compound of formula (XXXVIa) is treated with hydroxylamine hydrochloride, in a suitable solvent such as MeOH and the like, at a temperature of about 70° C. to provide an isoxazole compound of formula (XXXIXa), where n^{a} is 1.

Compounds of formulas (XXXVIIa), (XXXVIIb), (XXXVIIc), (XXXVIIIa), and (XXXIXa) where n^{a} is 0 or 2 may be prepared in a manner analogous to compounds of formulas (XXXVIIa), (XXXVIIb), (XXXVIIc), (XXXVIIIa), and (XXXIXa), where n^{a} is 1.

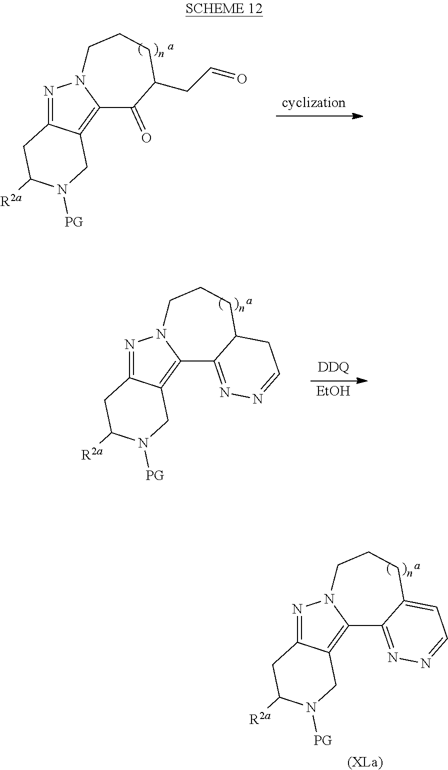

According to SCHEME 12, tert-butyl 11-oxo-10-(2-oxoethyl)-3,4,8,9,10,11-hexahydro-1H-pyrido[4′,3′:3,4]pyrazolo[1,5-a]azepine-2(7H)-carboxylate is treated with hydrazine hydrate to afford tert-butyl 4a,5,6,7,10,11-hexahydro-4H-pyridazino[3,4-c]pyrido[4′,3′:3,4]pyrazolo[1,5-a]azepine-12(13H)-carboxylate, tert-butyl 4a,5,6,7,10,11-hexahydro-4H-pyridazino[3,4-c]pyrido [4′,3′:3,4]pyrazolo[1,5-a]azepine-12(13H)-carboxylate is oxidized with a reagent such as DDQ, and the like; in a suitable solvent such as THF: at a temperature of about 0° C.; affords the aromatized compound of formula (XLa), where n^{a} is 1. R^{2a} is H, and PG is Boc.

A compound of formula (XLa), where n^{a} is 0 or 2, and R^{2a} is H or C₁₋₆alkyl, may be prepared in a manner analogous to a compound of formula (XLa), where n^{a} is 1.

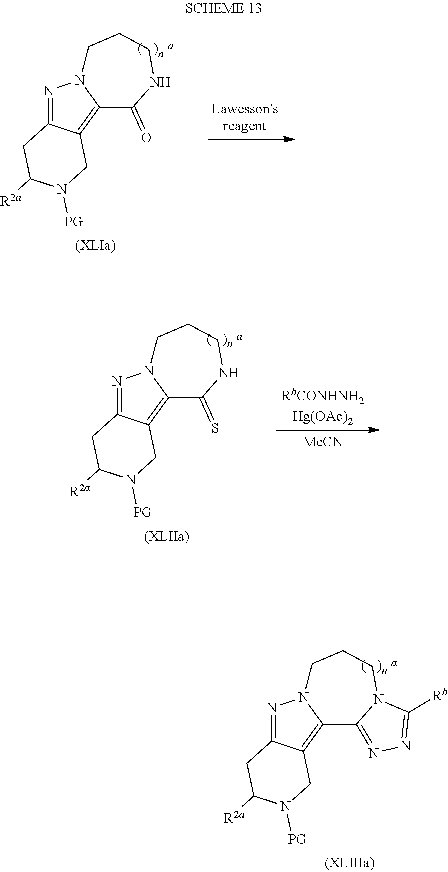

According to SCHEME 13, a compound of formula (XLIa) is converted to the thioamide compound of formula (XLIIa), employing Lawesson's reagent. For example, tert-butyl 11-oxo-3,4,8,9,10,11-hexahydro-1H-pyrido[4′,3′:3,4]pyrazolo[1,5-a][1,4]diazepine-2(7H)-carboxylate (as described in PCT Int. Appl. WO2018005883, Jan. 4, 2018) is treated with Lawesson's reagent; in a suitable solvent such as toluene, and the like; at a temperature of about 110° C.; to provide tert-butyl 11-thioxo-3,4,8,9,10,11-hexahydro-1H-pyrido[4′,3′:3,4]pyrazolo[1,5-a][1,4]diazepine-2(7H)-carboxylate. A compound of formula (XLIIa), is cyclized to form a compound of formula (XLIIIa). For example, tert-butyl 11-thioxo-3,4,8,9,10,11-hexahydro-1H-pyrido[4′,3′:3,4]-pyrazolo[1,5-a][1,4]diazepine-2(7H)-carboxylate is cyclized with an R^{b} substituted hydrazide (wherein R^{b} is hydrogen or CH₃); Hg(OAc)₂; in a suitable solvent such as ACN, and the like; to afford a compound of formula (XLIIIa), where R^{2a} is H or C₁₋₆alkyl, PG is Boc, n^{a} is 1, and R^{b} is H or CH₃.

A compound of formula (XLIII), where n is 0 or 2, and R² is H or C₁₋₆alkyl, may be prepared in a manner analogous to a compound of formula (XLIII), where n is 1.

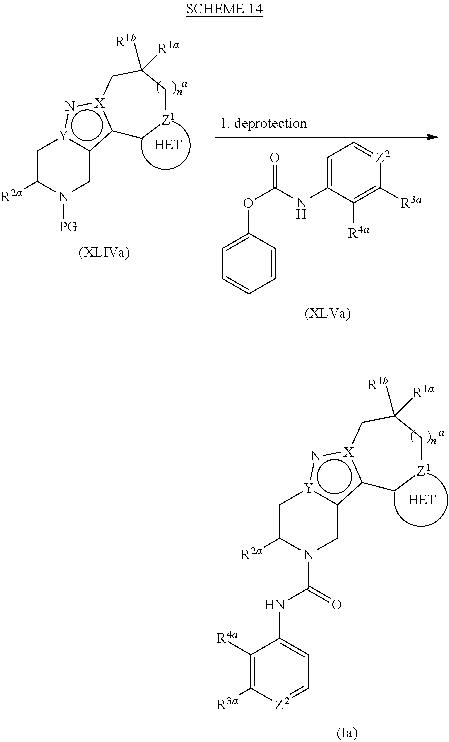

According to SCHEME 14, a compound of formula (XLIVa) (which encompasses compounds of formulas (XVa), (XVIa), (XVIIIa), (XXIa), (XXXIVa), (XXXVIIa), (XXXVIIb), XXXVIIc), (XXXVIIIa), (XXXIXa), (XLa), and (XLIa)), is deprotected employing conditions known to one skilled in the art. Subsequent reaction with a commercially available or synthetically accessible compound of formula (XLVa), where Z², R^{3a}, and R^{4a} are as defined above; a suitable base such as TEA, and the like; in a suitable solvent such as DCM, and the like; provides a compound of Formula (Ia).

### General Procedures

The following specific examples are provided to further illustrate the present disclosure and various preferred embodiments.

In obtaining the compounds described in the examples below and the corresponding analytical data, the following experimental and analytical protocols were followed unless otherwise indicated.

Unless otherwise stated, reaction mixtures were magnetically stirred at room temperature (rt) under a nitrogen atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure.

Normal-phase silica gel chromatography (FCC) was performed on silica gel (SiO₂) using prepacked cartridges.

Preparative reverse-phase high performance liquid chromatography (RP HPLC) was performed on either:

METHOD A. A Gilson GX-281 semi-prep-HPLC with Phenomenex Synergi C18 (10 μm, 150×25 mm), or Boston Green ODS C18 (5 μm, 150×30 mm), and mobile phase of 5-99% ACN in water (with 0.225% FA) over 10 min and then hold at 100% ACN for 2 min, at a flow rate of 25 mL/min. or
METHOD B. A Gilson GX-281 semi-prep-HPLC with Phenomenex Synergi C18 (10 μm, 150×25 mm), or Boston Green ODS C18 (5 μm, 150×30 mm), and mobile phase of 5-99% ACN in water (0.1% TFA) over 10 min and then hold at 100% ACN for 2 min, at a flow rate of 25 mL/min. or
METHOD C. A Gilson GX-281 semi-prep-HPLC with Phenomenex Synergi C18 (10 μm, 150×25 mm), or Boston Green ODS C18 (5 μm, 150×30 mm), and mobile phase of 5-99% ACN in water (0.05% HC) over 10 min and then hold at 100% ACN for 2 min, at a flow rate of 25 mL/min. or
METHOD D. A Gilson GX-281 semi-prep-HPLC with Phenomenex Gemini C18 (10 μm, 150×25 mm). AD (10 μm, 250 mm×30 mm), or Waters XBridge C18 column (5 μm, 150×30 mm), mobile phase of 0-99% ACN in water (with 0.05% ammonia hydroxide v/v) over 10 min and then hold at 100% ACN for 2 min, at a flow rate of 25 mL/min. or
METHOD E. A Gilson GX-281 semi-prep-HPLC with Phenomenex Gemini C18 (10 μm, 150×25 mm), or Waters XBridge C18 column (5 μm, 150×30 mm), mobile phase of 5-99% ACN in water (10 mM NH₄HCO₃) over 10 min and then hold at 100% ACN for 2 min, at a flow rate of 25 mL/min.
Preparative supercritical fluid high performance liquid chromatography (SFC) was performed either on a Thar 80 Prep-SFC system, or Waters 80Q Prep-SFC system from Waters. The ABPR was set to 100 bar to keep the CO₂ in SF conditions, and the flow rate may verify according to the compound characteristics, with a flow rate ranging from 50 g/min to 70 g/min. The column temperature was ambient temperature

Mass spectra (MS) were obtained on a SHIMADZU LCMS-2020 MSD or Agilent 1200\(G⁶¹¹⁰A MSD using electrospray ionization (ESI) in positive mode unless otherwise indicated. Calculated (calcd.) mass corresponds to the exact mass.

Nuclear magnetic resonance (NMR) spectra were obtained on Bruker model AVIII 400 spectrometers. Definitions for multiplicity are as follows: s=singlet, d=doublet, t=triplet, q=quartet, m=multiplet, br=broad. It will be understood that for compounds comprising an exchangeable proton, said proton may or may not be visible on an NMR spectrum depending on the choice of solvent used for running the NMR spectrum and the concentration of the compound in the solution.

Chemical names were generated using ChemDraw Ultra 12.0, ChemDraw Ultra 14.0 (CambridgeSoft Corp., Cambridge, Mass.) or ACD/Name Version 10.01 (Advanced Chemistry).

Compounds designated as R* or S* are enantiopure compounds where the absolute configuration was not determined.

### Intermediate 1: Tert-Butyl 5-methylene-5,6,9,10-tetrahydro-4H-isoxazolo[3,4-c]pyrido[4′,3′:3,4]-pyrazolo[1,5-a]azepine-11(12H)-carboxylate

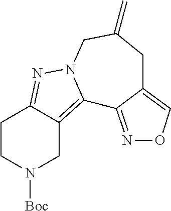 [SMILES: C=C1Cc2conc2-c2c3c(nn2C1)CCN(C(=O)OC(C)(C)C)C3]

### Step A. tert-Butyl 3-(3-ethoxy-3-oxopropanoyl)-6,7-dihydro-2H-pyrazolo[4,3-c]pyridine-5(4H)-carboxylate

To a solution of ethyl acetate (20.88 g, 237.02 mmol, 23.20 mL) in THF (120 mL) was added NaHMDS (1 M, 474.04 mL) at −65° C. under N2. A solution of 5-tert-butyl 3-ethyl 6,7-dihydro-2H-pyrazolo[4,3-c]pyridine-3,5(4H)-dicarboxylate (preparation as described in WO2018005881, publication date Jan. 4, 2018) (28 g, 94.81 mmol) in THF (200 mL) was added dropwise into the mixture over 1 h at −65° C. The mixture was stirred at 45° C. for 10 h. The mixture was quenched with HCl (1 M aq, 1500 mL) and diluted with ethyl acetate (1500 mL). The organic phases were separated and dried over Na₂SO₄, filtered and concentrated in vacuo. The residue was purified by column chromatography (SiO₂, petroleum ether/ethyl acetate=10/1 to 1/1) to give the title compound (28.4 g, 84.18 mmol, 88.79% yield, 100% purity) as a yellow solid. MS (ESI): mass calcd. for C₁₆H₂₃N₃O₅, 337.16: m/z found, 360.1 [M+Na]⁺.

### Step B. Mixture of di-tert-butyl 3-(3-ethoxy-3-oxopropanoyl)-6,7-dihydro-2H-pyrazolo[4,3-c]pyridine-2,5(4H)-dicarboxylate and di-tert-butyl 3-(3-ethoxy-3-oxopropanoyl)-6,7-dihydro-1H-pyrazolo[4,3-c]pyridine-1,5(4H)-dicarboxylate

To a solution of tert-butyl 3-(3-ethoxy-3-oxopropanoyl)-6,7-dihydro-2H-pyrazolo[4,3-c]pyridine-5(4H)-carboxylate (18 g, 53.35 mmol), TEA (16.20 g, 160.06 mmol, 22.28 mL) and DMAP (651.82 mg, 5.34 mmol) in DCM (200 mL) was added Boc₂O (11.64 g, 53.35 mmol, 12.26 mL), the mixture was stirred at 15° C. for 2 h. The mixture was poured into 1 M HCl aq (250 mL) and extracted with ethyl acetate (200 mL×2). The combined organic phases were washed with brine (200 mL), dried with anhydrous Na₂SO₄, filtered and the filtrate concentrated under reduced pressure. The residue was purified by silica flash column chromatography (eluent of 0-20% ethyl acetate/petroleum ether) to give the title compound (20 g, 22.86 mmol, 42.84% yield, 100% purity) as a colorless oil. MS (ESI): mass calcd. for C₂₁H₃₁N₃O₇, 437.22; m/z found, 460.1 [M+Na]⁺/897.2 [2M+23]⁺.

### Step C. Mixture of di-tert-butyl3-(4-(((tert-butyldiphenylsilyloxy)methyl)-2-(ethoxycarbonyl)-pent-4-enoyl)-6,7-dihydro-2H-pyrazolo[4,3-c]pyridine-2,5(4H)-dicarboxylate and di-tert-butyl3-(4-(((tert-butyldiphenylsilyloxy)methyl)-2-(ethoxycarbonyl)pent-4-enoyl)-6,7-dihydro-1H-pyrazolo[4,3-c]pyridine-1,5(4H)-dicarboxylate

To a mixture of di-tert-butyl 3-(3-ethoxy-3-oxopropanoyl)-6,7-dihydro-2H-pyrazolo[4,3-c]pyridine-2,5(4H)-dicarboxylate and di-tert-butyl 3-(3-ethoxy-3-oxopropanoyl)-6,7-dihydro-1H-pyrazolo[4,3-c]pyridine-1,5(4H)-dicarboxylate (14.00 g, 32.04 mmol) in acetone (150 mL) was added K₂CO₃ (6.64 g, 48.05 mmol), NaI (960.39 mg, 6.41 mmol) and 2-(bromomethyl)allyloxy-tert-butyl-diphenyl-silane (14.97 g, 38.44 mmol). The mixture was stirred at 55° C. for 4 h. The mixture was poured into HCl (400 mL, 1 M aq) at 0° C. and extracted with ethyl acetate (300 mL×3). The combined organic phases were washed with brine (500 mL), dried with anhydrous Na₂SO₄, filtered, and the filtrate concentrated under reduced pressure. The residue was purified by column chromatography (SiO₂, petroleum ether/ethyl acetate=30/1 to 201) to afford the title compound (13.5 g, 16.83 mmol, 52.53% yield, 93% purity) (TLC, petroleum ether/ethyl acetate=3/1) as a yellow oil. MS (ESI): mass calcd. for C₄₁H₅₅N₃O₃Si, 745.38; m/z found, 768.5 [M+Na]⁺.

### Step D. tert-Butyl 3-(4-(((tert-butyldiphenylsilyl)oxy)methyl)pent-4-enoyl)-6,7-dihydro-2H-pyrazolo[4,3-c]pyridine-5(4H)-carboxylate

To a mixture of di-tert-butyl 3-(4-(((tert-butyl-diphenylsilyl)oxy)methyl)-2-(ethoxycarbonyl)pent-4-enoyl)-6,7-dihydro-2H-pyrazolo[4,3-c]pyridine-2,5(4H)-dicarboxylate and di-tert-butyl 3-(4-(((tert-butyldiphenylsilyl)oxy)methyl)-2-(ethoxycarbonyl)pent-4-enoyl)-6,7-dihydro-1H-pyrazolo[4,3-c]pyridine-1,5(4H)-dicarboxylate (13.5 g, 16.83 mmol) in MeOH (50 mL) was added a solution of KOH (1.89 g, 33.66 mmol) in water (10 mL). The mixture was stirred at 65° C. for 3 h. The mixture was poured into HCl (1M, aq, 300 mL) and extracted with ethyl acetate (200 mL×3). The combined organic phases were washed with brine (200 mL), dried with anhydrous Na₂SO₄, filtered, and the filtrate concentrated under reduced pressure. The residue was purified by silica gel chromatography (SiO₂: petroleum ether/ethyl acetate=3/1) to afford the title compound (8.9 g, 15.51 mmol, 92.15% yield) as a yellow oil. MS (ESI): mass calcd. for C₃₃H₄₃N₃O₄Si, 573.3: m/z found, 574.4 [M+H]⁺.

### Step E. tert-Butyl 3-(4-(hydroxymethyl)pent-4-enoyl-6,7-dihydro-2H-pyrazolo [4,3-c]pyridine-5(4H)-carboxylate

To a solution of tert-butyl 3-(4-(((tert-butyldiphenylsilyl)oxy)methyl)pent-4-enoyl)-6,7-dihydro-2H-pyrazolo[4,3-c]pyridine-5(4H)-carboxylate (14 g, 21.96 mmol) in THF (50 mL) was added TBAF (1 M, 32.94 mL). The mixture was stirred at 30° C. for 12 h. The mixture was poured into water (100 mL) and extracted with ethyl acetate (80 mL×3). The combined organic phases were washed with brine (100 mL), dried with anhydrous Na₂SO₄, filtered and the filtrate concentrated under reduced pressure. The residue was purified by silica gel chromatography (petroleum ether/ethyl acetate=2/1 to 1/1) to afford the title compound (6.3 g, 18.41 mmol, 83.83% yield, 98% purity) as a white solid. MS (ESI)/mass calcd. for C₁₇H₂₅N₃O₄, 335.2: m/z found, 358.1 [M+Na]⁺; ¹H NMR (400 MHz, CDCl₃) δ=5.05 (s, 1H), 4.91 (s, 1H), 4.67 (s, 2H), 4.16 (s, 2H), 3.72 (t, J=5.4 Hz, 2H), 3.15 (s, 2H), 2.79 (t, J=5.6 Hz, 2H), 2.53 (t, J=7.2 Hz, 2H), 1.49 (s, 9H).

### Step F. tert-Butyl 3-(4-(((methylsulfonyl)oxy)methyl)pent-4-enoyl)-6,7-dihydro-2H-pyrazolo-[4,3-c]pyridine-5(4H)-carboxylate

To a solution of tert-butyl 3-(4-(hydroxymethyl)pent-4-enoyl)-6,7-dihydro-2H-pyrazolo[4,3-c]pyridine-5(4H)-carboxylate (6.3 g, 18.41 mmol) and TEA (5.59 g, 55.23 mmol, 7.69 mL) in DCM (30 mL) was added MsCl (4.73 g, 41.29 mmol, 3.20 mL) at 0° C. under N₂. The mixture was stirred at 0° C. for 1 h. The mixture was poured into water (60 mL) and extracted with ethyl acetate (60 mL×3). The combined organic phases were washed with brine (60 mL), dried with anhydrous Na₂SO₄, filtered, and the filtrate concentrated under reduced pressure to afford the title compound (8.2 g, crude) as a yellow oil. MS (ESI): mass calcd. for C₁₈H₂₇N₃O₆S, 413.2; m/z found, 414.1 [M+H]⁺.

### Step G. tert-Butyl 8-methylene-11-oxo-3,4,8,9,10,11-hexahydro-1H-pyrido[4′,3′:3,4]pyrazolo-[1,5-a]azepine-2(7H)-carboxylate

To a solution of tert-butyl3-(4-(((methylsulfonyl)oxy)methyl)-pent-4-enoyl)-6,7-dihydro-2H-pyrazolo[4,3-c]pyridine-5(4H)-carboxylate (8.2 g, crude) in THF (60 mL) was added DBU (7.06 g, 46.37 mmol, 6.99 mL) at 30° C. under N₂. The mixture was stirred at 30° C. for 1 h. The mixture was poured into water (50 mL), extracted with ethyl acetate (50 mL×3). The combined organic phases were washed with brine (50 mL), dried over anhydrous Na₂SO₄, filtered, and the filtrate concentrated under reduced pressure. The residue was purified by silica gel chromatography (silica gel, petroleum ether/ethyl acetate=10/1-8/1) to afford the title compound (4.2 g, 11.25 mmol, 85% purity) as a colorless oil. MS (ESI): mass calcd. for C₁₇H₂₃N₃O₃, 317.2; m/z found, 318.2 [M+H]⁺; ¹H NMR (400 MHz, CDCl₃) δ=5.22 (s, 1H), 5.09 (s, 1H), 5.03 (s, 2H), 4.62 (s, 2H), 3.68 (s, 2H), 2.93-2.87 (m, 2H), 2.74 (s, 4H), 1.47 (s, 9H).

### Step H. tert-Butyl 10-((dimethylamino)methylene-8-methylene-11-oxo-3,4,8,9,10,11-hexa-hydro-1H-pyrido[4′,3′:3,4]pyrazolo[1,5-a]azepine-2(7H)-carboxylate

A solution of tert-butyl 8-methylene-11-oxo-3,4,8,9,10,11-hexahydro-1H-pyrido[4′,3′:3,4]pyrazolo[1,5-a]azepine-2(7H)-carboxylate (4.2 g, 11.25 mmol) in DMF-DMA (15 mL) was stirred at 80° C. for 12 h. The mixture was concentrated under reduced pressure. The residue was poured into water (30 mL) and extracted with ethyl acetate (20 mL×2). The combined organic phases were washed with brine (20 mL×2), dried over anhydrous Na₂SO₄, filtered, and the filtrate concentrated under reduced pressure to afford the title compound (4.5 g, crude) as a yellow solid. MS (ESI): mass calcd. for C₂₀H₂₈N₄O₃, 372.2: m/z found, 395.1 [M+Na]⁺.

### Step I. tert-Butyl 5-methylene-5,6,9,10-tetrahydro-4H-isoxazolo[3,4-c]pyrido[4′,3′:3,4]pyrazolo-[1,5-a]azepine-11(12H)-carboxylate

To a solution of tert-butyl10-((dimethylamino)methylene)-8-methylene-11-oxo-3,4,8,9,10,11-hexahydro-1H-pyrido[4′,3′:3,4]pyrazolo[1,5-a]azepine-2(7H)-carboxylate (4.5 g, crude) in Py (50 mL) was added NH₂OH.HCl (5.04 g, 72.53 mmol). The mixture was stirred at 115° C. for 12 h. The mixture was concentrated under reduced pressure. The residue was poured into HCl (1N, aq. 40 mL) and stirred for 1 min. The aqueous phase was extracted with ethyl acetate (40 mL×2). The combined organic phases were washed with brine (30 mL×2), dried over anhydrous Na₂SO₄, filtered, and the filtrate concentrated under reduced pressure. The residue was purified by column chromatography (SiO₂, petroleum ether/ethyl acetate=10/1 to 5/1) to afford the title compound (2.1 g, 5.95 mmol, 97% purity) as a white solid. MS (ESI): mass calcd. for C₁₈H₂₂N₄O₃, 342.2: m/z found, 343.1 [M+H]⁺. ¹H NMR (400 MHz, CDCl₃) δ=8.32 (s, 1H), 5.34 (s, 1H), 5.26 (s, 1H), 4.93 (s, 2H), 4.68 (s, 2H), 3.75 (s, 2H), 3.64 (s, 2H), 2.79 (s, 2H), 1.50-1.47 (m, 9H).

### Intermediate 2: tert-Butyl 5-(hydroxymethyl)-5,6,9,10-tetrahydro-4H-isoxazolo[3,4-c]pyrido-[4′,3′:3,4]pyrazolo[1,5-a]azepine-11(12H)-carboxylate

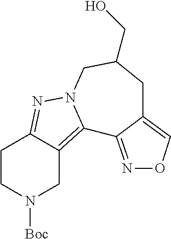 [SMILES: CC(C)(C)OC(=O)N1CCc2nn3c(c2C1)-c1nocc1CC(CO)C3]

To a solution of tert-butyl 5-methylene-5,6,9,10-tetrahydro-4H-isoxazolo[3,4-c]pyrido[4′,3′:3,4]-pyrazolo[1,5-a]azepine-11(12H)-carboxylate (Intermediate 1, product from Step 1, 480 mg, 1.40 mmol) in THF (5 mL) was added 1,9-BBN (0.5 M, 56.08 mL) at −10° C. The mixture was stirred at −10° C. for 2 h then a solution of NaOH (560.72 mg, 14.02 mmol) in water (5 mL) was added at −30° C., followed by H₂O₂ (3.18 g, 28.04 mmol, 2.69 mL, 30% purity). The mixture was stirred at 15° C. for 16 h. The mixture was quenched with sat.aq NaHSO₃ (50 mL) and extracted with EtOAc (80 mL×3), the combined organic layers were dried over Na₂SO₄, filtered, and the filtrate concentrated under reduced pressure. The residue was purified by column chromatography (SiO₂, petroleum ether/ethyl acetate=50%-100%) to afford the title compound (460 mg, 1.24 mmol, 88.31% yield, 97% purity) as a white solid. MS (ESI): mass calcd. for C₁₈H₂₄N₄O₄, 360.18; m/z found, 361.0 [M+H]⁺.

### Intermediate 3: (5S*)-tert-Butyl 5-((2,2-difluoroethoxy)methyl)-5,6,9,10-tetrahydro-4H-isoxazolo[3,4-c]pyrido[4′,3′:3,4]pyrazolo[1,5-a]azepine-1 (12H)-carboxylate

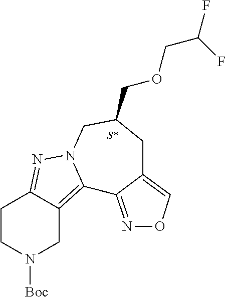 [SMILES: CC(C)(C)OC(=O)N1CCc2nn3c(c2C1)-c1nocc1C[C@H](COCC(F)F)C3]

### Step A. (5S*)-tert-Butyl 5-(hydroxymethyl)-5,6,9,10-tetrahydro-4H-isoxazolo[3,4-c]pyrido-[4′,3′:3,4]pyrazolo[1,5-a]azepine-11(12H)-carboxylate and (5R*)-tert-Butyl5-(hydroxymethyl)-5,6,9,10-tetrahydro-4H-isoxazolo[3,4-c]pyrido[4′,3′:3,4]pyrazolo[1,5-a]azepine-11(12H)-carboxylate

tert-Butyl 5-(hydroxymethyl)-5,6,9,10-tetrahydro-4H-isoxazolo[3,4-c]pyrido-[4′,3′:3,4]pyrazolo[1,5-a]azepine-11(12H)-carboxylate (Intermediate 2) was isolated by SFC (condition: column: IC (250 mm×30 mm, 10 um); mobile phase: [0.1% NH₃ H₂O IPA]; B %: 45%-45%, 6.1 min:100 min) to give (5S*)-tert-butyl 5-(hydroxymethyl)-5,6,9,10-tetrahydro-4H-isoxazolo[3,4-c]pyrido [4′,3′:3,4]pyrazolo[1,5-a]azepine-11(12H)-carboxylate (Peak 1 on SFC (IC-3S_4_40_3ML Column: Chiralpak IC-3 100×4.6 mm I.D., 3 um Mobile phase: 40% iso-propanol (0.05% DEA) in CO₂ Flow rate: 3 mL/min Wavelength: 220 nm), retention time=1.369 min, 136 mg, 97% purity) as a white solid and (5R*)-tert-butyl 5-(hydroxymethyl)-5,6,9,10-tetrahydro-4H-isoxazolo[3,4-c]pyrido[4′,3′:3,4]pyrazolo[1,5-a]azepine-11(12H)-carboxylate (Peak 2 on SFC (IC-3S_4_40_3ML Column: Chiralpak IC-3 100×4.6 mm I.D., 3 um Mobile phase: 40% iso-propanol (0.05% DEA) in CO₂ Flow rate: 3 mL/min Wavelength: 220 nm), retention time=1.627 min, 82 mg, 97% purity) as a white solid.

### Step B. (5S*)-tert-Butyl 5-((2,2-difluoroethoxy)methyl)-5,6,9,10-tetrahydro-4H-isoxazolo[3,4-c]pyrido[4′,3′:3,4]pyrazolo[1,5-a]azepine-11(12H)-carboxylate

To a solution of (5S*)-tert-butyl 5-(hydroxymethyl)-5,6,9,10-tetrahydro-4H-isoxazolo[3,4-c]pyrido[4′,3′:3,4]pyrazolo[1,5-a]-azepine-11(12H)-carboxylate (135.00 mg, 363.34 umol) in THF (2 mL) was added NaH (30 mg, 750.07 umol, 60% purity). The mixture was stirred at 0° C. for 0.5 h, and then 2,2-difluoroethyl trifluoromethanesulfonate (234 mg, 1.09 mmol) was added to the mixture. The mixture was stirred at 0° C. for 4 h, then poured into ice-water (20 mL) and extracted with ethyl acetate (20 mL×3). The combined organic phases were washed with brine (30 mL), dried with anhydrous Na₂SO₄, filtered, and the filtrate concentrated under reduced pressure to afford the title compound (140 mg, crude) as a colorless oil. MS (ESI): mass calcd. for C₂₀H₂₆F₂N₄O₄, 424.2: m/z found, 425.1 [M+H]⁺.

### Intermediate 4. (5R*)-tert-Butyl 5-((2,2-difluoroethoxy)methyl)-5,6,9,10-tetrahydro-4H-isoxazolo[3,4-c]pyrido[4′,3′:3,4]pyrazolo[1,5-a]azepine-11(12H)-carboxylate

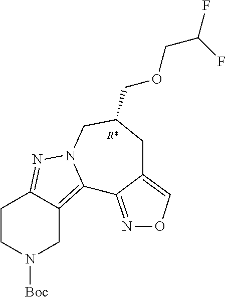 [SMILES: CC(C)(C)OC(=O)N1CCc2nn3c(c2C1)-c1nocc1C[C@@H](COCC(F)F)C3]

The title compound was prepared in a manner analogous to Intermediate 3, but substituting (5R*)-tert-butyl 5-(hydroxymethyl)-5,6,9,10-tetrahydro-4H-isoxazolo[3,4-c]pyrido[4′,3′:3,4]pyrazolo-[1,5-a]azepine-11(12H)-carboxylate for (5S*)-tert-butyl 5-(hydroxymethyl)-5,6,9,10-tetrahydro-4H-isoxazolo[3,4-c]pyrido[4′,3′:3,4]pyrazolo[1,5-a]azepine-11(12H)-carboxylate in Step B. MS (ESI): mass calcd. for C₂₀H₂₆F₂N₄O₄, 424.2: m/z found, 425.1 [M+H]⁺.

### Intermediate 5: tert-Butyl 5-methylene-5,6,9,10-tetrahydro-4H-isoxazolo[5,4-c]pyrido[4′,3′:3,4]-pyrazolo[1,5-a]azepine-11(12H)-carboxylate

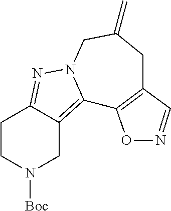 [SMILES: C=C1Cc2cnoc2-c2c3c(nn2C1)CCN(C(=O)OC(C)(C)C)C3]

To a solution of tert-butyl 10-((dimethylamino)methylene)-8-methylene-11-oxo-3,4,8,9,10,11-hexahydro-1H-pyrido[4′,3′:3,4]pyrazolo[1,5-a]azepine-2(7H)-carboxylate (Intermediate 1, product from Step H, 0.32 g, 859.15 umol) in MeOH (10 mL) was added NH₂OH.HCl (358.21 mg, 5.15 mmol). The mixture was stirred at 30° C. for 12 h. The mixture was poured into water (20 mL) and extracted with ethyl acetate (30 mL×3). The combined organic phases were washed with brine (50 mL), dried over anhydrous Na₂SO₄, filtered, and the filtrate concentrated under reduced pressure. The residue was purified by column chromatography (SiO₂, petroleum ether/ethyl acetate=20/1 to 2/1) to afford the title compound (200 mg, 519.87 umol, 89% purity) as a colorless oil. MS (ESI): mass calcd. for C₁₈H₂₂N₄O₃, 342.2; m/z found, 343.1 [M+H]⁺; ¹H NMR (400 MHz, CDCl₃) δ=8.17 (s, 1H), 5.39 (s, 1H), 5.33 (s, 1H), 4.87 (s, 2H), 4.75 (s, 2H), 3.74 (s, 2H), 3.57 (s, 2H), 2.78 (t, J=5.4 Hz, 2H), 1.49 (s, 9H).

### Intermediate 6: tert-Butyl 5-hydroxy-5,6,9,10-tetrahydro-4H-isoxazolo[3,4-c]pyrido[4′,3′:3,4]-pyrazolo[1,5-a]azepine-11(12H)-carboxylate

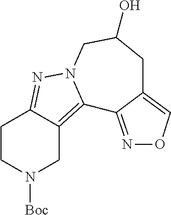 [SMILES: CC(C)(C)OC(=O)N1CCc2nn3c(c2C1)-c1nocc1CC(O)C3]

### Step A. tert-Butyl 5-hydroxy-5-(hydroxymethyl-5,6,9,10-tetrahydro-4H-isoxazolo[3,4-c]pyrido[4′,3′:3,4]pyrazolo[1,5-a]azepine-11(12H)-carboxylate

To a solution of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1, product from Step 1, 300 mg, 876.19 umol) in THF (20 mL) and H₂O (10 mL) were added NMO (153.97 mg, 1.31 mmol, 138.71 uL) and K₂OsO₄.2H₂O (32.28 mg, 87.62 umol) at 0° C. The mixture was stirred at 25° C. for 16 h. Additional NMO (153.97 mg) and K₂OsO₄.2H₂O (50 mg) were added and the mixture was stirred at 25° C. for 16 h. The mixture was diluted with water (20 mL) and extracted with ethyl acetate (20 mL-3), the combined organic layers were washed with sat. aq. NaHSO₃ (20 mL×2), dried over Na₂SO₄, filtered and the filtrate concentrated under reduced pressure to afford the title compound (334 mg, crude) as a white solid. MS (ESI): mass calcd. for C₁₈H₂₄N₄O₅, 376.2; m/z found, 377.1 [M+H]⁺.

### Step B. tert-Butyl 5-oxo-5,6,9,10-tetrahydro-4H-isoxazolo[3,4-c]pyrido[4′,3′:3,4]pyrazolo[1,5-a]azepine-11(12H)-carboxylate

To a solution of tert-butyl 5-hydroxy-5-(hydroxymethyl)-5,6,9,10-tetrahydro-4H-isoxazolo[3,4-c]pyrido[4′,3′:3,4]pyrazolo[1,5-a]azepine-11(12H)-carboxylate (330 mg) in THF (3.3 mL) and water (3.3 mL) was added NaIO₄ (562.56 mg, 2.63 mmol, 145.74 uL). The mixture was stirred at 25° C. for 2 h. The mixture was diluted with water (50 mL), extracted with EtOAc (40 mL×2), combined organic layers were dried over Na₂SO₄, filtered, and the filtrate concentrated under reduced pressure to afford the title compound (320 mg, crude) as a brown solid. LCMS indicated 60% of hydrate mass and 24% of desired mass. MS (ESI): mass calcd. for C₁₇H₂₀N₄O₄, 344.4: m/z found, 345.2 [M+H]⁺

### Step C. tert-Butyl 5-hydroxy-5,6,9,10-tetrahydro-4H-isoxazolo[3,4-c]pyrido[4′,3′:3,4]-pyrazolo[1,5-a]azepine-11(12H)-carboxylate

To a solution of tert-butyl 5-oxo-5,6,9,10-tetrahydro-4H-isoxazolo[3,4-c]pyrido[4′,3′:3,4]pyrazolo[1,5-a]azepine-11(12H)-carboxylate (300 mg) in EtOH (3 mL) was added NaBH₄ (65.92 mg, 1.74 mmol) at 0° C. The mixture was stirred at 25° C. for 5 h. The reaction was quenched with sat.aq NH₄Cl (20 mL) and extracted with EtOAc (40 mL×3). The combined organic layers were dried over Na₂SO₄, filtered, and the filtrate concentrated under reduced pressure to afford the title compound (230 mg, crude) as a yellow solid. MS (ESI): mass calcd. for C₁₇H₂₂N₄O₄, 346.4; m/z found, 347.3 [M+H]⁺.

### Intermediate 7: (10R)-tert-Butyl 10-methyl-5,6,9,10-tetrahydro-4H-isoxazolo[5,4-c]pyrido-[4′3′:3,4]pyrazolo[1,5-a]azepine-11(12H)-carboxylate

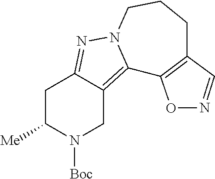 [SMILES: C[C@@H]1Cc2nn3c(c2CN1C(=O)OC(C)(C)C)-c1oncc1CCC3]

### Step A. (R)-5-tert-Butyl 3-ethyl 2-allyl-6-methyl-6,7-dihydro-2H-pyrazolo[4,3-c]pyridine-3,5(4H)-dicarboxylate

A mixture of (R)-5-tert-butyl 3-ethyl 6-methyl-6,7-dihydro-2H-pyrazolo[4,3-c]pyridine-3,5(4H)-dicarboxylate (preparation as described in PCT nt. Appl. WO 2018005883)(15 g, 48.49 mmol), 3-bromoprop-1-ene (8.80 g, 72.73 mmol). Cs₂CO₃ (39.50 g, 121.22 mmol) in anhydrous DMF (200 mL) was degassed and purged with N₂ 3 times, and then the mixture was stirred at 15° C. for 16 h under N₂ atmosphere. The mixture was poured into water (30 mL) and stirred for 5 min. The aqueous phase was extracted with ethyl acetate (20 mL). The organic phases were washed with brine (30 mL), dried with anhydrous Na₂SO₄, filtered and the filtrate concentrated under reduced pressure. The residue was purified by column chromatography (SiO₂, petroleum ether/ethyl acetate=100/1 to 1/1) to afford the title compound (9.7 g, 26.26 mmol, 54.16% yield, 94.6% purity) as a colorless oil. MS (ESI): mass calcd. for C₁₈H₂₇N₃O₄, 349.2; m/z found, 350.1 [M+H]⁺.

### Step B. (R)-tert-Butyl 2-allyl-3-(hydroxymethyl)-6-methyl-6,7-dihydro-2H-pyrazolo[4,3-c]pyridine-5(4H)-carboxylate

A solution of (R)-5-tert-butyl 3-ethyl 2-allyl-6-methyl-6,7-dihydro-2H-pyrazolo[4,3-c]pyridine-3,5(4H)-dicarboxylate (8 g, 22.89 mmol) in THF (80 mL) was added LiAlH₄ (1.30 g, 34.34 mmol) at −40° C. under N₂, and then the mixture was stirred at −40° C. for 2 h under N₂ atmosphere. Ice-NaOH (3 mL, 15% aq) was added to the mixture dropwise at −40° C. and stirred for 5 min. Then the mixture was warmed to 15° C. and filtered. The filtrate was poured into water (40 mL) and extracted with ethyl acetate (30 mL×2). The combined organic phases were washed with brine (100 mL), dried over anhydrous Na₂SO₄, filtered and the filtrate concentrated under reduced pressure. The residue was purified by column chromatography (SiO₂, petroleum ether/ethyl acetate=100/l to 0:1) to afford the title compound (6.3 g, 20.29 mmol, 88.62% yield, 99% purity) as a colorless oil. MS (ESI): mass calcd. for C₁₆H₂₅N₃O₃, 307.2; m/z found, 308.1 [M+H]⁺.

### Step C. (R)-tert-Butyl 2-allyl-3-formyl-6-methyl-6,7-dihydro-2H-pyrazolo[4,3-c]pyridine-5(4H)-carboxylate

To a solution of (COCl)2 (4.74 g, 37.33 mmol, 3.27 mL) in DCM (150 mL) was added DMSO (3.89 g, 49.77 mmol, 3.89 mL) in one portion under N₂ at −78° C. The mixture was stirred at −78° C. for 15 min. Then (R)-tert-butyl 2-allyl-3-(hydroxymethyl)-6-methyl-6,7-dihydro-2H-pyrazolo[4,3-c]pyridine-5(4H)-carboxylate (7.3 g, 24.88 mmol) was added followed by TEA (8.81 g, 87.09 mmol, 12.12 mL). The mixture was stirred at −78° C. for 2 h under a N₂ atmosphere, then the mixture was poured into water (200 mL) at −40° C., stirred for 1 min, then warmed to 15° C. The aqueous phase was extracted with DCM (100 mL×2). The combined organic phases were washed with brine (300 mL), dried over anhydrous Na₂SO₄, filtered and the filtrate concentrated under reduced pressure. The residue was purified by column chromatography (SiO₂, petroleum ether/ethyl acetate=100/1 to 501) to afford the title compound (6.4 g, 21.31 mmol, 85.63% yield, 97% purity) as a colorless oil. MS (ESI): mass calcd. for C₁₆H₂₃N₃O₃, 305.2; m/z found, 306.1 [M+H]⁺.

### Step D. (6R)-tert-Butyl 2-allyl-3-(1-hydroxybut-3-en-1-yl)-6-methyl-6,7-dihydro-2H-pyrazolo[4,3-c]pyridine-5(41H)-carboxylate

To a solution of (R)-tert-butyl 2-allyl-3-formyl-6-methyl-6,7-dihydro-2H-pyrazolo[4,3-c]pyridine-5(4H)-carboxylate (5.8 g, 18.99 mmol) in THF (60 mL) was added allyl(bromo)magnesium (0 M, 56.98 mL) dropwise at −40° C. under N2. The mixture was stirred at −40° C. for 30 min, then heated to 0° C. and stirred for 2 h. The mixture was quenched with ice-HCl (aq. 1 N, 50 mL) and stirred for 1 min. The aqueous phase was extracted with ethyl acetate (60 mL×2). The combined organic phases were washed with brine (100 mL), dried with anhydrous Na₂SO₄, filtered and the filtrate concentrated under reduced pressure. The residue was purified by column chromatography (SiO₂, petroleum ether/ethyl acetate=100/1 to 1:1) to afford the title compound (5.7 g, 15.70 mmol, 82.66% yield, 95.7% purity) as a colorless oil. MS (ESI): mass calcd. for C₁₉H₂₉N³O₃, 347.2: m/z found, 348.1 [M+H]⁺.

### Step E. (3R)-tert-Butyl 11-hydroxy-3-methyl-3,4,10,11-tetrahydro-1H-pyrido[4′,3′:3,4]pyrazolo-[1,5-a]azepine-2(7H)carboxylate

A mixture of (6R)-tert-butyl 2-allyl-3-(1-hydroxybut-3-en-1-yl)-6-methyl-6,7-dihydro-2H-pyrazolo[4,3-c]pyridine-5(4H)-carboxylate (2.2 g, 6.33 mmol), [1,3-bis(2,4,6-trimethylphenyl)imidazolidin-2-ylidene]-dichloro-[(2-isopropoxyphenyl)methylene] ruthenium (396.77 mg, 633.18 umol) in DCM (1.6 L) was degassed and purged with N₂ (3×), and then the mixture was stirred at 40° C. for 16 h under a N₂ atmosphere. [1,3-Bis(2,4,6-trimethylphenyl)imidazolidin-2-ylidene]-dichloro-[(2-isopropoxyphenyl)-methylene]ruthenium (198.38 mg, 316.59 umol) was added to the mixture at 15° C. under a nitrogen atmosphere. The mixture was stirred at 34° C. for another 32 h under N2, then the mixture was stirred at 40° C. for an additional 32 h. The mixture was concentrated under reduced pressure. The residue was purified by column chromatography (SiO₂, petroleum ether/ethyl acetate=100/1 to 3/1) to afford the title compound (1.8 g, 5.58 mmol, 88.11% yield, 99% purity) as a black brown solid. MS (ESI): mass calcd. for C₁₇H₂₅NiO₃, 319.2; m/z found, 320.1 [M+H]⁺.

### Step F. (3R tert-Butyl 11-hydroxy-3-methyl-3,4,8,9,10,11-hexahydro-1H-pyrido[4′,3′:3,4]-pyrazolo[1,5-a]azepine-2(7H)-carboxylate

To a solution of (3R)-tert-butyl 11-hydroxy-3-methyl-3,4,10,11-tetrahydro-1H-pyrido[4′,3′:3,4]pyrazolo[1,5-a]azepine-2(7H)-carboxylate (750 mg, 2.35 mmol) in MeOH (30 mL) was added Pd/C (75 mg, 10%) under N2. The suspension was degassed under reduced pressure and purged with H₂ several times. The mixture was stirred under H₂ (15 psi) at 15° C. for 16 h. The mixture was filtered and concentrated under reduced pressure to afford the title compound (680 mg, 2.12 mmol, 90.10% yield) as a black brown oil. MS (ESI): mass calcd. for C₁₇H₂₇N₃O₃, 321.2; m/z found, 322.1 [M+H]⁺.

### Step G. (R)-tert-Butyl 3-methyl-11-oxo-3,4,8,9,10,11-hexahydro-1H-pyrido[4′,3′:3,4]pyrazolo[1,5-a]azepine-2(7H)-carboxylate

A mixture of (3R)-tert-butyl 11-hydroxy-3-methyl-3,4,8,9,10,11-hexahydro-1H-pyrido-[4′,3′:3,4]pyrazolo[1,5-a]azepine-2(7H)-carboxylate (680 mg, 2.12 mmol), TPAP (148.70 mg, 423.13 umol) and NMO (991.36 mg, 8.46 mmol, 893.11 uL) in acetonitrile (ACN) (10 mL) was degassed and purged with N₂ (3×), and then the mixture was stirred at 15° C. for 16 h under a N₂ atmosphere. The mixture was concentrated under reduced pressure. The residue was purified by column chromatography (SiO₂, petroleum ether:ethyl acetate=10/1 to 1/1) to afford the title compound (600 mg, 1.84 mmol, 87.02% yield, 98% purity) as a yellow oil. MS(ESI): mass calcd. for C₁₇H₂₅N₃O₃, 319.2: m/z found, 320.1 [M+H]⁺.

### Step H. (R)-tert-Butyl 10-hydroxymethylene)-3-methyl-1-oxo-3,4,8,9,10,11-hexahydro-1H-pyrido[4′,3′:3,4]pyrazolo[1,5-a]azepine-2(7H)-carboxylate

A solution of (R)-tert-butyl 3-methyl-11-oxo-3,4,8,9,10,11-hexahydro-1H-pyrido [4′,3′:3,4]pyrazolo[1,5-a]azepine-2(7H)-carboxylate (400 mg, 1.25 mmol) in DMF-DMA (13.46 g, 112.91 mmol, 15 mL) was stirred at 75° C. for 16 h. The mixture was stirred at 75° C. for 16 h. The reaction mixture was concentrated under reduced pressure. The residue was poured into water (20 mL) and stirred for 2 min. The aqueous phase was extracted with ethyl acetate (20 mL/2). The combined organic phases were washed with brine (10 mL×2), dried with anhydrous Na₂SO₄, filtered and the filtrate concentrated under reduced pressure to afford the title compound (440 mg, crude) as a yellow solid. MS (ESI): mass calcd. for C₁₈H₂₃N₃O₄, 347.2; m/z found, 348.1 [M+H]⁺.

### Step 1. (10R)-tert-Butyl 10-methyl-5,6,9,10-tetrahydro-4H-isoxazolo[5,4-c]pyrido [4′3′:3,4]pyrazolo[1,5-a]azepine-11(12H)-carboxylate

To a solution of (R)-tert-butyl 10-(hydroxymethylene)-3-methyl-11-oxo-3,4,8,9,10,11-hexahydro-1H-pyrido[4′,3′:3,4]pyrazolo[1,5-a]azepine-2(7H)-carboxylate (200 mg, 575.69 umol) in MeOH (30 mL) was added NH₂OH.HCl (240.03 mg, 3.45 mmol) in one portion at 30° C. under N₂. The mixture was stirred at 30° C. for 16 h. The mixture was poured into water (100 mL) and stirred for 1 min. The aqueous phase was extracted with ethyl acetate (50 mL×2). The combined organic phases were washed with brine (00 mL×2), dried with anhydrous Na₂SO₄, filtered and the filtrate concentrated under reduced pressure. The residue was purified by prep-TLC (petroleum ether/ethyl acetate:=1/2) to afford the title compound (120 mg, 348.42 umol, 60.52% yield) as a light yellow oil. MS (ESI): mass calcd. for C₁₈H₂₄N₄O₃, 344.2; n/z found, 345.1 [M+H]⁺.

### Intermediate 8: (11R)-tert-Butyl 11-methyl-6,7,10,11-tetrahydro-5H-pyrido[2,3-c]pyrido-[4′,3′:3,4]pyrazolo[1,5-a]azepine-12(13H)-carboxylate

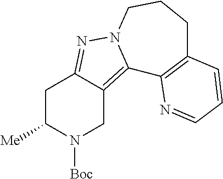 [SMILES: C[C@@H]1Cc2nn3c(c2CN1C(=O)OC(C)(C)C)-c1ncccc1CCC3]

To a solution of (R)-tert-butyl 3-methyl-11-oxo-3,4,8,9,10,11-hexahydro-1H-pyrido-[4′,3′:3,4]pyrazolo[1,5-a]azepine-2(7H)-carboxylate (160 mg, 500.94 umol, product of Step G in Intermediate 7) and prop-2-yn-1-amine (137.96 mg, 2.50 mmol, 160.41 uL) in EtOH (2 mL) was added NaAuCl₄.2H₂O (49.82 mg, 125.24 umol). The mixture was stirred at 80° C. for 72 h. The residue was diluted with water (10 mL) and the mixture was extracted with EtOAc (10 mL×3). The combined organic layers were washed with brine (10 mL), dried over Na₂SO₄, filtered and the filtrate concentrated under reduced pressure. The residue was purified by column chromatography (SiO₂, petroleum ether/ethyl acetate=101 to 2/1) to give the title compound (90 mg, 190.44 umol, 38.02% yield, 75% purity) as a yellow oil. MS (ESI): mass calcd. for C₂₀H₂₆N₄O₂, 354.2: m/z found, 355.1 [M+H]⁺.

### Intermediate 9: (10R)-tert-Butyl 10-methyl-5,6,9,10-tetrahydro-4H-isoxazolo[3,4-c]pyrido-[4′,3′:3,4]pyrazolo[1,5-a]azepine-11(12H)-carboxylate

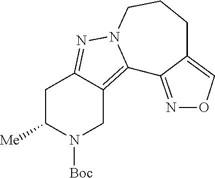 [SMILES: C[C@@H]1Cc2nn3c(c2CN1C(=O)OC(C)(C)C)-c1nocc1CCC3]

To a solution of (R)-tert-butyl 1-(hydroxymethylene)-3-methyl-11-oxo-3,4,8,9,10,11-hexahydro-1H-pyrido[4′,3′:3,4]pyrazolo[1,5-a]azepine-2(7H)-carboxylate (200 mg, 575.69 umol) in Py (30 mL) was added NH₂OH.HCl (240.03 mg, 3.45 mmol) in one portion under N₂. The mixture was stirred at 115° C. for 16 h. The mixture was concentrated under reduced pressure. The residue was poured into HCl (1N aq, 100 mL) and stirred for 1 min. The aqueous phase was extracted with ethyl acetate (50 mL×2). The combined organic phases were washed with brine (100 mL×2), dried over anhydrous Na₂SO₄, filtered, and the filtrate concentrated under reduced pressure. The residue was purified by prep-TLC (petroleum ether/ethyl acetate=1/2) to afford the title compound (90 mg, 261.32 umol, 45.39% yield) as a light yellow oil. MS (ESI): mass calcd. for C₁₈H₂₄N₄O, 344.2; m/z found, 345.1 [M+H]⁺.

### Intermediate 10: tert-Butyl 6,7,10,11-tetrahydro-5H-pyrido[4′,3′:3,4]pyrazolo[1,5-a][1,2,4]-triazolo[3,4-c][1,4]diazepine-12(13H)-carboxylate

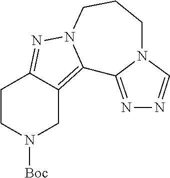 [SMILES: CC(C)(C)OC(=O)N1CCc2nn3c(c2C1)-c1nncn1CCC3]

### Step A. tert-Butyl 11-thioxo-3,4,8,9,10,11-hexahydro-1H-pyrido[4′,3′:3,4]pyrazolo[1,5-a][1,4]diazepine-2(7H)-carboxylate

To a solution of tert-butyl 11-oxo-3,4,7,8,9,10-hexahydro-1H-pyrido[2,3]pyrazolo[2,4-b][1,4]diazepine-2-carboxylate (preparation as described in PCT Int. Appl. WO2018005883, Jan. 4, 2018) (250.00 mg, 816.03 umol) in toluene (5 mL) was added Lawesson's reagent (165.03 mg, 408.02 umol). The mixture was heated to 110° C. for 3 h, then concentrated under reduced pressure. The residue was purified by prep-TLC (EtOAc) to afford the title compound (258.0 mg, 800.20 umol, 98.06% yield) as a yellow solid.

### Step B. tert-Butyl 6,7,10,11-tetrahydro-5H-pyrido[4′,3′:3,4]pyrazolo[1,5-a][1,2,4]triazolo[3,4-c][1,4]diazepine-12(13H)-carboxylate

To a solution of tert-butyl 11-thioxo-3,4,8,9,10,11-hexahydro-1H-pyrido[4′,3′:3,4]pyrazolo[1,5-a][1,4]diazepine-2(7H)-carboxylate (80.00 mg, 248.12 umol) and formohydrazide (74.51 mg, 1.24 mmol) in MeCN (3.00 mL) was added Hg(OAc)₂ (118.61 mg, 372.18 umol), then the mixture was stirred at 20° C. for 16 h. The mixture was diluted with water (50 mL) and extracted with EtOAc (20 mL×3). The combined organic layers were washed with brine (24) mL×2), dried over Na₂SO₄, filtered, and the filtrate concentrated under reduced pressure to afford the title compound (100.00 mg, crude) as a colorless oil.

### Intermediate 11: tert-Butyl 3-methyl-6,7,10,11-tetrahydro-5H-pyrido[4′,3′:3,4a]pyrazolo[1,5-a][1,2,4]triazolo[3,4-c][1,4]diazepine-12(13H)-carboxylate

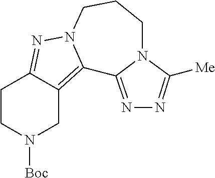 [SMILES: Cc1nnc2n1CCCn1nc3c(c1-2)CN(C(=O)OC(C)(C)C)CC3]

### Step A. tert-Butyl 11-thioxo-3,4,8,9,10,11-hexahydro-1H-pyrido[4′,3′:3,4]pyrazolo[1,5-a][1,4]diazepine-2(7H)-carboxylate

The title compound was prepared in a manner analogous to Intermediate 12, using tert-butyl 11-oxo-3,4,8,9,10,11-hexahydro-1H-pyrido[4′,3′:3,4]-pyrazolo[1,5-a][1.4]diazepine-2(7H)-carboxylate (preparation as described in PCT Int. Appl. WO2018005883) instead of (R)-tert-butyl 3-methyl-11-oxo-3,4,8,9,10,11-hexahydro-1H-pyrido[4′,3′:3,4]pyrazolo[1,5-a][1,4]diazepine-2(7H)-carboxylate in Step A. The title compound was used directly in the next step without further purification.

### Step B: tert-Butyl 3-methyl-6,7,10,11-tetrahydro-5H-pyrido[4′,3′:3,4]pyrazolo[1,5-a][1,2,4]-triazolo[3,4-c][1,4]diazepine-12(13H)-carboxylate

To a solution of tert-butyl 11-thioxo-3,4,8,9,10,11-hexahydro-1H-pyrido[4′,3′:3,4]pyrazolo[1,5-a][1,4]diazepine-2(7H)-carboxylate (80.0) mg, 248.12 umol) and acetohydrazide (91.90 mg, 1.24 mmol) in MeCN (3.00 mL) was added Hg(OAc)₂ (118.61 mg, 372.18 umol), then the mixture was stirred at 20° C. for 16 h. The mixture was extracted with EtOAc (20 mL×3) and water (30 mL). The combined organic layers were washed with brine (20 mL×2), dried over Na₂SO₄, filtered and the filtrate concentrated under reduced pressure to afford the title compound (100.00 mg, crude) as a colorless oil.

### Intermediate 12: (11R)-tert-Butyl 11-methyl-6,7,10,11-tetrahydro-5H-pyrido-[4′,3′:3,4]pyrazolo[1,5-a][1,2,4]triazolo[3,4-c][1,4]diazepine-12(13H)-carboxylate

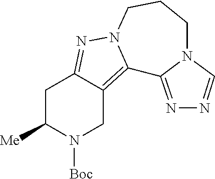 [SMILES: C[C@H]1Cc2nn3c(c2CN1C(=O)OC(C)(C)C)-c1nncn1CCC3]

### Step A. (R)-tert-Butyl 3-methyl-1-thioxo-3,4,8,9,10,11-hexahydro-1H-pyrido[4′,3′:3,4]-pyrazolo[1,5-a][1,4]diazepine-2(7H)-carboxylate

To a solution of (R)-tert-butyl 3-methyl-1-oxo-3,4,8,9,10,11-hexahydro-1H-pyrido[4′,3′:3,4]pyrazolo[1,5-a][1,4]diazepine-2(7H)-carboxylate (preparation as described in PCT Int. Appl. WO 2018005883) (300.00 mg, 936.36 umol) in toluene (3.00 mL) was added Lawesson reagent (189.36 mg, 468.18 umol). The mixture was heated to 110° C. for 3 h, then concentrated under reduced pressure. The residue was purified by column chromatography (petroleum ether/ethyl acetate=30% to 50%) to afford the title compound (270.00 mg, 650.02 umol, 69.42% yield, 81% purity) as a yellow solid.

### Step B. (11R)-tert-Butyl 11-methyl-6,7,10,11-tetrahydro-5H-pyrido[4′,3′:3,4]pyrazolo[1,5-a][1,2,4]triazolo[3,4-c][1,4]diazepine-12(13H)-carboxylate

To a suspension of (R)-tert-butyl 3-methyl-11-thioxo-3,4,8,9,10,11-hexahydro-1H-pyrido[4′,3′:3,4]pyrazolo[1,5-a][1,4]diazepine-2(7H)-carboxylate (100.00 mg, 297.22 umol) and formohydrazide (89.26 mg, 1.49 mmol) in MeCN (2.00 mL) was added Hg(OAc)₂ (142.08 mg, 445.83 umol). The mixture was stirred at 25° C. for 16 h, then diluted with water (50 mL) and extracted with EtOAc (50 mL×3). The combined organic layers were washed with brine (50 mL×2), dried over Na₂SO₄, filtered, and the filtrate concentrated under reduced pressure to afford the title compound (90.00 mg, crude) as a white solid. MS (ESI): mass calcd. for C₁₇H₂₄N₆O₂, 344.2; m/z found, 345.0 [M+H]⁺.

### Intermediate 13: (11R)-tert-Butyl 3,11-dimethyl-6,7,10,11-tetrahydro-5H-pyrido[4′,3′:3,4]-pyrazolo[1,5-a][1,2,4]triazolo[3,4-c][1,4]diazepine-12(13H)-carboxylate

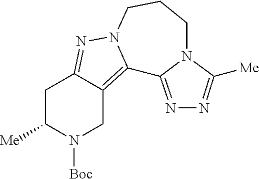 [SMILES: Cc1nnc2n1CCCn1nc3c(c1-2)CN(C(=O)OC(C)(C)C)[C@H](C)C3]

To a suspension of (R)-tert-butyl 3-methyl-11-thioxo-3,4,8,9,10,11-hexahydro-1H-pyrido-[4′ 3′:3,4]pyrazolo[1,5-a][1,4]diazepine-2(7H)-carboxylate (Intermediate 12, product from Step A, 80.00 mg, 237.78 umol) and acetohydrazide (88.07 mg, 1.19 mmol) in MeCN (3.00 mL) was added Hg(OAc)₂ (113.66 mg, 356.67 umol). The mixture was stirred at 25° C. for 16 h, then diluted with water (50 mL) and extracted with EtOAc (50 mL×3). The combined organic layers were washed with brine (50 mL×2), dried over Na₂SO₄, filtered, and the filtrate concentrated under reduced pressure to afford the title compound (90.00 mg, crude) as a white solid.

### Intermediate 14: tert-Butyl 11-oxo-3,4,8,9,10,11-hexahydro-1H-pyrido[4′,3′:3,4]pyrazolo[1,5-a]azepine-2(7H)-carboxylate

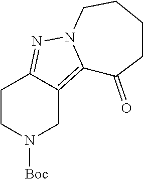 [SMILES: CC(C)(C)OC(=O)N1CCc2nn3c(c2C1)C(=O)CCCC3]

### Step A. 5-tert-Butyl 3-ethyl 2-allyl-6,7-dihydro-2H-pyrazolo[4,3-c]pyridine-3,5(4H)-dicarboxylate

To a mixture of 5-tert-butyl 3-ethyl 6,7-dihydro-2H-pyrazolo[4,3-c]pyridine-3,5(4H)-dicarboxylate (preparation as described in WO2018005881, publication date Jan. 4, 2018) (5.00 g, 16.93 mmol) and 3-bromoprop-1-ene (3.07 g, 25.40 mmol) in DMF (50.00 mL) was added Cs₂CO₃ (13.79 g, 42.33 mmol) in one portion under N₂. The mixture was stirred at 50° C. for 12 h. The mixture was poured into water (50 mL) and stirred for 1 min. The aqueous phase was extracted with ethyl acetate (50 mL×2). The combined organic phases were washed with brine (50 mL×2), dried with anhydrous Na₂SO₄, filtered, and the filtrate concentrated under reduced pressure. The residue was purified by silica gel chromatography (petroleum ether/ethyl acetate=15/1 to 5/1) to afford the title compound (2.70 g, 7.89 mmol, 46.60% yield, 98% purity) as a yellow solid. MS (ESI): mass calcd. for C₁₇H₂₅N₃O₄, 335.1: m/z found, 336.0 [M+H]⁺.

### Step B. tert-Butyl 2-allyl-3-(hydroxymethyl)-6,7-dihydro-2H-pyrazolo[4,3-c]pyridine-5(4H)-carboxylate

To a mixture of 5-tert-butyl 3-ethyl 2-allyl-6,7-dihydro-2H-pyrazolo[4,3-c]pyridine-3,5(4H)-dicarboxylate (1.00 g, 2.98 mmol) in THF (30.0) mL) was added LiAlH₄ (169.72 mg, 4.47 mmol) in one portion at −40° C. under N₂. The mixture was stirred at 20° C. for 1 h. The mixture was quenched with HCl (1N aq 10 mL). The aqueous phase was extracted with ethyl acetate (20 mL×2). The combined organic phases were washed with brine (20 mL×2), dried with anhydrous Na₂SO₄, filtered, and the filtrate concentrated under reduced pressure. The residue was purified by silica gel chromatography (dichloromethane/methanol=100/1˜20/1) to afford the title compound (780.00 mg, 2.66 mmol, 89.22% yield) as a yellow solid. MS (ESI): mass calcd. for C₁₅H₂₃N₃O₃, 293.1; m/z found, 294 [M+H]⁺.

### Step C. tert-Butyl 2-allyl-3-formyl-6,7-dihydro-2H-pyrazolo[4,3-c]pyridine-5(4H)-carboxylate

To a mixture of tert-butyl 2-allyl-3-(hydroxymethyl)-6,7-dihydro-2H-pyrazolo [4,3-c]pyridine-5(4H)-carboxylate (780.00 mg, 2.66 mmol) in DCM (30.0) mL) was added MnO₂ (2.31 g, 26.60 mmol) in one portion under N2. The mixture was stirred at 45° C. for 12 h. Additional MnO₂ (2.31 g, 26.60 mmol) was added and the mixture was stirred at 45° C. for another 24 h. At this time the mixture was filtered and concentrated under reduced pressure. The residue was purified by silica gel chromatography (petroleum ether/ethyl acetate=10/1 to 3/1) to afford the title compound (450.00 mg, 1.54 mmol, 58.07% yield, 100% purity) as a yellow solid. MS (ESI): mass calcd. for C₁₅H₂₁N₃O, 291.1; m/z found, 292 [M+H]⁺.

### Step D. tert-Butyl 2-allyl-3-(1-hydroxybut-3-en-1-yl)-6,7-dihydro-2H-pyrazolo[4,3-c]pyridine-5(4H)-carboxylate

To a mixture of tert-butyl 2-allyl-3-formyl-6,7-dihydro-2H-pyrazolo[4,3-c]pyridine-5(4H)-carboxylate (800.00 mg, 2.75 mmol) in THF (5.00 mL) was added allyl(bromo)magnesium (1 M, 8.24 mL) in one portion at −40° C. under N₂. The mixture was stirred at −40° C. for 2 h. The mixture was poured into water (20 mL) and stirred for 1 min. The aqueous phase was extracted with ethyl acetate (24) mL×2). The combined organic phases were washed with brine (10 mL 2), dried with anhydrous Na₂SO₄, filtered, and the filtrate concentrated under reduced pressure. The residue was purified by silica gel chromatography (petroleum ether/ethyl acetate=3/1 to 1/1) to afford the title compound (750.00 mg, 2.16 mmol, 78.53% yield. %% purity) as a yellow oil. MS (ESI): mass calcd. for C₁₈H₂₇N₃O₃, 333.2; m/z found, 334 [M+H]⁺.

### Step E. tert-Butyl 11-hydroxy-3,4,10,11-tetrahydro-1H-pyrido[4′,3′:3,4]pyrazolo[1,5-a]azepine-2(7H)-carboxylate

To a mixture of tert-butyl 2-allyl-3-(1-hydroxybut-3-en-1-yl)-6,7-dihydro-2H-pyrazolo[4,3-c]pyridine-5(4H)-carboxylate (750.00 mg, 2.25 mmol) in DCM (1.20 L) was added benzylidene-[1,3-bis(2,4,6-trimethylphenyl)imidazolidin-2-ylidene]-dichloro-ruthenium; tricyclohexylphosphane (381.94 mg, 449.88 umol) in one portion under N₂. The mixture was stirred at 30° C. for 12 h. The mixture was concentrated under reduced pressure. The residue was purified by silica gel chromatography (petroleum ether/ethyl acetate=4/1 to 1/l) to afford the title compound (650.00 mg, 2.02 mmol, 89.87% yield, 95% purity) as a yellow solid. MS (ESI): mass calcd. for C₁₆H₂₃N₃O₃, 305.1; m/z found, 306 [M+]⁺.

### Step F. tert-Butyl 11-hydroxy-3,4,8,9,10,11-hexahydro-1H-pyrido[4,3″3,4] pyrazolo[1,5-a]azepine-2(7H)-carboxylate

To a solution of tert-butyl 11-hydroxy-3,4,10,11-tetrahydro-1H-pyrido[4′,3′:3,4]pyrazolo[1,5-a]azepine-2(7H)-carboxylate (150.00 mg, 491.21 umol) in MeOH (5.00 mL) was added Pd/C (20.00 mg, 10%) under N₂. The suspension was degassed under reduced pressure and purged with H₂ several times. The mixture was stirred under Hz (15 psi) at 30° C. for 12 h. The reaction mixture was filtered, and the filtrate was concentrated to afford the title compound (140.0) mg, 455.45 umol, 92.72% yield) as a yellow solid. MS (ESI): mass calcd. for CH₁₆H₂₅N₃O₃, 307.1; m/z found, 308 [M+H]⁺.

### Step G. tert-Butyl 11-oxo-3,4,8,9,10,11-hexahydro-1H-pyrido[4′,3′:3,4]pyrazolo[1,5-a]azepine-2(7H)-carboxylate

To a mixture of tert-butyl 11-hydroxy-3,4,8,9,10,11-hexahydro-1H-pyrido [4′,3′:3,4]pyrazolo[1,5-a]azepine-2(7H)-carboxylate (2.00 g, 6.51 mmol) in MeCN (80.00 mL) was added NMO (3.05 g, 26.04 mmol, 2.75 mL) and TPAP (457.31 mg, 1.30 mmol) in one portion under N₂. The mixture was stirred at 30° C. for 12 h. The mixture was filtered and concentrated under reduced pressure. The residue was purified by silica gel chromatography (petroleum ether/ethyl acetate=4/1 to 1/1) to afford the title compound (1.60 g, 5.24 mmol, 80.48% yield) as a yellow oil. MS (ESI): mass calcd. for C₁₆H₂₅N₃O, 305.1; m/z found, 306 [M+H]⁺.

### Intermediate 15: tert-Butyl 6,7,10,11-tetrahydro-5H-pyridazino[3,4-c]pyrido[4′,3′:3,4]-pyrazolo[1,5-a]azepine-12(13H)-carboxylate

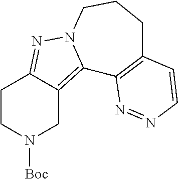 [SMILES: CC(C)(C)OC(=O)N1CCc2nn3c(c2C1)-c1nnccc1CCC3]

### Step A. tert-Butyl 10-allyl-11-oxo-3,4,8,9,10,11-hexahydro-1H-pyrido[4′,3′:3,4]pyrazolo[1,5-a]azepine-2(7H)-carboxylate

To a solution of tert-butyl 11-oxo-3,4,8,9,10,11-hexahydro-1H-pyrido[4′,3′:3,4]pyrazolo[1,5-a]azepine-2(7H)-carboxylate (Intermediate 14, 300.00 mg, 982.41 umol) and HMPA (440.12 mg, 2.46 mmol, 431.49 uL) in THF (8.00 mL) at −78° C. was added LDA (6 mL, 1.25 M, freshly prepared from N-isopropylpropan-2-amine (1.22 g, 12.05 mmol, 1.69 mL) in THF (3.00 mL) by adding n-BuLi (2.5 M, 5.00 mL) at −65° C.), then warm to −30° C. for 0.5 h. 3-Bromoprop-1-ene (594.26 mg, 4.91 mmol) was added at −78° C. The mixture was warmed to 30° C. and stirred for another 1 h. The reaction was quenched with HCl (1 N aq, 10 mL) and extracted with EtOAc (20 mL×3). The combined organic layers were dried over anhydrous Na₂SO₄, filtered, and the filtrate concentrated under reduced pressure. The residue was purified by prep-TLC (petroleum ether/ethyl acetate=1/l) and further purified by RP HPLC (Condition A) to afford tert-butyl 10-allyl-11-oxo-3,4,8,9,10,11-hexahydro-1H-pyrido[4′,3′:3,4]pyrazolo[1,5-a]azepine-2(7H)-carboxylate (31.00 mg, 89.74 umol, 9.14% yield) as a colorless oil. MS (ESI): mass calcd. for C₁₉H₂₇N₃O₃, 345.2: m/z found, 346.1 [M+H]⁺.

### Step B. tert-Butyl 11-oxo-10-(2-oxoethyl)-3,4,8,9,10,11-hexahydro-1H-pyrido [4′,3′: 3,4]-pyrazolo[1,5-a]azepine-2(7H)-carboxylate

To a mixture of tert-butyl 10-allyl-11-oxo-3,4,7,8,9,10-hexahydro-1H-pyrido[2,3]pyrazolo[2,4-]azepine-2-carboxylate (60.00 mg, 173.70 umol) in THF (4.0 mL) and H₂O (4.0 mL) was added OsO₄ (13.25 mg, 52.11 umol, 2.70 uL) and NaIO₄ (148.61 mg, 694.80 umol, 38.50 uL) in one portion at 0° C. under N₂. The mixture was stirred at 20° C. for 10 h. The mixture was poured into water (10 mL) and stirred for 1 min. The aqueous phase was extracted with ethyl acetate (10 mL×2). The combined organic phases were washed with brine (5 mL×2), dried over anhydrous Na₂SO₄, filtered and the filtrate concentrated under reduced pressure to afford the title compound (60.35 mg, crude) as a yellow oil. MS(ESI): mass calcd. for C₁₈H₂₅N₃O₄, 347.1; m/z found, 348.1 [M+H]⁺.

### Step C. tert-Butyl 4a,5,6,7,10,11-hexahydro-4H-pyridazino[3,4-c]pyrido[4′,3′:3,4]pyrazolo[1,5-a]azepine-12(13H)-carboxylate

To a mixture of tert-butyl 11-oxo-10-(2-oxoethyl)-3,4,7,8,9,10-hexahydro-1H-pyrido[2,3]pyrazolo[2,4-b]azepine-2-carboxylate (60.35 mg, 173.71 umol) in EtOH (10.00 mL) was added N₂H₄.H₂O (15.35 mg, 260.57 umol, 14.90 uL, 85% purity) in one portion at 0° C. under N₂. The mixture was stirred at 20° C. for 2 h. The reaction mixture was used in the next step directly. MS (ESI): mass calcd. for C₁₈H₂₅N₅O₂, 343.2; m/z found, 344.1 [M+H]⁺.

### Step D. tert-Butyl 6,7,10,11-tetrahydro-5H-pyridazino[3,4-c]pyrido[4′,3′:3,4] pyrazolo[1,5-a]azepine-12(13H)-carboxylate

To the reaction mixture from Step C was added DDQ (47.32 mg, 208.46 umol) under N₂. The mixture was stirred at 0° C. for 2 h. The mixture was concentrated under reduced pressure. The residue was purified by prep-TLC (DCM/MeOH=10/1) to afford the title compound (17.00 mg, 48.95 umol, 28.18% yield, 98.3% purity) as a yellow oil. MS(ESI): mass calcd. for C₁₈H₂₃N₅O₂, 341.1; m/z found, 342 [M+H]⁺.

### Intermediate 16: tert-Butyl 4,5,6,9,10,12-hexahydropyrazolo[3,4-c]pyrido[4′,3′:3,4]pyrazolo[1,5-a]azepine-1(2H)-carboxylate

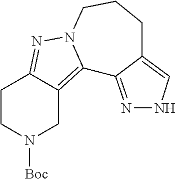 [SMILES: CC(C)(C)OC(=O)N1CCc2nn3c(c2C1)-c1n[nH]cc1CCC3]

### Step A tert-Butyl 10-(hydroxymethylene)-11-oxo-3,4,8,9,10,11-hexahydro-1H-pyrido[4′,3′:3,4]-pyrazolo[1,5-a]azepine-2(7H)-carboxylate

A mixture of tert-butyl 11-oxo-3,4,7,8,9,10-hexahydro-1H-pyrido[2,3]pyrazolo [2,4-a]azepine-2-carboxylate (200.00 mg, 654.94 umol, Intermediate 14) in DMF-DMA (18.00 g, 151.07 mmol, 20.00 mL) was stirred at 75° C. for 12 h. The mixture was stirred at 75° C. for another 24 h, then concentrated under reduced pressure. The residue was poured into water (20 mL) and stirred for 2 min. The aqueous phase was extracted with ethyl acetate (20 mL×2). The combined organic phases were washed with brine (10 mL×2), dried with anhydrous Na₂SO₄, filtered and the filtrate concentrated under reduced pressure to afford title compound (210.00 mg, 629.91 umol, 96.18% yield) as a yellow solid. MS(ESI): mass calcd. for C₁₇H₂₃N₃O₄, 333.1; m/z found, 334.1 [M+H]⁺.

### Step B. tert-Butyl 4,5,6,9,10,12-hexahydropyrazolo[3,4-c]pyrido[4′,3′:3,4]pyrazolo[1,5-a]azepine-11(2H)-carboxylate

To a mixture of tert-butyl 10-(hydroxymethylene)-11-oxo-3,4,8,9,10,11-hexahydro-1H-pyrido[4′,3′:3,4]pyrazolo[1,5-a]azepine-2(7H)-carboxylate (80.00 mg, 239.97 umol) in MeOH (5.00 mL) was added N₂H₄.H₂O (28.27 mg, 479.93 umol, 27.44 uL, 85% purity) in one portion at 30° C. under N₂. The mixture was stirred at 30° C. for 10 h. The mixture was concentrated under reduced pressure. The residue was purified by prep-TLC (petroleum ether/ethyl acetate:1/2) to afford the title compound (54.00 mg, 163.93 umol, 68.31% yield) as a yellow solid. MS (ESI): mass calcd. for C₁₇H₂₃N₅O₂, 329.1; m/z found, 330.1 [M+H]⁺.

### Intermediate 17: tert-Butyl 6,7,10,11-tetrahydro-5H-pyrido[2,3-c]pyrido[4′,3′:3,4]pyrazolo[1,5-a]azepine-12(13H)-carboxylate

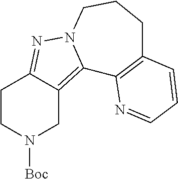 [SMILES: CC(C)(C)OC(=O)N1CCc2nn3c(c2C1)-c1ncccc1CCC3]

The title compound was prepared in a manner analogous to Intermediate 8, substituting tert-butyl 11-oxo-3,4,8,9,10,11-hexahydro-1H-pyrido[4′3′:3,4]pyrazolo[1,5-a]azepine-2(7H)-carboxylate for (R)-tert-butyl 3-methyl-11-oxo-3,4,8,9,10,11-hexahydro-1H-pyrido[4′,3′:3,4]pyrazolo[1,5-a]azepine-2(7H)-carboxylate. MS (ESI): mass calcd. for C₁₉H₂₄N₄O₂, 340.2; m/z found, 341.0 [M+H]⁺.

### Intermediate 18: tert-Butyl 2-methyl-4,5,6,9,10,12-hexahydropyrazolo[3,4-c]pyrido[4′,3′:3,4]-pyrazolo[1,5-a]azepine-11(2H)-carboxylate

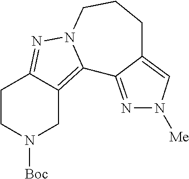 [SMILES: Cn1cc2c(n1)-c1c3c(nn1CCC2)CCN(C(=O)OC(C)(C)C)C3]

To a mixture of tert-butyl 10-(hydroxymethylene)-11-oxo-3,4,8,9,10,11-hexahydro-1H-pyrido[4′,3′:3,4]pyrazolo[1,5-a]azepine-2(7H)-carboxylate (130.00 mg, 389.95 umol, Intermediate 15 product from Step A) in MeOH (5.00 mL) was added methylhydrazine (89.82 mg, 779.90 umol, 102.07 uL) in one portion at 30° C. under N₂. The mixture was stirred at 30° C. for 10 h. The mixture was concentrated under reduced pressure, then purified by prep-TLC (petroleum ether/ethyl acetate=1/2) to afford 100 mg of crude product, which was further purified by RP HPLC (Condition A) to afford title compound tert-butyl 2-methyl-4,5,6,9,10,12-hexahydropyrazolo[3,4-c]pyrido[4′,3′:3,4]pyrazolo[1,5-a]azepine-11(2H)-carboxylate (70.00 mg, 203.83 umol, 52.27% yield) as a yellow solid, and another regioisomer tert-butyl 1-methyl-4,5,6,9,10,12-hexahydropyrazolo[3,4-c]pyrido[4′,3′:3,4]pyrazolo[1,5-a]azepine-11(1H)-carboxylate (20.00 mg, 58.24 umol, 14.93% yield) as a yellow solid. MS(ESI): mass calcd. for C₁₈H₂₅N₅O₂, 343.2: m/z found, 344.2 [M+H]⁺; ¹H NMR (400 MHz, CDCl₃) δ 7.19 (s, 1H), 4.63-4.77 (m, 2H), 4.38-4.53 (m, 2H), 4.06-4.20 (m. I3H), 3.84-3.94 (m, 3H), 3.84-3.94 (m, 3H), 3.64-3.67 (m, 1H), 3.72 (br s, 1H), 2.83-2.96 (m, 2H), 2.76 (br t, J=5.58 Hz, 2H), 2.13-2.30 (m, 2H), 1.50 (s, 9H).

### Intermediate 19: tert-Butyl 1-methyl-4,5,6,9,10,12-hexahydropyrazolo[3,4-c]pyrido[4′,3′:3,4]-pyrazolo[1,5-a]azepine-11(1H)-carboxylate

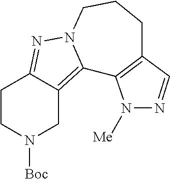 [SMILES: Cn1ncc2c1-c1c3c(nn1CCC2)CCN(C(=O)OC(C)(C)C)C3]

The title compound was isolated by RP HPLC (Condition A) from Intermediate 16. MS (ESI): mass calcd. for C₁₈H₂₅N₅O₂, 343.2; m/z found, 344.2 [M+H]⁺. ¹H NMR (400 MHz, CDCl₃) δ 7.43 (s, 1H), 4.43-4.65 (m, 2H), 4.11-4.25 (m, 2H), 3.91 (s, 3H), 3.76 (br s, 2H), 2.82 (br t, J=5.58 Hz, 2H), 2.71 (t, J=7.47 Hz, 2H), 2.22 (br dd, J=4.96, 6.71 Hz, 2H), 1.48 (s, 8H).

### Intermediate 20: tert-Butyl 5,6,9,10-tetrahydro-4H-isoxazolo[3,4-c]pyrido[4′,3:3,4]pyrazolo[1,5-a]azepine-11(12H)-carboxylate

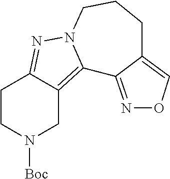 [SMILES: CC(C)(C)OC(=O)N1CCc2nn3c(c2C1)-c1nocc1CCC3]

To a mixture of tert-butyl 10-(hydroxymethylene)-11-oxo-3,4,8,9,10,11-hexahydro-1H-pyrido[4′,3′:3,4]pyrazolo[1,5-a]azepine-2(7H)-carboxylate (80.0) mg, 239.97 umol, Intermediate 15 product from Step A) in Py (5.00 mL) was added NH₂OH.HCl (100.05 mg, 1.44 mmol) in one portion under N₂. The mixture was stirred at 115° C. for 12 h, then concentrated under reduced pressure. The residue was poured into HCl (1 N aq, 10 mL) and stirred for 1 min. The aqueous phase was extracted with ethyl acetate (10 mL×2). The combined organic phases were washed with brine (10 mL×2), dried with anhydrous Na₂SO₄, filtered and the filtrate concentrated under reduced pressure. The residue was purified by prep-TLC (petroleum ether/ethyl acetate=1/2) to afford title compound (48.40 mg, 116.23 umol, 48.44% yield, 80% purity) as a yellow solid. MS(ESI): mass calcd. for C₁₇H₂₂N₄O₃, 330.1; m/z found, 331.1 [M+H]⁺; ¹H NMR (400 MHz, CDCl₃) δ 8.17-8.37 (m, 1H), 4.70 (br s, 2H), 4.38-4.57 (m, 2H), 3.74 (br s, 2H), 3.50 (s, 3H), 2.87-3.03 (m, 2H), 2.65-2.82 (m, 2H), 2.16-2.39 (m, 2H), 1.50 (s, 9H).

### Intermediate 21 tert-Butyl 5,6,9,10-tetrahydro-4H-isoxazolo[5,4-c]pyrido[4′,3′:3,4]pyrazolo[1,5-a]azepine-11(12H)-carboxylate

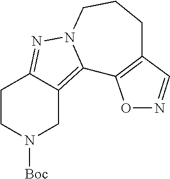 [SMILES: CC(C)(C)OC(=O)N1CCc2nn3c(c2C1)-c1oncc1CCC3]

To a mixture of tert-butyl 10-(hydroxymethylene)-11-oxo-3,4,8,9,10,11-hexahydro-1H-pyrido[4′,3′:3,4]pyrazolo[1,5-a]azepine-2(7H)-carboxylate (70.00 mg, 209.97 umol. Intermediate 15 product from Step A) in MeOH (5.00 mL) was added NH₂OH.HCl (87.55 mg, 1.26 mmol) in one portion at 30° C. under N₂. The mixture was stirred at 3° C. for 12 h. The mixture was poured into water (10 mL) and stirred for 1 min. The aqueous phase was extracted with ethyl acetate (10 mL×2). The combined organic phases were washed with brine (10 mL×2), dried with anhydrous Na₂SO₄, filtered and concentrated under reduced pressure. The residue was purified by prep-TLC (petroleum ether/ethyl acetate=L/2) to afford the title compound (40.0) mg, 106.54 umol, 50.74% yield, 88% purity) as yellow oil. MS (ESI): mass calcd. for C₁₇H₂₂N₄O₃, 330.1; m/z found, 331.1 [M+H]⁺. ¹H NMR (400 MHz, CDCl₃) δ 8.15 (s, 1H), 4.78 (br s, 2H), 4.40-4.57 (m, 2), 3.74 (br s, 2H), 2.86 (t, J=5.96 Hz, 2H), 2.77 (br s, 2H), 2.19-2.31 (m, 2H), 1.50 (s, 9H).

### Intermediate 22: tert-Butyl 11-oxo-3,4,8,9,10,11-hexahydro-1H-cyclohepta[3,4]pyrazolo-[1,5-a]pyrazine-2(7H)-carboxylate

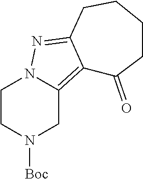 [SMILES: CC(C)(C)OC(=O)N1CCn2nc3c(c2C1)C(=O)CCCC3]

### Step A. Diethyl 1-(2-((tert-butoxycarbonyl)amino)ethyl)-1H-pyrazole-3,5-dicarboxylate

To a solution of diethyl 1H-pyrazole-3,5-dicarboxylate (45 g, 212.06 mmol) and Cs₂CO₃ (82.91 g, 254.47 mmol) in DMF (1000 mL) was added tert-butyl N-(2-bromoethyl)carbamate (50.85 g, 226.91 mmol). The mixture was stirred at 15° C. for 16 h under N₂ atmosphere. The reaction mixture was diluted with water (500 mL) and extracted with EtOAc (700 mL×3). The combined organic layers were washed with brine (1000 mL×3), dried over Na₂SO₄, filtered and the filtrate concentrated under reduced pressure to give the title compound (67 g, crude) as a white solid, which was used directly for the next step. ¹H NMR (400 MHz, CDCl₃) 7.35 (s, 1H), 4.82-4.74 (m, 3H), 4.42-4.33 (m, 4H), 3.63-3.62 (m, 2H), 1.46-1.38 (m, 15H).

### Step B. Ethyl 4-oxo-4,5,6,7-tetrahydropyrazolo[1,5-a]pyrazine-2-carboxylate

To a solution of diethyl 1-[2-(tert-butoxycarbonylamino)ethyl]pyrazole-3,5-dicarboxylate (67 g, 188.53 mmol) in MeOH (100 mL) was added HCL/MeOH (4 M, 100 mL). The mixture was stirred at 15° C. for 16 h. The reaction mixture was concentrated under reduced pressure to give crude product (54.9 g crude, HCl salt) as a white solid. To the resulting solid was added dioxane (560 mL), following by a solution of Na₂CO₃ (39.89 g, 376.36 mmol) in water (560 mL). The mixture was stirred at 15° C. for 16 h. The reaction mixture was extracted with EtOAc (500 mL×2), following by DCM/MeOH=20/1 (500 mL×2). The combined organic layers were dried over Na₂SO₄, filtered and the filtrate concentrated under reduced pressure. The residue was triturated in a mixture of petroleum ether/EtOAc (v/v=10/1, 150 mL) and then filtered. The collected solid was dried to give title compound (34 g, containing ˜60% mol methyl ester) as a white solid.

### Step C. tert-Butyl 2-(hydroxymethyl)-6,7-dihydropyrazolo[1,5-a]pyrazine-5(4H)-carboxylate

To a solution of tert-butyl 2-(hydroxymethyl)-6,7-dihydropyrazolo[1,5-a]pyrazine-5(4H)-carboxylate (32.00 g, containing ˜60% mol methyl ester) in THF (640 mL) was added LAH (6.6 g, 173.91 mmol) at −30° C. under a N₂ atmosphere, then the mixture was heated to 75° C. for 16 h. LAH (6.6 g, 173.89 mmol) was added to the mixture at −30° C. The reaction mixture was heated to 75° C. for 16 h. The reaction mixture was quenched by addition of saturated aqueous potassium sodium tartrate tetrahydrate (30 mL) and stirred for 1 h and filtered. To the filtrate was added Boc₂O (50.12 g, 229.67 mmol, 52.76 mL) and stirred at 15° C. for 16 h. The reaction mixture was diluted with water (600 mL) and extracted with EtOAc (3M) mL×2). The combined organic layers were washed with brine (400 mL), dried over Na₂SO₄, filtered and the filtrate concentrated under reduced pressure. The residue was purified by column chromatography to give title product (33 g, 130.28 mmol, 85.09% yield) as a white solid. ¹H NMR (400 MHz, CDCl₃) δ 6.04 (s, 1H), 4.62-4.61 (m, 4H), 4.13-4.10 (m, 2H), 3.86-3.84 (m, 2H), 1.47 (s, 9H).

### Step D. tert-Butyl 2-(hydroxymethyl)-3-iodo-6,7-dihydropyrazolo[1,5-a]pyrazine-5(4H)-carboxylate

A solution of tert-butyl 2-(hydroxymethyl)-(6,7-dihydro-4H-pyrazolo[1,5-a]pyrazine-5-carboxylate (23 g, 90.80 mmol) in MeCN (300 mL) was added NIS (30.64 g, 136.20 mmol) slowly, and the mixture was stirred at 15° C. for 16 h under a N₂ atmosphere. The mixture was diluted with water (400 mL) and extracted with EtOAc (400 mL). The organic phases were washed with saturated Na₂S₂O₃ (400 mL), dried over Na₂SO₄, filtered and the filtrate concentrated under reduced pressure. The residue was rinsed with petroleum ether/EtOAc=20/1 (300 mL) and stirred for 0.5 h. The mixture was filtered. The collected solid was dried under reduced pressure to give title compound (29.5 g, 77.80 mmol, 85.68% yield) as a yellow solid. ¹H NMR (400 MHz, CDCl₃) δ 4.61 (s, 2H), 4.48 (s, 2H), 4.14 (m, 2H), 3.86 (m, 2H).

### Step E. tert-Butyl 2-formyl-3-iodo-6,7-dihydropyrazolo[1,5-a]pyrazine-5(4H)-carboxylate

To a solution of tert-butyl 2-(hydroxymethyl)-3-iodo-6,7-dihydro-4H-pyrazolo[1,5-a]pyrazine-5-carboxylate (9 g, 23.73 mmol) in DCM (180 mL) was added Dess-Martin (15.10 g, 35.60 mmol, 11.02 mL) and the mixture was stirred at 15° C. for 2 h. The mixture was filtered, and the filtrate was diluted with DCM (300 mL) and washed with brine (300 mL). The organic phases were dried over Na₂SO₄, filtered and the filtrate concentrated under reduced pressure. The residue was purified by silica gel column chromatography to give title compound (7.5 g, 19.88 mmol, 83.78% yield) as a yellow solid.

### Step F. tert-Butyl 3-iodo-2-vinyl-6,7-dihydropyrazolo[1,5-a]pyrazine-5(4H)-carboxylate

To a solution of methyl(triphenyl)phosphonium bromide (9.23 g, 25.85 mmol) in THF (50 mL) was added NaHMDS (1 M, 25.85 mL) at −10° C. under a N₂ atmosphere, followed by a solution of tert-butyl 2-formyl-3-iodo-6,7-dihydro-4H-pyrazolo[1,5-a]pyrazine-5-carboxylate (7.5 g, 19.88 mmol) in THF (30 mL) after 0.5 h and the mixture was stirred at 15° C. for 2 h. The mixture was quenched with brine (120 mL) and extracted with EtOAc (120 ml). The organic phases were dried over Na₂SO₄, filtered and the filtrate concentrated under reduced pressure. The residue was purified by silica gel column chromatography to give pure title compound (2.8 g, 7.46 mmol) as a colorless oil.

### Step G. tert-Butyl 3-(1-hydroxypent-4-en-1-yl)-2-vinyl-6,7-dihydropyrazolo[1,5-a]pyrazine-5(4H)carboxylate

To a solution of tert-butyl 3-iodo-2-vinyl-6,7-dihydro-4H-pyrazolo[1,5-a]pyrazine-5-carboxylate (1.8 g, 4.80 mmol) in T-IF (30 ml) was added i-PrMgCl (2 M, 360) mL) at −10° C. under a N₂ atmosphere. The mixture was stirred at 10° C. for 1 h, then a solution of pent-4-enal (605.31 mg, 7.20 mmol) in THF (3 mL) was added. The reaction mixture was stirred at 15° C. for 1.5 h. The mixture was quenched with saturated NH₄Cl (100 mL) and extracted with EtOAc (100 mL). The organic phases were dried over Na₂SO₄, filtered and the filtrate concentrated under reduced pressure. The residue was purified by silica gel column chromatography to give title compound (1.0 g, 3.00 mmol, 62.52% yield) as a colorless oil.

### Step H. tert-Butyl 11-hydroxy-3,4,10,11-tetrahydro-1H-cyclohepta[3,4]pyrazolo[1,5-a]pyrazine-2(9H)-carboxylate

To a solution of tert-butyl 3-(1-hydroxypent-4-enyl)-2-vinyl-6,7-dihydro-4H-pyrazolo[1,5-a]pyrazine-5-carboxylate (1.3 g, 3.90 mmol) in DCM (800 ml) was added [1,3-bis(2,4,6-trimethylphenyl)imidazolidin-2-ylidene]-dichloro-[(2-isopropoxyphenyl)methylene]ruthenium (244.32 mg, 389.89 umol) under a N₂ atmosphere, and the mixture was stirred at 40° C. for 16 h. The mixture was concentrated under reduced pressure. The residue was purified by silica gel column chromatography to give title compound (0.79 g, 2.59 mmol, 66.35% yield) as a brown solid. MS (ESI): mass calcd. for C₁₆H₂₃N₃O₃ 305.2; m/z found, 306.1 [M+H]⁺.

### Step I. tert-Butyl 11-oxo-3,4,10,11-tetrahydro-1H-cyclohepta[3,4]pyrazolo[1,5-a]pyrazine-2(9H)-carboxylate

A mixture of tert-butyl 11-hydroxy-1,3,4,9,10,11-hexahydrocyclohepta-[2,3]pyrazolo[2,4-a]pyrazine-2-carboxylate (570 mg, 1.87 mmol). NMO (874.65 mg, 7.47 mmol, 787.97 uL) and TPAP (131.19 mg, 373.32 umol) in MeCN (10 mL) was degassed and purged with N₂ (3×), and then the mixture was stirred at 15° C. for 1.5 h under a N₂ atmosphere. The mixture was poured into ice-water (50 mL) and stirred for 1 min. The aqueous phase was extracted with ethyl acetate (30 mL×2). The combined organic phases were washed with brine (60 ml), dried with anhydrous Na₂SO₄, filtered and the filtrate concentrated under reduced pressure. The residue was purified by column chromatography to give title compound (405 mg, 1.34 mmol, 71.52% yield) as a black brown solid. MS (ESI): mass calcd. for C₁₆H₂₁N₃O₃; 303.2; m/z found, 304.1 [M+H]⁺.

### Step J. tert-Butyl 11-oxo-3,4,8,9,10,11-hexahydro-1H-cyclohepta[3,4]pyrazolo[1,5-a]pyrazine-2(7H)-carboxylate

To a solution of tert-butyl 11-oxo-3,4,9,10-tetrahydro-1H-cyclohepta[2,3]-pyrazolo[2,4-a]pyrazine-2-carboxylate (0.405 g, 1.34 mmol) in EtOH (30 mL)/MeOH (3 mL) was added Pd/C (0.08 g, 1.34 mmol, 10% purity) and the mixture was stirred at 15° C. under H₂ (15 Psi) atmosphere for 1 h. The mixture was filtered, the filtrate was concentrated under reduced pressure to give title compound (0.39 g, 1.28 mmol, 95.66% yield) as a brown solid, which was used directly for the next step. ¹H NMR (400 MHz, CDCl₃) δ 4.77 (s, 2H), 4.05-4.03 (m, 2H), 3.81-3.79 (m, 2H), 2.89-2.86 (m, 2H), 2.62-2.59 (m, 2H), 1.89-1.82 (m, 4H), 1.44 (s, 9H).

### Intermediate 23: tert-Butyl 5,6,9,10-tetrahydro-4H-isoxazolo[5″,4″:3′,4′]cyclohepta[1′,2′:3,4]-pyrazolo[1,5-a]pyrazine-11(12H)-carboxylate

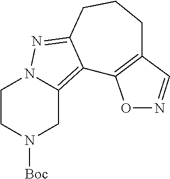 [SMILES: CC(C)(C)OC(=O)N1CCn2nc3c(c2C1)-c1oncc1CCC3]

### Step A. tert-Butyl 11-((dimethylamino)methylene)-11-oxo-3,4,8,9,10,11-hexahydro-1H-cyclohepta[3,4]pyrazolo[1,5-a]pyrazine-2(7H)-carboxylate

A solution of tert-butyl 11-oxo-3,4,7,8,9,10-hexahydro-1H-cyclohepta[2,3]pyrazolo[2,4-a]pyrazine-2-carboxylate (0.08 g, 261.98 umol) in DMF-DMA (3.59 g, 30.11 mmol, 4 mL) was heated to 115° C. for 56 h. The mixture was concentrated under reduced pressure. The residue was diluted with EtOAc (30 mL) and washed with brine (30 mL). The organic phases were dried over Na₂SO₄, filtered and the filtrate concentrated under reduced pressure to give title compound (0.09 g, crude) as a yellow solid, which was used directly for the next step.

### Step B. tert-Butyl 5,6,9,10-tetrahydro-4H-isoxazolo[5′,4″:3′,4′]cyclohepta[1′,2′:3,4]pyrazolo[1,5-a]pyrazine-11(12H)-carboxylate

A mixture of tert-butyl 10-((dimethylamino)methylene)-11-oxo-3,4,8,9,10,11-hexahydro-1H-cyclohepta[3,4]pyrazolo[1,5-a]pyrazine-2(7H)-carboxylate (0.09 g, 249.69 umol) and hydroxylamine hydrochloride (104.11 mg, 1.50 mmol) in MeOH (3 mL) was stirred at 20° C. for 16 h. The mixture was diluted with EtOAc (40 mL) and washed with brine (40 mL). The organic phases were dried over Na₂SO₄, filtered, and the filtrate concentrated under reduced pressure. The residue was purified by prep-TLC (Petroleum ether/EtOAc) to give title compound (0.051 g, 140.47 umol, 56.26% yield, 91% purity) as a colorless oil. MS (ESI): mass calcd. for C₁₇H₂₂N₄O₃ 330.2; m/z found, 331.1 [M+H]⁺.

¹H NMR (400 MHz, CDCl₃) δ 8.03 (s, 1H), 4.97 (s, 2H), 4.16-4.13 (m, 2H), 3.93-3.9) (m, 2H), 3.02-2.99 (m, 2H), 2.79-2.76 (m, 2H), 2.05-2.00 (m, 2H), 1.51 (s, 9H).

### Intermediate 24: tert-Butyl 5,6,9,10-tetrahydro-4H-isoxazolo [3″,4″:3′,4′] cyclohepta [1′,2′:3,4]pyrazolo[1,5-a]pyrazine-11(12H)-carboxylate

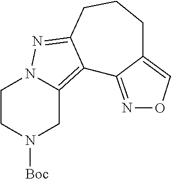 [SMILES: CC(C)(C)OC(=O)N1CCn2nc3c(c2C1)-c1nocc1CCC3]

### Step A. tert-Butyri 10-((dimethylamino)methylene)-11-oxo-3,4,8,9,10,11-hexahydro-1H-cyclohepta[3,4]pyrazolo[1,5-a]pyrazine-2(7H)-carboxylate

A solution of tert-butyl 11-oxo-3,4,7,8,9,10-hexahydro-1H-cyclohepta[2,3]pyrazolo[2,4-a]pyrazine-2-carboxylate (0.34 g, 1.11 mmol) and TDAM (1.29 g, 8.91 mmol, 1.54 mL) in toluene (15 mL) was heated to 115° C. for 16 h. TDAM (646.87 mg, 4.45 mmol) was added and the mixture was heated to 115° C. for another 16 h. Additional TDAM (323.43 mg, 2.23 mmol) was added and the mixture was heated to 115° C. for another 16 h. At that time, the mixture was diluted with EtOAc (60 mL) and washed with brine (50 mL×3). The organic phases were dried over Na₂SO₄, filtered and the filtrate concentrated under reduced pressure to give title compound (0.385 g, crude) as a yellow solid, which was used directly for the next step.

### Step B. tert-Butyl 5,6,9,10-tetrahydro-4H-isoxazolo[3″,4″:3′,4′]cyclohepta[1′,2′:3,4]pyrazolo[1,5-a]pyrazine-11(12H)-carboxylate

A mixture of tert-butyl 10-(dimethylamino)methylene)-11-oxo-3,4,8,9,10,11-hexahydro-1H-cyclohepta[3,4]pyrazolo[1,5-a]pyrazine-2(7H)-carboxylate (0.235 g, 651.96 umol) and hydroxylamine hydrochloride (271.83 mg, 3.91 mmol) in pyridine (12 mL) was stirred at 115° C. for 24 h. The mixture was concentrated to give a yellow residue, which was diluted with EtOAc (50 mL) and washed with HCl (1 M aq, 50 mL). The organic phases were dried over Na₂SO₄, filtered and the filtrate concentrated under reduced pressure. The residue was purified by prep-HPLC (Condition A) to give regioisomer compound tert-butyl 5,6,9,10-tetrahydro-4H-isoxazolo[5″,4″:3′,4′]cyclohepta[1′,2′:3,4] pyrazolo[1,5-a]pyrazine-11(12H)-carboxylate (Intermediate 2, 0.07 g, 211.88 umol, 32.50% yield) as a colorless oil, and title compound (0.037 g, 111.99 umol, 17.18% yield) as a colorless oil. ¹H NMR (400 MHz, CDCl₃) δ 8.11 (s, 1H), 4.90 (s, 2H), 4.18-4.15 (m, 2H), 3.93-3.90 (m, 2H), 3.07-3.04 (m, 2H), 2.85-2.83 (m, 2H), 2.01-1.98 (m, 2H), 1.51 (s, 9H).

### Example 1a: N-(3-Cyano-4-fluorophenyl-5-methylene-5,6,9,10-tetrahydro-4H-isoxazolo[3,4-c]pyrido[4′,3′:3,4]pyrazolo[1,5-a]azepine-11(12H)-carboxamide

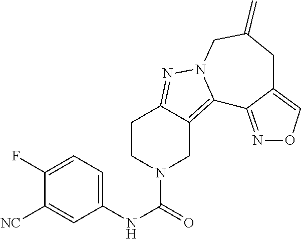 [SMILES: C=C1Cc2conc2-c2c3c(nn2C1)CCN(C(=O)Nc1ccc(F)c(C#N)c1)C3]

### Step A. 5-Methylene-5,6,9,10,11,12-hexahydro-4H-isoxazolo[3,4-c]pyrido[4′,3′:3,4]pyrazolo[1,5-a]azepine

To a solution of tert-butyl 5-methylene-5,6,9,10-tetrahydro-4H-isoxazolo[3,4-c]pyrido-[4′,3′:3,4]pyrazolo[1,5-a]azepine-11(12H)-carboxylate (Intermediate 1, 0.06 g, 175.24 umol) in DCM (5 mL) was added TFA (770.00 mg, 6.75 mmol, 0.5 mL). The mixture was stirred at 20° C. for 1 h. The reaction mixture was concentrated under reduced pressure to give the title compound (63 mg, crude, TFA salt) as a yellow oil. MS (ESI): mass calcd. for C₁₃H₁₄N₄O, 242.17; m/z found, 243.1 [M+H]⁺.

### Step B. N-(3-Cyano-4-fluorophenyl)-5-methylene-5,6,9,10-tetrahydro-4H-isoxazolo[3,4-c]-pyrido[4′,3′:3,4]pyrazolo[1,5-a]azepine-11(12H)-carboxamide

To a solution of 5-methylene-5,6,9,10,11,12-hexahydro-4H-isoxazolo[3,4-c]pyrido-[4′,3′:3,4]pyrazolo[1,5-a]azepine (63 mg, 182.43 umol, TFA salt) and phenyl N-(3-cyano-4-fluoro-phenyl)carbamate (44 mg, 154.55 umol) in DCM (5 mL) was added TEA (184.60 mg, 1.82 mmol, 253.92 uL). The mixture was stirred at 25° C. for 12 h. The reaction mixture was concentrated under reduced pressure. The residue was purified by RP HPLC (Condition A) to give the title compound (40.58 mg, 99.34 umol, 54.46% yield, 99% purity) as a white solid. MS (ESI): mass calcd. for C₂₁H₁₇FN₆O₂, 404.1: m/z found, 405.1 [M+H]⁺; ¹H NMR (400 MHz, CDCl₃) δ=8.36 (s, 1H), 7.77 (dd, J=2.8, 5.6 Hz, 1H), 7.65-7.61 (m, 1H), 7.13 (t, J=8.8 Hz, 1H), 6.82 (s, 1H), 5.39 (s, 1H), 5.31 (s, 1H), 4.97 (s, 2H), 4.73 (s, 2H), 3.90 (t, J=5.6 Hz, 2H), 3.66 (s, 2H), 2.89 (t, J=5.6 Hz, 2H).

### Example 2a: N-(4-Fluoro-3-(trifluoromethyl)phenyl)-5-methylene-5,6,9,10-tetrahydro-4H-isoxazolo[3,4-c]pyrido[4′,3′:3,4]pyrazolo[1,5-a]azepine-11(12H)-carboxamide

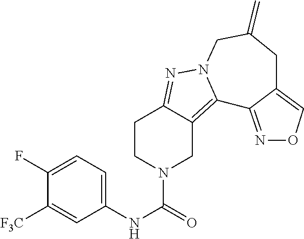 [SMILES: C=C1Cc2conc2-c2c3c(nn2C1)CCN(C(=O)Nc1ccc(F)c(C(F)(F)F)c1)C3]

The title compound was prepared in a manner analogous to Example 1, using phenyl (4-fluoro-3-(trifluoromethyl)phenyl)carbamate instead of phenyl (3-cyano-4-fluorophenyl)carbamate in Step B. MS (ESI): mass calcd. for C₂₁H₁₇F₄N₅O₂, 447.13; m/z found, 448.1 [M+H]⁺. ¹H NMR (400 MHz, CDCl₃) δ=8.36 (s, 1H), 7.68 (dd, J=2.4, 6.0 Hz, 1H), 7.64-7.59 (m, 1H), 7.13 (t, J=9.2 Hz, 1H), 6.72 (s, 1H), 5.39 (s, 1H), 5.31 (s, 1H), 4.98 (s, 2H), 4.73 (s, 2H), 3.91 (t, J=5.6 Hz, 2H), 3.66 (s, 2H), 2.89 (t, J=5.6 Hz, 2H).

### Example 3a N-(3-Cyano-4-fluorophenyl-5-(hydroxymethyl)-5,6,9,10-tetrahydro-4H-isoxazolo-[3,4-c]pyrido[4′,3′:3,4]pyrazolo[1,5-a]azepine-11(12H)carboxamide

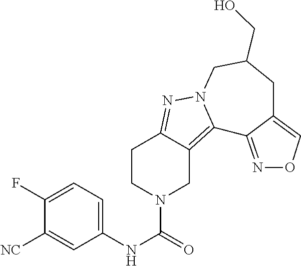 [SMILES: N#Cc1cc(NC(=O)N2CCc3nn4c(c3C2)-c2nocc2CC(CO)C4)ccc1F]

The title compound was prepared in a manner analogous to Example 1, using tert-butyl 5-(hydroxymethyl)-5,6,9,10-tetrahydro-4H-isoxazolo[3,4-c]pyrido[4′,3′:3,4]pyrazolo[1,5-a]-azepine-11(12H)-carboxylate (Intermediate 2) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A. MS (ESI): mass calcd. for C₁₂H₁₉FN₆O₃, 422.15; m/z found, 423.1 [M+H]⁺. ¹H NMR (400 MHz, CDCl₃) δ=8.35 (s, 1H), 7.78 (dd, J=2.8, 5.6 Hz, 1H), 7.67-7.63 (m, 1H), 7.14 (t, J=8.8 Hz, 1H), 6.90 (s, 1H), 4.75-4.68 (m, 3H), 4.46-4.37 (m, 1H), 3.93-3.87 (m, 2H), 3.74-3.66 (m, 2H), 3.14-3.08 (m, 1H), 2.90-2.80 (m, 3H), 2.45 (d, J=6.4 Hz, 1H).

### Example 4a: N-(4-Fluoro-3-(trifluoromethyl)phenyl)-5-(hydroxymethyl)-5,6,9,10-tetrahydro-4H-isoxazolo[3,4-c]pyrido[4′,3′:3,4]pyrazolo[1,5-a]azepine-11(12H)-carboxamide

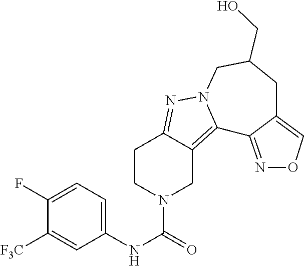 [SMILES: O=C(Nc1ccc(F)c(C(F)(F)F)c1)N1CCc2nn3c(c2C1)-c1nocc1CC(CO)C3]

The title compound was prepared in a manner analogous to Example 1, using tert-butyl 5-(hydroxymethyl)-5,6,9,10-tetrahydro-4H-isoxazolo[3,4-c]pyrido[4′,3′:3,4]pyrazolo[1,5-a]-azepine-11(12H)-carboxylate (Intermediate 2) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A, and using phenyl (4-fluoro-3-(trifluoromethyl)phenyl)carbamate instead of phenyl (3-cyano-4-fluorophenyl)carbamate in Step B. MS (ESI): mass calcd. for C₂₁H₁₉F₄N₅O₃, 465.1: m/z found, 466.1 [M+H]⁺. ¹H NMR (400 MHz, CDCl₃) δ=8.34 (s, 1H), 7.69 (dd, J=2.4, 6.0 Hz, 1H), 7.65-7.60 (m, 1H), 7.13 (t, J=9.6 Hz, 1H), 6.77 (s, 1H), 4.76-4.67 (m, 3H), 4.46-4.37 (m, 1H), 3.93-3.87 (m, 2H), 3.75-3.65 (m, 2H), 3.15-3.07 (m, 1H), 2.90-2.78 (m, 3H), 2.50-2.40 (m, 1H).

### Example 5a: (5S*)-N-3-Cyano-4-fluorophenyl)-5-(2,2-difluoroethoxy)methyl)-5,6,9,10-tetrahydro-4H-isoxazolo[3,4-c]pyrido[4′,3′:3,4]pyrazolo[1,5-a]azepine-11(12H)-carboxamide

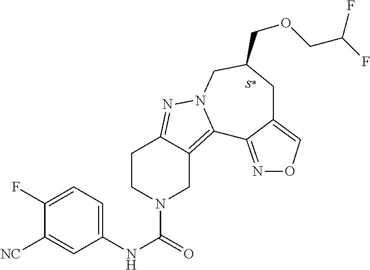 [SMILES: N#Cc1cc(NC(=O)N2CCc3nn4c(c3C2)-c2nocc2C[C@H](COCC(F)F)C4)ccc1F]

The title compound was prepared in a manner analogous to Example 1, using (5S*)-tert-butyl 5-((2,2-difluoroethoxy)methyl)-5,6,9,10-tetrahydro-4H-isoxazolo[3,4-c]pyrido[4′,3′:3,4]pyrazolo-[1,5-a]azepine-11(12H)-carboxylate (Intermediate 3) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A. MS (ESI): mass calcd. for C₂₄H₂₁F₃N₆O₃, 486.2; m/z found, 487.1 [M+H]⁺. ¹H NMR (400 MHz, CDCl₃) δ=8.35 (s, 1H), 7.78 (dd, J=2.8, 5.6 Hz, 1H), 7.66-7.62 (m, 1H), 7.14 (t, J=8.8 Hz, 1H), 6.80 (s, 1H), 6.03-5.70 (m, 1H), 4.79-4.72 (m, 2H), 4.70-4.66 (m, 1H), 4.40-4.34 (m, 1H), 3.93-3.88 (m, 2H), 3.73-3.63 (m, 2H), 3.60 (d, J=6.4 Hz, 2H), 3.10-3.05 (m, 1H), 2.91-2.84 (m, 3H), 2.59-2.48 (m, 1H).

### Example 6a: (5S*)-5-((2,2-Difluoroethyl)methyl-N-4-fluoro-3-(trifluoroethyl)phenyl)-5,6,9,10-tetrahydro-4H-isoxazolo[3,4-c]pyrido[4′,3′:3,4]pyrazolo[1,5-a]azepine-11(12H)-carboxamide

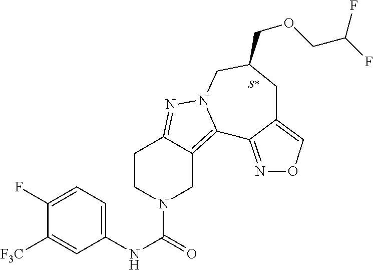 [SMILES: O=C(Nc1ccc(F)c(C(F)(F)F)c1)N1CCc2nn3c(c2C1)-c1nocc1C[C@H](COCC(F)F)C3]

The title compound was prepared in a manner analogous to Example 1, using (5S*)-tert-butyl 5-((2,2-difluoroethoxy)methyl)-5,6,9,10-tetrahydro-4H-isoxazolo[3,4-c]pyrido[4′,3′:3,4]pyrazolo-[1,5-a]azepine-11(12H)-carboxylate (Intermediate 3) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A, and using phenyl (4-fluoro-3-(trifluoromethyl)phenyl)carbamate instead of phenyl (3-cyano-4-fluorophenyl)carbamate in Step B. MS (ESI): mass calcd. for C₂₃H₂₁F₆N₅O₃, 529.2; m/z found, 530.1 [M+H]⁺. ¹H NMR (400 MHz, CDCl₃) δ=8.35 (s, 1H), 7.69 (dd, J=2.8, 6.0 Hz, 1H), 7.66-7.59 (m, 1H), 7.14 (t, J=9.2 Hz, 1H), 6.73 (s, 1H), 6.03-5.71 (m, 1H), 4.80-4.73 (m, 2H), 4.70-4.66 (m, 1H), 4.40-4.34 (m, 1H), 3.96-3.87 (m, 2H), 3.71-3.63 (m, 2H), 3.60 (d, J=6.4 Hz, 2H), 3.10-3.05 (m, 1H), 2.91-2.84 (m, 3H), 2.60-2.48 (m, 12).

### Example 7a: (5R*)-N-(3-Cyano-4-fluorophenyl)-5-((2,2-difluoroethoxy)methyl)-5,6,9,10-tetrahydro-4H-isoxazolo[3,4-c]pyrido[4′,3′:3,4]pyrazolo[1,5-a]azepine-11(12H)-carboxamide

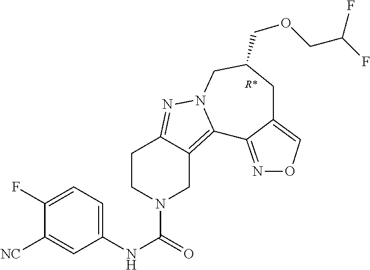 [SMILES: N#Cc1cc(NC(=O)N2CCc3nn4c(c3C2)-c2nocc2C[C@@H](COCC(F)F)C4)ccc1F]

The title compound was prepared in a manner analogous to Example 1, using (5R*)-tert-butyl 5-((2,2-difluoroethoxy)methyl)-5,6,9,10)-tetrahydro-4H-isoxazolo[3,4-c]pyrido[4′,3′:3,4]pyrazolo-[1,5-a]azepine-11(12H)-carboxylate (Intermediate 4) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A. MS (ESI): mass calcd. for C₂₃H₂₁F₃N₆O₃, 486.2, m/z found, 487.1 [M+H]⁺. ¹H NMR (400 MHz, CDCl₃) δ=8.36 (s, 1H), 7.79 (dd, J=2.8, 5.6 Hz, 1H), 7.68-7.64 (m, 1H), 7.14 (t, J=8.8 Hz, 1H), 6.95 (s, 1H), 6.02-5.71 (m, 1H), 4.76-4.66 (m, 3H), 4.40-4.34 (m, 1H), 3.91 (q, J=5.6 Hz, 2H), 3.71-3.63 (m, 2H), 3.60 (d, J=6.4 Hz, 2H), 3.10-3.06 (m, 1H), 2.92-2.84 (m, 3H), 2.59-2.49 (m, 1H).

### Example 8a: (5R*)-5-((2,2-Difluoroethoxy)methyl)-N-(4-fluoro-3-(trifluoromethyl)phenyl)-5,6,9,10-tetrahydro-4H-isoxazolo[3,4-c]pyrido[4′,3′:3,4]pyrazolo[1,5-a]azepine-11(12H)-carboxamide

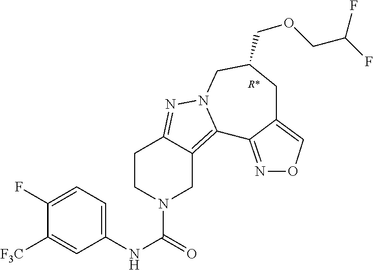 [SMILES: O=C(Nc1ccc(F)c(C(F)(F)F)c1)N1CCc2nn3c(c2C1)-c1nocc1C[C@@H](COCC(F)F)C3]

The title compound was prepared in a manner analogous to Example 1, except using (5R)-tert-butyl 5-((2,2-difluoroethoxy)methyl)-5,6,9,10-tetrahydro-4H-isoxazolo[3,4-c]pyrido[4′,3′:3,4]-pyrazolo[1,5-a]azepine-11(12H)-carboxylate (Intermediate 4) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A. and using phenyl (4-fluoro-3-(trifluoromethyl)-phenyl)carbamate instead of phenyl (3-cyano-4-fluorophenyl)carbamate in Step B. MS (ESI): mass calcd. for C₂₃H₂₁F₆N₅O₃, 529.2; m/z found, 530.1 [M+H]⁺. ¹H NMR (400 MHz, CDCl₃) δ=8.35 (s, 1H), 7.70 (dd, J=2.8, 6.0 Hz, 1H), 7.66-7.60 (m, 1H), 7.13 (t, J=9.2 Hz, 1H), 6.85 (s, 1H), 6.03-5.70 (m, 1H), 4.77-4.64 (m, 31H), 4.40-4.34 (m, 1H), 3.94-3.88 (m, 2H), 3.73-3.63 (m, 2H), 3.60 (d, J=6.4 Hz, 2H), 3.13-3.05 (m, 1H), 2.92-2.83 (m, 3H), 2.54 (s, 1H).

### Example 9a: N-(3-Cyano-4-fluorophenyl)-5-methylene-5,6,9,10-tetrahydro-4H-isoxazolo[5,4-c]-pyrido[4′,3′:3,4]pyrazolo[1,5-a]azepine-11(12H)-carboxamide

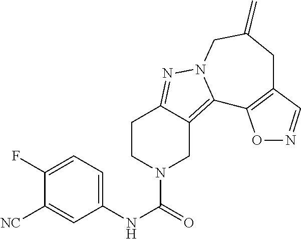 [SMILES: C=C1Cc2cnoc2-c2c3c(nn2C1)CCN(C(=O)Nc1ccc(F)c(C#N)c1)C3]

The title compound was prepared in a manner analogous to Example 1, except using tert-butyl 5-methylene-5,6,9,10-tetrahydro-4H-isoxazolo[5,4-c]pyrido[4′,3′:3,4]pyrazolo[1,5-a]azepine-11(12H)-carboxylate (Intermediate 5) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A. MS (ESI): mass calcd. for C₂₁H₁₇FN₆O₂, 404.1; m/z found, 405.1 [M+H]⁺. ¹H NMR (400 MHz, CDCl₃) δ=8.22 (s, 1H), 7.78 (dd, J=2.8, 5.4 Hz, 1H), 7.64-7.60 (m, 1H), 7.15 (t, J=8.8 Hz, 1H), 6.68 (s, 1H), 5.42 (s, 1H), 5.36 (s, 1H), 4.90 (s, 2H), 4.81 (s, 2H), 3.88 (t, J=5.6 Hz, 2H), 3.60 (s, 2H), 2.88 (t, J=5.6 Hz, 2H).

### Example 10a: N-(4-Fluoro-3-(trifluoromethyl)phenyl)-5-methylene-5,6,9,10-tetrahydro-4H-isoxazolo[5,4-c]pyrido[4′,3′:3,4]pyrazolo[1,5-a]azepine-11(12H)-carboxamide

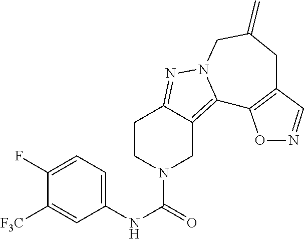 [SMILES: C=C1Cc2cnoc2-c2c3c(nn2C1)CCN(C(=O)Nc1ccc(F)c(C(F)(F)F)c1)C3]

The title compound was prepared in a manner analogous to Example 1, using tert-butyl 5-methylene-5,6,9,10-tetrahydro-4H-isoxazolo[5,4-c]pyrido[4′,3′:3,4]pyrazolo[1,5-a]azepine-11(12H)-carboxylate (Intermediate 5) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A and using phenyl (4-fluoro-3-(trifluoromethyl)phenyl)carbamate instead of phenyl (3-cyano-4-fluorophenyl)carbamate in Step B. MS (ESI): mass calcd. for C₂₁H₁₇F₄N₅O₂, 447.13; m/z found, 448.1 [M+H]⁺. ¹H NMR (400 MHz, CDCl₃) δ=8.22 (s, 1H), 7.69 (dd, J=2.8, 6.0 Hz, 1H), 7.64-7.59 (m, 1H), 7.14 (t, J=9.6 Hz, 1H), 6.63 (s, 1H), 5.42 (s, 1H), 5.36 (s, 1H), 4.90 (s, 2H), 4.82 (s, 2H), 3.89 (t, J=5.6 Hz, 2H), 3.60 (s, 2H), 2.88 (t, J=5.6 Hz, 2H).

### Example 11a: N-(3-Cyano-4-fluorophenyl)-5-hydroxy-5,6,9,10-tetrahydro-4H-isoxazolo[3,4-c]pyrido[4′,3′:3,4]pyrazolo[1,5-a]azepine-11(12H)-carboxamide

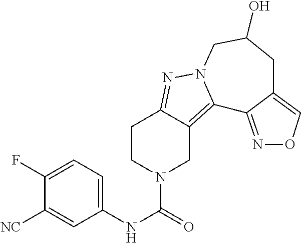 [SMILES: N#Cc1cc(NC(=O)N2CCc3nn4c(c3C2)-c2nocc2CC(O)C4)ccc1F]

The title compound was prepared in a manner analogous to Example 1, using tert-butyl 5-hydroxy-5,6,9,10-tetrahydro-4H-isoxazolo[3,4-c]pyrido[4′,3′:3,4]pyrazolo[1,5-a]azepine-11(12H)-carboxylate (Intermediate 6) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A. MS (ESI): mass calcd. for C₂₀H₁₇FN₆O₃, 408.1; m/z found, 409 [M+H]⁺. ¹H NMR (400 MHz, CD₃OD) δ=8.63 (s, 1H), 7.83 (dd, J=2.8, 5.6 Hz, 1H), 7.72 (ddd, J=2.8, 4.8, 9.2 Hz, 1H), 7.28 (t, J=9.2 Hz, 1H), 4.81 (s, 2H), 4.66-4.56 (m, 2H), 4.40 (q, J=5.2 Hz, 1H), 3.88 (t, J=5.6 Hz, 2H), 3.14 (d, J=5.2 Hz 2H), 2.86 (t, J=5.7 Hz, 2H).

### Example 12a: N-(4-Fluoro-3-(trifluoromethyl)phenyl)-5-methyl-5,6,9,10-tetrahydro-4H-isoxazolo[3,4-c]pyrido[4′,3′:3,4]pyrazolo[1,5-a]azepine-11(12H)-carboxamide

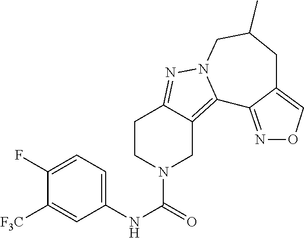 [SMILES: CC1Cc2conc2-c2c3c(nn2C1)CCN(C(=O)Nc1ccc(F)c(C(F)(F)F)c1)C3]

To a solution of N-(4-fluoro-3-(trifluoromethyl)phenyl)-5-methylene-5,6,9,10-tetrahydro-4H-isoxazolo[3,4-c]pyrido[4′,3′:3,4]pyrazolo[1,5-a]azepine-11(12H)-carboxamide (45 mg, 98.57 umol) in MeOH (2 mL) was added Pd—C (10%, 4 mg) under N₂. The suspension was degassed under reduced pressure and purged with H₂ several times. The mixture was stirred under H. (15 psi) at 25° C. for 10 min. The reaction mixture was filtered and concentrated in vacuo. The residue was purified by RP HPLC (Condition A) to give N-(4-fluoro-3-(trifluoromethyl)phenyl)-5-methyl-5,6,9,10-tetrahydro-4H-isoxazolo[3,4-c]pyrido[4′,3′:3,4]pyrazolo[1,5-a]azepine-11(12H)-carboxamide (24.11 mg, 53.11 umol, 53.88% yield, 99% purity) as a white solid. MS (ESI): mass calcd. for C₂₁H₁₉F₄N₅O₂, 449.2; m/z found, 450.2 [M+H]⁺. ¹H NMR (400 MHz, CDCl₃) δ=8.31 (s, 1H), 7.70 (dd, J=2.8, 6.0 Hz, 1H), 7.65-7.60 (m, 1H), 7.14 (t, J=9.6 Hz, 1H), 6.75 (s, 1H), 4.75 (d, J=3.2 Hz, 2H), 4.55-4.52 (m, 1H), 4.32-4.27 (m, 1H), 3.94-3.88 (m, 2H), 3.04-3.00 (m, 1H), 2.88 (t, J=5.6 Hz, 2H), 2.76-2.69 (m, 1H), 2.44 (d, J=6.8 Hz, 1H), 1.16 (d, J=7.2 Hz, 3H).

### Example 13a: N-(3-Cyano-4-fluorophenyl)-5-methyl-5,6,9,10-tetrahydro-4H-isoxazolo[3,4-c]pyrido[4′,3′:3,4]pyrazolo[1,5-a]azepine-11(12H)-carboxamide

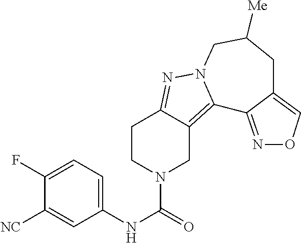 [SMILES: CC1Cc2conc2-c2c3c(nn2C1)CCN(C(=O)Nc1ccc(F)c(C#N)c1)C3]

The title compound was prepared in a manner analogous to Example 12, using N-(3-cyano-4-fluorophenyl)-5-methylene-5,6,9,10-tetrahydro-4H-isoxazolo[3,4-c]pyrido[4′,3′:3,4]pyrazolo-[1,5-a]azepine-11(12H)-carboxamide (Example 1) instead of N-(4-fluoro-3-(trifluoro-methyl)phenyl)-5-methylene-5,6,9,10-tetrahydro-4H-isoxazolo[3,4-c]pyrido[4′,3′:3,4]pyrazolo-[1,5-a]azepine-11(12H)-carboxamide. MS (ESI): mass calcd. for C₂₁H₁₉FN₆O₂, 406.2; m/z found, 407.1 [M+H]⁺. ¹H NMR (400 MHz, CDCl₃) δ=8.32 (s, 1H), 7.79 (dd, J=2.8, 5.6 Hz, 1H), 7.68-7.61 (m, 1H), 7.14 (t, J=8.8 Hz, 1H), 6.78 (s, 1H), 4.74 (d, J=3.2 Hz, 2H), 4.55-4.52 (m, 1H), 4.33-4.27 (m, 1H), 3.94-3.88 (m, 2H), 3.04-3.00 (m, 1H), 2.88 (t, J=5.6 Hz, 2H), 2.76-2.69 (m, 1H), 2.44 (d, J=5.6 Hz, 1H), 1.16 (d, J=7.2 Hz, 3H).

### Example 14a: (10R)-N-(3-Cyano-4-fluorophenyl)-10-methyl-5,6,9,10-tetrahydro-4H-isoxazolo[5,4-c]pyrido[4′,3′:3,4]pyrazolo[1,5-a]azepine-11(12H)-carboxamide

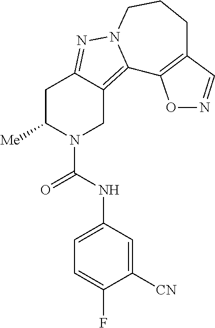 [SMILES: C[C@@H]1Cc2nn3c(c2CN1C(=O)Nc1ccc(F)c(C#N)c1)-c1oncc1CCC3]

The title compound was prepared in a manner analogous to Example 1, using (10R)-tert-butyl 10-methyl-5,6,9,10-tetrahydro-4H-isoxazolo[5,4-c]pyrido[4′,3′:3,4]pyrazolo[1,5-a]azepine-11(12H)-carboxylate (Intermediate 7) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A. MS (ESI): mass calcd. for C₂₁H₁₉FN₆O₂, 406.2; m/z found, 407.1 [M+H]⁺. ¹H NMR (400 MHz, CDCl₃) 8.20-8.19 (m, 1H), 7.84-7.79 (m, 1H), 7.67-7.59 (m, 1H), 7.17 (d, J=8.7 Hz, 1H), 6.62-6.58 (m, 1H), 5.17-5.11 (m, 1H), 4.94 (s, 1H), 4.64) (d, J=15.0 Hz, 1H), 4.51 (s, 2H), 3.09-3.00 (m, 1H), 2.91-2.86 (m 2H), 2.72-2.65 (m, 1H), 2.32-2.24 (m, 2H), 1.22-1.19 (m, 3H).

### Example 15a: (10R)-N-(4-Fluoro-3-(trifluoromethyl)phenyl)-10-methyl-5,6,9,10-tetrahydro-4H-isoxazolo[5,4-c]pyrido[4′,3′:3,4]pyrazolo[1,5-a]azepine-11(12H)-carboxamide

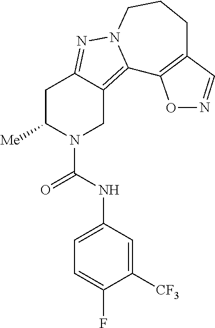 [SMILES: C[C@@H]1Cc2nn3c(c2CN1C(=O)Nc1ccc(F)c(C(F)(F)F)c1)-c1oncc1CCC3]

The title compound was prepared in a manner analogous to Example 1, using (10R)-tert-butyl 10-methyl-5,6,9,10-tetrahydro-4H-isoxazolo[5,4-c]pyrido[4′,3′:3,4]pyrazolo[1,5-a]azepine-11(12H)-carboxylate (Intermediate 7) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A and using phenyl (4-fluoro-3-(trifluoromethyl)phenyl)carbamate instead of phenyl (3-cyano-4-fluorophenyl)carbamate in Step B. MS (ESI): mass calcd. for C₂₁H₁₉F₄N₅O₂, 449.2; m/z found, 450.1 [M+H]⁺. ¹H NMR (400 MHz, CDCl₃) 8.20 (s, 1H), 7.75-7.70 (m, 1H), 7.68-7.60 (m, 1H), 7.20-7.12 (m, 1H), 6.63-6.57 (m, 1H), 5.22-5.11 (m, 1H), 4.96 (s, 1H), 4.62 (d, J=15.2 Hz, 1H), 4.52 (t, J=5.0 Hz, 2H), 3.11-3.01 (m, 1H), 2.90 (s, 2H), 2.74-2.65 (m, 1H), 2.35-2.23 (m, 2H), 1.21 (d, J=6.9 Hz, 3H).

### Example 16a: (11R)-N-(3-Cyano-4-fluorophenyl)-11-methyl-6,7,10,11-tetrahydro-5H-pyrido-[2,3-c]pyrido[4′,3′:3,4]pyrazolo[1,5-a]azepine-12(13H)-carboxamide

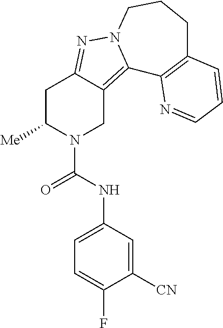 [SMILES: C[C@@H]1Cc2nn3c(c2CN1C(=O)Nc1ccc(F)c(C#N)c1)-c1ncccc1CCC3]

The title compound was prepared in a manner analogous to Example 1, using (11R)-tert-butyl 11-methyl-6,7,10,11-tetrahydro-5H-pyrido[2,3-c]pyrido[4′,3′:3,4]pyrazolo[1,5-a]azepine-12(13H)-carboxylate (Intermediate 8) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A. MS (ESI): mass calcd. for C₂₃H₂₁FN₆O, 416.2: m/z found, 417.2 [M+H]⁺. ¹H NMR (400 MHz, CDCl₃) δ=8.60 (dd, J=1.6, 4.8 Hz, 1H), 7.73 (dd, J=2.8, 5.4 Hz, 1H), 7.70-7.63 (m, 2H), 7.25 (dd, J=4.8, 7.6 Hz, 1H), 7.13 (t, =8.7 Hz, 1H), 6.83 (s, 1H), 5.19-5.05 (m, 1H), 4.97 (d, J=15.3 Hz, 1H), 4.54 (d, J=15.3 Hz, 1H), 4.32-4.22 (m, 2H), 3.10 (dd, J=5.9, 15.8 Hz, 1H), 2.81 (t, J=6.9 Hz, 2H), 2.73 (d, J=16.3 Hz, 1H), 2.49-2.38 (m, 2H), 1.27 (d, J=6.8 Hz, 3H).

### Example 17a: (11R)-N-(4-Fluoro-3-(trifluoromethyl)phenyl)-11-methyl-6,7,10,11-tetrahydro-5H-pyrido[2,3-c]pyrido[4′,3′:3,4]pyrazolo[1,5-a]azepine-12(13H)-carboxamide

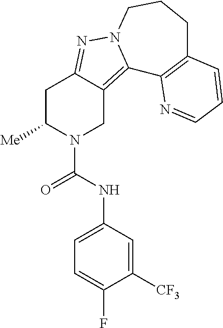 [SMILES: C[C@@H]1Cc2nn3c(c2CN1C(=O)Nc1ccc(F)c(C(F)(F)F)c1)-c1ncccc1CCC3]

The title compound was prepared in a manner analogous to Example 1, using (1 IR)-tert-butyl 11-methyl-6,7,10,11-tetrahydro-5H-pyrido[2,3-c]pyrido[4′,3′:3,4]pyrazolo[1,5-a]azepine-12(13H)-carboxylate (Intermediate 8) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A and using phenyl (4-fluoro-3-(trifluoromethyl)phenyl)carbamate instead of phenyl (3-cyano-4-fluorophenyl)carbamate in Step B. MS (ESI): mass calcd. for C₂₃H₂₁F₄N₅O, 459.2: m/z found, 460.1 [M+H]⁺. ¹H NMR (400 MHz, CDCl₃) δ=8.60 (dd, J=1.7, 4.8 Hz, 1H), 7.68-7.59 (m, 3H), 7.24 (dd, J=4.8, 7.6 Hz, 1H), 7.12 (t, J=9.4 Hz, 1H), 6.73 (s, 1H), 5.15-5.04 (m, 1H), 4.96 (d, J=15.3 Hz, 1H), 4.56 (d, J=15.4 Hz, 1H), 4.28 (t, J=6.8 Hz, 2H), 3.11 (dd, J=6.1, 15.5 Hz, 1H), 2.81 (t, J=6.9 Hz, 2H), 2.73 (d, J=15.7 Hz, 1H), 2.47-2.40 (m, 2H), 1.27 (d, J=7.0 Hz, 31H).

### Example 18a: (10R)-N-(3-Cyano-4-fluorophenyl)-10-methyl-5,6,9,10-tetrahydro-4H-isoxazolo-[3,4-c]pyrido[4′,3′:3,4]pyrazolo[1,5-a]azepine-11(12H)-carboxamide

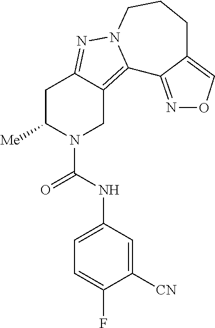 [SMILES: C[C@@H]1Cc2nn3c(c2CN1C(=O)Nc1ccc(F)c(C#N)c1)-c1nocc1CCC3]

The title compound was prepared in a manner analogous to Example 1, using (10R)-tert-butyl 10-methyl-5,6,9,10-tetrahydro-4H-isoxazolo[3,4-c]pyrido[4′,3′:3,4]pyrazolo[1,5-a]azepine-11(12H)-carboxylate (Intermediate 9) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A. MS (ESI): mass calcd. for C₂₁H₁₉FN₆O₂, 406.2; m/z found, 407.1 [M+H]⁺. ¹H NMR (400 MHz, CDCl₃) 8.32 (s, 1H), 7.81-7.77 (m, 1H), 7.68-7.61 (m, 1H), 7.18-7.10 (m, 1H), 6.76-6.65 (m, 1H), 5.26-5.12 ((m, 1H), 4.92-4.78 (m, 1H), 4.64-4.46 (m, 3H), 3.13-2.92 (m, 3H), 2.77-2.63 (m, 1H), 2.32-2.18 (m, 2H), 1.22-1.17 (m, 3H).

### Example 19a: (10R)-N-4-Fluoro-3-(trifluoromethyl)phenyl)-10-methyl-5,6,9,10-tetrahydro-4H-isoxazolo[3,4-c]pyrido[4′,3′:3,4]pyrazolo[1,5-a]azepine-11(12H)-carboxamide

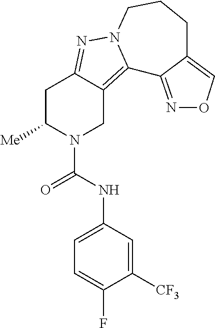 [SMILES: C[C@@H]1Cc2nn3c(c2CN1C(=O)Nc1ccc(F)c(C(F)(F)F)c1)-c1nocc1CCC3]

The title compound was prepared in a manner analogous to Example 1, using (10R)-tert-butyl 10-methyl-5,6,9,10-tetrahydro-4H-isoxazolo[3,4-c]pyrido[4′,3:3,4]pyrazolo[1,5-a]azepine-11(12H)-carboxylate (Intermediate 9) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A and using phenyl (4-fluoro-3-(trifluoromethyl)phenyl)carbamate instead of phenyl (3-cyano-4-fluorophenyl)carbamate in Step B. MS (ESI): mass calcd. for C₂₁H₁₉F₄N₅O₂, 449.2; m/z found, 450.1 [M+H]⁺; ¹H NMR (400 MHz, CDCl₃) 8.33 (s, 1H), 7.74-7.69 (m, 1H), 7.66-7.60 (m, 1H), 7.14 (t, J=9.5 Hz, 1H), 6.66 (s, 1H), 5.20 (t, J=7.0 Hz, 1H), 4.87 (d, J=15.3 Hz, 1H), 4.61-4.50 (m, 3H), 3.11-2.96 (m, 3H), 2.70 (d, J=15.6 Hz, 1H), 2.32-2.22 (m, 2H), 1.24 (d, J=6.9 Hz, 3H).

### Example 20a: N-(3-Chloro-4-fluorophenyl)-6,7,10,11-tetrahydro-5H-pyrido[4′,3′:3,4]pyrazolo-[1,5-a][1,2,4]triazolo[3,4-c][1,4]diazepine-12(13H)-carboxamide

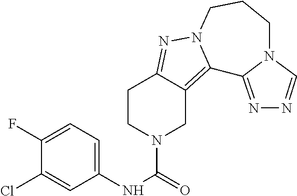 [SMILES: O=C(Nc1ccc(F)c(Cl)c1)N1CCc2nn3c(c2C1)-c1nncn1CCC3]

The title compound was prepared in a manner analogous to Example 1, using tert-butyl 6,7,10,11-tetrahydro-5H-pyrido[4′,3′:3,4]pyrazolo[1,5-a][1,2,4]triazolo[3,4-c][1,4]diazepine-12(13H)-carboxylate (Intermediate 10) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A and using phenyl (3-chloro-4-fluorophenyl)carbamate instead of phenyl N-(3-cyano-4-fluoro-phenyl)carbamate in Step B. MS (ESI): mass calcd. for C₁₈H₁₇ClFN₇O, 401.1: m/z found, 402 [M+H]⁺. ¹H NMR (400 MHz, CDCl₃) δ=8.19 (s, 1H), 7.65 (dd, J=2.6, 6.7 Hz, 1H), 7.29 (dd, =2.8, 4.1 Hz, 1H), 7.00-7.10 (m, 2H), 4.86 (s, 2H), 4.63-4.70 (m, 2H), 4.39-4.45 (m, 2H), 3.91 (t, J=5.8 Hz, 2H), 2.87 (t, J=5.8 Hz, 2H), 2.49-2.59 ((m, 2H).

### Example 21a: N-(3-Chloro-4-fluorophenyl)-3-methyl-6,7,10,11-tetrahydro-5H-pyrido[4′,3′:3,4]-pyrazolo[1,5-a][1,2,4]triazolo[3,4-c][1,4]diazepine-12(13H)-carboxamide

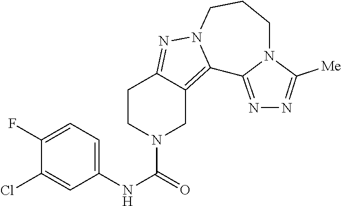 [SMILES: Cc1nnc2n1CCCn1nc3c(c1-2)CN(C(=O)Nc1ccc(F)c(Cl)c1)CC3]

The title compound was prepared in a manner analogous to Example 1, using tert-butyl 3-methyl-6,7,10,11-tetrahydro-5H-pyrido[4′,3′:3,4]pyrazolo[1,5-a][1,2,4]triazolo[3,4-c][1,4]diazepine-12(13H)-carboxylate (Intermediate 11) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A and using phenyl (3-chloro-4-fluorophenyl)carbamate instead of phenyl N-(3-cyano-4-fluoro-phenyl)carbamate in Step B. MS (ESI): mass calcd. for C₁₉H₁₉ClFN₇O, 415.1: m/z found, 416 [M+H]⁺. ¹H NMR (400 MHz, CDCl₃) δ=7.66 (dd, J=2.7, 6.6 Hz, 1H), 7.27-7.31 (m, 1H), 7.12 (s, 1H), 7.05 (t, J=8.8 Hz, 1H), 4.83 (s, 2H), 4.61-4.66 (m, 2H), 4.18-4.23 (m, 2H), 3.91 (t, J=5.8 Hz, 2H), 2.85 (t, J=5.7 Hz, 2H), 2.49-2.56 (m, 5H).

### Example 22a: (R)-N-(3-Chloro-4-fluorophenyl)-11-methyl-6,7,10,11-tetrahydro-5H-pyrido-[4′,3′:3,4]pyrazolo[1,5-a][1,2,4]triazolo[3,4-c][1,4]diazepine-12(13H)-carboxamide

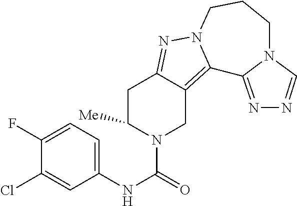 [SMILES: C[C@@H]1Cc2nn3c(c2CN1C(=O)Nc1ccc(F)c(Cl)c1)-c1nncn1CCC3]

The title compound was prepared in a manner analogous to Example 1, using (11R)-tert-butyl 11-methyl-6,7,10,11-tetrahydro-5H-pyrido[4′,3′:3,4]pyrazolo[1,5-a][1,2,4]triazolo[3,4-c][1,4]-diazepine-12(13H)-carboxylate (Intermediate 12) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A and using phenyl (3-chloro-4-fluorophenyl)carbamate instead of phenyl N-(3-cyano-4-fluoro-phenyl)carbamate in Step B. MS (ESI): mass calcd. for C₁₉H₁₉ClFN₇O, 415.1; m/z found, 416 [M+H]⁺. ¹H NMR (400 MHz, CDCl₃) δ=8.20 (s, 1H), 7.67 (dd, J=2.6, 6.5 Hz, 1H), 7.28-7.31 (m, 1H), 7.06 (1, J=8.8 Hz, 1H), 6.96 (br s, 1H), 5.25 (quin, J=6.5 Hz, 1H), 5.00 (d, =15.8 Hz, 1H), 4.58-4.74 (m, 3H), 4.40-4.49 (m, 2H), 3.06 (dd, =5, 15.9 Hz, 1H), 2.69 (d, J=15.8 Hz, 1H), 2.55 (br d, J=3.3 Hz, 2H), 1.18 (d, =7.0 Hz, 3H).

### Example 23a: (11R)-N-(3-Chloro-4-fluorophenyl)-11-methyl-6,7,10,11-tetrahydro-5H-pyrido-[4′,3′:3,4]pyrazolo[1,5-a][1,2,4]triazolo[3,4-c][1,4]diazepine-12(13H)-carboxamide

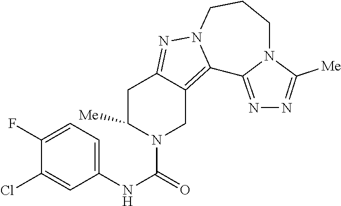 [SMILES: Cc1nnc2n1CCCn1nc3c(c1-2)CN(C(=O)Nc1ccc(F)c(Cl)c1)[C@H](C)C3]

The title compound was prepared in a manner analogous to Example 1, using (1 IR)-tert-butyl 3,11-dimethyl-6,7,10,11-tetrahydro-5H-pyrido[4′,3′:3,4]pyrazolo[1,5-a][1,2,4]triazolo[3,4-c][1,4]diazepine-12(13H)-carboxylate (Intermediate I3) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A and using phenyl (3-chloro-4-fluorophenyl)carbamate instead of phenyl N-(3-cyano-4-fluoro-phenyl)carbamate in Step B. MS (ESI): mass calcd. for C₂₆H₂₁ClFN₇O, 429.1; m/z found, 430 [M+H]⁺. ¹H NMR (400 MHz, CDCl₃) δ=7.68 (dd, J=2.7, 6.6 Hz, 1H), 7.28-7.32 (m, 1H), 7.06 (t, J=8.8 Hz, 1H), 6.98 (s, 1H), 4.97 (m, 1H), 4.57-4.68 (m, 3H), 4.20-4.25 (m, 2H), 3.05 (m, 1H), 2.68 (m, 1H), 2.54 (s, 5H), 1.17 (d, J=6.9 Hz, 3H).

### Example 24a: N-(3-Cyano-4-fluorophenyl)-6,7,10,11-tetrahydro-5H-pyridazino[3,4-c]pyrido-[4′,3′:3,4]pyrazolo[1,5-a]azepine-12(13H)-carboxamide

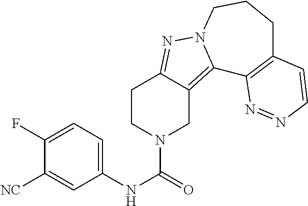 [SMILES: N#Cc1cc(NC(=O)N2CCc3nn4c(c3C2)-c2nnccc2CCC4)ccc1F]

The title compound was prepared in a manner analogous to Example 1, using tert-butyl 10-methyl-11-oxo-8-(1H-pyrazol-3-yl)-3,4,8,9,10,11-hexahydro-1H-pyrido[4′,3′:3,4]pyrazolo[1,5-a][1.4]diazepine-2(7H)-carboxylate (Intermediate 15) instead of tert-butyl 10-methyl-11-oxo-8-(1H-1,2,4-triazol-3-yl)-1,3,4,7,8,9-hexahydropyrido[2,3]pyrazolo[2,4-b][1,4]diazepine-2-carboxylate (Intermediate 1) in Step A. MS (ESI): mass calcd. for C₂₁H₁₈FN₇O, 403.1; m/z found, 404 [M+H]⁺. ¹H NMR (400 MHz, CDCl₃) 9.04 (d, J=5.1 Hz, 1H), 7.75-7.82 (m, 1H), 7.64 (ddd, J=2.8, 4.6, 9.2 Hz, 1H), 7.43 (d, J=5.1 Hz, 1H), 7.12 (t, J=8.7 Hz, 1H), 6.9) (s, 1H), 4.89 (s, 2H), 4.40 (t, J=6.5 Hz, 2H), 3.94 (t, J=5.8 Hz, 2H), 2.87-3.00 (m, 4H), 2.44 (t, J=6.5 Hz, 2H)

### Example 25a: N-(3-Chloro-4-fluorophenyl)-4,5,6,9,10,12-hexahydropyrazolo[3,4-c]pyrido-[4′3′:3,4]pyrazolo[1,5-a]azepine-11(2H)-carboxamide

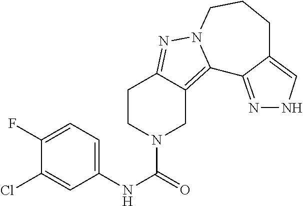 [SMILES: O=C(Nc1ccc(F)c(Cl)c1)N1CCc2nn3c(c2C1)-c1n[nH]cc1CCC3]

The title compound was prepared in a manner analogous to Example 1, using tert-butyl 4,5,6,9,10,12-hexahydropyrazolo[3,4-c]pyrido[4′,3*:3,4]pyrazolo[1,5-a]azepine-11(2H)-carboxylate (Intermediate I6) instead of tert-butyl 10-methyl-11-oxo-8-(1H-1,2,4-triazol-3-yl)-1,3,4,7,8,9-hexahydropyrido[2,3]pyrazolo[2,4-b][1,4]diazepine-2-carboxylate (Intermediate 1) in Step A and using phenyl (3-chloro-4-fluorophenyl)carbamate instead of phenyl N-(3-cyano-4-fluoro-phenyl)carbamate in Step B. MS(ESI): mass calcd. for C₁₉H₁₈ClFN₆O, 400.1; m/z found, 401 [M+H]+. ¹H NMR (400 MHz, CDCl₃) δ 7.54 (dd, J=2.6, 6.4 Hz, 1H), 7.45 (s, 1H), 7.21-7.26 (m, 1H), 7.00-7.08 (m, 1H), 6.63-6.70 (m, 1H), 4.76 (s, 2H), 4.44-4.57 (m, 2H), 3.87 (t, J=5.8 Hz, 2H), 2.92-3.03 (m, 2H), 2.86 (t, J=5.8 Hz, 2H), 2.16-2.30 (m, 2H).

### Example 26a: N-(3-Cyano-4-fluorophenyl-4,5,6,9,10,12-hexahydropyrazolo[3,4-c]pyrido-[1′,3′:3,4]pyrazolo[1,5-a]azepine-11(2H)-carboxamide

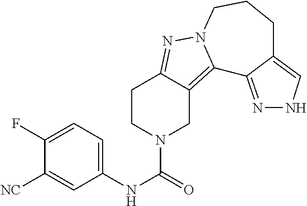 [SMILES: N#Cc1cc(NC(=O)N2CCc3nn4c(c3C2)-c2n[nH]cc2CCC4)ccc1F]

The title compound was prepared in a manner analogous to Example 1, using tert-butyl 4,5,6,9,10,12-hexahydropyrazolo[3,4-c]pyrido[4′,3′:3,4]pyrazolo[1,5-a]azepine-11(2H)-carboxylate (Intermediate 16) instead of tert-butyl 10-methyl-11-oxo-8-(1H-1,2,4-triazol-3-yl)-1,3,4,7,8,9-hexahydropyrido[2,3]pyrazolo[2,4-b][1,4]diazepine-2-carboxylate (Intermediate 1) in Step A. MS(ESI): mass calcd. for C₂₀H₁₈FN₇O, 391.1; m/z found, 392 [M+H]+. ¹H NMR (400 MHz, CD₃OD) δ 7.79-7.85 (m, 1H), 7.71 (ddd, J=2.8, 4.7, 9.2 Hz, 1H), 7.56 (s, 1H), 7.27 (t, J=9.0 Hz, 1H), 4.80 (s, 2H), 4.39-446 (m, 2H), 3.80-3.89 (m, 2H), 2.93-3.02 (m, 2H), 2.80 (t, J=5.7 Hz, 2H), 2.12-2.23 (m, 2H).

### Example 27a: N-(3-Cyano-4-fluorophenyl-6,7,10,11-tetrahydro-5H-pyrido[2,3-c]pyrido-[4′,3′:3,4]pyrazolo[1,5-a]azepine-12(13H)-carboxamide

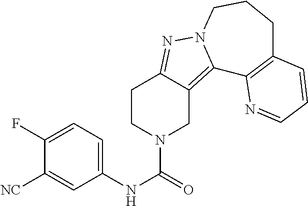 [SMILES: N#Cc1cc(NC(=O)N2CCc3nn4c(c3C2)-c2ncccc2CCC4)ccc1F]

The title compound was prepared in a manner analogous to Example 1, using tert-butyl 11-oxo-3,4,8,9,10,11-hexahydro-1H-pyrido[4′,3′:3,4]pyrazolo[1,5-a]azepine-2(7H)-carboxylate (Intermediate I7) instead of tert-butyl 5-methylene-5,6,9,10-tetrahydro-4H-isoxazolo[3,4-c]pyrido[4′,3′:3,4]pyrazolo[1,5-a]azepine-11(12H)-carboxylate (Intermediate 1) in Step A. MS (ESI): mass calcd. for C₂₂H₁₉FN₆O, 402.16; m/z found, 403.2 [M+H]⁺. ¹H NMR (400 MHz, CDCl₃) δ=8.59 (dd, J=1.6, 4.8 Hz, 1H), 7.72 (dd, J=2.8, 5.4 Hz, 1H), 7.69-7.63 (m, 2H), 7.27-7.23 (m, 1H), 7.13 (t, J=8.8 Hz, 1H), 6.87 (s, 1H), 4.79 (s, 2H), 4.26 (t, J=6.8 Hz, 2H), 3.91 (t, J=5.8 Hz, 2H), 2.92 (t, J=5.8 Hz, 2H), 2.81 (t, J=6.8 Hz, 2H), 2.46-2.39 (m, 2H).

### Example 28a: N-(4-Fluoro-3-(trifluoromethyl)phenyl)-6,7,10,11-tetrabydro-5H-pyrido[2,3-c]pyrido[4′,3′:3,4]pyrazolo[1,5-a]azepine-12(13H)-carboxamide

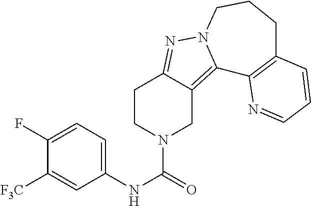 [SMILES: O=C(Nc1ccc(F)c(C(F)(F)F)c1)N1CCc2nn3c(c2C1)-c1ncccc1CCC3]

The title compound was prepared in a manner analogous to Example 1, using tert-butyl 11-oxo-3,4,8,9,10,11-hexahydro-1H-pyrido[4′,3′:3,4]pyrazolo[1,5-a]azepine-2(7H)-carboxylate (Intermediate 17) instead of tert-butyl 5-methylene-5,6,9,10-tetrahydro-4H-isoxaolo[3,4-c]pyrido[4′,3′:3,4]pyrazolo[1,5-a]azepine-11(12H)-carboxylate (Intermediate 1) in Step A and using phenyl (4-fluoro-3-trifluoromethyl)phenyl)carbamate instead of phenyl (3-cyano-4-fluorophenyl)carbamate in Step B. MS (ESI): mass calcd. for C₂₂H₁₉F₄N₅O, 445.2, m/z found, 446.1 [M+H]⁺. ¹H NMR (400 MHz, CDCl₃) δ=8.59 (dd, J=1.6, 4.8 Hz, 1H), 7.67-7.59 (m, 3H), 7.24 (dd, J=4.8, 7.6 Hz, 1H), 7.12 (t, J=9.4 Hz, 1H), 6.74 (s, 1H), 4.79 (s, 2H), 4.26 (t, J=6.8 Hz, 2H), 3.91 (t, J=6.0 Hz, 2H), 2.92 (t, J=6.0 Hz, 2H), 2.81 (t, J=6.8 Hz, 2H), 2.46-2.39 (m, 2H).

### Example 29a: N-(3-Chloro-4-fluorophenyl)-2-methyl-4,5,6,9,10,12-hexahydropyrazolo[3,4-c]-pyrido[4′,3′:3,4]pyrazolo[1,5-a]azepine-11(2H)-carboxamide

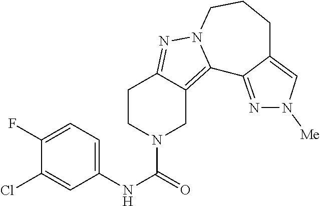 [SMILES: Cn1cc2c(n1)-c1c3c(nn1CCC2)CCN(C(=O)Nc1ccc(F)c(Cl)c1)C3]

The title compound was prepared in a manner analogous to Example 1, using tert-butyl 2-methyl-4,5,6,9,10,12-hexahydropyrazolo[3,4-c]pyrido[4′,3′:3,4]pyrazolo[1,5-a]azepine-11(2H)-carboxylate (Intermediate 18) instead of tert-butyl 10-methyl-11-oxo-8-(1H-1,2,4-triazol-3-yl)-1,3,4,7,8,9-hexahydropyrido[2,3]pyrazolo[2,4-b][1,4]diazepine-2-carboxylate (Intermediate 1) in Step A and using phenyl (3-chloro-4-fluorophenyl)carbamate instead of phenyl N-(3-cyano-4-fluoro-phenyl)carbamate in Step B. MS(ESI): mass calcd. for C₂₀H₂₀ClFN₆O, 414.1; m/z found, 415.1 [M+H]⁺. ¹H NMR (400 MHz, CDCl₃) δ 7.57 (dd, J=2.6, 6.5 Hz, 1H), 7.21-7.26 (m, 2H), 7.06 (t, J=8.8 Hz, 1H), 6.62 (s, 1H), 4.74 (s, 2H), 4.39-4.55 (m, 2H), 3.93 (s, 3H), 3.86 (s, 2H), 2.85 (s, 4H), 2.18 (br s, 2H).

### Example 30a: N-(3-Chloro-4-fluorophenyl)-1-methyl-4,5,6,9,10,12-hexahydropyrazolo[3,4-c]pyrido[4′,3′:3,4]pyrazolo[1,5-a]azepine-11(1H)-carboxamide

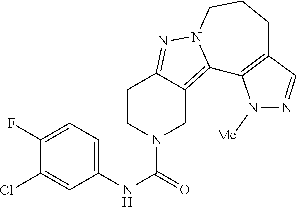 [SMILES: Cn1ncc2c1-c1c3c(nn1CCC2)CCN(C(=O)Nc1ccc(F)c(Cl)c1)C3]

The title compound was prepared in a manner analogous to Example 1, using tert-butyl 1-methyl-4,5,6,9,10,12-hexahydropyrazolo[3,4-c]pyrido[4′,3′:3,4]pyrazolo[1,5-a]azepine-11(1H)-carboxylate (Intermediate 19) instead of tert-butyl 10-methyl-11-oxo-8-(1H-1,2,4-triazol-3-yl)-1,3,4,7,8,9-hexahydropyrido[2,3]pyrazolo[2,4-b][1,4]diazepine-2-carboxylate (Intermediate 1) in Step A and using phenyl (3-chloro-4-fluorophenyl)carbamate instead of phenyl N-(3-cyano-4-fluoro-phenyl)carbamate in Step B. MS (ESI): mass calcd. for C₂₀H₂₀ClFN₆O, 414.1; m/z found, 415.1 [M+H]⁺. ¹H NMR (400 MHz, CDCl₃) δ 7.49-7.56 (m, 1H), 7.44 (s, 1H), 7.14-7.22 (m, 1H), 7.02-7.11 (m, 1H), 6.41 (s, 1H), 4.64 (s, 2H), 4.16-4.25 (m, 2H), 3.94 (s, 3H), 3.84 (s, 2H), 2.91-3.00 (m, 2H), 2.73 (s, 2H), 2.16-2.29 (m, 2H).

### Example 31a: N-(3-Chloro-4-fluorophenyl-5,6,9,10-tetrahydro-4H-isoxazolo[3,4-c]pyrido-[4′,3′:3,4]pyrazolo[1,5-a]azepine-11(2H)-carboxamide

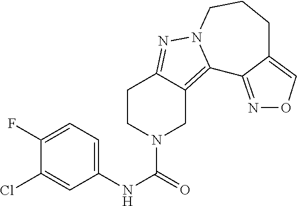 [SMILES: O=C(Nc1ccc(F)c(Cl)c1)N1CCc2nn3c(c2C1)-c1nocc1CCC3]

The title compound was prepared in a manner analogous to Example 1, using tert-butyl 5,6,9,10-tetrahydro-4H-isoxazolo[3,4-c]pyrido[4′,3′:3,4]pyrazolo[1,5-a]azepine-11(12H)-carboxylate (Intermediate 20) instead of tert-butyl 10-methyl-11-oxo-8-(1H-1,2,4-triazol-3-yl)-1,3,4,7,8,9-hexahydropyrido[2,3]pyrazolo[2,4-b][1,4]diazepine-2-carboxylate (Intermediate 1) in Step A and using phenyl (3-chloro-4-fluorophenyl)carbamate instead of phenyl N-(3-cyano-4-fluoro-phenyl)carbamate in Step B. MS (ESI): mass calcd. for C₁₉H₁₇ClFN₅O₂, 401.1; m/z found, 402.1 [M+H]⁺. ¹H NMR (400 MHz, CDCl₃) δ 8.32 (s, 1H), 7.60 (dd, J=2.2, 6.5 Hz, 1H), 7.24 (br d, J=3.3 Hz, 1H), 7.06 (t, J=8.7 Hz, 1H), 6.62 (s, 1H), 4.73 (s, 2H), 4.53-4.61 (m, 2H), 3.90 (t, J=5.7 Hz, 2H), 2.93-3.03 (m, 2H), 2.87 (t, J=5.7 Hz, 2H), 2.19-2.31 (m, 2H).

### Example 32a: N-(3-Chloro-4-fluorophenyl)-5,6,9,10-tetrahydro-4H-isoxazolo[5,4-c]pyrido-[4′,3′:3,4]pyrazolo[1,5-a]azepine-11(12H)-carboxamide

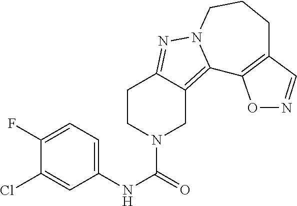 [SMILES: O=C(Nc1ccc(F)c(Cl)c1)N1CCc2nn3c(c2C1)-c1oncc1CCC3]

The title compound was prepared in a manner analogous to Example 1, using tert-butyl 5,6,9,10-tetrahydro-4H-isoxazolo[5,4-c]pyrido[4′,3′:3,4]pyrazolo[1,5-a]azepine-11(12H)-carboxylate (Intermediate 21) instead of tert-butyl 10-methyl-11-oxo-8-(1H-1,2,4-triazol-3-vi)-1,3,4,7,8,9-hexahydropyrido[2,3]pyrazolo[2,4-b][1,4]diazepine-2-carboxylate (Intermediate 1) in Step A and using phenyl (3-chloro-4-fluorophenyl)carbamate instead of phenyl N-(3-cyano-4-fluoro-phenyl)carbamate in Step B. MS (ESI): mass calcd. for C₁₉H₁₇ClFN₅O₂, 401.1; m/z found, 402.1 [M+H]⁺. ¹H NMR (400 MHz, CDCl₃) δ 8.19 (s, 1H), 7.60 (dd, J=2.7, 6.5 Hz, 1H), 7.21-7.26 (m, 1H), 7.08 (t, J=8.7 Hz, 1H), 6.54 (s, 1H), 4.83 (s, 2H), 4.46-4.53 (m, 2H), 3.88 (t, J=5.8 Hz, 2H), 2.87 (td, J=6.0, 8.2 Hz, 4H), 2.27 (br dd, J=3.8, 6.1 Hz, 2H).

### Example 33a: N-(3-Cyano-4-fluorophenyl)-5,6,9,10-tetrahydro-4H-isoxazolo[5″,4″:3′,4′]-cyclohepta[1′,2′:3,4]pyrazolo[1,5-a]pyrazine-11(12H)-carboxamide

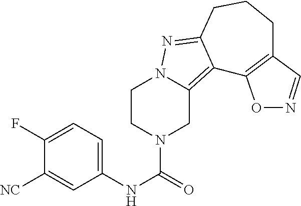 [SMILES: N#Cc1cc(NC(=O)N2CCn3nc4c(c3C2)-c2oncc2CCC4)ccc1F]

### Step A. 5,6,9,10,11,12-Hexahydro-4H-isoxazolo[5″,4″:3′,4′]cyclohepta[1′,2′:3,4]pyrazolo-[1,5-a]pyrazine

To a solution of tert-butyl 5,6,9,10-tetrahydro-4H-isoxazolo[5″,4″:3′,4′]-cyclohepta[1′,2′:3,4]pyrazolo[1,5-a]pyrazine-11(12H)-carboxylate (0.07 g, 211.88 umol) in DCM (2 mL) was added TFA (1.54 g, 13.51 mmol, 1 mL) and the mixture was stirred at 20° C. for 1 h. The mixture was concentrated under reduced pressure to give title compound (0.073 g, crude. TFA salt) as a yellow oil, which was used directly for the next step.

### Step B. N-(3-Cyano-4-fluorophenyl)-5,6,9,10-tetrahydro-4H-isoxazolo[5″,4″:3′,4]cyclohepta-[1′,2′:3,4]pyrazolo[1,5-a]pyrazine-11(12H)-carboxamide

A mixture of 5,6,9,10,11,12-hexahydro-4H isoxazolo[5″,4″:3′,4′]cyclohepta[1′,2′:3,4] pyrazolo [1,5-a]pyrazine (0.073 g, TFA salt), phenyl N-(3-cyano-4-fluoro-phenyl)carbamate (54.33 mg, 212.03 umol) and Et₃N (107.28 mg, 1.06 mmol, 147.56 uL) in DCM (4 mL) was stirred at 20° C. for 16 h. The mixture was concentrated under reduced pressure. The residue was purified by prep-HPLC (condition A) to give title compound (0.048 g, 120.86 umol, 57.00% yield, 98.8% purity) as a white solid. MS (ESI): mass calcd. for C₂₀H₁₇FN₆O₂ 392.1; m/z found, 393.1 [M+H]⁺. ¹H NMR (400 MHz, DMSO-d₆) δ 9.32 (s, 1H), 8.41 (s, 1H), 7.95-7.93 (m, 1H), 7.78-7.78 (m, 1H), 7.48-7.44 (m, 1H), 5.01 (s, 2H), 4.17-4.14 (m, 2H), 3.99-3.97 (m, 2H), 2.93-2.90 (m, 2H), 2.76-2.73 (m, 2H), 1.91-1.89 (m, 2H).

### Example 34a: N-(3-Cyano-4-fluorophenyl)-5,6,9,10-tetrahydro-4H-isoxazolo[5″,4″:3′,4′]-cyclohepta[1′,2′:3,4]pyrazolo[1,5-a]pyrazine-11(12H)-carboxamide

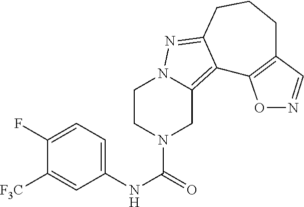 [SMILES: O=C(Nc1ccc(F)c(C(F)(F)F)c1)N1CCn2nc3c(c2C1)-c1oncc1CCC3]

The title compound was prepared in a manner analogous to Example 1, step 2, using phenyl (4-fluoro-3-(trifluoromethyl)phenyl)carbamate instead of phenyl N-(3-cyano-4-fluoro-phenyl)-carbamate. MS (ESI): mass calcd. for C₂₀H₁₇F₄N₅O₂ 435.1; m/z found, 436.1 [M+H]⁺. ¹H NMR (400 MHz, DMSO-d₆) δ 9.30 (s, 1H), 8.41 (s, 1H), 7.93-7.90 (m, 1H), 7.80-7.77 (m, 1H), 7.45-7.41 (m, 1H), 5.01 (s, 2H), 4.17-4.00 (m, 2H), 3.99-3.98 (m, 2H), 2.93-2.9) (m, 2H), 2.75-2.73 (m, 2H), 1.91-1.89 (m, 2H).

### Example 35a: N-(3-Cyano-4-fluorophenyl)-5,6,9,10-tetrahydro-4H-isoxazolo[3″,4″: 3′,4′]-cyclohept[1′,2′:3,4]pyrazolo[1,5-a]pyrazine-11(12H)-carboxamide

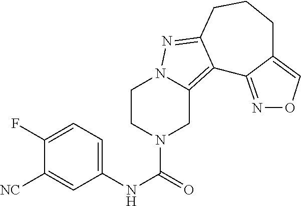 [SMILES: N#Cc1cc(NC(=O)N2CCn3nc4c(c3C2)-c2nocc2CCC4)ccc1F]

The title compound was prepared in a manner analogous to Example 1, using tert-butyl 5,6,9,10-tetrahydro-4H-isoxazolo[3″,4″:3′,4′]cyclohepta[1′,2′:3,4]pyrazolo[1,5-a]pyrazine-11(12H)-carboxylate instead of tert-butyl 5,6,9,10-tetrahydro-4H-isoxazolo(5″,4″:3′,4′-cyclohepta[1′,2′:3,4]pyrazolo[1,5-a]pyrazine-11(12H)-carboxylate. MS (ESI): mass calcd. for C₂₀H₁₇FN₆O₂ 392.1; m/z found, 393.1 [M+H]⁺. ¹H NMR (400 MHz, DMSO-d6) δ 9.31 (s, 1H), 8.65 (s, 1H), 7.94-7.92 (m, 1H), 7.78-7.77 (m, 1H), 7.48-7.43 (m, 1H), 4.90 (s, 2H), 4.18-4.15 (m, 2H), 3.99-3.95 (m, 2H), 2.97-2.95 (m, 2H), 2.82-2.79 (m, 2H), 1.88-1.86 (m, 2H).

### Example 36a: N-(4-Fluoro-3-(trifluoromethyl)phenyl)-5,6,9,10-tetrahydro-4H-isoxazolo-[3″,4″:3′,4′]cyclohepta[1′,2′:3,4]pyrazolo[1,5-a]pyrazine-11(12H)-carboxamide

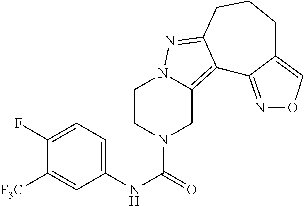 [SMILES: O=C(Nc1ccc(F)c(C(F)(F)F)c1)N1CCn2nc3c(c2C1)-c1nocc1CCC3]

The title compound was prepared in a manner analogous to Example 1, step 2, except using 5,6,9,10,11,12-hexahydro-4H-isoxazolo[3″,4″:3′,4′]cyclohepta[1′,2′:3,4]pyrazolo[1,5-a]pyrazine to react with phenyl (4-fluoro-3-(trifluoromethyl)phenyl)carbamate instead of 5,6,9,10,11,12-hexahydro-4H-isoxazolo[5″,4″:3′,4′]cyclohepta[1′,2′:3,4]pyrazolo[1,5-a]pyrazine to react with phenyl N-(3-cyano-4-fluoro-phenyl)carbamate. MS (ESI): mass calcd. for C₂₀H₁₇F₄N₅O₂ 435.1; m/z found, 436.1 [M+H]⁺. ¹H NMR (400 MHz, DMSO-d₆) δ 9.29 (s, 1H), 8.65 (s, 1H), 7.92-7.9) (m, 1H), 7.79-7.77 (m, 1H), 7.45-7.40 (m, 1H), 4.90 (s, 2H), 4.18-4.16 (m, 2H), 4.00-3.99 (m, 2H), 2.98-2.95 (m, 2H), 2.81-2.80 (m, 2H), 1.89-1.86 (m, 2H).

### 4. Anti-HBV Activity of Compounds of Formula (I)

The anti HBV activity was measured using the HepG2.117 cell line, a stable, inducibly HBV producing cell line, which replicates HBV in the absence of doxicycline (Tet-off system). The HepG2 cell line is available from ATCC® under number HB-8065. Transfection of the HepG2 cell line can be as described in Sun and Nassal 2006 Journal of Hepatology 45 (2006) 636-645 “Stable HepG2- and Huh7-based human hepatoma cell lines for efficient regulated expression of infectious hepatitis B virus”.

For the antiviral assay, HBV replication was induced, followed by a treatment with serially diluted compound in 96-well plates. After 3 days of treatment, the antiviral activity was determined by, quantification of intracellular HBV DNA using real-time PCR and an HBV specific primer set and probe.

Cytotoxicity of the compounds was tested using HepG2 or HepG2.117 cells, incubated for 3 days in the presence of compounds. The viability of the cells was assessed using the PERKIN ELMER ATPlite Luminescence Assay System.”

### Results:

TABLE 4

| | HBV-AVE-HepG2.117 | TOX-HepC2.117 |
|---|---|---|
| Compound | EC₅₀ | CC₅₀ |
| number | (μM, mean value) | (μM, mean value) |
| 1 | 1.134 | >50 |
| 2 | 0.921 | 41.93 |
| 3 | 1.440 | 46.97 |
| 4 | >10 | >10 |
| 5 | 0.279 | >50 |
| 6 | 0.208 | >50 |
| 7 | 1.289 | >50 |
| 8 | 2.550 | >50 |
| 9 | >50 | >50 |
| 10 | 8.056 | >10 |
| 11 | 0.300 | >46.20 |
| 12 | 0.835 | 31.11 |
| 13 | 2.942 | 26.60 |
| 14 | 0.213 | 39.22 |
| 15 | 0.934 | >43.47 |
| 16 | 1.623 | >50 |
| 17 | 0.248 | 24.66 |
| 18 | 1.051 | >50 |
| 19 | 0.263 | >50 |
| 20 | 0.300 | >50 |
| 22 | 0.130 | >50 |
| 23 | 0.172 | 21.16 |
| 24 | 1.590 | >50 |
| 26 | 1.150 | >50 |
| 27 | 0.393 | >50 |
| 29 | 15.462 | >50 |
| 30 | 0.181 | >50 |
| 31 | 0.047 | >50 |
| 32 | 0.090 | >50 |
| 33 | 0.154 | >50 |
| 34 | 0.358 | >50 |
| 35 | 0.395 | >50 |
| 36 | 0.686 | >50 |
| 37 | 0.343 | >50 |
| 38 | 1.435 | >50 |
| 39 | 1.338 | 43.57 |
| 40 | 0.499 | >50 |
| 41 | 0.749 | 25.57 |
| 42 | >48.834 | >50 |
| 43 | 2.320 | >50 |
| 44 | 0.574 | >50 |
| 45 | 4.967 | >50 |
| 46 | 0.236 | 27.09 |
| 47 | 0.265 | >50 |
| 48 | 0.019 | >50 |
| 49 | 4.637 | >50 |
| 50 | 8.432 | >50 |
| 51 | >10 | >50 |
| 52 | >10 | >50 |
| 53 | >10 | >50 |

Induction or non-induction of HBc speckling HepG2.117 cells were cultured in the presence of DMSO or test compound in absence of doxycycline. After formaldehyde fixation and Triton-X-100 permeabilization. Hepatitis B virus core protein (HBc) was immunolabeled with a primary anti-HBc antibody. ALEXA 488-conjugated secondary antibody was used for fluorescent detection of the primary HBV Core signal. CELLMASK Deep Red and HOECHST 33258 were used for the detection of cytoplasm and nucleus respectively, which allowed the segmentation of cellular compartments. An image analysis software that allows to detect different morphological phenotypes was used to determine the level of HBV core in the cytoplasm or nucleus (high content imaging assay).

### HBV Replication Inhibition Assay

HBV replication inhibition by the disclosed compounds were determined in cells infected or transfected with HBV or cells with stably integrated HBV, such as HepG2.2.15 cells (Sells et al. 1987). In this example, HepG2.2.15 cells were maintained in cell culture medium containing 10% fetal bovine serum (FBS), Geneticin, L-glutamine, penicillin and streptomycin. HepG2.2.15 cells were seeded in 96-well plates at a density of 40,000 cells/well and were treated with serially diluted compounds at a final DMSO concentration of 0.5% either alone or in combination by adding drugs in a checker box format. Cells were incubated with compounds for three days, after which medium was removed and fresh medium containing compounds was added to cells and incubated for another three days. At day 6, supernatant was removed and treated with DNase at 37° C. for 60 minutes, followed by enzyme inactivation at 75° C. for 15 minutes. Encapsidated HBV DNA was released from the virions and covalently linked HBV polymerase by incubating in lysis buffer (Affymetrix QS0010) containing 2.5 μg proteinase K at 50° C. for 40 minutes. HBV DNA was denatured by addition of 0.2 M NaOH and detected using a branched DNA (BDNA) QuantiGene assay kit according to manufacturer recommendation (Affymetrix). HBV DNA levels were also quantified using qPCR, based on amplification of encapsidated HBV DNA extraction with QuickExtraction Solution (Epicentre Biotechnologies) and amplification of HBV DNA using HBV specific PCR probes that can hybridize to HBV DNA and a fluorescently labeled probe for quantitation. In addition, cell viability of HepG2.2.15 cells incubated with test compounds alone or in combination was determined by using CellTitre-Glo reagent according to the manufacturer protocol (Promega). The mean background signal from wells containing only culture medium was subtracted from all other samples, and percent inhibition at each compound concentration was calculated by normalizing to signals from HepG2.2.15 cells treated with 0.5% DMSO using equation E1.

% inhibition=(DMSOave−Xi)/DMSOave×100% E1:

where DMSOave is the mean signal calculated from the wells that were treated with DMSO control (0% inhibition control) and Xi is the signal measured from the individual wells. EC₅₀ values, effective concentrations that achieved 50% inhibitory effect, were determined by non-linear fitting using Graphpad Prism software (San Diego, Calif.) and equation E2.

Y=Ymin+(Ymax−Ymin)/(1+10(Log EC50−X)×HillSlope) E2:

where Y represents percent inhibition values and X represents the logarithm of compound concentrations.

Selected disclosed compounds were assayed in the HBV replication assay (BDNA assay), as described above, and a representative group of these active compounds is shown in Table 5. Table 5 shows EC₅₀ values obtained by the BDNA assay for a group of select compounds.

TABLE 5
Activity in BDNA-assay (EC₅₀)

| Ex | | EC₅₀ |
|---|---|---|
| # | Compound name | (nM) |
| 1a | N-(3-Cyano-4-fluorophenyl)-5-methylene-5,6,9,10-tetrahydro-4H- | 64 |
| | isoxazolo[3,4-c]pyrido[4′,3′: 3,4]pyrazolo[1,5-′a]azepine-11(12H)- | |
| | carboxamide; | |
| 2a | N-(4-Fluoro-3-(trifluoromethyl)phenyl)-5-methylene-5,6,9,10- | 75 |
| | tetrahydro-4H-isoxazolo[3,4-c]pyrido[4′,3′: 3,4]pyrazolo[1,5- | |
| | a]azepine-11(12H)-carboxamide; | |
| 3a | N-(3-Cyano-4-fluorophenyl)-5-(hydroxymethyl)-5,6,9,10- | 23 |
| | tetrahydro-4H-isoxazolo[3,4-c]pyrido[4′,3′: 3,4]pyrazolo[1,5- | |
| | a]azepine-11(12H)-carboxamide; | |
| 4a | N-(4-Fluoro-3-(trifluoromethyl)phenyl)-5-(hydromethyl)- | 57 |
| | 5,6,9,10-tetrahydro-4H-isoxazolo[3,4c]pyrido[4′,3′: 3,4]- | |
| | pyrazolo[1,5-a]azepine-11(12H)-carboxamide; | |
| 5a | (5S*)-N-(3-Cyano-4-fluorophenyl)-5-((2,2-difluoroethoxy)- | 82 |
| | methyl)-5,6,9,10-tetrahydro-4H-isoxazolo[3,4-c]pyrido- | |
| | [4′,3′: 3,4]pyrazolo[1,5-a]azepine-11(12H)-carboxamide; | |
| 6a | (5S*)-5-((2,2-Difluoroethoxy]methyl-N-(4-fluoro-3- | 45 |
| | (trifluoromethyl)phenyl)-5,6,9,10-tetrahydro-4H-isoxazolo[3,4- | |
| | c]pyrido[4′,3′: 3,4]pyrazolo[1,5-a]azepine-11(12H)-carboxamide; | |
| 7a | (5R*)N-(3-Cyano-4-fluorophenyl)-5-((2,2-difluoroethoxy)- | 21 |
| | methyl)-5,6,9,10-tetrahydro-4H-isoxazolo[3,4-c]pyrido[4′,3′: 3,4]- | |
| | pyrazolo[1,5-a]azepine-11(12H)-carboxamide; | |
| 8a | (5R*)-5-((2,2-Difluoroethoxy)methyl)-N-(4-fluoro-3-(trifluoro- | 26 |
| | methyl)phenyl)-5,6,9,10-tetrahydro-4H-isoxazolo[3,4- | |
| | c]pyrido[4′,3′: 3,4]pyrazolo[1,5-a]azepine-11(12H)-carboxamide; | |
| 9a | N-(3-Cyano-4-fluorophenyl)-5-methylene-5,6,9,10-tetrahydro-4H- | 800 |
| | isoxazolo[5,4-c]pyrido[4′,3′: 3,4]pyrazolo[1,5-a]azepine-11(12H)- | |
| | carboxamide; | |
| 10a | N-(4-Fluoro-3-(trifluoromethyl)phenyl)-5-methylene-5,6,9,10- | 350 |
| | tetrahydro-4H-isoxazolo[5,4-c]pyrido[4′,3′: 3,4]pyrazolol[1,5- | |
| | a]azepine-11(12H)-carboxamide; | |
| 11a | N-(3-Cyano-4-fluorophenyl)-5-hydroxy-5,6,9,10-tetrahydro-4H- | 43 |
| | isoxazolo[3,4-c]pyrido[4′,3′: 3,4]pyrazolo[1,5-a]azepine-11(12H)- | |
| | carboxamide; | |
| 12a | N-(4-Fluoro-3-(trifluoromethyl)phenyl)-5-methyl-5,6,9,10- | 52 |
| | tetrahydro-4H-isoxazolo[3,4-c]pyrido[4′,3′: 3,4]pyrazolo[1,5- | |
| | a]azepine-11(12H)-carboxamide; | |
| 13a | N-(3-Cyano-4-fluorophenyl)-5-methyl-5,6,9,10-tetrahydro-4H- | 52 |
| | isoxazolo[3,4-c]pyrido[4′,3′: 3,4]pyrazolo[1,5-a]azepine-11(12H)- | |
| | carboxamide; | |
| 14a | (10R)-N-(3-Cyano-4-fluorophenyl)-10-methyl-5,6,9,10- | 59 |
| | tetrahydro-4H-isoxazolo[5,4-c]pyrido[4′,3′: 3,4]pyrazolo[1,5- | |
| | a]azepine-11(12H)-carboxamide; | |
| 15a | (10R)-N-(4-Fluoro-3-(trifluoromethyl)phenyl)-10-methyl- | 83 |
| | 5,6,9,10-tetrahydro-4H-isoxazolo[5,4- | |
| | c]pyrido[4′,3′: 3,4]pyrazolo[1,5-a]azepine-11(12H)-carboxamide; | |
| 16a | (11R)-N-(3-Cyano-4-fluorophenyl)-11-methyl-6,7,10,11- | 330 |
| | tetrahydro-5H-pyrido[2,3-c]pyrido[4′,3′: 3,4]pyrazolo[1,5- | |
| | a]azepine-12(13H)-carboxamide; | |
| 17a | (11R)-N-(4-Fluoro-3-(trifluoromethyl)phenyl)-11-methyl- | 220 |
| | 6,7,10,11-tetrahydro-5H-pyrido[2,3-c]pyrido[4′,3′: 3,4]- | |
| | pyrazolo[1,5-a]azepine-12(13H)-carboxamide; | |
| 18a | (10R)-N-(3-Cyano-4-fluorophenyl)-10-methyl-5,6,9,10- | 15 |
| | tetrahydro-4H-isoxazolo[3,4-c]pyrido[4′,3′: 3,4]pyrazolo[1,5- | |
| | a]azepine-11(12H)-carboxamide; | |
| 19a | (10R)-N-(4-Fluoro-3-(trifluoromethyl)-pheny)-10-methyl- | 27 |
| | 5,6,9,10-tetrahydro-4H-isoxazolo[3,4-c]pyrido[4′,3′: 3,4]- | |
| | pyrazolo[1,5-a]azepine-11(12H)-carboxamide; | |
| 20a | N-(3-Chloro-4-fluorophenyl)-6,7,10,11-tetrahydro-5H-pyrido- | 2000 |
| | [4′,3′: 3,4]pyrazolo[1,5-a][1,2,4]triazolo[3,4-c][1,4]diazepine- | |
| | 12(13H)-carboxamide; | |
| 21a | N-(3-Chloro-4-fluorophenyl)-3-methyl-6,7,10,11-tetrahydro-5H- | 2700 |
| | pyrido(4′,3′: 3,4]pyrazolo[1,5-a][1,2,4]triazolo[3,4-c][1,4]- | |
| | diazepine-12(13H)-carboxamide; | |
| 22a | (11R)-N-(3-Chloro-4-fluorophenyl)-11-methyl-6, 7,10,11- | >4000 |
| | tetrahydro-5H-pyrido[4′,3′: 3,4][1,5-a][1,2,4]triazolo[3,4- | |
| | c][1,4]diazepine-12(13H)-carboxamide; | |
| 23a | (11R)-N-(3-Chloro-4-fluorophenyl)-11-methyl-6,7,10,11- | >4000 |
| | tetrahydro-5H-pyrido[4′,3′: 3,4]pyrazolo[1,5-a][1,2,4]triazolo[3,4- | |
| | c][1,4]diazepine-12(13H)-carboxamide; | |
| 24a | N-(3-Cyano-4-fluorophenyl)-6,7,10,11 -tetrahydro-5H-pyridazino- | 910 |
| | [3,4-c]pyrido[4′,3′: 3,4]pyrazolo[1,5-a]azepine-12(13H)- | |
| | carboxamide; | |
| 25a | N-(3-Chloro-4-fluorophenyl)-4,5,6,9,10,12-hexahydropyrazolo- | 28 |
| | [3,4-c[pyrido[4′,3′: 3,4]pyrazolo[1,5-a]azepine-11(2H)-carboxamide; | |
| 26a | N-(3-Cyano-4-fluorophenyl)-4,5,6,9,10,12-hexahydropyrazolo- | 23 |
| | [3,4-c]pyrido[4′,3′: 3,4]pyrazolo[1,5-a]azepine-11(2H)-carboxamide; | |
| 27a | N-(3-Cyano-4-fluorophenyl)-6,7,10,11-tetrahydro-5H-pyrido[2,3- | 170 |
| | c]pyrido[4′,3′: 3,4]pyrazolo[1,5-a]azepine-12(13H)-carboxamide; | |
| 28a | N-(4Fluoro-3-(trifluoromethyl)phenyl)-6,7,10,11-tetrahydro-5H- | 1800 |
| | pyrido[2,3-c]pyrido[4′,3′: 3,4]pyrazolo[1,5-a]azepine-12(13H)- | |
| | carboxamide; | |
| 29a | N-(3-Chloro-4-fluorophenyl)-2-methyl-4,5,6,9,10,12-hexhahydro- | 3100 |
| | pyrazolo[3,4-c]pyrido[4′,3′: 3,4]pyrazolo[1,5-a]azepine-11(2H)- | |
| | carboxamide; | |
| 30a | N-(3-Chloro-4-fluorophenyl)-1-methyl-4,5,6,9,10,12- | 3400 |
| | hexahydropyrazdo[3,4-c]pyrido[4′,3′: 3,4]pyrazolo[1,5-a]azepine- | |
| | 11(1H)-carboxamide; | |
| 31a | N-(3-Chloro-4-fluorophenyl)-5,6,9,10-tetrahydro-4H- | 9 |
| | isoxazolo[3,4-c]pyrido[4′,3′: 3,4]pyrazolo[1,5-a]azepine-11(12H)- | |
| | carboxamide: | |
| 32a | N-(3Chloro-4-fluorophenyl)-5,6,9,10-tetrahydro-4H- | 12 |
| | isoxazolo[5,4-c]pyrido[4′,3′: 3,4]pyrazolo[1,5-a]azepine-11(12H)- | |
| | carboxamide; | |
| 33a | N-(3-Cyano-4-fluorophenyl)-5,6,9,10-tetrahydro-4H- | 790 |
| | isoxazolo[5″,4″: 3′,4′]cyclohepta[1′,2′: 3,4]pyrazolo[1,5-a]pyrazine- | |
| | 11(12H)carboxamide; | |
| 34a | N-(3-Cyano-4-fluorophenyl)-5,6,9,10-tetrahydro-4H- | 440 |
| | isoxazolol[5″,4″: 3,4]cyclohepta[1′,2′: 3,4]pyrazolo[1,5-a]pyrazine- | |
| | 11(12H)-carboxamide; | |
| 35a | N-(3-Cyano-4-fluorophenyl)-5,6,9,10-tethydra-4H- | 170 |
| | isoxazolo[3″,4″: 3′,4′]cyclohepta[1′,2′: 3,4]pyrazolo[1,5-a]pyrazine- | |
| | 11(12H)carboxamide; and | |
| 36a | N-(4-Fluoro-3-(trifluoromethyl)phenyl)-5,6,9,10-tetrahydro-4H- | 180 |
| | isoxazolo[3″,4″: 3′,4′]cyclohepta[1′,2′: 3,4]pyrazolo[1,5-a]pyrazine- | |
| | 11(12H)-carboxamide; | |

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A compound of Formula (I): 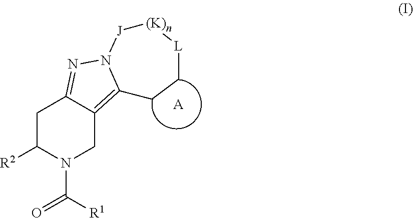
or a stereoisomer or tautomer thereof, wherein 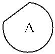
is a 5-membered heteroaryl comprising one, two or three heteroatoms, the heteroatoms being independently selected from the group consisting of N, O and S, wherein the 5-membered heteroaryl is substituted with one or more substituents each independently selected from the group consisting of H, C₁₋₄alkyl, CF₃, CF₂H, NH₂, NH(CH₃), N(CH₃)₂ and phenyl;
R¹ is a 5- to 10-membered monocyclic or bicyclic ring, wherein the 5- to 10-membered monocyclic or bicyclic ring:
optionally contains 1 to 3 heteroatoms, the heteroatoms each independently being selected from N, O and S; and/or
is optionally substituted with one or more substituents each independently selected from the group consisting of hydrogen, halogens, CN, CF₃, CF₂H, CFH₂, CF₂CH₃, C₁₋₆alkyl, OC₁₋₄alkyl, OCF₃, OCF₂H and C₃₋₄cycloalkyl;
R² is selected from the group consisting of H, C₁₋₄alkyl and C₁₋₄alkyl substituted with one or more F;
J is CHR³;
R³ is selected from the group consisting of H, CH₂OH, and C(═O)N(R⁴)(R⁵);
R⁴ and R⁵ are each independently selected from the group consisting of H, C₁₋₄alkyl, and C₃₋₄cycloalkyl, wherein the C₁₋₄alkyl is optionally substituted with one or more substituents each independently selected from the group consisting of OH and F;
K is selected from the group consisting of C(R⁶)(R⁷), C═CH₂ and C(═O);
R⁶ and R⁷ are each independently selected from the group consisting of H, F, OH, OCH₃, CH₂OH, C(═O)R⁸ and C(═O)N(R⁹)(R¹⁰);
R⁸ is OH or morpholine;
R⁹ and R¹⁰ are each independently selected from the group consisting of H, phenyl, C₁₋₄alkyl and C₃₋₄cycloalkyl, wherein the C₁₋₄alkyl is optionally substituted with one or more substituents each independently selected from the group consisting of OH and F;
n is an integer of 0 or 1;
L is C(R¹¹)(R¹²), NH, or O;
R¹¹ and R¹² are each independently selected from the group consisting of H and C(═O)N(R¹³)(R¹⁴); and
R¹³ and R¹⁴ are each independently selected from the group consisting of H, C₁₋₄alkyl and C₃₋₄cycloalkyl, wherein the C₁₋₄alkyl is optionally substituted with one or more substituents each independently selected from the group consisting of OH and F,
or a pharmaceutically acceptable salt thereof.

2. The compound of claim 1, wherein R¹ is phenyl substituted with one or more Cl substituents.

3. The compound of claim 1, wherein R² is H or methyl.

4. The compound of claim 1, wherein R³ is H.

5. The compound of claim 1, wherein K is C(R⁶)(R⁷) or C═CH₂.

6. The compound of claim 5, wherein R⁶ and R⁷ are independently selected from the group consisting of H, F, OH, CH₂OH and C(═O)N(R⁹)(R¹⁰).

7. The compound of claim 6, wherein R⁹ and R¹⁰ are independently selected from the group consisting of C₁₋₄alkyl and C₃₋₄cycloalkyl.

8. The compound of claim 1, wherein each of R¹¹ and R¹² is hydrogen.

9. The compound of claim 1, wherein 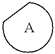
is selected from the group consisting of isoxazole, pyrazole, imidazole, oxazole and thiazole, and wherein 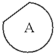
is optionally substituted with one or more substituents selected from the group consisting of H, C₁₋₄alkyl, CF₃, CF₂H, NH₂, NH(CH₃), N(CH₃)₂ and phenyl.

10. The compound of claim 1, wherein 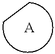
is an isoxazole, optionally substituted with a substituent selected from C₁₋₄alkyl and NH₂.

11. The compound of claim 1, wherein 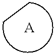
is a pyrazole.

12. A pharmaceutical composition, which comprises the compound or pharmaceutically acceptable salt of claim 1, and further comprises at least one pharmaceutically acceptable carrier.

13. A process for the preparation of the pharmaceutical composition according to claim 12, comprising combining an effective amount of the compound of Formula (I), in intimate admixture with a pharmaceutically acceptable carrier.

14. (canceled)

15. A method of preventing or treating an HBV infection or of an HBV-induced disease in mammal in need thereof, comprising administering to the mammal an effective amount of or the pharmaceutical composition of claim 12.

16. A method of preventing or treating chronic hepatitis B in a subject in need thereof, comprising administering to the subject an effective amount of the pharmaceutical composition of claim 12.

17. A method of treating an HBV infection or an HBV-induced disease in an individual in need thereof, comprising administering to the individual a therapeutically effective amount of the pharmaceutical composition of claim 12.

18. A product comprising a first compound and a second compound as a combined preparation for simultaneous, separate or sequential use in the prevention or treatment of an HBV infection or of an HBV-induced disease in mammal in need thereof, wherein said first compound is different from said second compound, wherein said first compound is the compound or pharmaceutically acceptable salt of claim 1 and wherein said second compound is another HBV inhibitor.

19. The product of claim 18, wherein said second compound is another HBV inhibitor which is selected from the group consisting of: therapeutic agents selected from HBV combination drugs, HBV vaccines, HBV DNA polymerase inhibitors, immunomodulators, toll-like receptor (TLR) modulators, interferon alpha receptor ligands, hyaluronidase inhibitors, hepatitis b surface antigen (HBsAg) inhibitors, cytotoxic T-lymphocyte-associated protein 4 (ipi4) inhibitors, cyclophilin inhibitors, HBV viral entry inhibitors, antisense oligonucleotide targeting viral mRNA, short interfering RNAs (siRNA) and ddRNAi endonuclease modulators, ribonucleotide reductase inhibitors, HBV E antigen inhibitors, covalently closed circular DNA (cccDNA) inhibitors, famesoid X receptor agonists, HBV antibodies, CCR2 chemokine antagonists, thymosin agonists, cytokines, nucleoprotein modulators, retinoic acid-inducible gene 1 simulators, NOD2 stimulators, phosphatidylinositol 3-kinase (PI3K) inhibitors, indoleamine-2,3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and other HBV drugs.

20. A method for the preparing a compound of Formula (I) according to claim 1, comprising at least one step from among steps a), b), c), d), e), f), g), h), i), j), k), l), m), n), o), p), q), r) and s):
a) reacting a compound of Formula (II), 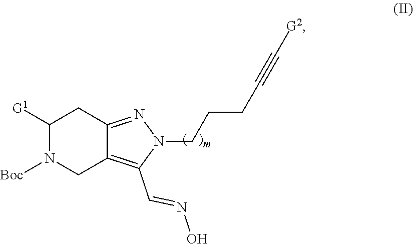
with NaOCl to form a compound of Formula (III), 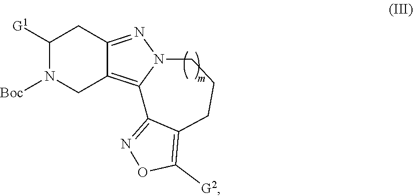
wherein
m is an integer of 0 or 1;
G¹ is H or CH₃;
G² is H, C₁₋₄alkyl, CF₃ or phenyl;
with the proviso that when m is 1, G¹ and G² are not both H;
b) reacting a compound of Formula (III), 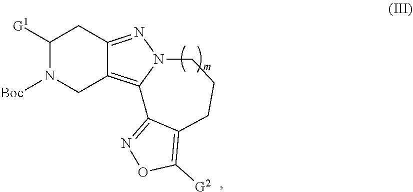
with a strong acid, such as hydrochloric acid (HCl), or TFA to form a compound of formula (IV), 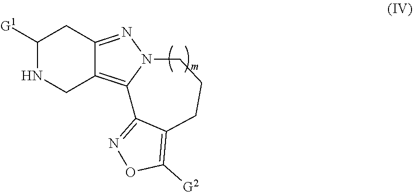
wherein
m is an integer of 0 or 1;
G¹ is H or CH₃;
G² is H, C₁₋₄alkyl, CF₃ or phenyl;
c) reacting a compound of Formula (IV), 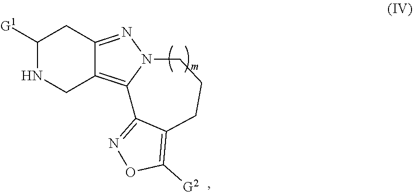
with a compound of formula (V), 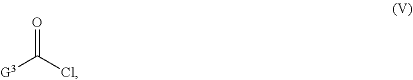
in the presence of a non-nucleophilic base, to form a compound of formula (VI), 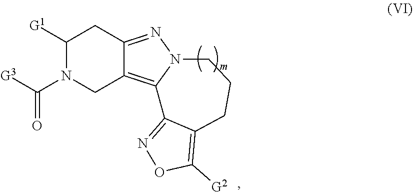
wherein
m is an integer of 0 or 1;
G¹ is H or CH₃;
G² is H, C₁₋₄alkyl, CF₃ or phenyl;
G³ is phenyl substituted with one or more substituents selected from the group consisting of Cl, F, CF₃, CF₂H, CN, and C₁₋₄alkyl;
d) reacting a compound of formula (VII), 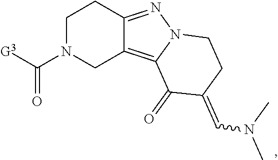
with a compound of formula (VIII), 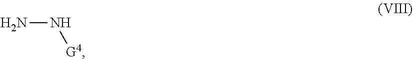
to form a compound of Formula (IX), 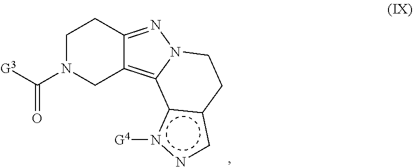
wherein [symbol]
represents a single or a double bond; 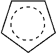
is an aromatic ring;
G³ is phenyl substituted with one or more substituents selected from the group consisting of Cl, F, CF₃, CF₂H, CN, and C₁₋₄alkyl;
G⁴ is H or CH₃;
e) reacting a compound of Formula (X), 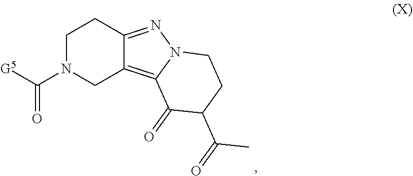
with hydrazine, to form a compound of Formula (XI), 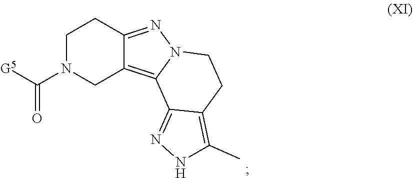
wherein G⁵ is phenyl substituted with one or more substituents selected from the group consisting of Cl, F, CF₃, CF₂H, CN, and C₁₋₄alkyl;
f) reacting a compound of Formula (XXV), 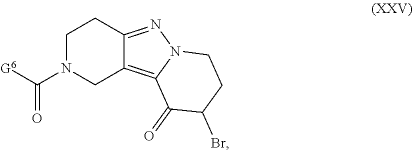
with thioacetamide, to form a compound of Formula (XXVI), 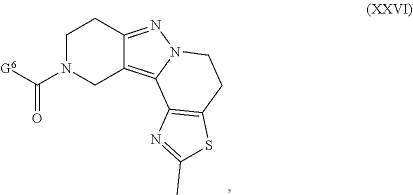
wherein G⁶ is phenyl substituted with one or more substituents selected from the group consisting of Cl, F, CF₃, CF₂H, CN, and C₁₋₄alkyl;
g) reacting a compound of Formula (XII), 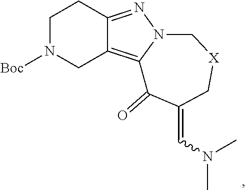
with a compound of Formula (XIII),
H₂N-G⁷ (XII),
to form a compound of Formula (XIV), 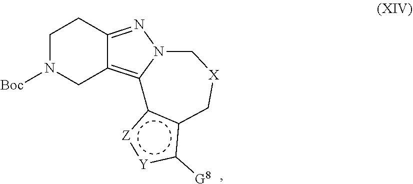
wherein [symbol]
represents a single or a double bond; 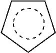
is an aromatic ring;
X is CH₂ or C═CH₂;
G⁷ is OH, NH₂ or NH(CH₃);
G⁸ is H or NH₂;
with the proviso that when G⁷ is NH₂ or NH(CH₃), then G⁸ is H; or when G⁷ is OH, then G⁸ is H or NH₂;
Y is O, NH, N or N(CH₃);
Z is N or O;
h) reacting a compound of Formula (XV), 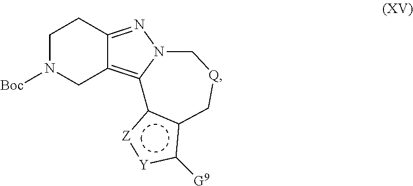
with a strong acid, to form a compound of Formula (XVI), 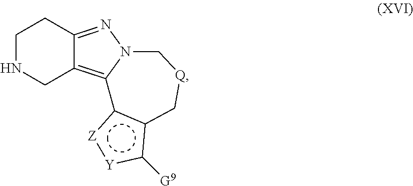
wherein [symbol]
represents a single or a double bond; 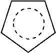
is an aromatic ring;
Q is C═CH₂ or CG¹⁰G¹¹;
G⁹ is H or NH₂;
G¹⁰ and G¹¹ are independently selected from the group consisting of H, OH, CONHMe, CH₂OH and CONH₂;
Y is O, N, NH or N(CH₃);
Z is N or O;
i) reacting a compound of Formula (XVI), 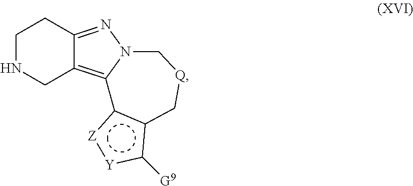
with a compound of Formula (XVII), 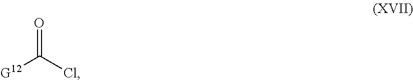
in the presence of a non-nucleophilic base, to form a compound of Formula (XVIII), 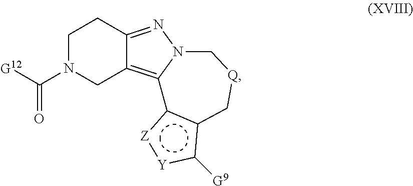
wherein [symbol]
represents a single or a double bond; 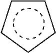
is an aromatic ring;
Q is C═CH₂ or CG¹⁰G¹¹;
G⁹ is H or NH₂;
G¹⁰ and G¹¹ are independently selected from H, OH, CONHMe, CH₂OH and CONH₂;
G² is phenyl substituted with one or more substituents selected from the group consisting of Cl, F, CF₃, CF₂H, CN, and C₁₋₄alkyl;
Y is O, N, NH or N(CH₃);
Z is N or O;
j) reacting a compound of Formula (XIX), 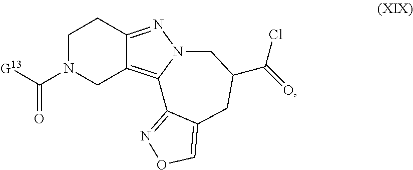
with a compound of Formula (XX), 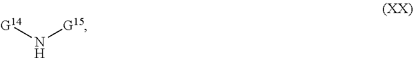
to form a compound of Formula (XXI), 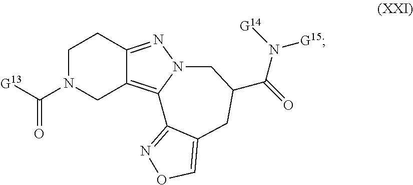
wherein
G³ is phenyl substituted with one or more substituents selected from the group consisting of Cl, F, CF₃, CF₂H, CN, and C₁₋₄alkyl;
G¹⁴ and G¹⁵ are independently selected from the group consisting of H, C₁₋₄alkyl, cyclopropyl, CH₂CH₂OH, CH₂CF₃ and phenyl;
or G¹⁴ and G¹⁵ are connected together to form a morpholine ring;
k) reacting a compound of Formula (XXVII), 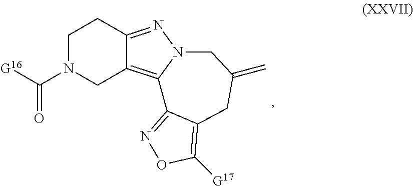
with potassium osmate (K₂OsO₄), in the presence of 4-Methylmorpholine N-oxide (NMO), to form a compound of Formula (XXVIII), 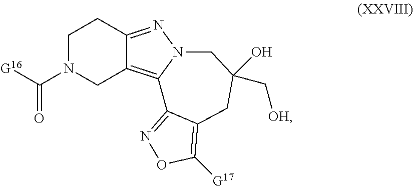
wherein
G¹⁷ is H or NH₂;
G¹⁶ is O-tert-butyl or phenyl substituted with one or more substituents selected from the group consisting of Cl, F, CF₃, CF₂H, CN, and C₁₋₄alkyl;
l) reacting a compound of Formula (XXIX), 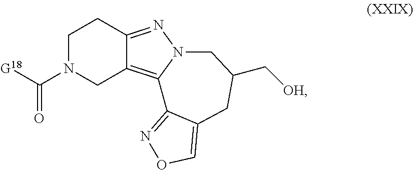
with an oxidizing agent, to form a compound of Formula (XXX); 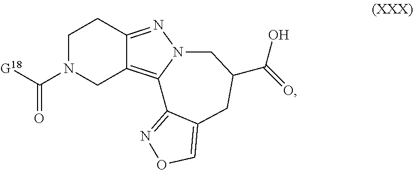
wherein G¹⁸ is O-tert-butyl or phenyl substituted with one or more substituents selected from the group consisting of Cl, F, CF₃, CF₂H, CN, and C₁₋₄alkyl;
m) reacting a compound of Formula XXXI), 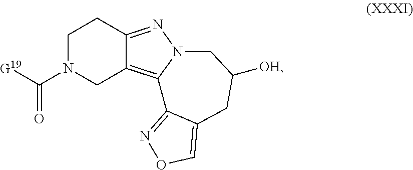
with a fluorinating reagent, to form a compound of Formula (XXXII), 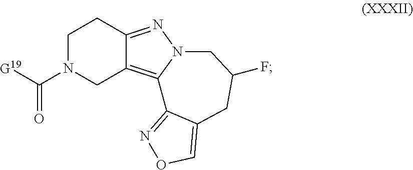
wherein G⁹ is phenyl substituted with one or more substituents selected from the group consisting of Cl, F, CF₃, CF₂H, CN, and C₁₋₄alkyl;
n) reacting a compound of Formula (XXXIII), 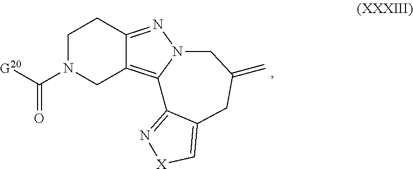
with hydrogen peroxide, in the presence of 9-BBN and sodium hydroxide, to form a compound of Formula (XXXIV), 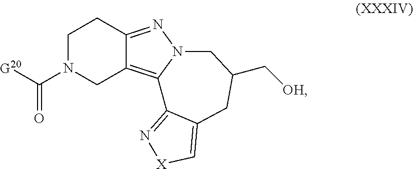
wherein
G²⁰ is O-tert-butyl or phenyl substituted with one or more substituents selected from the group consisting of Cl, F, CF₃, CF₂H, CN, and C₁₋₄alkyl;
X is NH or O;
o) reacting a compound of Formula XXXV 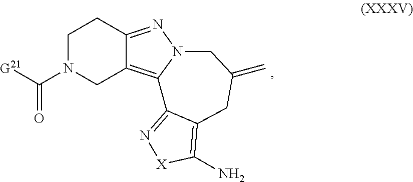
with a methylating agent, in the presence of a non-nucleophilic base, to form a compound of Formula (XXXVI), 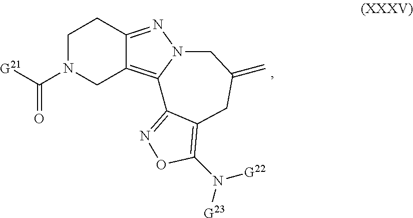
wherein
G²¹ is O-tert-butyl or phenyl substituted with one or more substituents selected from the group consisting of Cl, F, CF₃, CF₂H, CN, and C₁₋₄alkyl;
G²² and G²³ are independently selected from H and CH₃, with the proviso that at least one of G²² and G²³ is CH₃;
p) reacting a compound of Formula (XXXVII), 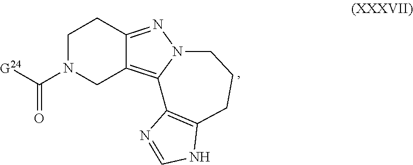
with a methylating agent, in the presence of a non-nucleophilic base, to form a compound of Formula (XXXVIII), 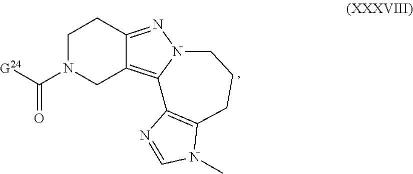
wherein G²⁴ is O-tert-butyl or phenyl substituted with one or more substituents selected from the group consisting of Cl, F, CF₃, CF₂H, CN, and C₁₋₄alkyl;
q) reacting a compound of Formula (XXXIX), 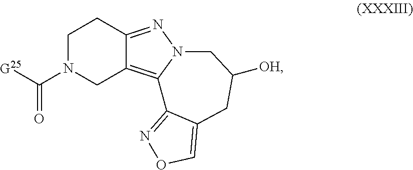
with a methylating agent, in the presence of a non-nucleophilic base, to form a compound of Formula (XL), 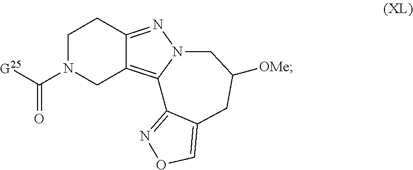
wherein G²⁵ is O-tert-butyl or phenyl substituted with one or more substituents selected from the group consisting of Cl, F, CF₃, CF₂H, CN, and C₁₋₄alkyl;
r) reacting a compound of Formula (XXII), 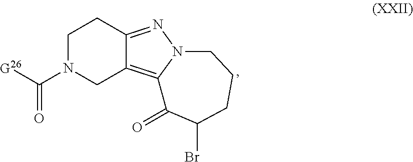
with a compound of Formula (XXIII), 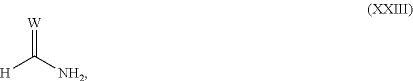
to form a compound of Formula (XXIV), 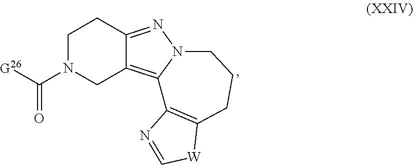
wherein
G²⁶ is phenyl substituted with one or more substituents selected from the group consisting of Cl, F, CF₃, CF₂H, CN, and C₁₋₄alkyl;
W is O or S;
W′ is O, NH, S;
s) reacting a compound of Formula (XLI), 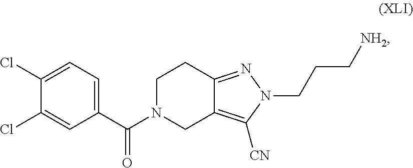 [SMILES: N#Cc1c2c(nn1CCCN)CCN(C(=O)c1ccc(Cl)c(Cl)c1)C2]
with magnesium ethoxide and chloroacetaldehyde, to form a compound of Formula (XLII),

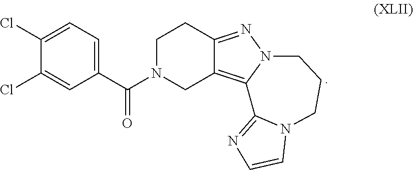 [SMILES: O=C(c1ccc(Cl)c(Cl)c1)N1CCc2nn3c(c2C1)-c1nccn1CCC3]

21.-29. (canceled)

30. A compound selected from the group consisting of: 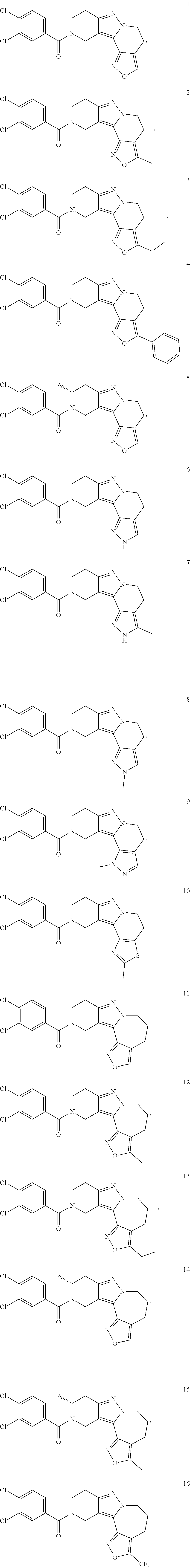 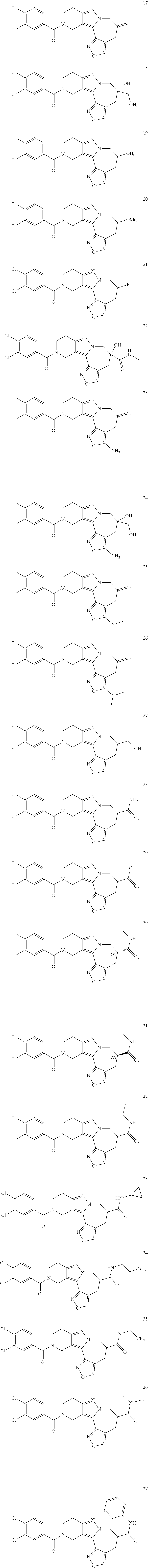 [chemical structure]
